(19) United States
(12) Patent Application Publication
(10) Pub. No.: US 2003/0022819 A1
(43) Pub. Date: Jan. 30, 2003

(54) Angiogenesis-modulating compositions and uses

(76) Inventors: Leona E. Ling, Winchester, MA (US); Michele Sanicola-Nadel, Winchester, MA (US)

Correspondence Address: ROPES & GRAY, ONE INTERNATIONAL PLACE, BOSTON, MA, 02110-2624, US

(21) Appl. No.: 09/883,848

(22) Filed: Jun. 18, 2001

Related U.S. Application Data
(60) Provisional application No. 60/211,919, filed on Jun. 16, 2000.

(51) Int. Cl. A61K 38/17; A61K 31/715

(52) U.S. Cl. 514/7; 514/12; 514/8; 514/54

(57) ABSTRACT

Hedgehog agonists and antagonists can be used to regulate angiogenesis, and have utility in treating tissue repair and cancer, and to prevent angiogenesis driven pathologies.

### RELATED APPLICATIONS

This application claims priority from U.S. Provisional patent application serial No. 60/211,919 filed Jun. 16, 2000, the specification of which is hereby incorporated by reference.

### BACKGROUND OF THE INVENTION

Hedgehog proteins act as morphogens in a wide variety of tissues during embryonic development (Ingham, 1995; Perrimon, 1995; Johnson and Tabin, 1997; Hammerschmidt et al., 1997). Vertebrate hedgehogs are crucial to a number of epithelial-mesenchymal inductive interactions during neuronal development, limb development, lung, bone, hair follicle and gut formation (Ericson et al., 1995; Roberts et al., 1995; Apelqvist et al., 1997; Ericson et al., 1997; Hammerschmidt et al., 1997; Johnson and Tabin, 1995; Pepicelli et al., 1998; Litingtung et al., 1998; Roberts et al., 1998; Dodd et al., 1998; Dockter, 2000). Mammalian hedgehog genes consist of sonic, indian and desert which are highly conserved between species (Zardoya, 1996). Sonic hedgehog (shh) is expressed widely during development and sonic null mice are embryonic lethal with multiple defects beginning early to midgestation (Bitgood and McMahon, 1995; Chiang et al., 1996; Litingtung et al., 1998; St-Jacques et al., 1998). Indian hedgehog (ihh) is expressed less widely and indian null mice survive till late gestation. However, Ihh null mice exhibit severe stunting of skeletal growth which correlates to the role of lhh in regulating bone growth plate (St-Jacques et al., 1999; Karp et al., 2000). Desert hedgehog (dhh) is the most restricted in expression and Dhh null mice are viable, but as expected from the expression pattern, male gonads do not develop completely and the peripheral nerves develop in a disorganized fashion (Bitgood et al., 1996; Parmantier et al., 1999).

Hedgehog signalling occurs through the interaction of hedgehog protein with the hedgehog receptor, patched (Ptc) and this interaction's modulation of the co-receptor smoothened (Smo). The mammalian genome contains 2 patched genes, ptcl and ptc2, both of which encode 12 transmembrane proteins containing a sterol sensing domain (Motoyama et al, 1998; Carpenter et al, 1998). The interaction of Hh and Ptc inactivates the repression of smoothened (Smo), a 7 transmembrane protein which then leads to activation of fused (Fu), a serine-threonine kinase, and the disassociation of a transcription factor, Gli, from the microtuble-associated Fu—Gli—Su(fu) complex. The uncomplexed Gli protein is transported to the nucleus where it activates downstream target genes of the hedgehog pathway including the ptcl and glil genes (Ding et al., 1999; Murone et al, 1999a; Murone et al, 1999b; Pearse et al., 1999; Stone et al., 1999; Hynes et al, 2000).

Hedgehog genes have so far not been implicated directly in embryonic or adult angiogenesis. No vascular defects have been reported in shh, ihh or dhh knockout mice. However, we show here that cells in the adult vasculature both express ptcl and can respond to exogenous hedgehog and, more importantly, hedgehog is able to induce robust neovascularization in the corneal pocket model of angiogenesis. The angiogenic response to hedgehog appears to occur through the activation of mesenchymal cells to produce VEGFs and Angiopoietins.

Angiogenesis, the process of sprouting new blood vessels from existing vasculature and arteriogenesis, the remodeling of small vessels into larger conduit vessels are both physiologically important aspects of vascular growth in adult tissues (Klagsbrun and D'Amore, 1991; Folkman and Shing, 1992; Beck and D'Amore, 1997; Yancopoulos et al., 1998; Buschman and Schaper, 2000). These processes of vascular growth are required for beneficial processes such as tissue repair, wound healing, recovery from tissue ischemia and menstrual cycling. They are also required for the development of pathological conditions such as the growth of neoplasias, diabetic retinopathy, rheumatoid arthritis, psoriasis, certain forms of macular degeneration, and certain inflammatory pathologies (Cherrington et al., 2000).

The ability to stimulate vascular growth has potential utility for treatment of ischemia-induced pathologies such as myocardial infarction, coronary artery disease, peripheral vascular disease, and stroke. The sprouting of new vessels and/or the expansion of small vessels in ischemic tissues prevents ischemic tissue death and induces tissue repair. Certain growth factors such as those in the vascular endothelial growth factor (VEGF) and fibroblast growth factor (FGF) families are able to stimulate vascular growth by acting on endothelial cells to induce angiogenesis. Other factors have also been shown to have angiogenic and arteriogenic activities such as MCPI (Buschman and Schaper, 2000) and angiopoietins. In preclinical models of myocardial infarction, both FGFs and VEGFs have been able to improve myocardial revascularization and function (Yanagisawa-Miwa et al, 1992; Battler et al., 1993; Harada et al., 1994; Banai et al., 1994; Unger et al., 1994; Mesri et al., 1995; Pearlman et al., 1995; Landau et al, 1995; Lazarous et al., 1996; Engler, 1996; Magovern et al., 1997; Shou et al., 1997). Also in models of peripheral vascular disease, VEGF and other angiogenic factors are able to induce angiogenesis and improve vascular perfusion of the ischemic limb (Majesky, 2000; Takeshita et al, 1996 and 1994; Rivard et al., 1998 and 1999, Isner et al, 1996).

A number of these factors are also implicated in vascular growth in pathological conditions such as tumor expansion, diabetic retinopathy and rhematoid arthritis. The inhibiton of vascular growth in these contexts has also shown beneficial effects in preclinical animal models (Klohs and Hamby, 1999; Zhu and Witte, 1999; Cherrington et al., 2000). For example, inhibition of angiogenesis by blocking vascular endothelial growth factor or its receptor has resulted in inhibition of tumor growth and in retinopathy (Fong et al., 1999; Wood et al., 2000; Ozaki et al., 2000). Also, the development of pathological pannus tissue in rheumatoid arthritis involves angiogenesis and can be blocked by inhibitors of angiogenesis (Peacock et al., 1995; Storgard et al., 1999).

Thus, the induction of angiogenesis and vascular growth is beneficial for tissue repair and would healing whereas inhibition of angiogenic growth factors can prevent angiogenesis driven pathologies. It would be useful to develop novel therapeutics that modulate angiogenesis.

### SUMMARY OF THE INVENTION

Hedgehog proteins are angiogenic growth factors which can have utility in treating tissue repair and ischemia and that inhibition of the hedgehog proteins and the hedgehog pathway can prevent angiogenesis driven pathologies.

### BRIEF DESCRIPTION OF THE DRAWINGS

FIG. 1: Alignment of N-terminal fragments of Human Hedgehog Proteins

FIG. 2: Consensus sequence of a hedgehog protein suitable for use in developing the conjugated proteins of the invention, antagonist, where “Xaa” indicates amino acids that differ between the Sonic, Indian and Desert hedgehog proteins.

### DETAILED DESCRIPTION OF THE INVENTION

The present invention relates to the use of hedgehog protein, DNA, or other hedgehog therapeutic as an agent to induce the growth of new blood vessels, ie angiogenesis, arteriogenesis or vascular growth in adult tissues where the induction of angiogenesis has therapeutic value. The present invention also relates to the use of inhibitors of hedgehog protein or signaling to prevent angiogenesis contributing to pathological conditions such as neoplasia (tumors and gliomas), diabetic retinopathy, rheumatoid arthritis, osteroarthritis, macular degeneration, psoriasis, ulcerative colitis, Chrohn's disease, and inflammation.

All references cited in the Detailed Description are incorporated herein by references, unless stipulated otherwise. The following terms are used herein:

I. Definitions

“Angiogenesis” is defined as any alteration of an existing vascular bed or the formation of new vasculature which benefits tissue perfusion. This includes the formation of new vessels by sprouting of endothelial cells from existing blood vessels or the remodeling of existing vessels to alter size, maturity. direction or flow properties to improve blood perfusion of tissue.

Mesenchymal cells are defined as cells of mesenchymal origin including fibroblasts, stromal cells, smooth muscle cells, skeletal muscle cells, cells of osteogenic origin such as chondrocytes, cells of hemaeopoietic origin such as monocytes, macrophages, lymphocytes, granulocytes and cells of adipose origin such as adipocytes.

A hedgehog therapeutic, whether it is a hedgehog angonist or hedgehog antagonist is said to have “therapeutic efficacy” in modulating angiogenesis and an amount of the therapeutic is said to be a “angiogenic modulatory amount”, if administration of that amount of the therapeutic is sufficient to cause a significant modulation (i.e., increase or decrease) in angiogenic activity when administered to a subject (e.g., an animal model or human patient) needing modulation of angiogenesis.

As used herein, a hedgehog therapeutic of the invention is an “agonist” if it “modulates” hedgehog biological activity (i.e., elicits, allows and/or enhances hedgehog biological activity). For the purposes of the invention an agonist also refers to an agent, e.g., a polypeptide such as an hedgehog or patched or a small organic molecule which can elicit, allow and/or enhance hedgehog and/or patched-mediated binding or which can otherwise modulate hedgehog and/or patched function, e.g., by activating hedgehog-ligand mediated hedgehog signal transduction. Such an agonist of the hedgehog/patched interaction is an agent which has one or more of the following properties: (1) it coats, or binds to, a hedgehog protein associated with an extracellular matrix, e.g., heparin, heparin proteoglycans, collagen, fibronectin, vitronectin, thrombospondin, or on the surface of a hedgehog bearing or secreting cell with sufficient specificity to modulate a hedgehog-ligand/hedgehog receptor interaction, e.g., the hedgehog/patched-smoothened interaction; (2) it coats, or binds to, a hedgehog on the surface of a hedgehog-bearing or secreting cell with sufficient specificity to modify, and preferably to modulate, transduction of a hedgehog-mediated signal e.g., hedgehog/patched-smoothened—mediated signaling; (3) it coats, or binds to, a hedgehog receptor or co-receptor, (e.g., patched, smoothened or a heparin proteoglycan) in or on cells with sufficient specificity to modulate the hedgehog/patched-smoothened interaction; (4) it coats, or binds to, a hedgehog receptor (e.g., patched or smoothened) in or on cells with sufficient specificity to modify, and preferably to modulate, transduction of hedgehog receptor mediated hedgehog signaling, e.g., patched, smoothened, fused or gli-mediated hedgehog signaling.

In preferred embodiments an agonist has one or both of properties 1 and 2. In other preferred embodiments the agonist has one or both of properties 3 and 4. Moreover, more than one agonist can be administered to a patient, e.g., an agent which binds to hedgehog can be combined with an agent which binds to patched. Moreover, a hedgehog therapeutic is an “agonist” if it modulates angiogenesis in such a way as to enhance, elicit, accelerate or increase angiogenesis, regardless of the mode of action of such therapeutic.

As used herein, a hedgehog therapeutic is an “antagonist” if it de-activates the hedgehog receptor or inhibits its activity or inhibits activity of the hedgehog protein. Such an antagonist may additionally have one or more of the following properties: (1) it coats, or binds to, a hedgehog protein on the surface of a hedgehog bearing or secreting cell with sufficient specificity to de-activate or inhibit a hedgehog-ligand/hedgehog interaction, e.g., the hedgehog/patched interaction; (2) it coats, or binds to, a hedgehog protein on the surface of a hedgehog-bearing or secreting cell with sufficient specificity to modify, and preferably to de-activate or inhibit, transduction of a hedgehog-mediated signal e.g., hedgehog/patched, smoothened, fused, or gli -mediated signaling; (3) it coats, or binds to, a hedgehog receptor or coreceptor (e.g., patched or smoothened) in or on cells with sufficient specificity to de-activate or inhibit the hedgehog/patched interaction; (4) it coats, or binds to, a hedgehog receptor or co-receptor (e.g., patched or smoothened) in or on cells with sufficient specificity to modify, and preferably to de-activate or inhibit transduction of hedgehog receptor mediated hedgehog signaling, e.g., patched-mediated hedgehog signaling. In preferred embodiments an antagonist has one or both of properties 1 and 2. In other preferred embodiments the antagonist has one or both of properties 3 and 4. Moreover, more than one antagonist can be administered to a patient, e.g., an agent which binds to hedgehog can be combined with an agent which binds to patched. Moreover, a hedgehog therapeutic is an “antagonist” if it modulates angiogenesis in such a way as to inhibit, decelerate, reverse or otherwise slow angiogenesis, regardless of the mode of action of such therapeutic. For example, antagonist molecules may be antibody homologs (defined below), certain fragments of hedgehog, or small organic molecules that may be administered and modulate hedgehog binding sites on cells.

As discussed herein, the hedgehog therapeutics (i.e., antagonists or agonists) that can be linked or otherwise conjugated to, for instance, an antibody homolog such as an immunoglobulin or fragment thereof are not limited to a particular type or structure of hedgehog or patched or other molecule so that, for purposes of the invention, any agent capable of forming a chimeric protein and capable of effectively modulating hedgehog is considered to be an equivalent of the therapeutics used in the examples herein.

As used herein, the term “antibody homolog” includes intact antibodies consisting of immunoglobulin light and heavy chains linked via disulfide bonds. The term “antibody homolog” is also intended to encompass a hedgehog therapeutic comprising one or more polypeptides selected from immunoglobulin light chains, immunoglobulin heavy chains and antigen-binding fragments thereof which are capable of binding to one or more antigens (i.e., hedgehog or patched). The component polypeptides of an antibody homolog composed of more than one polypeptide may optionally be disulfide-bound or otherwise covalently crosslinked. Accordingly, therefore, “antibody homologs” include intact immunoglobulins of types IgA, IgG, IgE, IgD, IgM (as well as subtypes thereof), wherein the light chains of the immunoglobulin may be of types kappa or lambda or portions of intact antibodies that retain antigen-binding specificity, for example, Fab fragments, Fab′ fragments, F(ab′)2 fragments, F(v) fragments, heavy chain monomers or dimers, light chain monomers or dimers, dimers consisting of one heavy and one light chain, and the like.

As used herein, a “humanized antibody homolog” is an antibody homolog, produced by recombinant DNA technology, in which some or all of the amino acids of a human immunoglobulin light or heavy chain that are not required for antigen binding have been substituted for the corresponding amino acids from a nonhuman mammalian immunoglobulin light or heavy chain. A “human antibody homolog” is an antibody homolog in which all the amino acids of an immunoglobulin light or heavy chain (regardless of whether or not they are required for antigen binding) are derived from a human source.

“amino acid”—a monomeric unit of a peptide, polypeptide, or protein. There are twenty amino acids found in naturally occurring peptides, polypeptides and proteins, all of which are L-isomers. The term also includes analogs of the amino acids and D-isomers of the protein amino acids and their analogs.

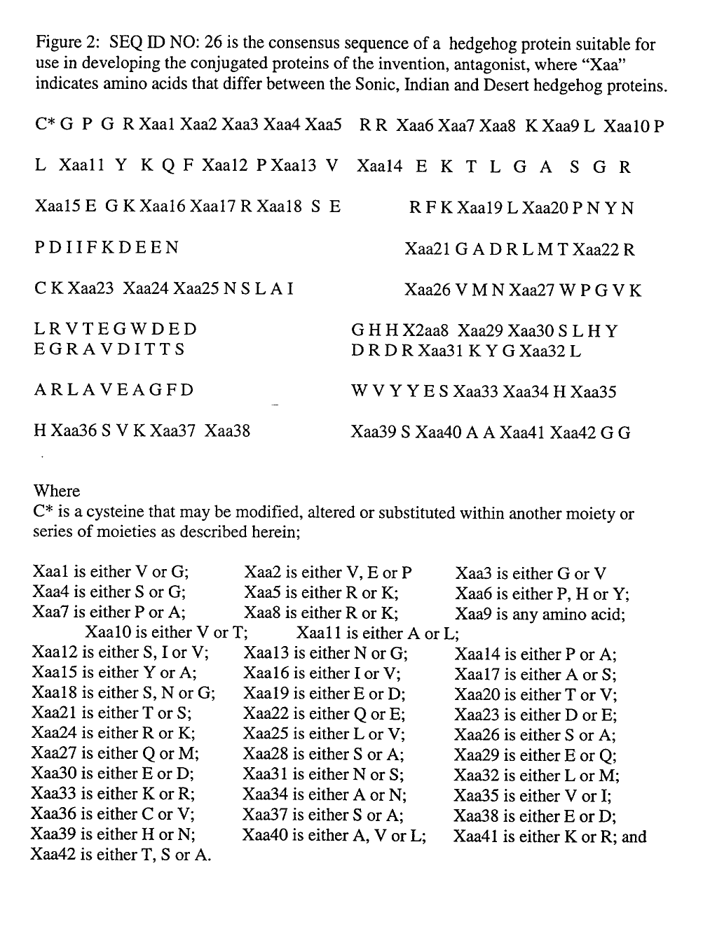

A hedgehog therapeutic has “biological activity” if it has at least one of the following properties: (i) it has the ability to bind to its receptor, patched or it encodes, upon expression, a polypeptide that has this characteristic; and/or (ii) it may induce alkaline phosphatase activity in C3H10T1/2 cells. The hedgehog therapeutic protein meeting this functional test of “biological activity” may meet the hedgehog consensus criteria as defined herein in FIG. 2 (SEQ ID NO: 26). This term “biological activity” includes antagonists and agonists.

The term “bioavailability” refers to the ability of a compound to be absorbed by the body after administration. For instance, a first compound has greater bioavailability than a second compound if, when both are administered in equal amounts, the first compound is absorbed into the blood to a greater extent than the second compound.

The term “chimeric” hedgehog therapeutic is a generic term referring to constructs X-A, where “X” is a polypeptide having the amino acid sequence or portion thereof, consisting of the amino acid sequence of a hedgehog protein and “A” is at least part of a polypeptide other than hedgehog. “A” may include a linker sequence (as defined below) and may be attached to either, or both, of the N- or C-terminii of the hedgehog moiety. Chimeric hedgehog therapeutics of the invention therefore include compounds in which the various moieties are chemically cross-linked or covalently “fused” (as defined below).

As used herein, the term “covalently coupled” means that the specified moieties of the hedgehog therapeutic are either directly covalently bonded to one another, or else are indirectly covalently joined to one another through an intervening moiety or moieties, such as a bridge, spacer, or linkage moiety or moieties. The intervening moiety or moieties are called a “coupling group”. The term “conjugated” is used interchangeably with “covalently coupled”.

“expression control sequence”—a sequence of polynucleotides that controls and regulates expression of genes when operatively linked to those genes.

“expression vector”—a polynucleotide, such as a DNA plasmid or phage (among other common examples) which allows expression of at least one gene when the expression vector is introduced into a host cell. The vector may, or may not, be able to replicate in a cell.

The phrase “extracellular signaling protein” means any protein that is either secreted from a cell, or is associated with the cell membrane, and upon binding to the receptor for that protein on a target cell, triggers a response in the target cell.

“functional equivalent” of an amino acid residue is (i) an amino acid having similar reactive properties as the amino acid residue that was replaced by the functional equivalent; (ii) an amino acid of a ligand of a polypeptide of the invention, the amino acid having similar properties as the amino acid residue that was replaced by the functional equivalent; (iii) a non-amino acid molecule having similar properties as the amino acid residue that was replaced by the functional equivalent.

A first polynucleotide encoding hedgehog protein is “functionally equivalent” compared with a second polynucleotide encoding hedgehog protein if it satisfies at least one of the following conditions:

(a) the “functional equivalent” is a first polynucleotide that hybridizes to the second polynucleotide under standard hybridization conditions and/or is degenerate to the first polynucleotide sequence. Most preferably, it encodes a mutant hedgehog having the activity of an hedgehog therapeutic;

(b) the “functional equivalent” is a first polynucleotide that codes on expression for an amino acid sequence encoded by the second polynucleotide.

The term “hedgehog therapeutic” includes, but is not limited to, the agonist and/or antagonist agents listed herein as well as their functional equivalents. As used herein, the term “functional equivalent” therefore refers to, for example, an hedgehog protein or a polynucleotide encoding the hedgehog protein that has the same or an improved beneficial effect on the mammalian recipient as the hedgehog of which it is deemed a functional equivalent. As will be appreciated by one of ordinary skill in the art, a functionally equivalent protein can be produced by recombinant techniques, e.g., by expressing a “functionally equivalent DNA”. Accordingly, the instant invention embraces hedgehog therapeutics encoded by naturally-occurring DNAs, as well as by non-naturally-occurring DNAs which encode the same protein as encoded by the naturally-occurring DNA. Due to the degeneracy of the nucleotide coding sequences, other polynucleotides may be used to encode hedgehog protein. These include all, or portions of the above sequences which are altered by the substitution of different codons that encode the same amino acid residue within the sequence, thus producing a silent change. Such altered sequences are regarded as equivalents of these sequences. For example, Phe (F) is coded for by two codons, TTC or TTT, Tyr (Y) is coded for by TAC or TAT and His (H) is coded for by CAC or CAT. On the other hand, Trp (W) is coded for by a single codon, TGG. Accordingly, it will be appreciated that for a given DNA sequence encoding a particular hedgehog there will be many DNA degenerate sequences that will code for it. These degenerate DNA sequences are considered within the scope of this invention.

The term “fusion” or “fusion protein” is a species of chimeric hedgehog therapeutic and refers to a co-linear, covalent linkage of two or more proteins or fragments thereof via their individual peptide backbones, most preferably through genetic expression of a polynucleotide molecule encoding those proteins. It is preferred that the proteins or fragments thereof are from different sources (e.g., a ‘chimeric’ protein). Thus, preferred fusion therapeutics include an hedgehog protein or fragment covalently linked to a second moiety that is not a hedgehog protein. In certain embodiments, the non-hedgehog moiety may be a protein having a domain or region which is homologous to a member of the immunoglobulin gene superfamily. Members of this superfamily inlcude class I and class II major histocompatability antigens, CD4 and T cell receptor chains. Further examples of members of this family and fusion proteins containing them are found in U.S. Pat. No. 5,565,335 (Genentech), incorporated herein by reference.

Non-hedgehog proteins of this type are useful if they contain one or more amino acid sequences at least 20, 50, 75 or 150 residues in length, that are at least 40% homologous to a sequence of an immunoglobulin constant or variable region. A non-hedgehog protein meeting these requirements is said to possess an “Ig-like domain” which may be an “Ig-like constant domain” or an “Ig-like variable domain”. Thus, one embodiment of the present invention is a chimeric hedgehog therapeutic in which the non-hedgehog moiety contains at least one Ig-like domain, or portion thereof.

Other embodiments are possible. Specifically, a “hedgehog/Ig fusion” is a hedgehog therapeutic comprising a biologically active hedgehog molecule of the invention (i.e., Sonic hedgehog), or a biologically active fragment thereof (i.e., the N-terminal portion) linked to an N-terminus of an immunoglobulin chain wherein a portion of the N-terminus of the immunoglobulin is replaced with the hedgehog. A species of hedgehog/Ig fusion is an “hedgehog/Fc fusion” which is a protein comprising an hedgehog molecule of the invention (i.e., hedgehog—) linked to at least a part of the constant domain of an immunoglobulin. Also, the term “fusion protein” means an hedgehog protein chemically linked via a mono- or hetero- functional molecule to a second moiety that is not an hedgehog protein and is made de novo from purified protein as described below. Thus, this invention features a hedgehog therapeutic molecule which includes: (1) a hedgehog moiety, (2) a second peptide, e.g., one which increases solubility or in vivo life time of the hedgehog moiety, e.g., a member of the immunoglobulin super family or fragment or portion thereof, e.g., a portion or a fragment of IgG, e.g., the human IgGl heavy chain constant region, e.g., CH2, CH3, and hinge regions; and a toxin moiety.

“Heterologous promoter”—as used herein is a promoter which is not naturally associated with a gene or a purified nucleic acid.

“Homology” and “identity” each refer to sequence similarity between two polypeptide sequences, and both homology and ‘identity’ are used interchangeably in this disclosure. Homology can be determined by comparing a position in each sequence which may be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology between sequences is a function of the number of matching or homologous positions shared by the sequences. An “unrelated” or “non-homologous” sequence shares less than 40 percent identity, though preferably less than 25 percent identity, with a sequence of the present invention.

For instance, if 6 of 10 of the positions in two sequences are matched or are homologous, then the two sequences are 60% homologous. By way of example, the DNA sequences CTGACT and CAGGTT share 50% homology (3 of the 6 total positions are matched). Generally, a comparison is made when two sequences are aligned to give maximum homology. Such alignment can be provided using, for instance, the method of Needleman et al., J. Mol Biol. 48: 443-453 (1970), implemented conveniently by computer programs described in more detail below. Homologous sequences share identical or similar amino acid residues, where similar residues are conservative substitutions for, or “allowed point mutations” of, corresponding amino acid residues in an aligned reference sequence. In this regard, a “conservative substitution” of a residue in a reference sequence are those substitutions that are physically or functionally similar to the corresponding reference residues, e.g., that have a similar size, shape, electric charge, chemical properties, including the ability to form covalent or hydrogen bonds, or the like. Particularly preferred conservative substitutions are those fulfilling the criteria defined for an “accepted point mutation” in Dayhoff et al., 5: Atlas of Protein Sequence and Structure, 5: Suppl. 3, chapter 22: 354-352, Nat. Biomed. Res. Foundation, Washington, D.C. (1978).

“Percent homology/identity” of two amino acids sequences or two nucleic acid sequences is determined using the alignment algorithm of Karlin and Altschul (Proc. Nat. Acad. Sci., USA 87: 2264 (1990) as modified in Karlin and Altschul (Proc. Nat. Acad. Sci., USA 90: 5873 (1993). Such an algorithm is incorporated into the NBLAST or XBLAST programs of Altschul et al., J. Mol. Biol. 215: 403 (1990). BLAST searches are performed with the NBLAST program, score=100, wordlength=12, to obtain nucleotide sequences homologous to a nucleic acid of the invention. BLAST protein searches are performed with the XBLAST program, score=50, wordlength=3, to obtain amino acid sequences homologous to a reference polypeptide. To obtain gapped alignments for comparisons, gapped BLAST is used as described in Altschul et al., Nucleic Acids Res., 25: 3389 (1997). When using BLAST and Gapped BLAST, the default parameters of the respective programs (XBLAST and NBLAST) are used. See http://www/n cbi.nlm.nih.gov.

The term “hedgehog N-terminal fragment” may be used interchangeably with “Hedgehog” and refers to the active mature sequence that is proteolytically cleaved from the hedgehog precursor.

The term “hydrophobic” refers to the tendency of chemical moieties with nonpolar atoms to interact with each other rather than water or other polar atoms. Materials that are “hydrophobic” are, for the most part, insoluble in water. Natural products with hydrophobic properties include lipids, fatty acids, phospholipids, sphingolipids, acylglycerols, waxes, sterols, steroids, terpenes, prostaglandins, thromboxanes, leukotrienes, isoprenoids, retenoids, biotin, and hydrophobic amino acids such as tryptophan, phenylalanine, isoleucine, leucine, valine, methionine, alanine, proline, and tyrosine. A chemical moiety is also hydrophobic or has hydrophobic properties if its physical properties are determined by the presence of nonpolar atoms.

The phrase “internal amino acid” means any amino acid in a peptide sequence that is neither the N-terminal amino acid nor the C-terminal amino acid.

“Isolated” (used interchangeably with “substantially pure”) when applied to nucleic acid i.e., polynucleotide sequences that encode polypeptides, means an RNA or DNA polynucleotide, portion of genomic polynucleotide, cDNA or synthetic polynucleotide which, by virtue of its origin or manipulation: (i) is not associated with all of a polynucleotide with which it is associated in nature (e.g., is present in a host cell as an expression vector, or a portion thereof); or (ii) is linked to a nucleic acid or other chemical moiety other than that to which it is linked in nature; or (iii) does not occur in nature. By “isolated” it is further meant a polynucleotide sequence that is: (i) amplified in vitro by, for example, polymerase chain reaction (PCR); (ii) synthesized chemically; (iii) produced recombinantly by cloning; or (iv) purified, as by cleavage and gel separation.

“Isolated” (used interchangeably with “substantially pure”) when applied to polypeptides means a polypeptide or a portion thereof which, by virtue of its origin or manipulation: (i) is present in a host cell as the expression product of a portion of an expression vector; or (ii) is linked to a protein or other chemical moiety other than that to which it is linked in nature; or (iii) does not occur in nature, for example, a protein that is chemically manipulated by appending, or adding at least one hydrophobic moiety to the protein so that the protein is in a form not found in nature. By “isolated” it is further meant a protein that is: (i) synthesized chemically; or (ii) expressed in a host cell and purified away from associated and contaminating proteins. The term generally means a polypeptide that has been separated from other proteins and nucleic acids with which it naturally occurs. Preferably, the polypeptide is also separated from substances such as antibodies or gel matrices (polyacrylamide) which are used to purify it.

“multivalent protein complex” refers to a plurality of hedgehog therapeutics (i.e., one or more).

“mutant” is any change in the genetic material of an organism, in particular any change (i.e., deletion, substitution, addition, or alteration) in a wild type polynucleotide sequence or any change in a wild type protein. The term “mutein” is used interchangeably with “mutant”.

“N-terminal end” refers to the first amino acid residue (amino acid number 1) of the mature form of a protein.

“N-terminal cysteine” refers to the amino acid number 1 as shown in SEQ ID NOS. 23-26. In certain embodiments of the hedgehog therapeutic, the N-terminal cysteine has been “modified”. The term “modified” in this regard refers to chemical modifications of the N-terminal cysteine such as linkage thereof to another moiety such as a hydrophobic group and/or replacement of the N-terminal cysteine with another moiety, such as a hydrophobic group.

“operatively linked”: A polynucleotide sequence (DNA, RNA) is operatively linked to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

“protein” is any polymer consisting essentially of any of the 20 amino acids. Although “polypeptide” is often used in reference to relatively large polypeptides, and “peptide” is often used in reference to small polypeptides, usage of these terms in the art overlaps and is varied. The term “protein” as used herein refers to peptides, proteins and polypeptides, unless otherwise noted.

The terms “peptide(s)”, “protein(s)” and “polypeptide(s)” are used interchangeably herein. The terms “polynucleotide sequence” and “nucleotide sequence” are also used interchangeably herein.

“Recombinant,” as used herein, means that a protein is derived from recombinant, mammalian expression systems. Since hedgehog is not glycosylated nor contains disulfide bonds, it can be expressed in most prokaryotic and eukaryotic expression systems.

“Spacer” sequence refers to a moiety that may be inserted between an amino acid to be modified with an antibody homolog or fragment and the remainder of the protein. A spacer is designed to provide separation between the modification and the rest of the protein so as to prevent the modification from interfering with protein function and/or make it easier for the modification to link with an antibody homolog moiety or any other moiety.

Thus, “substantially pure nucleic acid” is a nucleic acid which is not immediately contiguous with one or both of the coding sequences with which it is normally contiguous in the naturally occurring genome of the organism from which the nucleic acid is derived. Substantially pure DNA also includes a recombinant DNA which is part of a hybrid gene encoding additional hedgehog sequences.

The phrase “surface amino acid” means any amino acid that is exposed to solvent when a protein is folded in its native form.

“standard hybridization conditions” refer to salt and temperature conditions substantially equivalent to 0.5 X SSC to about 5 X SSC and 65° C. for both hybridization and wash. The term “standard hybridization conditions” as used herein is therefore an operational definition and encompasses a range of hybridization conditions. Nevertheless, for the purposes of this present disclosure “high stringency” conditions include hybridizing with plaque screen buffer (0.2% polyvinylpyrrolidone, 0.2% Ficoll 400; 0.2% bovine serum albumin, 50 mM Tris—HCl (pH 7.5); 1 M NaCl; 0.1% sodium pyrophosphate; 1% SDS); 10% dextran sulfate, and 100 ug/ml denatured, sonicated salmon sperm DNA at 65° C. for 12-20 hours, and washing with 75 mM NaCl/7.5 mM sodium citrate (0.5 x SSC)/1% SDS at 65° C. “Low stringency” conditions include hybridizing with plaque screen buffer, 10% dextran sulfate and 110 ug/ml denatured, sonicated salmon sperm DNA at 55° C. for 12-20 hours, and washing with 300 mM NaCl/30 mM sodium citrate (2.0 X SSC)/1% SDS at 55° C. See also Current Protocols in Molecular Biology, John Wiley & Sons, Inc. New York, Sections 6.3.1-6.3.6, (1989).

A “therapeutic composition” as used herein is defined as comprising the therapeutics of the invention and other biologically compatible ingredients. The therapeutic composition may contain excipients such as water, minerals and carriers such as protein.

“wild type”—the naturally-occurring polynucleotide sequence of an exon of a protein, or a portion thereof, or protein sequence, or portion thereof, respectively, as it normally exists in vivo.

Practice of the present invention will employ, unless indicated otherwise, conventional techniques of cell biology, cell culture, molecular biology, microbiology, recombinant DNA, protein chemistry, and immunology, which are within the skill of the art. Such techniques are described in the literature. Unless stipulated otherwise, all references cited in the Detailed Description are incorporated herein by reference.

II. General Properties of Isolated Hedgehog Proteins

Hedgehogs are a family of genes which begin expression early in development and are involved in the morphogenesis of a number of organs in the developing embryo (Ingharn, 1995, Perrimon, 1995; Johnson and Tabin, 1995; Hammerschmidt et al., 1997).

However, there is currently no evidence that hedgehogs are directly involved in the development of the mammalian vasculature. Knockouts of each of the mammalian hedgehog genes, sonic (Chiang et al., 1996; Litingtung et al., 1998; St-Jacques et al., 1998), indian (St-Jacques et al., 1999; Karp et al., 2000) and desert (Bitgood et al., 1996; Parmantier et al., 1999) hedgehog have not been reported to have defects in vascular development, but do show defects in tissues where they are known to function in development.

The adult functions of the hedgehog proteins are not well understood. Hedgehog is known to be expressed in adult bone/cartilage, central and peripheral nervous system, kidney, eye and several other tissues (Valentine et al., 1997; Traiffort et al., 1998 and 1999; Iwamoto et al., 1999; Jensen et al., 1997; Parmantier et al., 1999). The adult function of the hedgehog pathway is perhaps best understood in bone and cartilage where it regulates the differentiation of chondrocytes by modulating PTHrp (Iwamoto et al., 1999; Karp et al., 2000). Administration of hedgehog locally in the skin also can induce hair growth in adult animals (Sato et al., 1999; Wang et al., 2000).

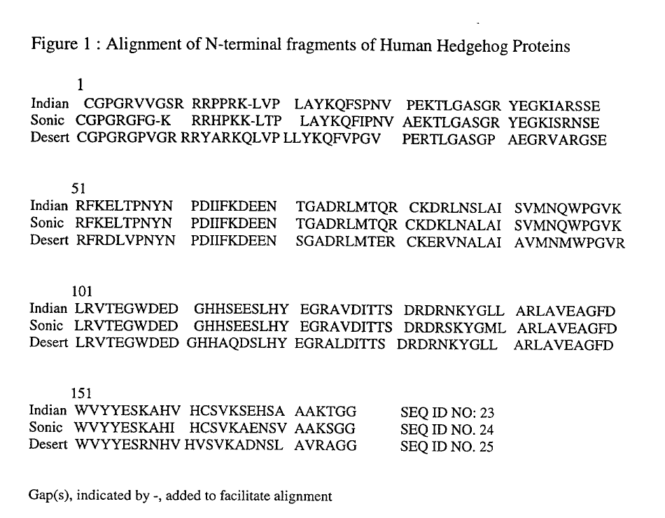

The various naturally-occurring hedgehog proteins from which the subject therapeutics can be derived are characterized by a signal peptide, a highly conserved N-terminal region (see FIG. 1), and a more divergent C-terminal domain. In addition to signal sequence cleavage in the secretory pathway (Lee, J.J. et al. (1992) Cell 71:33-50; Tabata, T. et al. (1992) Genes Dev. 2635-2645; Chang, D.E. et al. (1994) Development 120:3339-3353), hedgehog precursor proteins naturally undergo an internal autoproteolytic cleavage which depends on conserved sequences in the C-terminal portion (Lee et al. (1994) Science 266:1528-1537; Porter et al. (1995) Nature 374:363-366). This autocleavage leads to a 19 kD N-terminal peptide and a C-terminal peptide of 26-28 kD. The N-terminal peptide stays tightly associated with the surface of cells in which it was synthesized, while the C-terminal peptide is freely diffusible both in vitro and in vivo. Cell surface retention of the N-terminal peptide is dependent on autocleavage, as a truncated form of hedgehog encoded by an RNA which terminates precisely at the normal position of internal cleavage is diffusible in vitro (Porter et al. (1995) supra) and in vivo (Porter, J.A. et al. (1996) Cell 86, 21-34). Biochemical studies have shown that the autoproteolytic cleavage of the hedgehog precursor protein proceeds through an internal thioester intermediate, which subsequently is cleaved in a nucleophilic substitution. It is suggested that the nucleophile is a small lipophilic molecule, more particularly cholesterol, which becomes covalently bound to the C-terminal end of the N-peptide (Porter et al. (1996) supra), tethering it to the cell surface.

The vertebrate family of hedgehog genes includes at least four members, e.g., paralogs of the single drosophila hedgehog gene (reference). Three of these members, herein referred to as Desert hedgehog (Dhh), Sonic hedgehog (Shh) and Indian hedgehog (Ihh), apparently exist in all vertebrates, including fish, birds, and mammals. A fourth member, herein referred to as tiggie-winkle hedgehog (Thh), appears specific to fish. Isolated hedgehog proteins used in the methods of this invention are naturally occurring or recombinant proteins of the hedgehog family and may be obtainable from either invertebrate or from vertebrate sources (see references below). Members of the vertebrate hedgehog protein family share homology with proteins encoded by the Drosophila hedgehog (hh) gene (Mohler and Vani, (1992) Development 115, 957-971). Other members continue to be identified.

Mouse and chicken Shh and mouse Ihh genes (see, for example, U.S. Pat. No. 5,789,543) encode glycoproteins which undergo cleavage, yielding an amino terminal fragment of about 2OkDa and a carboxy terminal fragment of about 25kDa. The most preferred 2OkDa fragment has the consensus sequence SEQ ID NO: 26 which includes the amino acid sequences of SEQ ID NOS: 23-25. Various other fragments that encompass the 2OkDa moiety are considered within the presently claimed invention. Publications disclosing these sequences, as well as their chemical and physical properties, include Hall et al., (1995) Nature 378, 212-216; Ekker et al., (1995) Current Biology 5, 944-955; Fan et al., (1995) Cell 81, 457-465, Chang et al., (1994) Development 120, 3339-3353; Echelard et al., (1993) Cell 75, 1414-1430 34-38; PCT Patent Application WO 95/23223 (Jessell, Dodd, Roelink and Edlund); PCT Patent Publication WO 95/18856 (Ingham, McMahon and Tabin). U.S. Pat. No. 5,759,811 lists the Genbank accession numbers of a complete mRNA sequence encoding human Sonic hedgehog; a partial sequence of human Indian hedgehog mRNA, 5′ end; and a partial sequence of human Desert hedgehog mRNA. The hedgehog therapeutic compositions of the subject method can be generated by any of a variety of techniques, including purification of naturally occurring proteins, recombinantly produced proteins and synthetic chemistry. Polypeptide forms of the hedgehog therapeutics are preferably derived from vertebrate hedgehog proteins, e.g., have sequences corresponding to naturally occurring hedgehog proteins, or fragments thereof, from vertebrate organisms. However, it will be appreciated that the hedgehog polypeptide can correspond to a hedgehog protein (or fragment thereof) which occurs in any metazoan organism.

The vertebrate family of hedgehog genes includes at least four members, e.g., paralogs of the single drosophila hedgehog gene (SEQ ID No. 19). Three of these members, herein referred to as Desert hedgehog (Dhh), Sonic hedgehog (Shh) and Indian hedgehog (Ihh), apparently exist in all vertebrates, including fish, birds, and mammals. A fourth member, herein referred to as tiggie-winkle hedgehog (Thh), appears specific to fish. According to the appended sequence listing, (see also Table 1) a chicken Shh polypeptide is encoded by SEQ ID No: 1; a mouse Dhh polypeptide is encoded by SEQ ID No:2; a mouse Ihh polypeptide is encoded by SEQ ID No:3; a mouse Shh polypeptide is encoded by SEQ ID No:4 a zebrafish Shh polypeptide is encoded by SEQ ID No:5; a human Shh polypeptide is encoded by SEQ ID No:6; a human Ihh polypeptide is encoded by SEQ ID No:7; a human Dhh polypeptide is encoded by SEQ ID No. 8; and a zebrafish Thh is encoded by SEQ ID No. 9.

TABLE 1
Guide to hedgehog sequences in Sequence Listing

| | Nucleotide | Amino Acid |
|---|---|---|
| Chicken Shh | SEQ ID No. 1 | SEQ ID No. 10 |
| Mouse Dhh | SEQ ID No. 2 | SEQ ID No. 11 |
| Mouse Ihh | SEQ ID No. 3 | SEQ ID No. 12 |
| Mouse Shh | SEQ ID No. 4 | SEQ ID No. 13 |
| Zebrafish Shh | SEQ ID No. 5 | SEQ ID No. 14 |
| Human Shh | SEQ ID No. 6 | SEQ ID No. 15 |
| Human Ihh | SEQ ID No. 7 | SEQ ID No. 16 |
| Human Dhh | SEQ ID No. 8 | SEQ ID No. 17 |
| zebrafish Thh | SEQ ID No. 9 | SEQ ID No. 18 |
| Drosophila HH | SEQ ID No. 19 | SEQ ID No. 20 |

In addition to the sequence variation between the various hedgehog homologs, the hedgehog proteins are apparently present naturally in a number of different forms, including a pro-form, a full-length mature form, and several processed fragments thereof. The pro-form includes an N-terminal signal peptide for directed secretion of the extracellular domain, while the full-length mature form lacks this signal sequence.

As described above, further processing of the mature form occurs in some instances to yield biologically active fragments of the protein. For instance, sonic hedgehog undergoes additional proteolytic processing to yield two peptides of approximately 19 kDa and 27 kDa, the 19kDa fragment corresponding to an proteolytic N-terminal portion of the mature protein.

In addition to proteolytic fragmentation, the vertebrate hedgehog proteins can also be modified post-translationally, such as by glycosylation and/or addition of lipophilic moieties, such as stents, fatty acids, etc., though bacterially produced (e.g. unmodified) forms of the proteins still maintain certain of the bioactivities of the native protein. Bioactive fragments of hedgehog polypeptides of the present invention have been generated and are described in great detail in, e.g., PCT publications WO 95/18856 and WO 96/17924.

A “hedgehog therapeutic” of the invention is defined in terms of having at least a portion that consists of the consensus amino acid sequence of SEQ ID NO: 26 or at least a portion that consists of SEQ ID NOS: 10-18 or 23-25. The term also means a hedgehog polypeptide, or a functional variant of a hedgehog polypeptide, or homolog of a hedgehog polypeptide, or functional variant, which has biological activity and can modulate angiogenesis.

Members useful in the methods of the invention include any of the naturally-occurring native hedgehog proteins including allelic, phylogenetic counterparts or other variants thereof, whether naturally-sourced or produced chemically including muteins or mutant proteins, as well as recombinant forms and new, active members of the hedgehog family. Particularly useful hedgehog polypeptides have portions that include all or part of SEQ ID NOS: 23-26.

Hedgehog therapeutics may also include polypeptides having an amino acid sequence at least 60%, 80%, 90%, 95%, 98%, or 99% homologous to an amino acid sequence from SEQ ID NOS 10-18 or 23-26. The polypeptide can also include an amino acid sequence essentially the same as an amino acid sequence in SEQ ID NOS: 10-18 or 23-26. The polypeptide is at least 5, 10, 20, 50, 100, or 150 amino acids in length and includes at least 5, preferably at least 10, more preferably at least 20, most preferably at least 50, 100, or 150 contiguous amino acids from SEQ ID NOS: 10-18 or 23-26.

Polypeptides of the invention include those which arise as a result of the existence of multiple genes, alternative transcription events, alternative RNA splicing events, and alternative translational and posttranslational events. The polypeptide can be made entirely by synthetic means or can be expressed in systems, e.g., cultured cells, which result in substantially the same posttranslational modifications present when the protein is expressed in a native cell, or in systems which result in the omission of posttranslational modifications present when expressed in a native cell.

Moreover, mutagenesis can be used to create modified hh polypeptides, e.g., for such purposes as enhancing therapeutic or prophylactic efficacy, or stability (e.g., ex vivo shelf life and resistance to proteolytic degradation in vivo). Such modified peptides can be produced, for instance, by amino acid substitution, deletion, or addition. Modified hedgehog polypeptides can also include those with altered post-translational processing relative to a naturally occurring hedgehog protein, e.g., altered glycosylation, cholesterolization, prenylation and the like.

In one embodiment, a hedgehog therapeutic is a hedgehog polypeptide with one or more of the following characteristics:

(i) it has at least 30, 40, 42, 50, 60, 70, 80, 90 or 95% sequence identity with amino acids of SEQ ID NOS: 23-26;

(ii) it has a cysteine or a functional equivalent as the N-terminal end;

(iii) it may induce alkaline phosphatase activity in C3HlOTl/2 cells;

(iv) it has an overall sequence identity of at least 50%, preferably at least 60%, more preferably at least 70, 80, 90, or 95%, with a polypeptide of SEQ ID NOS: 10-18;

(v) it can be isolated from natural sources such as mammalian cells;

(vi) it can bind or interact with patched; and

(vii) it may be modified at at least one amino acid residue by a polyalkylene glycol polymer attached to the residue or, optionally, via a linker molecule to the amino acid residue.

Preferred nucleic acids encode a polypeptide comprising an amino acid sequence at least 60% homologous or identical, more preferably 70% homologous or identical, and most preferably 80% homologous or identical with an amino acid sequence selected from the group consisting of SEQ ID NOS: 10-18 or 23-26. Nucleic acids which encode polypeptides at least about 90%, more preferably at least about 95%, and most preferably at least about 98-99% homology or identity with an amino acid sequence represented in one of SEQ ID Nos: 23-26 are also within the scope of the invention.

In another embodiment, the hedgehog therapeutic is a polypeptide encodable by a nucleotide sequence that hybridizes under stringent conditions to a hedgehog coding sequence represented in one or more of SEQ ID NOS: 1-9, 19 or 23-26.

Preferred nucleic acids encode a hedgehog polypeptide comprising an amino acid sequence at least 60% homologous, more preferably 70% homologous and most preferably 80% homologous with an amino acid sequence selected from the group consisting of SEQ ID Nos:8-14. Nucleic acids which encode polypeptides at least about 90%, more preferably at least about 95%, and most preferably at least about 98-99% homology with an amino acid sequence represented in one of SEQ ID Nos: 10-18 or 20 are also within the scope of the invention.

Hedgehog therapeutics, in addition to native hedgehog proteins, are at least 60% homologous, more preferably 70% homologous and most preferably 80% homologous with an amino acid sequence represented by any of SEQ ID Nos: 10-18 or 20. Polypeptides which are at least 90%, more preferably at least 95%, and most preferably at least about 98-99% homologous with a sequence selected from the group consisting of SEQ ID Nos: 10-18 or 20 are also within the scope of the invention.

With respect to fragments of hedgehog polypeptide, preferred hedgehogs moieties include at least 50 amino acid residues of a hedgehog polypeptide, more preferably at least 100, and even more preferably at least 150.

Another preferred hedgehog polypeptide which can be included in the hedgehog therapeutic is an N-terminal fragment of the mature protein having a molecular weight of approximately 19 kDa.

Preferred human hedgehog proteins include N-terminal fragments corresponding approximately to residues 24-197 of SEQ ID No. 15, 28-202 of SEQ ID No. 16, and 23-198 of SEQ ID No. 17. By “corresponding approximately” it is meant that the sequence of interest is at most 20 amino acid residues different in length to the reference sequence, though more preferably at most 5, 10 or 15 amino acid different in length.

Still other preferred hedgehog therapeutics include an amino acid sequence represented by the formula A-B wherein: (i) A represents all or the portion of the amino acid sequence designated by residues 24-193 of SEQ ID No: 15; and B represents at least one amino acid residue of the amino acid sequence designated by residues 194-250 of SEQ ID No: 15; (ii) A represents all or the portion of the amino acid sequence designated by residues 25-193 of SEQ ID No: 13; and B represents at least one amino acid residue of the amino acid sequence designated by residues 194-250 of SEQ ID No: 13; (iii) A represents all or the portion of the amino acid sequence designated by residues 23-193 of SEQ ID No: 11; and B represents at least one amino acid residue of the amino acid sequence designated by residues 194-250 of SEQ ID No: 1; (iv) A represents all or the portion of the amino acid sequence designated by residues 28-197 of SEQ ID No: 12; and B represents at least one amino acid residue of the amino acid sequence designated by residues 198-250 of SEQ ID No: 12; (v) A represents all or the portion of the amino acid sequence designated by residues 29-197 of SEQ ID No: 16; and B represents at least one amino acid residue of the amino acid sequence designated by residues 198-250 of SEQ ID No: 16; or (vi) A represents all or the portion of the amino acid sequence designated by residues 23-193 of SEQ ID No. 17, and B represents at least one amino acid residue of the amino acid sequence designated by residues 194-250 of SEQ ID No. 17. In certain preferred embodiments, A and B together represent a contiguous polypeptide sequence designated sequence, A represents at least 25, 50, 75, 100, 125 or 150 amino acids of the designated sequence, and B represents at least 5, 10, or 20 amino acid residues of the amino acid sequence designated by corresponding entry in the sequence listing, and A and B together preferably represent a contiguous sequence corresponding to the sequence listing entry. Similar fragments from other hedgehog also contemplated, e.g., fragments which correspond to the preferred fragments from the sequence listing entries which are enumerated above.

III. Production of Recombinant Polypeptides

Isolated hedgehog polypeptides described herein can be produced by any suitable method known in the art. Such methods range from direct protein synthetic methods to constructing a DNA sequence encoding isolated polypeptide sequences and expressing those sequences in a suitable transformed host.

In one embodiment of a recombinant method, a DNA sequence is constructed by isolating or synthesizing a DNA sequence encoding a wild type protein of interest. Optionally, the sequence may be mutagenized by site-specific mutagenesis to provide functional analogs thereof. See, e.g., U.S. Pat. No. 4,588,585. Another method of constructing a DNA sequence encoding a polypeptide of interest would be by chemical synthesis using an oligonucleotide synthesizer. Such oligonucleotides may be preferably designed based on the amino acid sequence of the desired polypeptide, and preferably selecting those codons that are favored in the host cell in which the recombinant polypeptide of interest will be produced.

Standard methods may be applied to synthesize an isolated polynucleotide sequence encoding an isolated polypeptide of interest. For example, a complete amino acid sequence may be used to construct a back-translated gene. See Maniatis et al., supra. Further, a DNA oligomer containing a nucleotide sequence coding for the particular isolated polypeptide may be synthesized. For example, several small oligonucleotides coding for portions of the desired polypeptide may be synthesized and then ligated. The individual oligonucleotides typically contain 5′ or 3′ overhangs for complementary assembly.

Once assembled (by synthesis, site-directed mutagenesis, or by another method), the mutant DNA sequences encoding a particular isolated polypeptide of interest will be inserted into an expression vector and operatively linked to an expression control sequence appropriate for expression of the protein in a desired host. Proper assembly may be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host.

The choice of expression control sequence and expression vector will depend upon the choice of host. A wide variety of expression host/vector combinations may be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from Esherichia coli, including pCRI, pBR322, pMB9 and their derivatives, wider host range plasmids, such as M13 and filamentous single-stranded DNA phages. Preferred E. coli vectors include pL vectors containing the lambda phage pL promoter (U.S. Patent 4,874,702), pET vectors containing the T7 polymerase promoter (Studier et al., Methods in Enzymology 185: 60-89,1990 1) and the pSP72 vector (Kaelin et al., supra). Useful expression vectors for yeast cells, for example, include the 2 g and centromere plasmids. Further, within each specific expression vector, various sites may be selected for insertion of these DNA sequences. These sites are usually designated by the restriction endonuclease which cuts them. They are well-recognized by those of skill in the art. It will be appreciated that a given expression vector useful in this invention need not have a restriction endonuclease site for insertion of the chosen DNA fragment. Instead, the vector may be joined by the fragment by alternate means.

The expression vector, and the site chosen for insertion of a selected DNA fragment and operative linking to an expression control sequence, is determined by a variety of factors such as: the number of sites susceptible to a particular restriction enzyme, the size of the polypeptide, how easily the polypeptide is proteolytically degraded, and the like. The choice of a vector and insertion site for a given DNA is determined by a balance of these factors.

To provide for adequate transcription of the recombinant constructs of the invention, a suitable promoter/enhancer sequence may preferably be incorporated into the recombinant vector, provided that the promoter/expression control sequence is capable of driving transcription of a nucleotide sequence encoding a hedgehog protein. Any of a wide variety of expression control sequences may be used in these vectors. Such useful expression control sequences include the expression control sequences associated with structural genes of the foregoing expression vectors. Examples ofuseful expression control sequences include, for example, the-early and late promoters of SV40 or adenovirus, the lac system, the trp system, the TAC or TRC system, the major operator and promoter regions of phage lambda, for example pL, the control regions of fd coat protein, the promoter for 3-phosphoglycerate kinase or other glycolytic enzymes, the promoters of acid phosphatase, e.g., Pho5, the promoters of the yeast alpha-mating system and other sequences known to control the expression of genes of prokaryotic or eukaryotic cells and their viruses, and various combinations thereof.

Promoters which may be used to control the expression of immunoglobulin-based fusion protein include, but are not limited to, the SV40 early promoter region (Benoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:144-1445), the regulatory sequences of the metallothionine gene (Brinster et al., 1982, Nature 296:39-42); plant expression vectors comprising the nopaline synthetase promoter region (Herrera-Estrella et al., Nature 303:209-213) or the cauliflower mosaic virus 35S RNA promoter (Gardner, et al., 1981, Nucl. Acids Res. 9:2871), and the promoter for the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phophatase promoter, and the following animal transcriptional control regions, which exhibit tissue specificity and have been utilized in transgenic animals: elastase I gene control region which is active in pancreatic cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene enhancers or promoters which are active in pancreatic cells (Hanahan, 1985, Nature 315:115-122); immunoglobulin gene enhancers or promoters which are active in lymphoid cells (Grosschedl et al., 1984, Cell 38:647-658; Adames et al., 1985, Nature 318:533-538; Alexander et al., 1987, Mol. Cell. Biol. 7:1436-1444); the cytomegalovirus early promoter and enhancer regions (Boshart et al., 1985, Cell 41:521-530); mouse mammary tumor virus control region which is active in testicular, breast, lymphoid and mast cells (Leder et al., 1986, Cell 45:485-495); albumin gene control region which is active in liver (Pinkert et al., 1987, Genes and Devel. 1:268-276); alpha-fetoprotein gene control region which is active in liver (Krumlauf et al., 1985, Mol. Cell. Biol. 5:1639-1648; Hammer et al., 1987, Science 235:53-58); alphantitrypsin gene control region which is active in the liver (Kelsey et al, 1987, Genes and Devel. 1:161-171); -globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); and gonadotropic releasing hormone gene control region which is active in the hypothalamus (Mason et al., 1986, Science 234:1372-1378).

Any suitable host may be used to produce in quantity the isolated hedgehog polypeptides described herein, including bacteria, fungi (including yeasts), plants, insects, mammals, or other appropriate animal cells or cell lines, as well as transgenic animals or plants. More particularly, these hosts may include well known eukaryotic and prokaryotic hosts, such as strains of E. coli, Pseudomonas, Bacillus, Streptomyces, fungi, yeast (e.g., Hansenula ), insect cells such as Spodoptera fi rugiperda (SF9), and High Five TM, animal cells such as Chinese hamster ovary (CHO), mouse cells such as NS/O cells, African green monkey cells, COS 1, COS 7, BSC 1, BSC 40, and BMT 10, and human cells, as well as plant cells.

It should be understood that not all vectors and expression control sequences will function equally well to express a given isolated polypeptide. Neither will all hosts function equally well with the same expression system. However, one of skill in the art may make a selection among these vectors, expression control systems and hosts without undue experimentation. For example, to produce isolated polypeptide of interest in large-scale animal culture, the copy number of the expression vector must be controlled. Amplifiable vectors are well known in the art. See, for example, Kaufman and Sharp, (1982) Mol. Cell. Biol., 2, 1304-1319 and U.S. Pat. Nos. 4,470,461 and 5,122,464.

Such operative linking of a DNA sequence to an expression control sequence includes the provision of a translation start signal in the correct reading frame upstream of the DNA sequence. If the particular DNA sequence being expressed does not begin with a methionine, the start signal will result in an additional amino acid (methionine) being located at the N-terminus of the product. If a hydrophobic moiety is to be linked to the N-terminal methionyl-containing protein, the protein may be employed directly in the compositions of the invention. Neverthless, since the preferred N-terminal end of the protein is to consist of a cysteine (or functional equivalent) the methionine must be removed before use. Methods are available in the art to remove such N-terminal methionines from polypeptides expressed with them. For example, certain hosts and fermentation conditions permit removal of substantially all of the N-terminal methionine in vivo. Other hosts require in vitro removal of the N-terminal methionine. Such in vitro and in vivo methods are well known in the art.

Successful incorporation of these polynucleotide constructs into a given expression vector may be identified by three general approaches: (a) DNA-DNA hybridization, (b) presence or absence of “marker” gene functions, and (c) expression of inserted sequences. In the first approach, the presence of the hedgehog gene inserted in an expression vector can be detected by DNA-DNA hybridization using probes comprising sequences that are homologous to the inserted fusion protein gene. In the second approach, the recombinant vector/host system can be identified and selected based upon the presence or absence of certain “marker” gene functions (e.g., thymidine kinase activity, resistance to antibiotics such as G4 18, transformation phenotype, occlusion body formation in baculovirus, etc.) caused by the insertion of foreign genes in the vector. For example, if the polynucleotide is inserted so as to interrupt a marker gene sequence of the vector, recombinants containing the insert can be identified by the absence of the marker gene function. In the third approach, recombinant expression vectors can be identified by assaying the foreign gene product expressed by the recombinant vector. Such assays can be based, for example, on the physical or functional properties of the gene product in bioassay systems.

Recombinant nucleic acid molecules which encode chimeric hedgehog therapeutics may be obtained by any method known in the art (Maniatis et al., 1982, Molecular Cloning; A Laboratory Manual, Cold Spring Harbor Laboratory, Cold Spring Harbor, N.Y.) or obtained from publicly available clones. Methods for the preparation of genes which encode the heavy or light chain constant regions of immunoglobulins are taught, for example, by Robinson, R. et al., PCT Application, Publication No. W087-02671. The cDNA sequence encoding the hedgehog molecule or fragment may be directly joined to the cDNA encoding the heavy Ig contant regions or may be joined via a linker sequence. In further embodiments of the invention, a recombinant vector system may be created to accommodate sequences encoding hedgehog in the correct reading frame with a synthetic hinge region. Additionally, it may be desirable to include, as part of the recombinant vector system, nucleic acids corresponding to the 3′ flanking region of an immunoglobulin gene including RNA cleavage/polyadenylation sites and downstream sequences. Furthermore, it may be desirable to engineer a signal sequence upstream of the immunoglobulin fusion protein-encoding sequences to facilitate the secretion of the fused molecule from a cell transformed with the recombinant vector.

The proteins produced by a transformed host can be purified according to any suitable method. Such standard methods include chromatography (e.g., ion exchange, affinity, and sizing column chromatography), centrifugation, differential solubility, or by any other standard technique for protein purification. For immunoaffinity chromatography (See Example ), a protein such as Sonic hedgehog may be isolated by binding it to an affinity column comprising of antibodies that were raised against Sonic hedgehog, or a related protein and were affixed to a stationary support. For example, the hedgehog proteins and fragments may be purified by passing a solution thereof through a column having an hedgehog receptor immobilized thereon (see U.S. Pat. No. 4,725,669). The bound hedgehog molecule may then be eluted by treatment with a chaotropic salt or by elution with aqueous acetic acid. Specific immunoglobulin fusion proteins may be purified by passing a solution containing the fusion protein through a column which contains immobilized protein A or protein G which selectively binds the Fc portion of the fusion protein. See, for example, Reis, K. J., et al., J. Immunol. 132:3098-3102 (1984); PCT Application, Publication No. W087/00329.

Alternatively hedgehog proteins and chimeric molecules may be purified on anti-hedgehog antibody columns, or on anti-immunoglobulin antibody columns to give a substantially pure protein. By the term “substantially pure” is intended that the protein is free of the impurities that are naturally associated therewith. Substantial purity may be evidenced by a single band by electrophoresis. Alternatively, affinity tags such as hexahistidine, maltose binding domain, influenza coat sequence, and glutathione-S-transferase can be attached to the protein to allow easy purification by passage over an appropriate affinity column. Isolated proteins can also be characterized physically using such techniques as proteolysis, nuclear magnetic resonance, and X-ray crystallography.

An example of a useful hedgehog/Ig chimeric protein of this invention is that protein encoded by the nucleotide sequence of SEQ ID NOS: 31-34, which are secreted into the cell culture by eukaryotic cells containing the expression plasmids pUB55, pUB 114, pUB 115 and pUB 116, respectively (See Examples). These proteins consist of the mature human hedgehog fused to a portion of the hinge region and the CH2 and CH3 constant domains of murine or human Ig. Proteins of this group contains a sufficient portion of the immunoglobulin to be recognized by the Fc binding protein, Protein A.

A. Production of Fragments and Analogs

Fragments of an isolated protein (e.g., fragments of SEQ ID NOS: 10-18 or 23-26) can also be produced efficiently by recombinant methods, by proteolytic digestion, or by chemical synthesis using methods known to those of skill in the art. In recombinant methods, internal or terminal fragments of a polypeptide can be generated by removing one or more nucleotides from one end (for a terminal fragment) or both ends (for an internal fragment) of a DNA sequence which encodes for the isolated hedgehog polypeptide. Expression of the mutagenized DNA produces polypeptide fragments. Digestion with “end nibbling” endonucleases can also generate DNAs which encode an array of fragments. DNAs which encode fragments of a protein can also be generated by random shearing, restriction digestion, or a combination of both. Protein fragments can be generated directly from intact proteins. Peptides can be cleaved specifically by proteolytic enzymes, including, but not limited to plasmin, thrombin, trypsin, chymotrypsin, or pepsin. Each of these enzymes is specific for the type of peptide bond it attacks. Trypsin catalyzes the hydrolysis of peptide bonds in which the carbonyl group is from a basic amino acid, usually arginine or lysine. Pepsin and chymotrypsin catalyse the hydrolysis of peptide bonds from aromatic amino acids, such as tryptophan, tyrosine, and phenylalanine. Alternative sets of cleaved protein fragments are generated by preventing cleavage at a site which is susceptible to a proteolytic enzyme. For instance, reaction of the E-amino acid group of lysine with ethyltrifluorothioacetate in mildly basic solution yields blocked amino acid residues whose adjacent peptide bond is no longer susceptible to hydrolysis by trypsin. Proteins can be modified to create peptide linkages that are susceptible to proteolytic enzymes. For instance, alkylation of cysteine residues with (3-haloethylamines yields peptide linkages that are hydrolyzed by trypsin (Lindley, (1956) Nature 178, 647). In addition, chemical reagents that cleave peptide chains at specific residues can be used. For example, cyanogen bromide cleaves peptides at methionine residues (Gross and Witkip, (1961) J. Am. Chem. Soc. 83, 1510). Thus, by treating proteins with various combinations of modifiers, proteolytic enzymes and/or chemical reagents, the proteins may be divided into fragments of a desired length with no overlap of the fragments, or divided into overlapping fragments of a desired length.

Fragments can also be synthesized chemically using techniques known in the art such as the Merrifield solid phase F moc or t-Boc chemistry. Merrifield, Recent Progress in Hormone Research 23: 451 (1967).

Examples of prior art methods which allow production and testing of fragments and analogs are discussed below. These, or analogous methods may be used to make and screen fragments and analogs of an isolated polypeptide (e.g., hedgehog) which can be shown to have biological activity. An exemplary method to test whether fragments and analogs of hedgehog have biological activity is found in Example

B. Production of Altered DNA and Peptide Sequences: Random Methods

Amino acid sequence variants of a protein can be prepared by random mutagenesis of DNA which encodes the protein or a particular portion thereof. Useful methods include PCR mutagenesis and saturation mutagenesis. A library of random amino acid sequence variants can also be generated by the synthesis of a set of degenerate oligonucleotide sequences. Methods of generating amino acid sequence variants of a given protein using altered DNA and peptides are well-known in the art. The following examples of such methods are not intended to limit the scope of the present invention, but merely serve to illustrate representative techniques. Persons having ordinary skill in the art will recognize that other methods are also useful in this regard.

PCR Mutagenesis: See, for example Leung et al., (1989) Technique 1, 11-15.

Saturation Mutagenesis: One method is described generally in Mayers et al., (1989) Science 229, 242.

Degenerate Oligonucleotide Mutagenesis: See for example Harang, S.A., (1983) Tetrahedron 39, 3; Itakura et al., (1984) Ann. Rev. Biochem. 53, 323 and Itakura et al., Recombinant DNA, Proc. 3rd Cleveland Symposium on Macromolecules, pp. 273-289 (A.G. Walton, ed.), Elsevier, Amsterdam, 1981.

C. Production of Altered DNA and Peptide Sequences: Directed Methods

Non-random, or directed, mutagenesis provides specific sequences or mutations in specific portions of a polynucleotide sequence that encodes an isolated polypeptide, to provide variants which include deletions, insertions, or substitutions of residues of the known amino acid sequence of the isolated polypeptide. The mutation sites may be modified individually or in series, for instance by: (1) substituting first with conserved amino acids and then with more radical choices depending on the results achieved; (2) deleting the target residue; or (3) inserting residues of the same or a different class adjacent to the located site, or combinations of options 1-3.

Clearly, such site-directed methods are one way in which an N-terminal cysteine (or a functional equivalent) can be introduced into a given polypeptide sequence to provide the attachment site for a hydrophobic moiety.

Alanine scanning Mutagenesis: See Cunningham and Wells, (1989) Science 244, 1081-1085).

Oligonucleotide-Mediated Mutagenesis: See, for example, Adelman et al., (1983) DNA 2, 183.

Cassette Mutagenesis: See Wells et al., (1985) Gene 34, 315.

Combinatorial Mutagenesis: See, for example, Ladner et al., WO 88/06630

Indeed, it is plain from the combinatorial mutagenesis art that large scale mutagenesis of hedgehog proteins, without any preconceived ideas of which residues were critical to the biological function, and generate wide arrays of variants having equivalent biological activity. Indeed, it is the ability of combinatorial techniques to screen billions of different variants by high throughout analysis that removes any requirement of a priori understanding or knowledge of critical residues.

D. Other Variants of Isolated Polypeptides

Included in the invention are isolated molecules that are: allelic variants, natural mutants, induced mutants, and proteins encoded by DNA that hybridizes under high or low stringency conditions to a nucleic acid which encodes a polypeptide such as the N-terminal fragment of Sonic hedgehog (SEQ ID NO: 23) and polypeptides bound specifically by antisera to hedgehog peptides, especially by antisera to an active site or binding site of hedgehog. All variants described herein are expected to: (i) retain the biological function of the original protein and (ii) retain the ability to link to form a chimeric molecule with a non-hedgehog moiety.

The methods of the invention also feature uses of fragments, preferably biologically active fragments, or analogs of an isolated peptide such as hedgehog. Specifically, a biologically active fragment or analog is one having any in vivo or in vitro activity which is characteristic of the peptide shown in SEQ lD NOS: 10-18 or 23-26 or of other naturally occurring isolated hedgehog. Most preferably, the hydrophobically-modified fragment or analog has at least 10%, preferably 40% or greater, or most preferably at least 90% of the activity of Sonic hedgehog in any in vivo or in vitro assay.

Analogs can differ from naturally occurring isolated protein in amino acid sequence or in ways that do not involve sequence, or both. The most preferred polypeptides of the invention have preferred non-sequence modifications that include in vivo or in vitro chemical derivatization (e.g., of their N-terminal end). Hedgehog polypeptides may also be chemically modified to create hedgehog derivatives by forming covalent or aggregate conjugates with other chemical moieties, such as glycosyl groups, cholesterol, isoprenoids, lipids, phosphate, acetyl groups and the like. Covalent derivatives of hedgehog proteins can be prepared by linking the chemical moieties to functional groups on amino acid sidechains of the protein or at the N-terminus or at the C-terminus of the polypeptide.

For instance, hedgehog proteins can be generated to include a moiety, other than sequence naturally associated with the protein, that binds a component of the extracellular matrix and enhances localization of the analog to cell surfaces. For example, sequences derived from the fibronectin “type-III repeat”, such as a tetrapeptide sequence R—G—D—S (Pierschbacher et al. (1984) Nature 309:30-3; and Kornblihtt et al. (1985) EMBO 4:1755-9) can be added to the hedgehog polypeptide to support attachment of the chimeric molecule to a cell through binding ECM components (Ruoslahti et al. (1987) Science 238:491-497; Pierschbacheret al. (1987) J. Biol. Chem. 262:17294-8.; Hynes (1987) Cell 48:549-54; and Hynes (1992) Cell 69:11-25).

Other analogs include a protein such as Sonic hedgehog or its biologically active fragments whose sequences differ from the wild type consensus sequence (e.g., SEQ ID NO: 26) by one or more conservative amino acid substitutions or by one or more non conservative amino acid substitutions, or by deletions or insertions which do not abolish the isolated protein's biological activity. Conservative substitutions typically include the substitution of one amino acid for another with similar characteristics such as substitutions within the following groups: , valine, alanine and glycine; leucine and isoleucine; aspartic acid and glutamic acid; asparagine and glutamine; serine and threonine; lysine and arginine; and phenylalanine and tyrosine. The non-polar hydrophobic amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Other conservative substitutions can be readily known by workers of ordinary skill. For example, for the amino acid alanine, a conservative substitution can be taken from any one of D-alanine, glycine, beta-alanine, L-cysteine, and D-cysteine. For lysine, a replacement can be any one of D-lysine, arginine, D-arginine, homo-arginine, methionine, D-methionine, omithine, or D-ornithine.

Other analogs used within the methods of the invention are those with modifications which increase peptide stability. Such analogs may contain, for example, one or more non-peptide bonds (which replace the peptide bonds) in the peptide sequence. Also included are: analogs that include residues other than naturally occurring L-amino acids, such as D-amino acids or non-naturally occurring or synthetic amino acids such as beta or gamma amino acids and cyclic analogs. Incorporation of D-instead of L-amino acids into the isolated hedgehog polypeptide may increase its resistance to proteases. See, U.S. Pat. No. 5,219,990 supra. The term “fragment”, as applied to an isolated hedgehog analog, can be as small as a single amino acid provided that it retains biological activity. It may be at least about 20 residues, more typically at least about 40 residues, preferably at least about 60 residues in length. Fragments can be generated by methods known to those skilled in the art. The ability of a candidate fragment to exhibit isolated hedgehog biological activity can be also assessed by methods known to those skilled in the art as described herein.

IV. Antagonists of Hedgehog Activity

A preferred antagonist has at least the following properties: (i) the isolated protein binds the receptor patched-i with an affinity that may be less than, but is preferably at least the same as, the binding of mature hedgehog protein to patched-1; and (ii) the isolated protein blocks alkaline phosphatase (AP) induction by mature hedgehog protein when tested in an in vitro CH310T l/2 cell-based AP induction assay. Antagonists of the invention may also have the additional properties of being (iii) unable to induce ptc-1 and gli-1 expression.

Persons having ordinary skill in the art can easily test any putative hedgehog antagonist for these properties. In particular, the mouse embryonic fibroblast line C3HlOT I/2 is a mesenchymal stem cell line that is hedgehog responsive. Hedgehog treatment of the cells causes an upregulation of gli-1 and patched-I (known indicators of hedgehog dependent signaling) and also causes induction of alkaline phosphatase activity, an indicator that the cells have differentiated down the chondrocyte/bone osteoblast lineage. Several hedgehog variants are unable to elicit a hedgehog-dependent response on C3HlOT1/2 cells, but they competed with mature hedgehog for function and therefore serve as functional antagonists. The synthesis and use of such hedgehog antagonist moieties are briefly described below.

A. N-Modified Hedgehog Polypeptides as Antagonists

Certain hedgehog variants that contain N-terminal modifications can block hedgehog function because they lack the ability to elicit a hedgehog-dependent response but retain the ability to bind to hedgehog receptor, patched-1. The critical primary amino acid sequence that defines whether a hedgehog polypeptide (i.e., a Sonic, Indian or Desert hedgehog) is a functional hedgehog antagonist is the N-terminal cysteine residue which corresponds to Cys-1 of the mature hedgehog. So long as the hedgehog polypeptide either lacks this N-termninal cysteine completely or contains this N-terminal cysteine in a modified form (e.g. chemically modified or included as part of an N-terminal extension moiety), the resulting polypeptide can act as a functional hedgehog antagonist. In this regard, the fact that an N-terminal cysteine “corresponds to Cys-1” means: (a) the N-terminal cysteine is the Cys-1 of mature Sonic, Indian or Desert hedgehog; or (b) the N-terminal cysteine occupies the same position as Cys-1 of mature Sonic, Indian or Desert hedgehog. Provided that, for example, a Sonic hedgehog has an N-terminal cysteine corresponding to Cys-1 that is altered or otherwise modified as described herein, it can antagonize the action of any other member of the hedgehog family. Therefore, persons having ordinary skill in the art will understand that it is possible for an Indian hedgehog protein to antagonize the activity of Sonic, Desert or Indian hedgehogs.

Examples of these antagonists with N-terminal modifications are included below and one skilled in the art can alter the disclosed structure of the antagonist, e.g., by producing fragments or analogs, and test the newly produced structures for antagonist activity. These examples in no way limit the structure of any related hedgehog antagonists, but are merely provided for further description. These, or analogous methods, can be used to make and screen fragments and analogs of a antagonist polypeptides. There are several variants that are able to function as antagonists.

1. N-terminal extensions

Antagonist polypeptides of the invention may include a hedgehog polypeptide sequence in which the N-terminal cysteine is linked to an N-terminal extension moiety. The isolated antagonist polypeptide can therefore be, as but one example, a recombinant fusion protein having: (a) a first N-terminal polypeptide portion that can be 5′ to the hedgehog polypeptide itself, and that contains at least one element (e.g., an amino acid residue) that may be unrelated to hedgehog, linked to (b) an N-terminal cysteine corresponding to Cys-1 of Sonic hedgehog that is part of a hedgehog antagonist of the invention, or a portion of hedgehog antagonist. This N-terminal extension moiety (e.g., the first N-terminal polypeptide portion) can be a histidine tag, a maltose binding protein, glutathione-S-transferase, a DNA binding domain, or a polymerase activating domain. The functional antagonist may include an N-terminal extension moiety that contains an element which replaces the Cys-1 of mature hedgehog or an N-terminal cysteine that corresponds to Cys-1 of a mature Sonic hedgehog.

2. N-terminal deletions

Another variation of a functional antagonist is a hedgehog protein that is missing no greater than about 12 amino acids beginning from that N-terminal cysteine corresponding to Cys-1 of a mature hedgehog. Deletions in more than the about the first 12 contiguous amino acid residues do not generate functional antagonists. Preferably, deletions of about 10 contiguous amino acids will provide suitable functional antagonists. One can, however, remove fewer than 10 contiguous residues and still maintain antagonist function. Moreover, one can delete various combinations of non-contiguous residues provided that there are at least about 3 deleted residues in total.

These structures highlight the importance of the N-terminus of hedgehog proteins for function and indeed, underscore the need to conjugate a hedgehog protein at a site other than the N-terminal cysteine. All of the N-terminal deletion variants were indistinguishable from mature Sonic hedgehog (Shh) in their ability to bind patched-1, but were inactive in the in vitro C3H10T1/2 AP induction assay. All these N-terminal variants are unable to promote hedgehog-dependent signaling.

3. N-terminal mutations

Yet another functional antagonist has a mutation of the N-terminal cysteine to another amino acid residue. Any non-hydrophobic amino acid residue may be acceptable and persons having ordinary skill in the art following the teachings described herein will be able to perform the mutations and test the effects of such mutations. One example is Shh in which the N-terminal cysteine is replaced with a serine residue. This mutated form is indistinguishable from mature Shh in its ability to bind patched-1, but it blocks AP induction by mature Shh when tested for function in the C3H10T1/2 AP induction assay. Replacements with aspartic acid, alanine and histidine have also shown to serve as antagonists.

4. N-terminal cysteine modifications

Because the primary amino acid sequence of hedgehog contains the Cys-1 that is important for biological activity, certain other modifications will result in inactive antagonist variants of hedgehog protein. Another antagonist is an isolated functional antagonist of a hedgehog polypeptide, comprising a hedgehog polypeptide containing an N-terminal cysteine that corresponds to Cys-1 of a mature Sonic hedgehog, except that the cysteine is in a modified form. Antagonist polypeptides of hedgehog may have non-sequence modifications that include in vivo or in vitro chemical derivatization of their N-terminal cysteine, as well as possible changes in acetylation, methylation, phosphorylation, amidation, or carboxylation. As an example, the functional antagonist can have an N-terminal cysteine in an oxidized form. Thus, a functional antagonist can have an N-terminal cysteine that is effectively modified by including it as part of an N-terminal extension moiety.

The functional antagonist polypeptides can include amino acid sequences that are at least 60% homologous to a hedgehog protein. The antagonist must exhibit at least the following functional antagonist properties: (i) the isolated protein binds the receptor patched-1 with an affinity that may be less than, but is preferably at least the same as, the binding of mature hedgehog protein to patched-i; and (ii) the isolated protein blocks alkaline phosphatase (AP) induction by mature hedgehog protein when tested in an in vitro CH310T1/2 cell-based AP induction assay.

Antagonists useful in the present invention also include those which arise as a result of the existence of multiple genes, alternative transcription events, alternative RNA splicing events, and alternative translational and posttranslational events. The polypeptide can be made entirely by synthetic means or can be expressed in systems, e.g., cultured cells, which result in substantially the same posttranslational modifications present when the protein is expressed in a native cell, or in systems which result in the omission of posttranslational modifications present when expressed in a native cell.

In a preferred embodiment, isolated antagonist is a polypeptide with one or more of the following characteristics:

(i) it has at least 60, more preferably 90 and most preferably 95% sequence identity with amino acids of SEQ ID NOS: 10-18 and 23-26;

(ii) it either has a modified N-terminal cysteine or lacks an N-terminal cysteine or has an N-terminal cysteine in a position different from the N-terminal cysteine corresponding to Cys-1 of the hedgehog;

(iii) it blocks alkaline phosphatase induction by mature hedgehog in CH310T1/2 cells;

(iv) it binds or interacts with its receptor patched-1 with an affinity that may be less than, but is preferably at least the same as, the binding of mature hedgehog protein to patched-1;

(v) it is unable to induce ptc-1 and gli-1 expression in vitro in CH310T1/2 cells; or

(vi) it is unable to induce AP in CH310T1/2 assays.

B. Antibody Homologs as Antagonists

In other embodiments, the antagonists used in the method of the invention to bind to, including block or coat, cell-surface hedgehog (such as vertebrate Sonic, Indian or Desert) and/or cell surface ligand for said hedgehog proteins (such as patched) is an anti-hedgehog and/or anti patched monoclonal antibody or antibody homolog, as defined previously. Preferred antibodies and homologs for treatment, in particular for human treatment, include human antibody homologs, humanized antibody homologs, chimeric antibody homologs, Fab, Fab′, F(ab′)2 and F(v) antibody fragments, and monomers or dimers of antibody heavy or light chains or mixtures thereof. Monoclonal antibodies against VLA-4 are a preferred binding agent in the method of the invention.

The technology for producing monoclonal antibodies is well known. The preferred antibody homologs contemplated herein can be expressed from intact or truncated genomic or cDNA or from synthetic DNAs in prokaryotic or eukaryotic host cells. The dimeric proteins can be isolated from the culture media and/or refolded and dimerized in vitro to form biologically active compositions. Heterodimers can be formed in vitro by combining separate, distinct polypeptide chains. Alternatively, heterodimers can be formed in a single cell by co-expressing nucleic acids encoding separate, distinct polypeptide chains. See, for example, W093/09229, or U.S. Pat. No. 5,411,941, for several exemplary recombinant heterodimer protein production protocols. Currently preferred host cells include, without limitation, prokaryotes including E. coli, or eukaryotes including yeast, Saccharomyces, insect cells, or mammalian cells, such as CHO, COS or BSC cells. One of ordinary skill in the art will appreciate that other host cells can be used to advantage. For example, anti-hedgehog antibodies may be identified by immunoprecipitation of 1251-labeled cell lysates from hedgehog -expressing cells. Anti-hedgehog antibodies may also be identified by flow cytometry, e.g., by measuring fluorescent staining of cells incubated with an antibody believed to recognize hedgehog protein. The lymphocytes used in the production of hybridoma cells typically are isolated from immunized mammals whose sera have already tested positive for the presence of anti-hedgehog antibodies using such screening assays.

Typically, the immortal cell line (e.g., a myeloma cell line) is derived from the same mammalian species as the lymphocytes. Preferred immortal cell lines are mouse myeloma cell lines that are sensitive to culture medium containing hypoxanthine, arninopterin and thymidine (“HAT medium”). Typically, HAT-sensitive mouse myeloma cells are fused to mouse splenocytes using 1500 molecular weight polyethylene glycol (“PEG 1500”). Hybridoma cells resulting from the fusion are then selected using HAT medium, which kills unfused and unproductively fused myeloma cells (unfused splenocytes die after several days because they are not transformed). Hybridomas producing a desired antibody are detected by screening the hybridoma culture supernatants. For example, hybridomas prepared to produce anti-hedgehog or patched antibodies may be screened by testing the hybridoma culture supernatant for secreted antibodies having the ability to bind to a recombinant hedgehog or patched expressing cell line.

To produce antibody homologs that are intact immunoglobulins, hybridoma cells that tested positive in such screening assays were cultured in a nutrient medium under conditions and for a time sufficient to allow the hybridoma cells to secrete the monoclonal antibodies into the culture medium. Tissue culture techniques and culture media suitable for hybridoma cells are well known. The conditioned hybridoma culture supernatant may be collected and the anti-hedgehog or patched antibodies optionally further purified by well-known methods.

Alternatively, the desired antibody may be produced by injecting the hybridoma cells into the peritoneal cavity of an unimmunized mouse. The hybridoma cells proliferate in the peritoneal cavity, secreting the antibody which accumulates as ascites fluid. The antibody may be harvested by withdrawing the ascites fluid from the peritoneal cavity with a syringe. Several anti-hedgehog or patched monoclonal antibodies have been previously described. These anti-hedgehog or patched monoclonal antibodies and others will be useful in the methods of treatment according to the present invention.

Fully human monoclonal antibody homologs against hedgehog or patched are another preferred binding agent which may block or coat hedgehog ligands in the method of the invention. In their intact form these may be prepared using in vitro-primed human splenocytes, as described by Boerner et al., 1991, J. Immunol., 147, 86-95. Alternatively, they may be prepared by repertoire cloning as described by Persson et al., 1991, Proc. Nat. Acad. Sci. USA, 88: 2432-2436 or by Huang and Stollar, 1991, J. Immunol. Methods 141, 227-236. U.S. Pat. No. 5,798,230 (Aug. 25, 1998, “Process for the preparation of human monoclonal antibodies and their use”) who describe preparation of human monoclonal antibodies from human B cells. According to this process, human antibody-producing B cells are immortalized by infection with an Epstein-Barr virus, or a derivative thereof, that expresses Epstein-Barr virus nuclear antigen 2 (EBNA2). EBNA2 function, which is required for immortalization, is subsequently shut off, which results in an increase in antibody production.

In yet another method for producing fully human antibodies, U.S. Pat. No. 5,789,650 (Aug. 4, 1998, “Transgenic non-human animals for producing heterologous antibodies”) describes transgenic non-human animals capable of producing heterologous antibodies and transgenic non-human animals having inactivated endogenous immunoglobulin genes. Endogenous immunoglobulin genes are suppressed by aritisense polynucleotides and/or by antiserum directed against endogenous immunoglobulins. Heterologous antibodies are encoded by immunoglobulin genes not normally found in the genome of that species of non-human animal. One or more transgenes containing sequences ofunrearranged heterologous human immunoglobulin heavy chains are introduced into a non-human animal thereby forming a transgenic animal capable of functionally rearranging transgenic immunoglobulin sequences and producing a repertoire of antibodies of various isotypes encoded by human immunoglobulin genes. Such heterologous human antibodies are produced in B-cells which are thereafter immortalized, e.g., by fusing with an immortalizing cell line such as a myeloma or by manipulating such B-cells by other techniques to perpetuate a cell line capable of producing a monoclonal heterologous, fully human antibody homolog.

Large nonimmunized human phage display libraries may also be used to isolate high affinity antibodies that can be developed as human therapeutics using standard phage technology (Vaughan et al, 1996).

Yet another preferred binding agent which may block or coat hedgehog ligands in the method of the invention is a humanized recombinant antibody homolog having anti-hedgehog or patched specificity. Following the early methods for the preparation of true “chimeric antibodies” (where the entire constant and entire variable regions are derived from different sources), a new approach was described in EP 0239400 (Winter et al.) whereby antibodies are altered by substitution (within a given variable region) of their complementarity determining regions (CDRs) for one species with those from another. This process may be used, for example, to substitute the CDRs from human heavy and light chain Ig variable region domains with alternative CDRs from murine variable region domains. These altered Ig variable regions may subsequently be combined with human Ig constant regions to create antibodies which are totally human in composition except for the substituted murine CDRs. Such CDR-substituted antibodies would be predicted to be less likely to elicit an immune response in humans compared to true chimeric antibodies because the CDR-substituted antibodies contain considerably less non-human components. The process for humanizing monoclonal antibodies via CDR “grafting” has been termed “reshaping”. (Riechmann et al., 1988, Nature 332, 323-327; Verhoeyen et al., 1988, Science 239, 1534-1536).

Typically, complementarity determining regions (CDRs) of a murine antibody are transplanted onto the corresponding regions in a human antibody, since it is the CDRs (three in antibody heavy chains, three in light chains) that are the regions of the mouse antibody which bind to a specific antigen. Transplantation of CDRs is achieved by genetic engineering whereby CDR DNA sequences are determined by cloning of murine heavy and light chain variable (V) region gene segments, and are then transferred to corresponding human V regions by site directed mutagenesis. In the final stage of the process, human constant region gene segments of the desired isotype (usually gamma I for CH and kappa for CL) are added and the humanized heavy and light chain genes are co-expressed in mammalian cells to produce soluble humanized antibody.

The transfer of these CDRs to a human antibody confers on this antibody the antigen binding properties of the original murine antibody. The six CDRs in the murine antibody are mounted structurally on a V region “framework” region. The reason that CDR-grafting is successful is that framework regions between mouse and human antibodies may have very similar 3-D structures with similar points of attachment for CDRS, such that CDRs can be interchanged. Such humanized antibody homologs may be prepared, as exemplified in Jones et al., 1986, Nature 321, 522-525; Riechmann, 1988, Nature 332, 323-327; Queen et al., 1989, Proc. Nat. Acad. Sci. USA 86, 10029; and Orlandi et al., 1989, Proc. Nat. Acad. Sci. USA 86, 3833.

Nonetheless, certain amino acids within framework regions are thought to interact with CDRs and to influence overall antigen binding affinity. The direct transfer of CDRs from a murine antibody to produce a recombinant humanized antibody without any modifications of the human V region frameworks often results in a partial or complete loss of binding affinity. In a number of cases, it appears to be critical to alter residues in the framework regions of the acceptor antibody in order to obtain binding activity.

Queen et al., 1989 (supra) and WO 90/07861 (Protein Design Labs) have described the preparation of a humanized antibody that contains modified residues in the framework regions of the acceptor antibody by combining the CDRs of a murine MAb (anti-Tac) with human immunoglobulin framework and constant regions. They have demonstrated one solution to the problem of the loss of binding affinity that often results from direct CDR transfer without any modifications of the human V region framework residues; their solution involves two key steps. First, the human V framework regions are chosen by computer analysts for optimal protein sequence homology to the V region framework of the original murine antibody, in this case, the anti-Tac MAb. In the second step, the tertiary structure of the murine V region is modelled by computer in order to visualize framework amino acid residues which are likely to interact with the murine CDRs and these murine amino acid residues are then superimposed on the homologous human framework. See also U.S. Pat. Nos. 5,693,762; 5,693,761; 5,585,089; and 5,530,101 (Protein Design Labs).

One may use a different approach (Tempest et al.,1991, Biotechnology 9, 266-271) and utilize, as standard, the V region frameworks derived from NEWM and REI heavy and light chains respectively for CDR-grafting without radical introduction of mouse residues. An advantage ofusing the Tempest et al., approach to construct NEWM and REI based humanized antibodies is that the 3dimensional structures of NEWM and REI variable regions are known from x-ray crystallography and thus specific interactions between CDRs and V region framework residues can be modeled.

Regardless of the approach taken, the examples of the initial humanized antibody homologs prepared to date have shown that it is not a straightforward process. However, even acknowledging that such framework changes may be necessary, it is not possible to predict, on the basis of the available prior art, which, if any, framework residues will need to be altered to obtain functional humanized recombinant antibodies of the desired specificity. Results thus far indicate that changes necessary to preserve specificity and/or affinity are for the most part unique to a given antibody and cannot be predicted based on the humanization of a different antibody.

C. Small Organic Molecules as Antagonists

In other embodiments, a hedgehog antagonist may be a small organic molecule. Such a small organic molecule may antagonize hedgehog signal transduction via an interaction with but not limited to hedgehog, patched (ptc), gli, and/or smoothened. It is, therefore, specifically contemplated that these small molecules which intefere with aspects of hedgehog, ptc, or smoothened signal transduction activity will likewise be capable of inhibiting angiogenesis (or other biological consequences) in normal cells and/or mutant cells. Thus, it is contemplated that in certain embodiments, these compounds may be useful for inhibiting hedgehog activity in normal cells. In other embodiments, these compounds may be useful for inhibitng hedgehog activity in abnormal cells. In preferred embodiments, the subject inhibitors are organic molecules having a molecular weight less than 2500 amu, more preferably less than 1500 amu, and even more preferably less than 750 amu, and are capable of antagonizing hedgehog signaling, preferably specifically in target cells.

For example, compounds useful in the subject methods include compounds may be represented by general forumla (I):

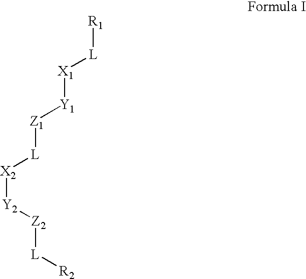

wherein, as valence and stability permit,

R₁ and R₂, independently for each occurrence, represent H, lower alkyl, aryl (e.g., substituted or unsubstituted), aralkyl (e.g., substituted or unsubstituted, e.g., —(CH₂)ₙaryl), or heteroaryl (e.g., substituted or unsubstituted), or heteroaralkyl (e.g., substituted or unsubstituted, e.g., —(CH2)nheteroaralkyl—);

L, independently for each occurrence, is absent or represents —(CH₂)ₙ-alkyl, -alkenyl-, -alkynyl-, —(CH₂)ₙalkenyl-, —(CH₂)ₙalkynyl-, —(CH₂)ₙO(CH₂)ₚ—, —(CH₂)ₙNR₂(CH₂)ₚ—, —(CH₂)ₙS(CH₂)ₚ—, —(CH₂)ₙalkenyl(CH₂)ₚ—, (CH₂)ₙalkynyl(CH₂)ₚ—, —O(CH₂)ₙ—, —NR₂(CH₂)ₙ—, or —S(CH₂)ₙ—;

X₁ and X₂ can be selected, independently, from —N(R₈)—, —O—, —S—, —Se—, —N═N—, —ON═CH—, —(R₈)N—N(R₈)—, —ON(R₈)—, a heterocycle, or a direct bond between L and Y₁ or Y₂, respectively;

Y₁ and Y₂ can be selected, independently, from —C(═O)—, —C(═S)—, —S(O₂)—, —S(O)—, —C(═NCN)—, —P(═O)(OR₂)—, a heteroaromatic group, or a direct bond between X₁ and Z₁ or X₂ and Z₂, respectively;

Z₁ and Z₂ can be selected, independently, from —N(R₈)—, —O—, —S—, —Se—, —N═N—, —ON═CH—, —R₈N—NR₈—, —ONR₈—, a heterocycle, or a direct bond between Y₁ or Y2, respectively, and L;

R₈, independently for each occurrence, represents H, lower alkyl, —(CH₂)naryl (e.g., substituted or unsubstituted), —(CH₂)ₙheteroaryl (e.g., substituted or unsubstituted), or two R₈ taken together may form a 4- to 8-membered ring, e.g., with X₁ and Z₁ or X₂ and Z₁, which rin g may include one or more carbonyls;

p represents, independently for each occurrence, an integer from 0 to 10, preferably from 0 to 3; and

n, individually for each occurence, represents an integer from 0 to 10, preferably from 0 to 5.

In certain embodiments, R₁ represents a substituted or unsubstituted heteroaryl group.

In certain embodiments, X₁ and X₂ can be selected from —N(R₈)—, —O—, —S—, a direct bond, and a heterocycle, Y₁ and Y₂ can be selected from —C(═O)—, —C(═S)—, and —S(O₂)—, and Z₁ or Z₂ can be selected from —N(R₈)—, —O—, —S—, a direct bond, and a heterocycle.

In certain related embodiments, X₁—Y₁—Z₁ or X₂—Y₂—Z₂ taken together represents a urea (N—C(O)—N) or an amide (N—C(O) or C(O)—N).

In certain embodiments, X₁ or X₂ represents a diazacarbocycle, such as a piperazine.

In certain embodiments, R₁ represents a fused cycloalkyl-aryl or cycloalkyl-heteroaryl system, for example:

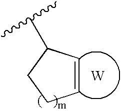

wherein W is a substituted or unsubstituted aryl or heteroaryl ring fused to the cycloalkyl ring and m is an integer from 1-4 inclusive, e.g., from 1-3, or from 1-2. The fused system may be bound to L from any carbon of the fused system, including the position depicted above. In certain embodiments, R₁ may represent a tetrahydronaphthyl group, and preferably Y₁—X₁—L—R , taken together represent a tetrahydronaphthyl amide group, such as:

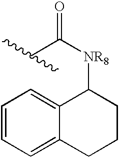

In embodiments wherein Y₁ and Z₁ are absent and X₁ comprises a pyrimidone, compounds useful in the present invention may be represented by general formula (II):

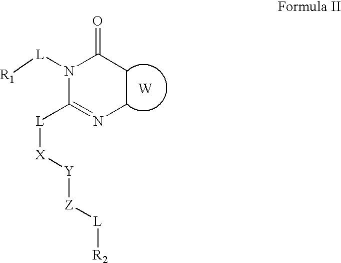

wherein, as valence and stability permit,

R₁ and R₂, independently for each occurrence, represent H, lower alkyl, —(CH₂)ₙaryl (e.g., substituted or unsubstituted), or —(CH₂)ₙheteroaryl (e.g., substituted or unsubstituted);

L, independently for each occurrence, is absent or represents —(CH₂)ₙ-alkyl, -alkenyl-, -alkynyl-, —(CH₂)ₙalkenyl-, —(CH₂)ₙalkynyl-, —(CH₂)ₙO(CH₂)ₚ—, —(CH₂)ₙNR₂(CH₂)ₚ—, —(CH₂)ₙS(CH₂)ₚ—, —(CH₂)ₙalkenyl(CH₂)ₚ—, —(CH₂)ₙalkynyl(CH₂)ₚ—, —O(CH₂)ₙ—, —NR₂(CH₂)ₙ—, or —S(CH₂)ₙ—;

X can be selected from —N(R₈)—, —O—, —S—, —Se—, —N═N—, —ON═CH—, —(R₈)N—N(R₈)—, —ON(R₈)—, a heterocycle, or a direct bond between L and Y;

Y can be selected from —C(═O)—, —C(═S)—, —S(O₂)—, —S(O)—, —C(═NCN)—, —P(═O)(OR₂)—, a heteroaromatic group, or a direct bond between X and Z;

Z can be selected from —N(R₈)—, —O—, —S—, —Se—, —N═N—, —ON═CH—, —R₈N—NR₈—, —ONR₈—, a heterocycle, or a direct bond between Y and L;

R₈, independently for each occurrence, represents H, lower alkyl, —(CH₂)ₙaryl (e.g., substituted or unsubstituted), —(CH₂)ₙheteroaryl (e.g., substituted or unsubstituted), or two R₈ taken together may form a 4- to 8-membered ring, e.g., with X and Z, which ring may include one or more carbonyls;

W represents a substituted or unsubsituted aryl or heteroaryl ring fused to the pyrimidone ring;

p represents, independently for each occurrence, an integer from 0 to 10, preferably from 0 to 3; and

n, individually for each occurence, represents an integer from 0 to 10, preferably from 0 to 5.

In embodiments wherein Y₁ and Z₁ are absent and X₁ comprises a pyrimidone, compounds useful in the present invention may be represented by general formula (III):

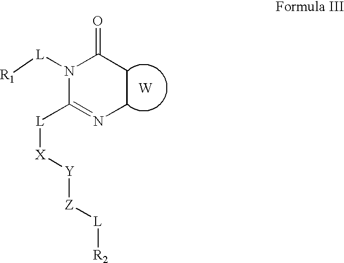

wherein, as valence and stability permit,

R₁ and R₂, independently for each occurrence, represent H, lower alkyl, aryl (e.g., substituted or unsubstituted), aralkyl (e.g., substituted or unsubstituted, e.g., —(CH₂)ₙaryl), or heteroaryl (e.g. , substituted or unsubstituted), or heteroaralkyl (e.g., substituted or unsubstituted, e.g., —(CH₂)ₙheteroaralkyl-);

L, independently for each occurrence, is absent or represents —(CH₂)ₙ-alkyl, -alkenyl-, -alkynyl-, —(CH₂)ₙalkenyl-, —(CH₂)ₙalkynyl-, —(CH₂)ₙO(CH₂)ₚ—, —(CH₂)ₙNR₂(CH₂)ₚ—, —(CH₂)ₙS(CH₂)ₚ—, —(CH₂)ₙalkenyl(CH₂)ₚ—, —(CH₂)ₙalkynyl(CH₂)ₚ—, —O(CH₂)ₙ—, —NR₂(CH₂)ₙ—, or —S(CH₂)ₙ—, which may optionally be substitued with a group selected from H, substituted or unsubstituted lower alkyl, alkenyl, or alkynyl, cycloalkylalkyl (e.g., substituted or unsubstituted, e.g., —(CH₂)ₙcycloalkyl), (e.g., substituted or unsubstituted), aryl (e.g., substituted or unsubstituted), aralkyl (e.g., substituted or unsubstituted, e.g., —(CH₂)ₙaryl), or heteroaryl (e.g., substituted or unsubstituted), or heteroaralkyl (e.g., substituted or unsubstituted, e.g., —(CH₂)ₙheteroaralkyl-), preferably from H, lower alkyl, —(CH₂)ₙaryl (e.g., substituted or unsubstituted), or —(CH₂)ₙheteroaryl (e.g., substituted or unsubstituted);

X can be selected from —N(R₈)—, —O—, —S—, —Se—, —N═N—, —ON═CH—, —(R₈)N—N(R₈)—, —ON(R₈)—, a heterocycle, or a direct bond between L and Y;

Y can be selected from —C(═O)—, —C(═S)—, —S(O₂)—, —S(O)—, —C(═NCN)—, —P(═O)(OR₂)—, a heteroaromatic group, or a direct bond between X and Z;

Z can be selected from —N(R₈)—, —O—, —S—, —Se—, —N═N—, —ON═CH—, —R₈N—NR₈—, —ONR₈—, a heterocycle, or a direct bond between Y and L;

R₈, independently for each occurrence, represents H, lower alkyl, aryl (e.g., substituted or unsubstituted), aralkyl (e.g., substituted or unsubstituted, e.g., —(CH₂)ₙaryl), or heteroaryl (e.g., substituted or unsubstituted), or heteroaralkyl (e.g., substituted or unsubstituted, e.g., —(CH₂)ₙheteroaralkyl-), or two R₈ taken together may form a 4- to 8-membered ring, e.g., with X and Z, which ring may include one or more carbonyls;

W represents a substituted or unsubsituted aryl or heteroaryl ring fused to the pyrimidone ring;

p represents, independently for each occurrence, an integer from 0 to 10, preferably from 0 to 3; and

n, individually for each occurrence, represents an integer from 0 to 10, preferably from 0 to 5.

In certain embodiments, R₁ represents a substituted or unsubstituted aryl or heteroaryl group, e.g., a phenyl ring, a pyridine ring, etc. In certain embodiments wherein —LR₁ represents a substituted aryl or heteroaryl group, R₁ is preferably not substituted with an isopropoxy (Me₂CHO—) group. In certain embodiments wherein —LR₁ represents a substituted aryl or heteroaryl group, R₁ is preferably not substituted with an ether group. In certain embodiments, substituents on R₁ (e.g., other than hydrogen) are selected from halogen, cyano, alkyl, alkenyl, alkynyl, aryl, hydroxyl, (unbranched alkyl-O—), silyloxy, amino, nitro, thiol, amino, imino, amido, phosphoryl, phosphonate, phosphine, carbonyl, carboxyl, carboxamide, anhydride, silyl, thioether, alkylsulfonyl, arylsulfonyl, sulfoxide, selenoether, ketone, aldehyde, ester, or —(CH₂)ₘ—R₈. In certain embodiments, non-hydrogen substituents are selected from halogen, cyano, alkyl, alkenyl, alkynyl, aryl, nitro, thiol, imino, amido, carbonyl, carboxyl, anhydride, thioether, alkylsulfonyl, arylsulfonyl, ketone, aldehyde, and ester. In certain embodiments, non-hydrogen substituents are selected from halogen, cyano, alkyl, alkenyl, alkynyl, nitro, amido, carboxyl, anhydride, alkylsulfonyl, ketone, aldehyde, and ester.

In certain embodiments, X can be selected from —N(R₈)—, —O—, —S—, a direct bond, and a heterocycle, Y can be selected from —C(═O)—, —C(═S)—, and —S(O₂)—, and Z can be selected from —N(R₈)—, —O—, —S—, a direct bond, and a heterocycle. In certain such embodiments, at least one of Z and X is present.

In certain related embodiments, X-Y-Z taken together represents a urea (NC(O)N) or an amide (NC(O) or C(O)N).

In certain embodiments, W is a substituted or unsubstituted benzene ring.

In certain embodiments, X represents a diazacarbocycle, such as a piperazine, e.g., substituted or unsubstituted.

In certain embodiments, X can be selected from —N(R₈)—, —O—, —S—, and a direct bond, Y can be selected from —C(═O)—, —C(═S)—, and —S(O₂)—, and Z can be selected from —N(R₈)—, —O—, —S—, and a direct bond, such that at least one of X and Z is present.

In certain embodiments R₈ represents H, lower alkyl, aralkyl, heteroaralkyl, aryl, or heteroaryl, e.g., H or lower alkyl.

In certain embodiments, X represents —NH—.

In certain embodiments, —L—X— represents —(unbranched lower alkyl)—NH—, e.g., —CH₂—NH—, —CH₂CH₂—NH—, etc.

In certain other embodiments, compounds useful in the subject methods include compounds may be represented by general forumla (IV):

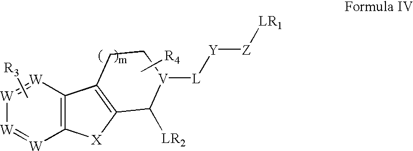

wherein, as valence and stability permit,

R₁ and R₂, independently for each occurrence, represent H, substituted or unsubstituted lower alkyl, alkenyl, or alkynyl, —(CH₂)ₙcycloalkyl (e.g., substituted or unsubstituted), —(CH₂)ₙaryl (e.g., substituted or unsubstituted), or —(CH₂)ₙheterocyclyl (e.g., substituted or unsubstituted);

L, independently for each occurrence, is absent or represents —(CH₂)ₙ-alkyl, -alkenyl-, -alkynyl-, —(CH₂)ₙalkenyl-, —(CH₂)ₙalkynyl-, —(CH₂)ₙO(CH₂)ₚ—, —(CH₂)ₙNR₂(CH₂)ₚ—, —(CH₂)ₙS(CH₂)ₚ—, —(CH₂)ₙalkenyl(CH₂)ₚ—, —(CH₂)ₙalkynyl(CH₂)ₚ—, —O(CH₂)ₙ—, —NR₂(CH₂)ₙ—, or —S(CH₂)ₙ—;

V represents N or CH;

W, independently for each occurrence, represents N or CH, such that preferably no more than one occurrence of W represents N;

X and Z, independently, can be selected from —CH—, —N(R₈)—, —O—, —S—, or —Se—;

Y can be selected from —C(═O)—, —C(═S)—, —S(O₂)—, —S(O)—, —C(═NCN)—, or —P(═O)(OR₂)—;

R₈, independently for each occurrence, represents H, substituted or unsubstituted lower alkyl, —(CH₂)ₙcycloalkyl (e.g., substituted or unsubstituted), —(CH₂)ₙaryl (e.g., substituted or unsubstituted), —(CH₂)ₙheterocyclyl (e.g., substituted or unsubstituted), or two R₈ taken together may form a 4- to 8-membered ring, e.g., with X₁ and Z₁ or X₂ and Z₁, which ring may include one or more carbonyls;

R₃ and R₄, independently represent from 1-4 substituents on the ring to which they are attached, selected from, independently for each occurrence, hydrogen, halogens, alkyls, alkenyls, alkynyls, aryls, hydroxyl, ═O, ═S, alkoxyl, silyloxy, amino, nitro, thiol, amines, imines, amides, phosphoryls, phosphonates, phosphines, carbonyls, carboxyls, carboxamides, anhydrides, silyls, ethers, thioethers, alkylsulfonyls, arylsulfonyls, selenoethers, ketones, aldehydes, esters, or —(CH₂)ₘ—R₈;

m represents an integer from 0-3;

p represents, independently for each occurrence, an integer from 0 to 10, preferably from 0 to 3; and

n, individually for each occurence, represents an integer from 0 to 10, preferably from 0 to 5.

In certain embodiments, R₁ and R₂ are independently selected from substituted or unsubstituted aryl, heterocyclyl, branched or unbranched alkyl, or cycloalkyl. In embodiments wherein R₁ or R₂ is aryl or heterocyclyl, substituents are preferably selected from H, alkyl, acyl, carboxy, ester, amide, cyano, ether, thioether, amino, halogen, nitro, and trihalomethyl.

In certain embodiments, R₃ is absent or represents one or two substituents selected from alkyl, acyl, carboxy, ester, amide, cyano, ether, thioether, amino, acyl, halogen, nitro, and trihalomethyl.

In certain embodiments, R₄ is absent or represents one or two substituents selected from ether, amino, thioether, alkyl, aryl, (═O), or carbonyl (e.g., carboxy, ester, ketone, aldehyde, etc.).

In certain embodiments, L is absent for each occurrence, or represents —CH₂— or —CH₂CH₂—.

In certain embodiments, X represents NR₈. R₈ preferably represents H.

In certain embodiments, Z represents NR₈. R₈ preferably represents H.

In certain embodiments, Y represents —C(═O)—, —C(═S)—, or —S(O₂)—.

In certain embodiments, m is 1.

In certain embodiments, W represents CH in all occurrences.

In certain embodiments, V represents N.

In certain embodiments, compounds useful in the present invention may be represented by general formula (V):

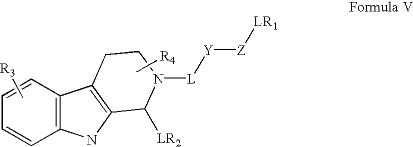

wherein, as valence and stability permit,

R₁ and R₂, independently for each occurrence, represent H, substituted or unsubstituted lower alkyl, alkenyl, or alkynyl, —(CH₂)ₙcycloalkyl (e.g., substituted or unsubstituted), —(CH₂)ₙaryl (e.g., substituted or unsubstituted), or —(CH₂)ₙheterocyclyl (e.g., substituted or unsubstituted);

L, independently for each occurrence, is absent or represents —(CH₂)ₙ-alkyl, -alkenyl-, -alkynyl-, —(CH₂)ₙalkenyl-, —(CH₂)ₙalkynyl-, —(CH₂)ₙO(CH₂)ₚ—, (CH₂)ₙNR₂(CH₂)ₚ—, —(CH₂)ₙS(CH₂)ₚ—, —(CH₂)ₙalkenyl(CH₂)ₚ—, —(CH₂)ₙalkynyl(CH₂)ₚ—, —O(CH₂)ₙ—, —NR₂(CH₂)ₙ—, or —S(CH₂)ₙ—;

X and Z, independently, can be selected from —CH—, —N(R₈)—, —O—, —S—, or —Se—;

Y can be selected from —C(═O)—, —C(═S)—, —S(O₂)—, —S(O)—, —C(═NCN)—, or —P(═O)(OR₂)—;

R₈, independently for each occurrence, represents H, substituted or unsubstituted lower alkyl, —(CH₂)ₙcycloalkyl (e.g., substituted or unsubstituted), —(CH₂)ₙaryl (e.g., substituted or unsubstituted), —(CH₂)ₙheterocyclyl (e.g., substituted or unsubstituted), or two R₈ taken together may form a 4- to 8-membered ring, e.g., with X₁ and Z₁ or X₂ and Z₁, which ring may include one or more carbonyls;

R₃ and R₄, independently represent from 1-4 substituents on the ring to which they are attached, selected from, independently for each occurrence, hydrogen, halogens, alkyls, alkenyls, alkynyls, aryls, hydroxyl, ═O, ═S, alkoxyl, silyloxy, amino, nitro, thiol, amines, imines, amides, phosphoryls, phosphonates, phosphines, carbonyls, carboxyls, carboxamides, anhydrides, silyls, ethers, thioethers, alkylsulfonyls, arylsulfonyls, selenoethers, ketones, aldehydes, esters, or —(CH₂) ,—R₈;

p represents, independently for each occurrence, an integer from 0 to 10, preferably from 0 to 3; and

n, individually for each occurence, represents an integer from 0 to 10, preferably from 0 to 5.

In certain embodiments, R₁ and R₂ are independently selected from substituted or unsubstituted aryl, heterocyclyl, branched or unbranched alkyl, or cycloalkyl. In embodiments wherein R₁ or R₂ is aryl or heterocyclyl, substituents are preferably selected from H, alkyl, acyl, carboxy, ester, amide, cyano, ether, thioether, amino, halogen, nitro, and trihalomethyl.

In certain embodiments, R₃ is absent or represents one or two substituents selected from alkyl, acyl, carboxy, ester, amide, cyano, ether, thioether, amino, acyl, halogen, nitro, and trihalomethyl.

In certain embodiments, R₄ is absent or represents one or two substituents selected from ether, amino, thioether, alkyl, aryl, (═O), or carbonyl (e.g., carboxy, ester, ketone, aldehyde, etc.).

In certain embodiments, L is absent for each occurrence, or represents —CH₂— or —CH₂CH₂—.

In certain embodiments, X represents NR₈. R₈ preferably represents H.

In certain embodiments, Z represents NR₈. R₈ preferably represents H.

In certain embodiments, Y represents —C(═O)—, —C(═S)—, or —S(O₂)—.

In still other embodiments, compounds which may be useful in the subject methods include compounds may be represented by general formula (VI):

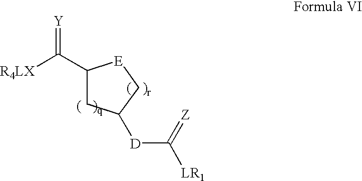

wherein, as valence and stability permit,

R₁, R₂, R₃, and R₄, independently for each occurrence, represent H, lower alkyl, —(CH₂)ₙaryl (e.g., substituted or unsubstituted), or —(CH₂)ₙheteroaryl (e.g., substituted or unsubstituted);

L, independently for each occurrence, is absent or represents —(CH₂)ₙ—, -alkenyl-, -alkynyl-, —(CH₂)ₙalkenyl-, —(CH₂)ₙalkynyl-, —(CH₂)ₙO(CH₂)ₚ—, —(CH₂)ₙNR₈(CH₂)ₚ—, —(CH₂)ₙS(CH₂)ₚ—, —(CH₂)ₙalkenyl(CH₂)ₚ—, —(CH₂)ₙalkynyl(CH₂)ₚ—, —O(CH₂)ₙ—, —NR₈(CH₂)ₙ—, or —S(CH₂)ₙ—;

X and D, independently, can be selected from —N(R₈)—, —O—, —S—, —(R₈)N—N(R₈)—, —ON(R₈)—, or a direct bond;

Y and Z, independently, can be selected from O or S;

E represents O, S, or NR₅, wherein R₅ represents LR₈ or —(C═O)LR₈.

R₈, independently for each occurrence, represents H, lower alkyl, —(CH₂)ₙaryl (e.g., substituted or unsubstituted), —(CH₂)ₙheteroaryl (e.g., substituted or unsubstituted), or two R₈ taken together may form a 4- to 8-membered ring;

p represents, independently for each occurrence, an integer from 0 to 10, preferably from 0 to 3;

n, individually for each occurrence, represents an integer from 0 to 10, preferably from 0 to 5; and

q and r represent, independently for each occurrence, an integer from 0-2.

In certain embodiments, D does not represent N-lower alkyl. In certain embodiments, D represents an aralkyl- or heteroaralkyl-substituted amine.

In certain embodiments, R₁ represents a lower alkyl group, such as a branched alkyl, a cycloalkyl, or a cycloalkylalkyl, for example, cyclopropyl, cyclopropylmethyl, neopentyl, cyclobutyl, isobutyl, isopropyl, sec-butyl, cyclobutylmethyl, etc.

In certain embodiments, Y and Z are O.

In certain embodiments, the sum of q and r is less than 4, e.g., is 2 or 3.

In certain embodiments, XLR₄, taken together, include a cyclic amine, such as a piperazine, a morpholine, a piperidine, a pyrrolidine, etc.

In certain embodiments, at least one of R₁, R₂, and R₃ includes an aryl or heteroaryl group. In certain related embodiments, at least two of R₁, R₂, and R₃ include an aryl or heteroaryl group. In certain embodiments, R₁ is lower alkyl.

In certain embodiments, L attached to R₁ represents O, S, or NR₈, such as NH.

In certain embodiments, E is NR₈. In certain embodiments, E represents an aralkyl- or heteroaralkyl-substituted amine, e.g., including polycyclic R₈.

In certain embodiments, X is not NH. In certain embodiments, X is included in a ring, or, taken together with —C(═Y)—, represents a tertiary amide.

In certain embodiments, compounds useful in the present invention may be represented by general formula (VII):

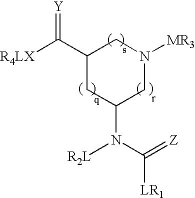

Formula VII

wherein, as valence and stability permit,

R₁, R₂, R₃, R₄, R₈, L, X, Y, Z, n, p, q, and r are as defined above;

M is absent or represents L, —SO₂L—, or —(C═O)L—; and

s represents, independently for each occurrence, an integer from 0-2.

In certain embodiments, Y and Z are O.

In certain embodiments, R₁ represents a lower alkyl group, such as a branched alkyl, a cycloalkyl, or a cycloalkylalkyl, for example, cyclopropyl, cyclopropylmethyl, neopentyl, cyclobutyl, isobutyl, isopropyl, sec-butyl, cyclobutylmethyl, etc.

In certain embodiments, the sum of q, r, and s is less than 5, e.g., is 2, 3, or 4.

In certain embodiments, XLR₄, taken together, include a cyclic amine, such as a piperazine, a morpholine, a piperidine, a pyrrolidine, etc.

In certain embodiments, L attached to R₁ represents O, S, or NR₈, such as NH.

In certain embodiments, at least one of R₁, R₂, and R₃ includes an aryl or heteroaryl group. In certain related embodiments, at least two of R₁, R₂, and R₃ include an aryl or heteroaryl group.

In certain embodiments, M is absent.

In certain embodiments, X is not NH. In certain embodiments, X is included in a ring, or, taken together with —C(═Y)—, represents a tertiary amide.

In certain embodiments, compounds useful in the present invention may be represented by general formula (VIII):

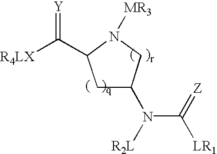

Formula VIII

wherein, as valence and stability permit,

R₁, R₂, R₃, R₄, R₈, L, M, X, Y, Z, n, p, q, and r are as defined above.

In certain embodiments, Y and Z are O.

In certain embodiments, R₁ represents a lower alkyl group, preferably a branched alkyl, a cycloalkyl, or a cycloalkylalkyl, for example, cyclopropyl, cyclopropylmethyl, neopentyl, cyclobutyl, isobutyl, isopropyl, sec-butyl, cyclobutylmethyl, etc.

In certain embodiments, the sum of q and r is less than 4, e.g., is 2 or 3.

In certain embodiments, XLR₄, taken together, include a cyclic amine, such as a piperazine, a morpholine, a piperidine, a pyrrolidine, etc.

In certain embodiments, at least one of R₁, R₂, and R₃ includes an aryl or heteroaryl group. In certain related embodiments, at least two of R₁, R₂, and R₃ include an aryl or heteroaryl group. In certain embodiments, R₁ is lower alkyl.

In certain embodiments, L attached to R₁ represents O, S, or NR₈, such as NH.

In certain embodiments, M is absent.

In certain embodiments, X is not NH. In certain embodiments, X is included in a ring, or, taken together with —C(═Y)—, represents a tertiary amide.

In certain embodiments, compounds useful in the present invention may be represented by general formula (IX):

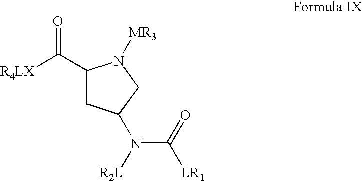

wherein, as valence and stability permit,

R₁, R₂, R₃, R₄, R₈, L, M, X, n, and p are as defined above.

In certain embodiments, XLR₄, taken together, include a cyclic amine, such as a piperazine, a morpholine, a piperidine, a pyrrolidine, etc.

In certain embodiments, R₁ represents a lower alkyl group, preferably a branched alkyl, a cycloalkyl, or a cycloalkylalkyl, for example, cyclopropyl, cyclopropylmethyl, neopentyl, cyclobutyl, isobutyl, isopropyl, sec-butyl, cyclobutylmethyl, etc.

In certain embodiments, at least one of R₁, R₂, and R₃ includes an aryl or heteroaryl group. In certain related embodiments, at least two of R₁, R₂, and R₃ include an aryl or heteroaryl group. In certain embodiments, R₁ is lower alkyl.

In certain embodiments, L attached to R₁ represents O, S, or NR₈, such as NH.

In certain embodiments, M is absent.

In certain embodiments, X is not NH. In certain embodiments, X is included in a ring, or, taken together with —C(═Y)—, represents a tertiary amide.

In certain embodiments L represents a direct bond for all occurrences.

In certain embodiments, compounds useful in the present invention may be represented by general formula (X):

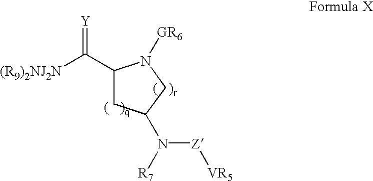

wherein, as valence and stability permit,

Y, n, p, q, and r are as defined above;

Z′ represents —C(═O)—, —C(═S)—, —C(═NH)—, SO₂, or SO, preferably —C(′O)—, —C(═S)—;

V is absent or represents O, S, or NR₈;

G is absent or represents —C(═O)— or —SO₂—;

J, independently for each occurrence, represents H or substituted or unsubstituted lower alkyl or alkylene, such as methyl, ethyl, methylene, ethylene, etc., attached to NC(═Y), such that both occurrences of N adjacent to J are linked through at least one occurrence of J, and

R₉, independently for each occurrence, is absent or represents H or lower alkyl, or two occurrences of J or one occurrence of J taken together with one occurrence of R g, forms a ring of from 5 to 7 members, which ring includes one or both occurrences of N;

R₅ represents substituted or unsubstituted alkyl (e.g., branched or unbranched), alkenyl (e.g., branched or unbranched), alkynyl (e.g., branched or unbranched), cycloalkyl, or cycloalkylalkyl;

R₆ represents substituted or unsubstituted aryl, aralkyl, heteroaryl, heteroaralkyl, heterocyclyl, heterocyclylalkyl, cycloalkyl, or cycloalkylalkyl, including polycyclic groups; and

R₇ represents substituted or unsubstituted aryl, aralkyl, heteroaryl, or heteroaralkyl.

In certain embodiments, Y is O. In certain embodiments, Z′ represents SO₂, —C(═O)—, or—C(═S)—.

In certain embodiments, the sum of q and r is less than 4.

In certain embodiments, NJ₂N, taken together, represent a cyclic diamine, such as a piperazine, etc., which may be substituted or unsubstituted, e.g., with one or more substitutents such as oxo, lower alkyl, lower alkyl ether, etc. In certain other embodiments, NJ₂ or NJR₉ taken together represent a substituted or unsubstituted heterocyclic ring to which the other occurrence of N is attached. In certain embodiments, one or both occurrences of J are substituted with one or more of lower alkyl, lower alkyl ether, lower alkyl thioether, amido, oxo, etc. In certain embodiments, a heterocyclic ring which comprises an occurrence of J has from 5 to 8 members.

In certain embodiments, R₅ represents a branched alkyl, cycloalkyl, or cycloalkylalkyl.

In certain embodiments, R₆ includes at least one heterocyclic ring, such as a thiophene, furan, oxazole, benzodioxane, benzodioxole, pyrrole, indole, etc.

In certain embodiments, R₇ represents a phenyl alkyl, such as a benzyl group, optionally substituted with halogen, hydroxyl, lower alkyl, nitro, cyano, lower alkyl ether (e.g., optionally substituted, such as CHF₂CF₂O), or lower alkyl thioether (e.g., optionally substituted, such as CF₃S).

In certain embodiments, R₈, when it occurs in V, represents H or lower alkyl, preferably H.

In certain embodiments, compounds useful in the present invention may be represented by general formula (XI):

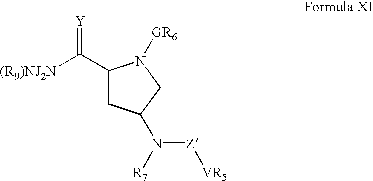

wherein, as valence and stability permit,

R₅, R₆, R₇, R₈, R₉, R₁₀, G, J, V, Y, Z′, n, and p are as defined above.

In certain embodiments, Y is O. In certain embodiments, Z′ represents SO₂, —C(═O)—, or —C(═S)—.

In certain embodiments, NJ₂N, taken together, represent a heterocyclic ring, such as a piperazine, etc., which may be substituted or unsubstituted, e.g., with one or more substitutents such as oxo, lower alkyl, lower alkyl ether, etc. In certain other embodiments, NJ₂ or NJR₉ taken together represent a substituted or unsubstituted heterocyclic ring to which the other occurrence of N is attached. In certain embodiments, one or both occurrences of J are substituted with one or more of lower alkyl, lower alkyl ether, lower alkyl thioether, amido, oxo, etc. In certain embodiments, a heterocyclic ring which comprises an occurrence of J has from 5 to 8 members.

In certain embodiments, R₅ represents a branched alkyl, cycloalkyl, or cycloalkylalkyl.

In certain embodiments, R₆ includes at least one heterocyclic ring, such as a thiophene, furan, oxazole, benzodioxane, benzodioxole, pyrrole, indole, etc.

In certain embodiments, R₇ represents a phenyl alkyl, such as a benzyl group, optionally substituted with halogen, hydroxyl, lower alkyl, nitro, cyano, lower alkyl ether (e.g., optionally substituted, such as CHF₂CF₂O), or lower alkyl thioether (e.g., optionally substituted, such as CF₃S).

In certain embodiments, R₈, when it occurs in V, represents H or lower alkyl, preferably H.

In certain preferred embodiments, the subject inhibitors inhibit hedgehog-mediated signal transduction with an IC₅₀ of 1 mM or less, more preferably of 1 μM or less, and even more preferably of 1 nM or less.

Moreover, the subject methods can be performed on cells which are provided in culture (in vitro), or on cells in a whole animal (in vivo). See, for example, PCT publications WO 95/18856 and WO 96/17924 (the specifications of which are expressly incorporated by reference herein).

V. Ago n ists of Hedgehog Biological Activity

Preferred hedgehog therapeutics useful in methods of the invention are agonists that are derived from several sources of hedgehog protein. In one embodiment, the agonist is not N-terminally clipped (as described above). Other embodiments of a hedgehog therapeutic suitable for the present methods are based, in part, on the discovery disclosed in U.S. patent application No. 60/067,423 (Dec. 3, 1997:PCT Publication that human Sonic hedgehog, expressed as a full-length construct in either insect or in mammalian cells, has a hydrophobic palmitoyl group appended to the alpha-amine of the N-terminal cysteine. This is the first example of an extracellular signaling protein being modified in such a manner, and, in contrast to thiol-linked palmitic acid modifications whose attachment is readily reversible, this novel N-linked palmitoyl moiety is likely to be very stable by analogy with myristic acid modifications.

The agonists have at least one of the following properties: (i) the isolated protein binds the receptor patched-I with an affinity that is at similar to, but is preferably higher than, the binding of mature hedgehog protein to patched-1; or (ii) the isolated protein binds to a hedgehog protein in such a way as to increase the proteins binding affinity to patched-i when tested in an in vitro CH310T1/2 cell-based AP induction assay. Agonists of the invention may also have the additional properties of being (iii) able to solely induce ptc-1 and gli-1 expression.

The preferred agonists for use in conjugation with a non-hedgehog conjugate (e.g., immunoglobulin or fragment thereof) include a derivitized hedgehog polypeptide sequence as well as other N-terminal and/or C-terminal amino acid sequence or it may include all or a fragment of a hedgehog amino acid sequence. Agonist polypeptides of the invention include those that arise as a result of the existence of multiple genes, alternative transcription events, alternative RNA splicing events, and alternative translational and posttranslational events. The polypeptide can be made entirely by synthetic means or can be expressed in systems, e.g., cultured cells, which result in substantially the same posttranslational modifications present when the protein is expressed in a native cell, or in systems which result in the omission of posttranslational modifications present when expressed in a native cell.

In one embodiment, the agonist is a hedgehog polypeptide with one or more of the following characteristics:

(i) it has at least 30, 40, 42, 50, 60, 70, 80, 90 or 95% sequence identity with a hedgehog sequence such as SEQ ID NOS: 10-18 or 23-26;

(ii) it has a cysteine or a functional equivalent as the N-terminal end;

(iii) it may induce alkaline phosphatase activity in C3H10T1/2 cells;

(iv) it has an overall sequence identity of at least 50%, preferably at least 60%, more preferably at least 70, 80, 90, or 95%, with a polypeptide of a hedgehog sequence;

(v) it can be isolated from natural sources such as mammalian cells;

(vi) it can bind or interact with patched; and

(vii) it may be hydrophobically-modified (i.e., it has at least one hydrophobicmoiety attached to the polypeptide).

Increasing the overall hydrophobic nature of a hedgehog protein increases the biological activity of the protein. The potency of a signaling protein such as hedgehog can be increased by: (a) chemically modifying, such as by adding a hydrophobic moiety to, the sulfhydryl and/or to the alpha-amine of the N-terminal cysteine (see U.S. Pat. No. 60/067,423); (b) replacing the N-terminal cysteine with a hydrophobic amino acid (see U.S. Pat. No. 60/067,423); or (c) replacing the N-terminal cysteine with a different amino acid and then chemically modifying the substituted residue so as to add a hydrophobic moiety at the site of the substitution.

Additionally, modification of a hedgehog protein at an internal residue on the surface of the protein with a hydrophobic moiety by: (a) replacing the internal residue with a hydrophobic amino acid; or (b) replacing the internal residue with a different amino acid and then chemically modifying the substituted residue so as to add a hydrophobic moiety at the site of the substitution will retain or enhance the biological activity of the protein.

Additionally, modification of a protein such as a hedgehog protein at the C-terminus with a hydrophobic moiety by: (a) replacing the C-terminal residue with a hydrophobic amino acid; or (b) replacing the C-terminal residue with a different amino acid and then chemically modifying the substituted residue so as to add a hydrophobic moiety at the site of the substitution, will retain or enhance the biological activity of the protein.

For hydrophobically-modified hedgehog obtained by chemically modifying the soluble, unmodified protein, palmitic acid and other lipids can be added to soluble Shh to create a lipid-modified forms with increased potency in the C3HIOT1/2 assay. Another form of protein encompassed by the invention is a protein derivatized with a variety of lipid moieties. The principal classes of lipids that are encompassed within this invention are fatty acids and sterols (e.g., cholesterol). Derivatized proteins of the invention contain fatty acids which are cyclic, acyclic (i.e., straight chain), saturated or unsaturated, mono-carboxylic acids. Exemplary saturated fatty acids have the generic formula: CH3 (CH₂)n COON. Table 2 below lists examples of some fatty acids that can be derivatized conveniently using conventional chemical methods.

TABLE 2
Exemplary Saturated and Unsaturated Fatty Acids
Saturated Acids: CH3 (CH2)n COOH:

| | Common Name |
|---|---|
| Value of n | |
| 2 | butyric acid |
| 4 | caproic acid |
| 6 | caprylic acid |
| 8 | capric acid |
| 10 | lauric acid |
| 12 | myristic acid* |
| 14 | palmitic acid* |
| 16 | stearic acid* |
| 18 | arachidic acid* |
| 20 | behenic acid |
| 22 | lignoceric acid |
| Unsaturated Acids: | |
| CH3CH═CHCOOH | crotonic acid |
| CH3(CH2)3CH═CH(CH2)7COOH | myristoleic acid* |
| CH3(CH2)5CH═CH(CH2)7COOH | palmitoleic acid* |
| CH3(CH2)7CH═CH(CH2)7COOH | oleic acid* |
| CH3(CH2)3(CH2CH═CH)2(CH2)7COOH | linoleic acid |
| CH3(CH2CH═CH)3(CH2)7COOH | linolenic acid |
| CH3(CH2)3(CH2CH═CH)4(CH2)3COOH | arachidonic acid |

The asterisk (*) denotes fatty acids detected in recombinant hedgehog protein secreted from a soluble construct (Pepinsky et al., supra).

Other lipids that can be attached to the protein include branched-chain fatty acids and those of the phospholipid group such as the phosphatidylinositols (i.e., phosphatidylinositol 4-monophosphate and phosphatidylinositol 4,5- biphosphate), phosphatidycholine, phosphatidylethanolamine, phosphatidylserine, and isoprenoids such as farnesyl or geranyl groups. Lipid-modified hedgehog proteins can be purified from either a natural source, or can be obtained by chemically modifying the soluble, unmodified protein.

For protein purified from a natural source, we showed that when full-length human Sonic hedgehog (Shh) was expressed in insect cells and membrane-bound Shh purified from the detergent-treated cells using a combination of SP—Sepharose chromatography and immunoaffinity chromatography, that the purified protein migrated on reducing SDS-PAGE gels as a single sharp band with an apparent mass of 20 kDa. See PCT The soluble and membrane-bound Shh proteins were readily distinguishable by reverse phase HPLC, where the tethered forms eluted later in the acetonitrile gradient. We then demonstrated that human Sonic hedgehog is tethered to cell membranes in two forms, one form that contains a cholesterol, and therefore is analogous to the data reported previously for Drosophila hedgehog, and a second novel form that contains both a cholesterol and a palmitic acid modification. Both modified forms were equally as active in the C3H10T1/2 alkaline phosphatase assay, but both were about 30-times more potent than soluble human Shh lacking the tether(s). The hydrophobic modifications did not significantly affect the apparent binding affinity of Shh for its receptor, patched.

For specific lipid-modified hedgehog obtained by chemically modifying the soluble, unmodified protein, palmitic acid and other lipids can be added to soluble Shh to create a lipid-modified forms with increased potency in the C3H10T1/2 assay. Generally, therefore, the reactive lipid moiety can be in the form of thioesters of saturated or unsaturated carboxylic acids such as a Coenzyme A thioesters. Such materials and their derivatives may include, for example, commercially available Coenzyme A derivatives such as palmitoleoyl Coenzyme A, arachidoyl Coenzyme A, arachidonoyl Coenzyme A, lauroyl Coenzyme A and the like. These materials are readily available from Sigma Chemical Company (St. Louis, Mo., 1998 catalog pp. 303-306).

There are a wide range of hydrophobic moieties with which hedgehog polypeptides can be derivatived. A hydrophobic group can be, for example, a relatively long chain alkyl or cycloalkyl (preferably n-alkyl) group having approximately 7 to 30 carbons. The alkyl group may terminate with a hydroxy or primary amine “tail”. To further illustrate, such molecules include naturally-occurring and synthetic aromatic and non-aromatic moieties such as fatty acids, esters and alcohols, other lipid molecules, cage structures such as adamantane and buckminsterfullerenes, and aromatic hydrocarbons such as benzene, perylene, phenanthrene, anthracene, naphthalene, pyrene, chrysene, and naphthacene.

Particularly useful as hydrophobic molecules are alicyclic hydrocarbons, saturated and unsaturated fatty acids and other lipid and phospholipid moieties, waxes, cholesterol, isoprenoids, terpenes and polyalicyclic hydrocarbons including adamantane and buckminsterfullerenes, vitamins, polyethylene glycol or oligoethylene glycol, (C1-C18)-alkyl phosphate diesters, —O—CH₂—CH(OH)—O—(C12-C18)-alkyl, and in particular conjugates with pyrene derivatives. The hydrophobic moiety can be a lipophilic dye suitable for use in the invention include, but are not limited to, diphenylhexatriene, Nile Red, N-phenyl-l-naphthylamine, Prodan, Laurodan, Pyrene, Perylene, rhodamine, rhodamine B, tetramethylrhodamine, Texas Red, sulforhodamine, 1,1′-didodecyl-3,3,3′,3′tetramethylindocarbocyanine perchlorate, octadecyl rhodamine B and the BODIPY dyes available from Molecular Probes Inc.

Other exemplary lipophilic moieties include aliphatic carbonyl radical groups include 1- or 2-adamantylacetyl, 3-methyladamant-1-ylacetyl, 3-methyl-3-bromo-1-adamantylacetyl, 1-decalinacetyl, camphoracetyl, camphaneacetyl, noradamantylacetyl, norbomaneacetyl, bicyclo[2.2.2.]-oct-5-eneacetyl, 1-methoxybicyclo[2.2.2.]-oct-5-ene-2-carbonyl, cis-5-norbomene-endo-2,3-dicarbonyl, 5-norbornen-2-ylacetyl, (1R)—(−)-myrtentaneacetyl, 2-norbomaneacetyl, anti-3-oxo-tricyclo[2.2. 1.0<2,6>]-heptane-7-carbonyl, decanoyl, dodecanoyl, dodecenoyl, tetradecadienoyl, decynoyl or dodecynoyl.

1. Chemical Modifications of the N-terminal cysteine of hedgehog

If an appropriate amino acid is not available at a specific position, site-directed mutagenesis can be used to place a reactive amino acid at that site. Reactive amino acids include cysteine, lysine, histidine, aspartic acid, glutamic acid, serine, threonine, tyrosine, arginine, methionine, and tryptophan. Mutagenesis could also be used to place the reactive amino acid at the N- or C-terminus or at an internal position.

For example, it is possible to chemically modify an N-terminal cysteine of a biologically active protein, such as a hedgehog protein, or eliminate the N-terminal cysteine altogether and still retain the protein's biological activity. The replacement or modification of the N-terminal cysteine of hedgehog with a hydrophobic amino acid results in a protein with increased potency in a cell-based signaling assay. By replacing the cysteine, this approach eliminates the problem of suppressing other unwanted modifications of the cysteine that can occur during the production, purification, formulation, and storage of the protein. The generality of this approach is supported by the finding that three different hydrophobic amino acids, phenylalanine, isoleucine, and methionine, each give a more active form of hedgehog, and thus, an agonist.

This is also important for conjugation with non-hedgehog moieties (e.g., immunoglobulin) as described below in which we introduce two isoleucine residues to the N-terminal cysteine end of Sonic and Desert hedgehog. This effectively allows us to use the thiol of C-terminal cysteine as the reactive site for covalent coupling. Thus, replacement of the N-terminal cysteine with any other hydrophobic amino acid should result in an active protein. Furthermore, since we have found a correlation between the hydrophobicity of an amino acid or chemical modification and the potency of the corresponding modified protein in the C3H10T1/2 assay (e.g. Phe>Met, long chain length fatty acids>short chain length), it could be envisioned that adding more than one hydrophobic amino acid to the hedgehog sequence would increase the potency of the agonist beyond that achieved with a single amino acid addition. Indeed, addition of two consecutive isoleucine residues to the N-terminus of human Sonic hedgehog results in an increase in potency in the C3H10T1/2 assay as compared to the mutant with only a single isoleucine added. Thus, adding hydrophobic amino acids at the N- or C-terminus of a hedgehog protein, in a surface loop, or some combination of positions would be expected to give a more active form of the protein. The substituted amino acid need not be one of the 20 common amino acids. Methods have been reported for substituting unnatural amino acids at specific sites in proteins and this would be advantageous if the amino acid was more hydrophobic in character, resistant to proteolytic attack, or could be used to further direct the hedgehog protein to a particular site in vivo that would make its activity more potent or specific. Unnatural amino acids can be incorporated at specific sites in proteins during in vitro translation, and progress is being reported in creating in vivo systems that will allow larger scale production of such modified proteins.

There are many modifications of the N-terminal cysteine which protect the thiol and append a hydrophobic moiety. One of skill in the art is capable of determining which modification is most appropriate for a particular therapeutic use. Factors affecting such a determination include cost and ease of production, purification and formulation, solubility, stability, potency, pharmacodynamics and kinetics, safety, immunogenicity, and tissue targeting.

2. Chemical modification of other amino acids.

There are specific chemical methods for the modification of many other amino acids. Therefore, another route for synthesizing a more active form of hedgehog would be to chemically attach a hydrophobic moiety to an amino acid in hedgehog other than to the N-terminal cysteine. If an appropriate amino acid is not available at the desired position, site-directed mutagenesis could be used to place the reactive amino acid at that site in the hedgehog structure, whether at the N- or C-terminus or at another position. Reactive amino acids would include cysteine, lysine, histidine, aspartic acid, glutamic acid, serine, threonine, tyrosine, arginine, methionine, and tryptophan. Thus the goal of creating a better hedgehog agonist could be attained by many chemical means and we do not wish to be restricted by a particular chemistry or site of modification since our results support the generality of this approach.

The hedgehog polypeptide can be linked to the hydrophobic moiety in a number of ways including by chemical coupling means, or by genetic engineering. To illustrate, there are a large number of chemical cross-linking agents that are known to those skilled in the art. For the present invention, the preferred cross-linking agents are heterobifunctional cross-linkers, which can be used to link the hedgehog polypeptide and hydrophobic moiety in a stepwise manner. Heterobifunctional cross-linkers provide the ability to design more specific coupling methods for conjugating to proteins, thereby reducing the occurrences ofunwanted side reactions such as homo-protein polymers. A wide variety of heterobifunctional cross-linkers are known in the art. These include: succinimidyl 4-(N-maleimidomethyl) cyclohexane-1-carboxylate (SMCC), m-Maleimidobenzoyl-N-hydroxysuccinimide ester (MBS); N-succinimidyl (4-iodoacetyl) aminobenzoate (SIAB), succinimidyl 4—(p-maleimidophenyl) butyrate (SMPB), 1-ethyl-3—(3-dimethylaminopropyl) carbodiimide hydrochloride (EDC); 4-succinimidyloxycarbonyl-a-methyl-a-(2-pyridyldithio)-tolune (SMPT), N-succinimidyl 3-(2-pyridyldithio) propionate (SPDP), succinimidyl 6-[3—(2-pyridyldithio) propionate] hexanoate (LC—SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product.

Another reactive group useful as part of a heterobifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodoacetyl functions) react with —SH groups at physiological pH's. Both of these reactive groups result in the formation of stable thioether bonds.

Generally, the structure of an agonistic hedgehog therapeutic useful in this invention is a chimeric molecule that has the general formula: X—Y—Z, where wherein X is a polypeptide having the amino acid sequence, or portion thereof, consisting of the amino acid sequence of hedgehog; Y is an optional linker moiety; and Z is a polypeptide comprising at least a portion of a polypeptide other than hedgehog . Preferably, X includes at least a biologically active N-terminal fragment of is human Sonic, Indian or Desert hedgehog. In the more preferred embodiments, Z is a protein with an 19-like constant and/or variable domain. Most preferably, Z is at least a portion of a constant region of an immunoglobulin and can be derived from an immunoglobulin of the class selected from IgM, IgG, IgD, IgA, and IgE. If the class is IgG, then it is selected from one of IgG1, IgG2, IgG3 and IgG4. The constant region of human IgM and IgE contain 4 constant regions (CHI, (hinge), CH_{2,} CH3 and CH4, whereas the constant region of human IgG, IgA and IgD contain 3 constant regions (CHI, (hinge), CH₂ and CH3. In the most preferred fusion proteins of the invention, the constant region contains at least the hinge, CH₂ and CH3 domains.

In another embodiment, the chimeric molecule has the structure D-[Sp]-B-[Sp]-C, where D is a non-hedgehog moiety such as described herein; [Sp] is an optional spacer peptide sequence; B is a hedgehog protein (which optionally may be a mutein as described herein); and C is an optional hydrophobic moiety linked (optionally by way of the spacer peptide) to the hedgehog protein D or another residue such as a surface site of the protein.

The present invention provides for multimeric hedgehog therapeutic molecules. Such multimers may be generated by using those Fc regions, or portions thereof, of Ig molecules which are usually multivalent such as IgM pentamers or IgA dimers. It is understood that a J chain polypeptide may be needed to form and stabilize IgM pentamers and IgA dimers. Alternatively, multimers of hedgehog therapeutic proteins may be formed using a protein with an affinity for the Fc region of Ig molecules, such as Protein A. For instance, a plurality of hedgehog/immunoglobulin fusion proteins may be bound to Protein A-agarose beads.

These multivalent forms are useful since they possess multiple hedgehog receptor binding sites. For example, a bivalent soluble hedgehog therapeutic may consist of two tandem repeats of those amino acids encoded by nucleic acids of SEQ. ID NOS: 1-9 or 21, 22 or 27 (moiety X in the generic formula) separated by a linker region (moiety Y), the repeats bound to at least a portion of an immunoglobulin constant domain (moiety Z). Alternate polyvalent forms may also be constructed, for example, by chemically coupling chimeric hedgehog therapeutics of the invention to any clinically acceptable carrier molecule, a polymer selected from the group consisting of Ficoll, polyethylene glycol or dextran using conventional coupling techniques. Alternatively, hedgehog may be chemically coupled to biotin, and the biotin-hedgehog chimera then allowed to bind to avidin, resulting in tetravalent avidin/biotin/hedgehog molecules. Chimeric hedgehog proteins may also be covalently coupled to dinitrophenol (DNP) or trinitrophenol (TNP) and the resulting conjugate precipitated with anti-DNP or anti-TNP-IgM, to form decameric conjugates with a valency of 10 for hedgehog receptor binding sites

Polymer Conjugates of Hedgehog Therapeutics

One unique property of polyalkylene glycol-derived polymers of value for therapeutic applications of the present invention is their general biocompatibility. These polymers have various water solubility properties and are not toxic. They are believed non-immunogenic and non-antigenic and do not interfere with the biological activities of the hedgehog protein moiety when conjugated under the conditions described herein. They have long circulation in the blood and are easily excreted from living organisms.

Hedgehog therapeutics are conjugated most preferably via a terminal reactive group on the polyalkylene glycol polymer although conjugations can also be branched from non-terminal reactive groups. The polymer with the reactive group(s) is designated herein as “activated polymer”. The reactive group would be expected to selectively react with free amino or other reactive groups on the hedgehog protein. In theory, the activated polymer(s) are reacted so that attachment could occur at any available hedgehog amino group such as alpha amino groups or the epsilon-amino groups of lysines, or —SH groups of cysteines. Free carboxylic groups, suitably activated carbonyl groups, hydroxyl, guanidyl, oxidized carbohydrate moieties and mercapto groups of the hedgehog protein (if available) can also be used as attachment sites.

In particular, the chemical modification of any N-terminal cysteine to protect the thiol, with concomitant conjugation with a polyalkylene glycol moiety (i.e., PEG), can be carried out in numerous ways by someone skilled in the art. See United States Patent 4,179,337. The sulfhydryl moiety, with the thiolate ion as the active species, is the most reactive functional group in a protein. There are many reagents that react faster with the thiol than any other groups. See Chemistry of Protein Conjugation and Cross-Linking (S. S. Wong, CRC Press, Boca Raton, Fla., 1991). The thiol of an N-terminal cysteine, such as found in all hedgehog proteins, would be expected to be more reactive than internal cysteines within the sequence. This is because the close proximity to the alpha-amine will lower the pKa of the thiol resulting in a greater degree of proton dissociation to the reactive thiolate ion at neutral or acid pH. In addition, the cysteine at the N-terminus of the structure is more likely to be exposed than the other two cysteines in the hedgehog sequence that are found buried in the protein structure.

Other examples of methods that provide linkage between a polyalkylene glycol and the N-terminal cysteine would be reactions with other alpha-haloacetyl compounds, organomercurials, disulfide reagents, and other N-substituted maleimides. Numerous derivatives of these active species are available commercially (e.g., ethyl iodoacetate (Aldrich, Milwaukee Wis.), phenyl disulfide (Aldrich), and N-pyrenemaleimide (Molecular Probes, Eugene Oreg.)) or could be synthesized readily (e.g., N-alkyliodoacetamides, N-alkylmaleimides, and organomercurials). Another aspect to the reactivity of an N-terminal cysteine is that it can take part in reaction chemistries unique to its 1,2-aminothiol configuration. One example is the reaction with thioester groups to form an N-terminal amide group via a rapid S to N shift of the thioester. This reaction chemistry can couple together synthetic peptides and can be used to add single or multiple, natural or unnatural, amino acids or other hydrophobic groups via the appropriately activated peptide. Another example, is the reaction with aldehydes to form the thiazolidine adduct. Numerous hydrophobic derivatives of thiol esters (e.g., C2—C24 saturated and unsaturated fatty acyl Coenzyme A esters (Sigma Chemical Co., St. Louis Mo.)), aldehydes (e.g., butyraldehyde, n-decyl aldehyde, and n-myristyl aldehyde (Aldrich)), and ketones (e.g., 2-, 3-, and 4-decanone (Aldrich)) are available commercially or could be synthesized readily. In a similar manner, thiomorpholine could be prepared from a variety of alpha-haloketone starting materials.

Several observations suggest that the C-terminus or amino acids near the C-terminus would be preferred targets for modification with a polyalkylene glycol moiety. Briefly, we have shown that: (i) The wild-type protein is naturally modified with cholesterol at the C-terminus, indicating that it is exposed and available for modification. Indeed, we showed that treatment with thrombin results in selective release of the C-terminal 3 amino acids (See U.S. Ser. No. 60/106,703, filed Nov. 2, 1998, now PCT Number -incorporated herein by reference); (ii) We performed extensive SAR analyses and discovered that the C-terminal 11 amino acids could be deleted without harmful effects on folding or function; (iii) We have made hedgehog/Ig fusion proteins by attaching an Ig moiety to the C-terminus of hedgehog without harmful effects on folding or function (data not presented here).

While there is no simple chemical strategy for targeting a polyalkylene glycol polymer such as PEG to the C-terminus of hedgehog, it is straightforward to genetically engineer a site that can be used to target the polymer moiety, as discussed above with regard to site-directed mutagenesis. For example, incorporation of a Cys at a site that is at or near the C-terminus allows specific modification using a maleimide, vinylsulfone or haloacetate-activated polyalkylene glycol (e.g., PEG). As discussed above in Section A, these derivatives can be used specifically for modification of the engineered C-terminal cysteines due to the high selectively of these reagents for Cys. Other strategies such as incorporation of a histidine tag which can be targeted (Fancy et al., (1996) Chem. & Biol. 3: 551) or an additional glycosylation site, represent other alternatives for modifying the C-terminus of hedgehog. A single polymer molecule may be employed for conjugation with the hedgehog protein and modified versions thereof as discussed above, although it is also contemplated that more than one polymer molecule can be attached as well. Conjugated hedgehog compositions of the invention may find utility in both in vivo as well as non-in vivo applications. Additionally, it will be recognized that the conjugating polymer may utilize any other groups, moieties, or other conjugated species, as appropriate to the end use application. By way of example, it may be useful in some applications to covalently bond to the polymer a functional moiety imparting UV-degradation resistance, or antioxidation, or other properties or characteristics to the polymer. As a further example, it may be advantageous in some applications to functionalize the polymer to render it reactive or cross-linkable in character, to enhance various properties or characterisics of the overall conjugated material. Accordingly, the polymer may contain any functionality, repeating groups, linkages, or other constitutent structures which do not preclude the efficacy of the conjugated hedgehog composition for its intended purpose. Other objectives and advantages of the present invention will be more fully apparent from the ensuing disclosure and appended claims.

Illustrative polymers that may usefully be employed to achieve these desirable characteristics are described herein below in exemplary reaction schemes. In covalently bonded peptide applications, the polymer may be functionalized and then coupled to free amino acid(s) of the peptide(s) to form labile bonds.

Generally from about 1.0 to about 10 moles of activated polymer per mole of protein is employed, depending on the particular reaction chemistry and the protein concentration. The final amount is a balance between maximizing the extent of the reaction while minimizing non-specific modifications of the product and, at the same time, defining chemistries that will maintain optimum activity, while at the same time optimizing, if possible, the half-life of the protein. Preferably, at least about 50% of the biological activity of the protein is retained, and most preferably 100% is retained.

The reactions may take place by any suitable method used for reacting biologically active materials with inert polymers. Generally the process involves preparing an activated polymer (that may have at least one terminal hydroxyl group) and thereafter reacting the protein with the activated polymer to produce the soluble protein suitable for formulation. The above modification reaction can be performed by several methods, which may involve one or more steps.

Suitable methods of attaching a polyalkylene glycol moiety to a C-terminal cysteine involve using such moieties that are activated with a thiol reactive group, as generally discussed above. Common thiol reactive groups include maleimides, vinylsulfones or haloacetates. These derivatives can be used specifically for modification of cysteines due to the high selectively of these reagents for —SH. Maleimides react specifically with free sulfhydryls (cysteine residues) in minutes, under slightly acidic to neutral (pH 6.0-7.5) conditions. This pH range is preferred although the reaction will proceed, albeit slowly, at pH 5.0. Halogens (iodoacetyl functions) react with —SH groups at physiological pH's to slightly basic conditions. Both of these reactive groups result in the formation of stable thioether bonds.

In the practice of the methods of the present invention, polyalkylene glycol residues of C1-C4 alkyl polyalkylene glycols, preferably polyethylene glycol (PEG), or poly(oxy)alkylene glycol residues of such glycols are advantageously incorporated in the polymer systems of interest. Thus, the polymer to which the protein is attached can be a homopolymer of polyethylene glycol (PEG) or is a polyoxyethylated polyol, provided in all cases that the polymer is soluble in water at room temperature. Non-limiting examples of such polymers include polyalkylene oxide homopolymers such as PEG or polypropylene glycols, polyoxyethylenated glycols, copolymers thereof and block copolymers thereof, provided that the water solubility of the block copolymer is maintained. Examples of polyoxyethylated polyols include, for example, polyoxyethylated glycerol, polyoxyethylated sorbitol, polyoxyethylated glucose, or the like. The glycerol backbone of polyoxyethylated glycerol is the same backbone occurring naturally in, for example, animals and humans in mono-, di-, and triglycerides. Therefore, this branching would not necessarily be seen as a foreign agent in the body.

As an alternative to polyalkylene oxides, dextran, polyvinyl pyrrolidones, polyacrylamides, polyvinyl alcohols, carbohydrate-based polymers and the like may be used. Moreover, heteropolymers (i.e., polymers consisting of more than one species of monomer such as a copolymer) as described in U.S. Pat. No. 5,359,030 may be used (e.g., proteins conjugated to polymers comprising a polyalkylene glycol moiety and one or more fatty acids) Those of ordinary skill in the art will recognize that the foregoing list is merely illustrative and that all polymer materials having the qualities described herein are contemplated. The polymer need not have any particular molecular weight, but it is preferred that the molecular weight be between about 300 and 100,000, more preferably between 10,000 and 40,000. In particular, sizes of 20,000 or more are best at preventing protein loss due to filtration in the kidneys. Moreover, in another aspect of the invention, one can utilize hedgehog covalently bonded to the polymer component in which the nature of the conjugation involves cleavable covalent chemical bonds. This allows for control in terms of the time course over which the polymer may be cleaved from the hedgehog. This covalent bond between the hedgehog protein drug and the polymer may be cleaved by chemical or enzymatic reaction. The polymer-hedgehog protein product retains an acceptable amount of activity. Concurrently, portions of polyethylene glycol are present in the conjugating polymer to endow the polymer-hedgehog protein conjugate with high aqueous solubility and prolonged blood circulation capability. As a result of these improved characteristics the invention contemplates parenteral, aerosol, and oral delivery of both the active polymer-hedgehog protein species and, following hydrolytic cleavage, bioavailability of the hedgehog protein per se, in in vivo applications.

It is to be understood that the reaction schemes described herein are provided for the purposes of illustration only and are not to be limiting with respect to the reactions and structures which may be utilized in the modification of the hedgehog protein, e.g., to achieve solubility, stabilization, and cell membrane affinity for parenteral and oral administration. Generally speaking, the concentrations of reagents used are not critical to carrying out the procedures provided hererin except that the molar amount of activated polymer should be at least equal to, and preferably in excess of, the molar amount of the reactive group (e.g., thiol) on the hedgehog amino acid(s). The reaction of the polymer with the hedgehog to obtain the most preferred conjugated products is readily carried out using a wide variety of reaction schemes. The activity and stability of the hedgehog protein conjugates can be varied in several ways, by using a polymer of different molecular size. Solubilities of the conjugates can be varied by changing the proportion and size of the polyethylene glycol fragment incorporated in the polymer composition.

3. Small Molecule Agonists

In other embodiments, a hedgehog agonist may be a small organic molecule. Such a small organic molecule may agonize hedgehog signal transduction via an interaction with but not limited to hedgehog, patched (ptc), gli, and/or smoothened. It is, therefore, specifically contemplated that these small molecules which enhance or potentiate aspects of hedgehog, ptc, or smoothened signal transduction will likewise be capable of enhancing angiogenesis (or other biological consequences) in normal cells and/or mutant cells. Thus, it is contemplated that in certain embodiments, these compounds may be useful for enhancing or potentiating hedgehog activity. In other embodiments, these compounds may be useful for inhibitng hedgehog activity in abnormal cells. In preferred embodiments, the subject agonists are organic molecules having a molecular weight less than 2500 amu, more preferably less than 1500 amu, and even more preferably less than 750 amu, and are capable of agonizing hedgehog signaling, preferably specifically in target cells.

For example, agonist compounds useful in the subject methods include compounds represented by general formula (XII):

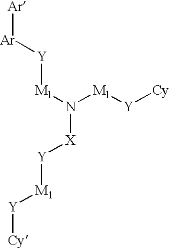

Formula XII

wherein, as valence and stability permit,

Ar and Ar′ independently represent substituted or unsubstituted aryl or heteroaryl rings;

Y, independently for each occurrence, may be absent or represent —N(R)—, —O—, —S—, or —Se—;

X can be selected from —C(═O)—, —C(═S)—, —S(O₂)—, —S(O)—, —C(═NCN)—, —P(═O)(OR₂)—, and a methylene group optionally substituted with 1-2 groups such as lower alkyl, alkenyl, or alkynyl groups;

M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;

R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;

Cy and Cy′ independenly represent substituted or unsubstituted aryl, heterocyclyl, heteroaryl, or cycloalkyl, including polycyclic groups;

i represents, independently for each occurrence, an integer from 0 to 5, preferably from 0 to 2; and

n, individually for each occurence, represents an integer from 0 to 10, preferably from 0 to 5.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Ar and Ar′ represent phenyl rings, e.g., unsubstituted or substituted with one or more groups including heteroatoms such as O, N, and S. In certain embodiments, at least one of Ar and Ar′ represents a phenyl ring. In certain embodiments, at least one of Ar and Ar′ represents a heteroaryl ring, e.g., a pyridyl, thiazolyl, thienyl, pyrimidyl, etc. In certain embodiments, Y and Ar′ are attached to Ar in a meta and/or 1,3-relationship.

In certain embodiments, Y is absent from all positions. In embodiments wherein Y is present in a position, i preferably represents an integer from 1-2 in an adjacent Mᵢ if i=0 would result in two occurrences of Y being directly attached, or an occurrence of Y being directly attached to N.

In certain embodiments, Cy′ is a substituted or unsubstituted aryl or heteroaryl. In certain embodiments, Cy′ is directly attached to X. In certain embodiments, Cy′ is a substituted or unsubstituted bicyclic or heteroaryl ring, preferably both bicyclic and heteroaryl, such as benzothiophene, benzofuiran, benzopyrrole, benzopyridine, etc. In certain embodiments, Cy′ is a monocyclic aryl or heteroaryl ring substituted at least with a substituted or unsubstituted aryl or heteroaryl ring, e.g., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system.

In certain embodiments, X is selected from —C(═O)—, —C(═S)—, and —S(O₂)—.

In certain embodiments, Cy represents a substituted or unsubstituted non-aromatic carbocyclic or heterocyclic ring, i.e., including at least one sp³ hybridized atom, and preferably a plurality of sp³ hybridized atoms. In certain embodiments, Cy includes an amine within the atoms of the ring or on a substitutent of the ring, e.g., Cy is pyridyl, imidazolyl, pyrrolyl, piperidyl, pyrrolidyl, piperazyl, etc., and/or bears an amino substituent. In certain embodiments, Cy is a 5- to 7-membered ring. In certain embodiments, Cy is directly attached to N. In embodiments wherein Cy is a six-membered ring directly attached to N and bears an amino substituent at the 4 position of the ring relative to N, the N and amine substituents may be disposed trans on the ring.

In certain embodiments, substituents on Ar or Ar′ are selected from halogen, lower alkyl, lower alkenyl, aryl, heteroaryl, carbonyl, thiocarbonyl, ketone, aldehyde, amino, acylamino, cyano, nitro, hydroxyl, azido, sulfonyl, sulfoxido, sulfate, sulfonate, sulfamoyl, sulfonamido, phosphoryl, phosphonate, phosphinate, —(CH₂)palkyl, —(CH₂)palkenyl, —(CH₂)palkynyl, —(CH₂)paryl, —(CH₂)paralkyl, —(CH₂)pOH, —(CH₂)pO-lower alkyl, —(CH₂)pO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above, wherein p, individually for each occurence, represents an integer from 0 to 10, preferably from 0 to 5.

In certain embodiments, compounds useful in the present invention may be represented by general formula (XIII):

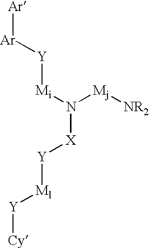

Formula XIII

wherein, as valence and stability permit,

Ar and Ar′ independently represent substituted or unsubstituted aryl or heteroaryl rings;

Y, independently for each occurrence, may be absent or represent —N(R)—, —O—, —S—, or —Se—;

X can be selected from —C(═O)—, —C(═S)—, —S(O₂)—, —S(O)—, —C(═NCN)—, —P(═O)(OR₂)—, and a methylene group optionally substituted with 1-2 groups such as lower alkyl, alkenyl, or alkynyl groups;

M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne, wherein some or all occurrences of M in M , form all or part of a cyclic structure;

R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;

Cy′ represents a substituted or unsubstituted aryl, heterocyclyl, heteroaryl, or cycloalkyl, including polycyclic groups;

j represents, independently for each occurrence, an integer from 0 to 10, preferably from 2 to 7;

i represents, independently for each occurrence, an integer from 0 to 5, preferably from 0 to 2; and

n, individually for each occurence, represents an integer from 0 to 10, preferably from 0 to 5.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Ar and Ar′ represent phenyl rings, e.g., unsubstituted or substituted with one or more groups including heteroatoms such as O, N, and S. In certain embodiments, at least one of Ar and Ar′ represents a phenyl ring. In certain embodiments, at least one of Ar and Ar′ represents a heteroaryl ring, e.g., a pyridyl, thiazolyl, thienyl, pyrimidyl, etc. In certain embodiments, Y and Ar′ are attached to Ar in a meta and/or 1,3-relationship.

In certain embodiments, Y is absent from all positions. In embodiments wherein Y is present in a position, i preferably represents an integer from 1-2 in an adjacent Mᵢ if i=0 would result in two occurrences of Y being directly attached, or an occurrence of Y being directly attached to N or NR₂.

In certain embodiments, Cy′ is a substituted or unsubstituted aryl or heteroaryl. In certain embodiments, Cy′ is directly attached to X. In certain embodiments, Cy′ is a substituted or unsubstituted bicyclic or heteroaryl ring, preferably both bicyclic and heteroaryl, such as benzothiophene, benzofuran, benzopyrrole, benzopyridine, etc. In certain embodiments, Cy′ is a monocyclic aryl or heteroaryl ring substituted at least with a substituted or unsubstituted aryl or heteroaryl ring, e.g., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system.

In certain embodiments, X is selected from —C(═O)—, —C(′S)—, and —S(O₂)—.

In certain embodiments, NR₂ represents a primary amine or a secondary or tertiary amine substituted with one or two lower alkyl groups, aryl groups, or aralkyl groups, respectively, preferably a primary amine.

In certain embodiments, substituents on Ar or Ar′ are selected from halogen, lower alkyl, lower alkenyl, aryl, heteroaryl, carbonyl, thiocarbonyl, ketone, aldehyde, amino, acylamino, cyano, nitro, hydroxyl, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)pOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above, wherein p, individually for each occurence, represents an integer from 0 to 10, preferably from 0 to 5.

In certain embodiments, compounds useful in the present invention may be represented by general formula (XIV):

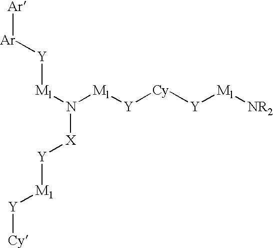

Formula XIV

wherein, as valence and stability permit,

Ar and Ar′ independently represent substituted or unsubstituted aryl or heteroaryl rings;

Y, independently for each occurrence, may be absent or represent —N(R)—, —O—, —S—, or —Se—;

X can be selected from —C(═O)—, —C(═S)—, —S(O₂)—, —S(O)—, —C(═NCN)—, —P(═O)(OR₂)—, and a methylene group optionally substituted with 1-2 groups such as lower alkyl, alkenyl, or alkynyl groups;

M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;

R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;

Cy and Cy′ independenly represent substituted or unsubstituted aryl, heterocyclyl, heteroaryl, or cycloalkyl, including polycyclic groups;

represents, independently for each occurrence, an integer from 0 to 5, preferably from 0 to 2; and

n, individually for each occurence, represents an integer from 0 to 10, preferably from 0 to 5.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Ar and Ar′ represent phenyl rings, e.g., unsubstituted or substituted with one or more groups including heteroatoms such as O, N, and S. In certain embodiments, at least one of Ar and Ar′ represents a phenyl ring. In certain embodiments, at least one of Ar and Ar′ represents a heteroaryl ring, e.g., a pyridyl, thiazolyl, thienyl, pyrimidyl, etc. In certain embodiments, Y and Ar′ are attached to Ar in a meta and/or 1,3-relationship.

In certain embodiments, Y is absent from all positions. In embodiments wherein Y is present in a position, i preferably represents an integer from 1-2 in an adjacent Mᵢ if i=0 would result in two occurrences of Y being directly attached, or an occurrence of Y being directly attached to N or NR₂.

In certain embodiments, Cy′ is a substituted or unsubstituted aryl or heteroaryl. In certain embodiments, Cy′ is directly attached to X. In certain embodiments, Cy′ is a substituted or unsubstituted bicyclic or heteroaryl ring, preferably both bicyclic and heteroaryl, such as benzothiophene, benzofuran, benzopyrrole, benzopyridine, etc. In certain embodiments, Cy′ is a monocyclic aryl or heteroaryl ring substituted at least with a substituted or unsubstituted aryl or heteroaryl ring, e.g., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system.

In certain embodiments, X is selected from —C(═O)—, —C(═S)—, and —S(0₂)-.

In certain embodiments, NR₂ represents a primary amine or a secondary or tertiary amine substituted with one or two lower alkyl groups, aryl groups, or aralkyl groups, respectively, preferably a primary amine.

In certain embodiments, Cy represents a substituted or unsubstituted non-aromatic carbocyclic or heterocyclic ring, i.e., including at least one sp³ hybridized atom, and preferably a plurality of sp³ hybridized atoms. In certain embodiments, Cy is directly attached to N and/or to NR₂. In certain embodiments, Cy is a 5- to 7-membered ring. In embodiments wherein Cy is a six-membered ring directly attached to N and bears an amino substituent at the 4 position of the ring relative to N, the N and amine substituents may be disposed trans on the ring.

In certain embodiments, substituents on Ar or Ar′ are selected from halogen, lower alkyl, lower alkenyl, aryl, heteroaryl, carbonyl, thiocarbonyl, ketone, aldehyde, amino, acylamino, cyano, nitro, hydroxyl, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)pOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above, wherein p, individually for each occurence, represents an integer from 0 to 10, preferably from 0 to 5.

In certain embodiments, compounds useful in the subject methods include compounds represented by general forumla (XV):

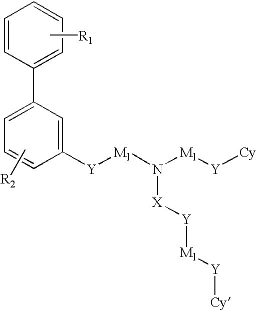

Formula XV

wherein, as valence and stability permit,

Cy′ represents a substituted or unsubstituted aryl or heteroaryl ring, including polycyclics;

Y, independently for each occurrence, may be absent or represent —N(R)—, —O—, —S—, or —Se—;

X can be selected from —C(═O)—, —C(═S)—, —S(O₂)—, —S(O)—, —C(═NCN)—, —P(═O)(OR₂)—, and a methylene group optionally substituted with 1-2 groups such as lower alkyl, alkenyl, or alkynyl groups;

M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;

R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;

R₁ and R₂ represent, independently and as valency permits, from 0-5 substituents on the ring to which it is attached, selected from halogen, lower alkyl, lower alkenyl, aryl, heteroaryl, carbonyl, thiocarbonyl, ketone, aldehyde, amino, acylamino, amido, amidino, cyano, nitro, hydroxyl, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above;

Cy represents substituted or unsubstituted aryl, heterocyclyl, heteroaryl, or cycloalkyl, including polycyclic groups;

i represents, independently for each occurrence, an integer from 0 to 5, preferably from 0 to 2; and

p and n, individually for each occurrence, represent integers from 0 to 10, preferably from 0 to 5.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Cy′ represents a substituted or unsubstituted bicyclic or heterocyclic ring system, preferably both bicyclic and heteroaryl, such as benzothiophene, benzofuran, benzopyrrole, benzopyridine, etc. In certain embodiments, Cy′ is directly attached to X. In certain embodiments, Cy′ is a monocyclic aryl or heteroaryl ring substituted at least with a substituted or unsubstituted aryl or heteroaryl ring, e.g., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system.

In certain embodiments, Y is absent from all positions. In embodiments wherein Y is present in a position, i preferably represents an integer from 1-2 in an adjacent Mᵢ if i=0 would result in two occurrences of Y being directly attached, or an occurrence of Y being directly attached to N.

In certain embodiments, X is selected from —C(═O)—, —C(═S)—, and —S(O₂)—.

In certain embodiments, Cy represents a substituted or unsubstituted non-aromatic carbocyclic or heterocyclic ring, i.e., including at least one Sp³ hybridized atom, and preferably a plurality of sp3 hybridized atoms. In certain embodiments, Cy includes an amine within the atoms of the ring or on a substitutent of the ring, e.g., Cy is pyridyl, imidazolyl, pyrrolyl, piperidyl, pyrrolidyl, piperazyl, etc., and/or bears an amino substituent. In certain embodiments, Cy is directly attached to N. In certain embodiments, Cy is a 5- to 7-membered ring. In embodiments wherein Cy is a six-membered ring directly attached to N and bears an amino substituent at the 4 position of the ring relative to N, the N and amine substituents may be disposed trans on the ring.

In certain embodiments, R₁ and R₂ represent, independently and as valency permits, from 0-5 substituents on the ring to which it is attached, selected from halogen, lower alkyl, lower alkenyl, carbonyl, thiocarbonyl, ketone, aldehyde, amino, acylamino, cyano, nitro, hydroxyl, azido, sulfonyl, sulfoxido, sulfate, sulfonate, sulfamoyl, sulfonamido, phosphoryl, phosphonate, phosphinate, —(CH₂)palkyl, —(CH₂)palkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above, wherein p, individually for each occurence, represents an integer from 0 to 10, preferably from 0 to 5.

In certain embodiments, compounds useful in the present invention may be represented by general formula (XVI):

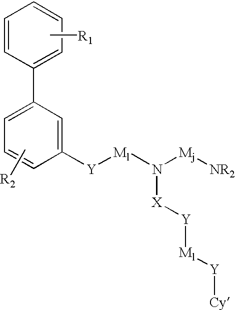

Formula XVI

wherein, as valence and stability permit,

Cy′ represents a substituted or unsubstituted aryl or heteroaryl ring, including polycyclics;

Y, independently for each occurrence, may be absent or represent —N(R)—, —O—, —S—, or —Se—;

X can be selected from —C(═O)—, —C(═S)—, —S(O₂)—, —S(O)—, —C(═NCN)—, —P(═O)(OR₂)—, and a methylene group optionally substituted with 1-2 groups such as lower alkyl, alkenyl, or alkynyl groups;

M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;

R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;

R₁ and R₂ represent, independently and as valency permits, from 0-5 substituents on the ring to which it is attached, selected from halogen, lower alkyl, lower alkenyl, aryl, heteroaryl, carbonyl, thiocarbonyl, ketone, aldehyde, amino, acylamino, amido, amidino, cyano, nitro, hydroxyl, azido, sulfonyl, sulfoxido, sulfate, sulfonate, sulfamoyl, sulfonamido, phosphoryl, phosphonate, phosphinate, —(CH₂)palkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above;

Cy′ representsa substituted or unsubstituted aryl, heterocyclyl, heteroaryl, or cycloalkyl, including polycyclic groups;

j represents, independently for each occurrence, an integer from 0 to 10, preferably from 2 to 7;

i represents, independently for each occurrence, an integer from 0 to 5, preferably from 0 to 2; and

p and n, individually for each occurrence, represent integers from 0 to 10, preferably from 0 to 5.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Cy′ represents a substituted or unsubstituted bicyclic or heterocyclic ring system, preferably both bicyclic and heteroaryl, such as benzothiophene, benzofuran, benzopyrrole, benzopyridine, etc. In certain embodiments, Cy′ is directly attached to X. In certain embodiments, Cy′ is a monocyclic aryl or heteroaryl ring substituted at least with a substituted or unsubstituted aryl or heteroaryl ring, e.g., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system.

In certain embodiments, Y is absent from all positions. In embodiments wherein Y is present in a position, i preferably represents an integer from 1-2 in an adjacent Mᵢ if i=0 would result in two occurrences of Y being directly attached, or an occurrence of Y being directly attached to N or NR₂.

In certain embodiments, X is selected from —C(═O)—, —C(═S)—, and —S(O₂)—.

In certain embodiments, NR₂ represents a primary amine or a secondary or tertiary amine substituted with one or two lower alkyl groups, aryl groups, or aralkyl groups, respectively, preferably a primary amine.

In certain embodiments, R₁ and R₂ represent, independently and as valency permits, from 0-5 substituents on the ring to which it is attached, selected from halogen, lower alkyl, lower alkenyl, carbonyl, thiocarbonyl, ketone, aldehyde, amino, acylamino, cyano, nitro, hydroxyl, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alky l —(CH₂)ₚNR-lower alkenyl, —NR(CH₂)ₙR, and protected forms of the above, wherein p, individually for each occurence, represents an integer from 0 to 10, preferably from 0 to 5.

In certain embodiments, compounds useful in the present invention may be represented by general formula (XVII):

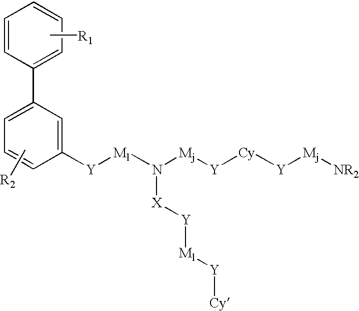

Formula XVII

wherein, as valence and stability permit,

Cy′ represents a substituted or unsubstituted aryl or heteroaryl ring, including polycyclics;

Y, independently for each occurrence, may be absent or represent —N(R)—, —O—, —S—, or —Se—;

X can be selected from —C(═O)—, —C(═S)—, —S(O₂)—, —S(O)—, —C(═NCN)—, —P(═O)(OR₂)—, and a methylene group optionally substituted with 1-2 groups such as lower alkyl, alkenyl, or alkynyl groups;

M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;

R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;

Cy represents substituted or unsubstituted aryl, heterocyclyl, heteroaryl, or cycloalkyl, including polycyclic groups;

i represents, independently for each occurrence, an integer from 0 to 5, preferably from 0 to 2; and

n and p, individually for each occurrence, represent integers from 0 to 10, preferably from 0 to 5.

In certain embodiments, M represents, independently for each occurrence, a substituted or unsubstituted methylene group, such as —CH₂—, —CHF—, —CHOH—, —CH(Me)—, —C(═O)—, etc.

In certain embodiments, Cy′ represents a substituted or unsubstituted bicyclic or heteroaryl ring system, preferably both bicyclic and heteroaryl, e.g., benzothiophene, benzofuran, benzopyrrole, benzopyridyl, etc. In certain embodiments, Cy′ is directly attached to X. In certain embodiments, Cy′ is a monocyclic aryl or heteroaryl ring substituted at least with a substituted or unsubstituted aryl or heteroaryl ring, e.g., forming a biaryl system. In certain embodiments, Cy′ includes two substituted or unsubstituted aryl or heteroaryl rings, e.g., the same or different, directly connected by one or more bonds, e.g., to form a biaryl or bicyclic ring system.

In certain embodiments, Y is absent from all positions. In embodiments wherein Y is present in a position, i preferably represents an integer from 1-2 in an adjacent Mᵢ if i=0 would result in two occurrences of Y being directly attached, or an occurrence of Y being directly attached to N or NR₂.

In certain embodiments, X is selected from —C(═O)—, —C(═S)—, and —S(O₂)—.

In certain embodiments, NR₂ represents a primary amine or a secondary or tertiary amine substituted with one or two lower alkyl groups, aryl groups, or aralkyl groups, respectively, preferably a primary amine.

In certain embodiments, Cy represents a substituted or unsubstituted non-aromatic carbocyclic or heterocyclic ring, i.e., including at least one sp³ hybridized atom, and preferably a plurality of sp³ hybridized atoms. In certain embodiments, Cy is directly attached to N and/or to NR₂. In certain embodiments, Cy is a 5- to 7-membered ring. In embodiments wherein Cy is a six-membered ring directly attached to N and bears an amino substituent at the 4 position of the ring relative to N, the N and amine substituents may be disposed trans on the ring.

In certain embodiments, R₁ and R₂ represent, independently and as valency permits, from 0-5 substituents on the ring to which it is attached, selected from halogen, lower alkyl, lower alkenyl, carbonyl, thiocarbonyl, ketone, aldehyde, amino, acylamino, cyano, nitro, hydroxyl, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above, wherein p, individually for each occurence, represents an integer from 0 to 10, preferably from 0 to 5.

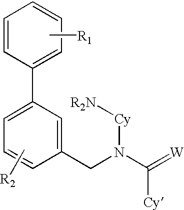

In certain embodiments, a subject compound has the structure of Formula XVIII: wherein, as valence and stability permit,

Cy represents a substituted or unsubstituted heterocyclyl or cycloalkyl;

Cy′ is a substituted or unsubstituted aryl or heteroaryl ring;

W is O or S;

R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;

R₁ and R₂ represent, independently and as valency permits, from 0-5 substituents on the ring to which it is attached, selected from halogen, lower alkyl, lower alkenyl, aryl, heteroaryl, carbonyl, thiocarbonyl, ketone, aldehyde, amino, acylamino, amido, amidino, cyano, nitro, hydroxyl, azido, sulfonyl, sulfoxido, sulfate, sulfonate, sulfamoyl, sulfonamido, phosphoryl, phosphonate, phosphinate, —(CH₂)ₚalkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above;

n and p, individually for each occurrence, represent integers from 0 to 10.

In certain embodiments, Cy′ represents a substituted or unsubstituted bicyclic or heteroaryl ring system, preferably both bicyclic and heteroaryl, e.g., benzothiophene, benzofuran, benzopyrrole, benzopyridyl, etc. In certain embodiments, Cy′ is directly attached to X.

In certain embodiments, NR₂ represents a primary amine or a secondary or tertiary amine substituted with one or two lower alkyl groups, aryl groups, or aralkyl groups, respectively, preferably a primary amine.

In certain embodiments, Cy represents a substituted or unsubstituted saturated carbocyclic or heterocyclic ring, i.e., composed of a plurality of sp³ hybridized atoms. In certain embodiments, Cy is a 5- to 7-membered ring. In embodiments wherein Cy is a six-membered ring directly attached to N and bears an amino substituent at the 4 position of the ring relative to N, the N and amine substituents may be disposed trans on the ring.

In certain embodiments, R₁ and R₂ represent, independently and as valency permits, from 0-5 substituents on the ring to which it is attached, selected from halogen, lower alkyl, lower alkenyl, car bonyl, thiocarbonyl, ketone, aldehyde, amino, acylamino, cyano, nitro, hydroxyl, sulfonyl, sulfoxido, sulfate, sulfonate, sulfamoyl, sulfonamido, —(CH₂)ₚalkyl, —(CH₂)ₚalkenyl, —(CH₂)ₚalkynyl, —(CH₂)ₚaryl, —(CH₂)ₚaralkyl, —(CH₂)ₚOH, —(CH₂)ₚO-lower alkyl, —(CH₂)ₚO-lower alkenyl, —O(CH₂)ₙR, —(CH₂)ₚSH, —(CH₂)ₚS-lower alkyl, —(CH₂)ₚS-lower alkenyl, —S(CH₂)ₙR, —(CH₂)ₚN(R)₂, —(CH₂)ₚNR-lower alkyl, —(CH₂)ₚNR-lower alkenyl, —NR(CH₂)ₙR, and protected forms of the above.

In certain embodiments, a subject compound has a structure of Formula XIX:

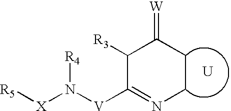

wherein, as valence and stability permit,

U represents a substituted or unsubstituted aryl or heteroaryl ring fused to the nitrogen-containing ring;

V represents a lower alkylene group, such as methylene, 1,2-ethylene, 1,1-ethylene, 1,1-propylene, 1,2-propylene, 1,3-propylene, etc.;

W represents S or O, preferably O;

X represents C═O, C═S, or SO₂;

R₃ represents substituted or unsubstituted aryl, heteroaryl, lower alkyl, lower alkenyl, lower alkynyl, carbocyclyl, carbocyclylalkyl, heterocyclyl, heterocyclylalkyl, aralkyl, or heteroaralkyl;

R₄ represents substituted or unsubstituted aralkyl or lower alkyl, such as phenethyl, benzyl, or aminoalkyl, etc.;

R₅ represents substituted or unsubstituted aryl, heteroaryl, aralkyl, or heteroaralkyl, including polycyclic aromatic or heteroaromatic groups.

In certain embodiments, U represents a phenyl ring fused to the nitrogen-containing ring.

In certain embodiments, R₃ is selected from substituted or unsubstituted aryl, heteroaryl, lower alkyl, lower alkenyl, aralkyl, and heteroaralkyl.

In certain embodiments, R₄ is an unsubstituted lower alkyl group, or is a lower alkyl group substituted with a secondary or tertiary amine.

In certain embodiments, R₅ is selected from substituted or unsubstituted phenyl or naphthyl, or is a diarylalkyl group, such as 2,2-diphenylethyl, diphenylmethyl, etc.

Moreover, the subject methods can be performed on cells which are provided in culture (in vitro), or on cells in a whole animal (in vivo). See, for example, PCT publications WO 95/18856 and WO 96/17924 (the specifications of which are expressly incorporated by reference herein).

VI. Testing for Biological Activity

While many bioassays have been used to demonstrate hedgehog activity, the C3H10T1/2 cell line provides a simple system for assessing hedgehog function without the complication of having to work with primary cell cultures or organ explants. The mouse embryonic fibroblast line C3HlOT I/2 is a mesenchymal stem cell line that, under defined conditions, can differentiate into adipocytes, chondrocytes, and bone osteoblasts (Taylor, S. M., and Jones, P. A., Cell 17: 771-779 (1979) and Wang, E. A., et al., Growth Factors 9: 57-71 (1993)). Bone morphogenic proteins drive the differentiation of C3H10T 1/2 cells into the bone cell lineage and alkaline phosphatase induction has been used as a marker for this process (Wang et al., supra). Shh has a similar effect on C3H10T I/2 cells (Kinto, N. et al., FEBS Letts. 404: 319-323 (1997)) and we routinely use the alkaline phosphatase induction by Shh as a quantitative measure of its in vitro potency. Shh treatment also produces a dose-dependent increase in gli-1 and ptc-1 expression, which can be readily detected by a PCR-based analysis.

We found that hedgehog protein can upregulate fibroblast expression of angiogenic growth factors, including VEGF121, VEGF165, VEGF189, Ang-1, and Ang-2 (Example 4). Thus, the procedure outlined in Example 4 provides a new method of measuring the in vitro angiogenic potential of hedgehog. Without wishing to be bound by any particular theory, this upregulation may explain the mechanism whereby hedgehog exerts its angiogenic effect.

Similarly, this cell line provides a simple bioassay to test the agonistic or antagonistic properties of the hedgehog therapeutics of the present invention. In preferred embodiments, agonists would be expected to induce alkaline phosphatase in CSH10T1/2 cells. In other embodiments, antagonists would be expected to inhibit the induction of alkaline phosphatase by exogenous hedgehog.

Further, persons having ordinary skill in the art will recognize means for determining if the hedgehog agents used in the present methods are efficacious in vivo. For instance, clinicians have available to them a variety of non-invasive tests such as echograms, electrocardiograms, CAT scans, MRI to determine vascular and cardiac functioning. Other methods include angiography and other more invasive physiological testing methods. For patients with neuropathies, nerve conduction velocity tests may be routinely performed. To test for the anti-angiogenic function of hedgehog antagonists, persons of ordinary skill in the art way use a variety of imaging methods such as CAT and MRI scans, as well as more invasive tests to look at blood chemistry and tumor metabolism.

VII. Subjects for Treatment

As a general matter, the methods of the present invention may be utilized for any mammalian subject needing modulation of angiogenesis. Mammalian subjects which may be treated according to the methods of the invention include, but are not limited to, human subjects or patients. In addition, however, the invention may be employed in the treatment of domesticated mammals which are maintained as human companions (e.g., dogs, cats, horses), which have significant commercial value (e.g., dairy cows, beef cattle, sporting animals), which have significant scientific value (e.g., captive or free specimens of endangered species), or which otherwise have value. In addition, as a general matter, the subjects for treatment with the methods of the present invention need not present indications for treatment with the agents of the invention other than those indications associated with need for modulation of angiogenesis. That is, the subjects for treatment are expected to be otherwise free of indications for treatment with the hedgehog therapeutic agents of the invention.

One of ordinary skill in the medical or veterinary arts is trained to recognize subjects which may need modulation of angiogenesis . In particular, clinical and non-clinical trials, as well as accumulated experience, relating to the presently disclosed and other methods of treatment, are expected to inform the skilled practitioner in deciding whether a given subject is in need of modulation and whether any particular treatment is best suited to the subject's needs, including treatment according to the present invention.

VIII. Utilities, Formulations and Methods of Treatment

A. General

We show that hedgehog receptor (ptcl) is normally expressed in the vasculature. We used a mouse which carries the lacZ reporter gene under the control of the endogenous ptc 1 promotor to determine the expression of ptcl in normal adult animals (Example 1). We further determined that mice injected with hedgehog protein for 3 days showed no obvious physical or behavioral differences compared to vehicle-treated or untreated littermates. The vascular and cardiovascular staining pattern for ptcl seen in normal animals intensifies significantly in animals injected with increasing doses of hedgehog protein. Our data show that systemic administration of hedgehog can induce ptcl upregulation and indicate that these vascular tissues are responsive to hedgehog protein.

We further determined that hedgehog induces neovascularization in a corneal model of angiogenesis (Example 3) as well as a matrigel plug model of angiogenesis (Example 2) . We further found that there was a striking qualitative difference in the appearance of vessels induced by hedgehog compared to VEGF. VEGF induced a fine mesh of capillaries which are short tortous sprouts from the extended branches of the preexisting limbus vessels at the base of the eye. In contrast, hedgehog induced much larger vessels which extended all the way to the pellet and contained numerous anastamoses between the venous and arterial circulation

Moreover, we employed surgical ligation of the femoral artery and removal of a segment of the artery distal to the ligation in mice to induce limb ischemia (Example 5). We found that hedgehog improves recovery from such ischemic limb injury.

In yet another clinically relevant animal model, we placed an ameroid constrictor around the left circumflex coronary artery of pigs. We determined that hedgehog protein or gene therapy can also improve these measures of cardiac perfusion, viability and function following ischemia in this model (Example 6). We determined that hedgehog protein is overexpressed in several human gastrointestinal tumor cell lines compared to normal human gastrointestinal epithelial cells or fibroblasts (Example 7) and that inhibition of hedgehog using, for example, anti-hedgehog blocking antibody, may decrease tumor growth rate and/or tumor angiogenesis (Example 7).

Accordingly, the methods of this invention may employ hedgehog therapeutics or biologically active portions thereof, to promote angiogenesis, such as, to repair damage of myocardial tissue as a result of myocardial infarction. Such methods may also include the repair of the cardiac vascular system after ischemia including the growth of collateral vasculature. Methods utilizing hedgehog therapeutics may be employed to stimulate the growth of transplanted tissue and collateral vasculature where coronary bypass surgery is performed. Methods may also treat damaged vascular tissue as a result of coronary artery disease and peripheral or central nervous system vascular disease or ischemia.

Methods of the invention may also promote wound healing, particularly to re-vascularize damaged tissues or stimulate collateral blood flow during ischemia and where new capillary angiogenesis is desired. Other methods of the invention may be employed to treat full-thickness wounds such as dermal ulcers, including pressure sores, venous ulcers, and diabetic ulcers. In addition, methods employing hedgehog therapeutics may be employed to treat full-thickness bums and injuries where a skin graft or flap is used to repair such burns and injuries. Such hedgehog therapeutics may also be employed for use in plastic surgery, for example, for the repair of lacerations, burns, or other trauma. In urology, methods of the invention may assist in recovery of erectile function. In the field of female reproductive health, methods of the invention may assist in the modulation of menstruation, ovulation, endometrial lining formation and maintanence, and placentation.

Since angiogenesis is important in keeping wounds clean and non-infected, methods may be employed in association with surgery and following the repair of cuts. They may also be employed for the treatment of abdominal wounds where there is a high risk of infection. Methods using hedgehog therapeutics described herein may be employed for the promotion of endothelialization in vascular graft surgery. In the case of vascular grafts using either transplanted or synthetic material, hedgehog therapeutics can be applied to the surface of the graft or at the junction to promote the growth of vascular smooth muscle and adventitial cells in conjunction with endothelial cells.

Methods of the invention may also be employed to coat artificial prostheses or natural organs which are to be transplanted in the body to minimize rejection of the transplanted material and to stimulate vascularization of the transplanted materials and may also be employed for vascular tissue repair, for example, that occurring during arteriosclerosis and required following balloon angioplasty where vascular tissues are damaged. Specifically, methods of the invention may be employed to promote recovery from arterial wall injury and thereby inhibit restenosis.

Nucleic acid sequences encoding hedgehog therapeutics may also be employed for in vitro purposes related to scientific research, synthesis of DNA and manufacture of DNA vectors, and for the production of diagnostics and therapeutics to treat human disease. For example. methods of the invention may involve in vitro culturing of vascular smooth muscle cells, fibroblasts, hematopoietic cells, muscle, myotendonous junction, bone or cartilage-derived cells and other mesenchymal cells, where a hedgehog therapeutic is added to the conditional medium in a concentration from 10 ng/ml to 20 ug/ml.

Antagonistic hedgehog therapeutics may be employed to limit angiogenesis necessary for solid tumor metastasis. The identification of antagonists can be used for the generation of certain inhibitors of vascular endothelial growth factor. Since angiogenesis and neovascularization are essential steps in solid tumor growth, inhibition of angiogenic activity of the vascular endothelial growth factor is very useful to prevent the further growth, retard, or even regress solid tumors. Gastrointestinal tumors and gliomas are also a type of neoplasia which may be treated with the antagonists of the present invention.

In addition to these disorders, the antagonists may also be employed to treat retinopathy associated with diabetes, rheumatoid arthritis, osteoarthritis, macular degeneration, glaucoma, Keloid formation, ulcerative colitis, Krohn's disease, psoriasis, and other conditions caused are exacerbated by increased angiogenic activity. The antagonists may be employed in a composition with a pharmaceutically acceptable carrier, e.g., as described herein.

These therapeutic agents may be administered by any route which is compatible with the particular agent employed. The hedgehog therapeutic agents of the invention may be provided to an individual by any suitable means, preferably directly (e.g., locally, as by injection or topical administration to a tissue locus) or systemically (e.g., parenterally or orally). Where the agent is to be provided parenterally, such as by intravenous, intraarterial, subcutaneous, or intramuscular, administration, the agent preferably comprises part of an aqueous solution. The solution is physiologically acceptable so that in addition to delivery of the desired agent to the subject, the solution does not otherwise adversely affect the subject's electrolyte and/or volume balance. The aqueous medium for the hedgehog therapeutic may comprise normal physiologic saline (e.g., 9.85% NaCl, 0.15M, pH 7-7.4).

The hedgehog therapeutics are preferably administered as a sterile pharmaceutical composition containing a pharmaceutically acceptable carrier, which may be any of the numerous well known carriers, such as water, saline, phosphate buffered saline, dextrose, glycerol, ethanol, and the like, or combinations thereof. The compounds of the present invention may be used in the form of pharmaceutically acceptable salts derived from inorganic or organic acids and bases. Included among such acid salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, tris(hydroxymethyl)methylamine and salts with amino acids such as arginine, lysine, and so forth. Also, the basic nitrogen-containing groups can be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

Pharmaceutical compositions of hedgehog therapeutics comprise any of the compounds of the present invention, or pharmaceutically acceptable derivatives thereof, together with any pharmaceutically acceptable carrier. The term “carrier” as used herein includes acceptable adjuvants and vehicles. Pharmaceutically acceptable carriers that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

According to this invention, the pharmaceutical compositions may be in the form of a sterile injectable preparation, for example a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as do natural pharmaceutically-acceptable oils, such as olive oil or castor oil, especially in their polyoxyethylated versions. These oil solutions or suspensions may also contain a long-chain alcohol diluent or dispersant.

Controlled release administration of a particular hedgehog therapeutic may be useful. For example, the therapeutic may be administered using intravenous infusion, an implantable osmotic pump, a transdermal patch, liposomes, or other modes of administration. In one embodiment, a pump may be used [Langer et al., eds., Medical Applications of Controlled Release, CRC Pres., Boca Raton, Fla. (1974); Sefton, CRC Crit. Ref. Biomed. Eng., 14:201 (1987); Buchwald et al., Surgery, 88:507 (1980); Saudek et al., N. Engl. J. Med., 321:574 (1989)]. In another embodiment, polymeric materials can be used [see, Langer, 1974, supra; Sefton, 1987, supra; Smolen et al., eds., Controlled Drug Bioavailability, Drug Product Design and Performance, Wiley, N.Y. (1984); Ranger et al., J. Macromol. Sci. Rev. Macromol. Chem., 23:61 (1983); see also Levy et al., Science, 228:190 (1985); During et al., Ann. Neurol., 25:351 (1989); Howard et al., J. Neurosurg., 71:105 (1989)]. In yet another embodiment, a controlled release system can be placed in proximity of the therapeutic target, e.g., a tumor, thus requiring only a fraction of the systemic dose [see. e.g., Goodson, in Medical Applications of Controlled Release, vol. 2, pp. 115-138 (1984)]. Other controlled release systems are discussed in the review by Langer, Science, 249:1527-1533 (1990). In another embodiment, the therapeutic compound can be delivered in a vesicle, in particular a liposome (see Langer, 1990, supra); Treat et al., in Liposomes in the Therapy of Infectious Disease and Cancer, Lopez-Berestein and Fidler (eds.), Liss, New York, pp. 353-365 (1989); Lopez-Berestein, pp. 317-327; see generally id.).

B. Oral Delivery

Contemplated for use herein are oral solid dosage forms, which are described generally in Martin, Chapter 89, 1990, supra, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, in Mode r n Pharmaceutics, Chapter 10, Banker and Rhodes ed., (1979), herein incorporated by reference. In general, the formulation will include the therapeutic (or chemically modified form), and inert ingredients which allow for protection against the stomach environment, and release of the biologically active material in the intestine.

For the protein (or derivative) the location of release may be the stomach, the small intestine (the duodenum, the jejunem, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the protein (or derivative) or by release of the biologically active material beyond the stomach environment, such as in the intestine. To ensure full gastric resistance, a coating impermeable to at least pH. 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films. A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multiparticulates in the form of granules or pellets of particle size about 1 mm. The formulation of the material for capsule administration could also be as a powder, lightly compressed plugs or even as tablets. The therapeutic could be prepared by compression. Colorants and flavoring agents may all be included. For example, the protein (or derivative) may be formulated (such as by liposome or microsphere encapsulation) and then further contained within an edible product, such as a refrigerated beverage containing colorants and flavoring agents. One may dilute or increase the volume of the therapeutic with an inert material. These diluents could include carbohydrates, especially mannitol, alpha -lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell. Disintegrants may be included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants. Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic. An antifrictional agent may be included in the formulation of the therapeutic to prevent sticking during the formulation process. Lubricants may be used as a layer between the therapeutic and the die wall, and these can include but are not limited to: stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, and Carbowax 4000 and 6000. Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment, a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios. Additives which potentially enhance uptake of the protein (or derivative) are for instance the fatty acids oleic acid, linoleic acid and linolenic acid.

C. Pulmonary Delivery

Also contemplated herein is pulmonary delivery of the present proteins (or derivatives thereof). The protein (or derivative) is delivered to the lungs of a mammal while inhaling and traverses across the lung epithelial lining to the blood-stream. Other reports of this include Adjei et al., Pharmaceutical Research, 7(6):565-569 (1990); Adjei et al., International Journal of Pharmaceutics, 63:135-144 (1990) (leuprolide acetate); Braquet et al., Journal of Cardiovascular Pharmacology, 13(suppl. 5):143-146 (1989) (endothelia-1); Hubbard et al., Annals of Internal Medicine, 3(3):206-212 (1989) ( alpha 1-antitrypsin); Smith et al., J. Clin. Invest., 84:1145-1146 (1989) ( alpha 1-proteinase); Os wein et al., “Aerosolization of Proteins”, Proceedings of Symposium on Respiratory Drug Delivery II, Keystone, Colo., (March 1990) (recombinant human growth hormone); Debs et al., J. Immunol., 140:3482-3488 (1988) (interferon-gamma and tumor necrosis factor alpha) and Platz et al., U.S. Pat. No. 5,284,656 (granulocyte colony stimulating factor). Contemplated for use in the practice of this invention are a wide range of mechanical devices designed for pulmonary delivery of therapeutic products, including but not limited to nebulizers, metered-dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered-dose inhaler, manufactured by Glaxo Inc., Research Triangle Park, N.C.; and the Spinhaler powder inhaler, manufactured by Fisons Corp., Bedford, Mass. All such devices require the use of formulations suitable for the dispensing of protein (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified protein may also be prepared in different formulations depending on the type of chemical modification or the type of device employed.

Formulations suitable for use with a nebulizer, either jet or ultrasonic, will typically comprise protein (or derivative) dissolved in water at a concentration of about 0.1 to 25 mg of biologically active protein per ml of solution. The formulation may also include a buffer and a simple sugar (e.g., for protein stabilization and regulation of osmotic pressure). The nebulizer formulation may also contain a surfactant, to reduce or prevent surface induced aggregation of the protein caused by atomization of the solution in forming the aerosol.

Formulations for use with a metered-dose inhaler device will generally comprise a finely divided powder containing the protein (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing protein (or derivative) and may also include a bulking agent, such as lactose, sorbitol, sucrose, or mannitol in amounts which facilitate dispersal of the powder from the device, e.g., 50 to 90% by weight of the formulation. The protein (or derivative) should most advantageously be prepared in particulate form with an average particle size of less than 10 mum (or microns), most preferably 0.5 to 5 mum, for most effective delivery to the distal lung.

D. Dosages

For all of the above molecules, as further studies are conducted, information will emerge regarding appropriate dosage levels for treatment of various conditions in various patients, and the ordinary skilled worker, considering the therapeutic context, age and general health of the recipient, will be able to ascertain the proper dosage. Generally, for injection or infusion, dosage will be between 0.01 mu. g of biologically active protein/kg body weight, (calculating the mass of the protein alone, without chemical modification), and 10 mg/kg (based on the same). The dosing schedule may vary, depending on the circulation half-life of the protein or derivative used, whether the polypeptide is delivered by bolus dose or continuous infusion, and the formulation used.

E. Administration with Other Compounds

For therapy associated with modulating angiogenesis, one may administer the present hedgehog therapeutics (or derivatives) in conjunction with one or more pharmaceutical compositions used for treating other clinical complications of the need for angiogenic modulation, such as those used for treatment of cancer (e.g., chemotherapeutics), cachexia, high blood pressure, high cholesterol, and other adverse conditions. Administration may be simultaneous or may be in seriatim. Similarly, one may administer more than one hedgehog therapeutic (or derivatives), having the same or differing mode of action, to attain an additive or synergistic effect on angiogenesis.

F. Nucleic Acid-Based Therapeutic Treatment

Nucleic acid sequences encoding an antagonisitic hedgehog therapeutic could be introduced into human tumor or blood vessel cells to develop gene therapy. Similarly, nucleic acid sequences encoding an agonistic hedgehog therapeutic could be introduced into human cells as a gene therapy based treatment.

In one embodiment, a nucleic acid sequence encoding a hedgehog therapeutic is introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral geries, are preferred. Defective virus is not infective after introduction into a cell. Use of defective viral vectors allows for administration to cells in a specific, localized area, without concern that the vector can infect other cells. Thus, adipose tissue can be specifically targeted. Examples of particular vectors include, but are not limited to, a defective herpes virus 1 (HS V 1) vector [Kaplitt et al., Molec. Cell. Neurosci., 2:320-330 (1991)], an attenuated adenovirus vector, such as the vector described by Stratford-Perricaudet et al., J. Clin. Invest., 90:626-630 (1992), and a defective adeno-associted virus vector [Samulski et al., J. Virol., 61:3096-3101 (1987); Samulski et al., J. Virol., 63:3822-3828 (1989)]. In another embodiment, the nucleic acid can be introduced in a retroviral vector, e.g., as described in Anderson et al., U.S. Pat. No. 5,399,346; Mann et al., Cell, 33:153 (1983); Temin et al., U.S. Pat. No. 4,650,764; Temin et al., U.S. Pat. No. 4,980,289; Markowitz et al., J. Virol., 62:1120 (1988); Temin et al., U.S. Pat. No. 5,124,263; International Patent Publication No. WO 95/07358, published Mar. 16, 1995, by Dougherty et al.; and Kuo et al., Blood, 82:845 (1993).

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker [Felgner et al., Proc. Natl. Acad. Sci. USA, 84:7413-7417 (1987); see Mackey et al., Proc. Natl. Acad. Sci. USA, 85:8027-8031 (1988)]. The use of cationic lipids may promote encapsulation of negatively charged nucleic acids, and also promote fusion with negatively charged cell membranes [Felgner et al., Science, 337:387-388 (1989)]. The use of lipofection to introduce exogenous genes into specific organs in vivo has certain practical advantages. Molecular targeting of liposomes to specific cells represents one area of benefit. It is clear that directing transfection to particular cell types would be particularly advantageous in a tissue with cellular heterogeneity, such as the pancreas, liver, kidney, and brain. Lipids may be chemically coupled to other molecules for the purpose of targeting (see Mackey et al., 1988, supra). Targeted peptides, e.g., hormones or neurotransmitters, and proteins such as antibodies, or non-peptide molecules could be coupled to liposomes chemically.

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wu et al., J. Biol. Chem., 267:963-967 (1992); Wu et al., J. Biol. Chem., 263:14621-14624 (1988); Hartmut et al., Canadian Patent Application No. 2,012,311, filed Mar. 15, 1990).

It is also possible to introduce the vector in vivo in conjuction with a catheter or other device. See Vale et al., 1999: Komowski et al., 2000.

H. Diagnostics

A diagnostic method useful in the present invention comprises examining a cellular sample or medium by means of an assay including an effective amount of an antagonist to a hedgehog protein, such as an anti-hedgehog antibody homolog, preferably an affinity-purified polyclonal antibody, and more preferably a mAb. In addition, it is preferable for the anti-hedgehog antibody molecules used herein be in the form of Fab, Fab′, F(ab)2 or F(v) portions or whole antibody molecules. As previously discussed, patients capable of benefiting from this method include those suffering from cancer or other conditions where abnormal angiogenesis is a characteristic or factor. Methods for isolating hedgehog protein and inducing anti-hedgehog antibodies and for determining and optimizing the ability of anti-hedgehog antibodies to assist in the examination of the target cells are all well-known in the art.

The present invention will be illustrated by the following, non-limiting examples. These are described in further detail in the pending publication, Pola et al., 2001, Nature Medicine, incorporated herein.

### EXAMPLE 1

Hedgehog Responsive Cells in Normal Vasculature

The Expression of Hedgehog Receptor in Normal Vasculature

The hedgehog receptor which is coupled directly to the hedgehog signalling pathway is patched 1 (ptcl). In addition to being the primary hedgehog receptor in the signalling pathway, ptcl gene expression is also induced by signalling through the hedgehog pathway. The expression of the ptcl gene in cells can thus indicate that the cell is potentially responsive to hedgehog proteins and can also show that the cell is in the process of responding to hedgehog stimulation. We used a mouse which carries the lacZ reporter gene under the control of the endogenous ptcl promotor to determine the expression of ptcl in normal adult animals Ptcl-lacZ mice carry a non disruptive insertion of the lacZ reporter gene containing a nuclear localization signal upstream of the ptcl coding region. LacZ expression corresponds to ptcl expression (Goodrich et al., 1997; M. Scott, Ontogeny, personal communication). Ptcl expression does not appear to be altered by LacZ insertion and expression corresponds to ptcl expression in embryos (M. Scott, Ontogeny, personal communication). Heterozygous Ptcl-lacZ mice and their wild type littermate controls are generated by mating heterozygote lacZ positive males with standard C57BL/6J female mice (Taconic, Germantown, N.Y.). Adult Ptcl-lacZ mice were fixed by cardiac perfusion followed by drop fixation of heart or vascular tissues for 1-2 hours in 0.2% gluteraldehyde, 5mM EDTA, 2mM MgCl₂, 0.1M sodium phosphate, pH8. Pup tissues and small tissues were directly drop fixed in gluteraldehyde for 1-2 hours. Following fixation, the tissues were washed 3 times for 20-30 min in 2 mM MgCl₂, 0.01 deoxycholate, 0.02% NP40, 5OmM sodium phosphate pH8. The tissues were then stained overnight at 37° C. in lmg/ml 5-Bromo-4-chloro-3-indolyl-D-galactopyranoside (Xgal) (Sigma, St. Louis, Mo.), 5 mM potassium ferricyanide, 5 mM potassium ferrocyanide, 2 mM MgCl₂, 0.01 % deoxycholate, 0.02% NP40, 50 mM sodium phosphate pH8. The tissues were visualized either as whole mounts or embedded in paraffin and prepared as light eosin-stained 5 micron sections.

Patched 1 is expressed in the endothelial cells of the aorta, some vascular smooth muscle cells (vSMC) and adventitial fibroblasts of the aorta (photomicrographs not presented here). In addition, coronary vasculature and cardiomyocytes of the atria and ventricles also express ptcl. These expression patterns suggest that cells in normal vascular and cardiovascular tissues may be responsive to or responding to hedgehog.

Normal Vasculature and Cardiovascular Tissues are Hedgehog Responsive

We determined that normal vascular and cardiovascular tissues are indeed responsive to exogenous hedgehog administration by injecting Ptcl-lacZ mice systemically with hedgehog. Ptcl-lacZ mice were injected daily subcutaneously with the indicated amounts of polyethylene glycol 20,000-conjugated A192C sonic hedgehog n-terminal protein (PEG—Shh) (Pepinsky et al, 2000) or its vehicle (PBS). This form of the protein also contains a mutation of the n-terminal cysteine residue to isoleucine-isoleucine which significantly improves the specific activity of hedgehog protein (Pepinsky et al, 1998; Taylor et al, in prep).

Mice injected with hedgehog protein for 3 days showed no obvious physical or behavioural differences compared to vehicle-treated or untreated littermates. Specifically, Ptcl-lacZ mice were injected (s.c.) once daily with PEG—Shh for 3 days starting at postnatal day 6 then sacrificed at postnatal day 9; selected organs were dissected and whole mount stained by X-Gal histochemistry. Mice were treated with vehicle, 3mg/kg PEG—Shh or 6mg/kg PEG—Shh for 3 days and were sacrificed on the fourth day. Vascular and cardiovascular tissues were dissected and whole-mount stained with Xgal. The vascular and cardiovascular staining pattern for ptcl seen in normal animals intensifies significantly in animals injected with increasing doses of hedgehog protein (data not presented here). Whole mount Xgal staining of the coronary arteries, atria and ventricles are increased in a dose dependent manner in the hearts and in the aortic wall of the Ptcl-lacZ mice injected with hedgehog. In contrast, wild type littermate mice injected with the highest dose of hedgehog (6mg/kg) show no staining suggesting that the staining seen in the Ptcl-lacZ animals is not due to endogenous betagalactosidase. Histological sections of these tissues show that the lacZ positive cells in the Ptcl-lacZ mice treated with hedgehog are similar to those which are positive in the vehicle-injected group and in normal adult hearts and aortas from untreated animals. Though the same type of cells appear to stain with Xgal in the treated animals, there appears to be an increase in the number of these cells especially in the adventitia. These data show that systemic administration of hedgehog can induce ptcl upregulation and indicate that these vascular tissues are responsive to hedgehog protein.

### EXAMPLE 2

Hedgehog Induces Neovascularization in Matrigel Plug Model of Angiogenesis

Hedgehog was also found to induce angiogenesis in the subcutaneous matrigel plug assay (Passaniti et al., 1992). Doses of 2 to 10 ug/ml of octyl, myr, PEG II or II-Fc fusion forms of human recombinant Shh were prepared in 0.5ml of matrigel containing 40 IU/ml of heparin and injected subcutaneously into C57BL6 mice (3-5mo. old, 5 mice/treatment group). The mice were sacrificed between 6-7 days after injection and the matrigel plug was dissected for visual inspection and histological analysis. Plugs containing hedgehog induced significant angiogenesis in the plug and surrounding tissue in 4 of 6 plugs at 2ug/ml and 5 of 6 plugs at lOug/ml whereas only 2 of 9 vehicle containing plugs showed any evidence of angiogenesis (data not presented here). Recombinant human bFGF, a known angiogenic protein, also showed significant hemoglobin content in 3 of 5 implants (data not shown). The results of the matrigel plug support the finding that hedgehog can induce angiogenesis in vivo.

### EXAMPLE 3

Hedgehog Induces Neovascularization in Corneal Model of Angiogenesis

The mouse cornea is avascular and can be used to demonstrate angiogenic activity by measuring the amount of vessel growth into this avascular tissue after surgical placement of a polymer pellet containing an angiogenic substance or growth factor into the cornea (Kenyon et al., 1996; Asahara et al., 1997). To confirm the angiogenic activity of hedgehog in another well accepted model of angiogenesis, we tested the ability of hedgehog protein to induce neovascularization in the mouse corneal model pocket model of angiogenesis.

Animals were anesthetized by pentobarbital intraperitoneal injection (160 mg/kg). Corneal pockets were created in the eyes of each mouse and a 0.34 X 0.34 mm sucrose albumin sulfate (Bukh Meditec, Vaerlose, DK) pellet coated with hydron polymer type NCC (Interferon Sciences, New Brunswick, N.J.) containing 1 of the agents indicated below was implanted into the corneal pocket. C57BL/fJ mice were divided into 5 groups: control buffer alone; VEGF 300 ng/pellet; Myr—Shh vehicle alone; Myr—Shh 1.5 microg/pellet 39; Myr—Shh+VEGF (1.5 microg/pellet +300 ng/pellet, respectively). Pellets were positioned 1.0 nun from the corneal limbus, and erythromycin ophthalmic ointment (E. Fourera) was applied to each operated eye. The corneas of all mice were routinely examined by slit-lamp biomicroscopy on postoperative day 6 after pellet implantation.

On the same day vessel length and corneal circumferential neovascularity (in degrees) were measured. After completing these measurements, C57BL/6J mice received an intravenous injection of 500 pg of BS-1 lectin FITC-conjugated (Vector Laboratories, Burlingame, Calif.). Thirty minutes later, the animals were sacrificed. The eyes were enucleated and fixed in 1 % paraformaldehyde solution. After fixation, the corneas were placed on glass slides and examined by fluorescence microscopy. Several C57BL/6J mice in each group did not receive BS-1 lectin injection; instead, the eyes were excised and fixed in 100% methanol solution for immunohistochemical staining.

There was significant neovascular growth in the Shh and in the VEGF groups but not the vehicle-containing pellet groups. There was a striking qualitative difference in the appearance of vessels induced by hedgehog compared to VEGF (photomicrographs not presented here). VEGF induced a fine mesh of capillaries which are short tortous sprouts from the extended branches of the preexisting limbus vessels at the base of the eye. In contrast, hedgehog induced much larger vessels which extended all the way to the pellet and contained numerous anastamoses between the venous and arterial circulation. Histological analysis confirmed that hedgehog induced larger diameter vessels than VEGF. Hedgehog induced vessels often were filled with red blood cells whereas VEGF induced vessels had few or no red blood cells.

Measurements (mean istandard error of the mean) of the VEGF and hedgehog vessels confirmed that hedgehog-induced vessel diameters (mean 33±17um) were significantly larger than VEGF vessel diameters (mean 8±3um) (p<0.0001)). The maximum vessel lengths induced by hedgehog (1020+200um) were also significantly greater than the maximum length of vessels induced by VEGF (700±70um) (p<0.000l). The density of vessels induced by hedgehog was slightly lower than the density of vessels in the corneal tissue exposed to VEGF as may be expected from the large number of small capillaries formed by VEGF (p<0.0001). All group differences were analysed by ANOVA and differences with p<0.05 were considered statistically significant.

In summary, neovascularization induced by Shh was characterized by a statistically significant increase in vessel length, circumferential neovascularity and diameter of the lumens; the mean number of vascular lumens per cross section was higher in the VEGF-treated corneas. Neovascularization induced by Shh+VEGF showed a large variability in the lumen diameter of these vessels ranging from small capillaries (6-7 gm) to large diameter vessels (80 gm). The combination of VEGF and Shh appears to create a composite of characteristics of both VEGF and Shh neovascular growth. These results confirm hedgehog protein can induce angiogenesis in vivo and suggest that hedgehog either alone or in combination with VEGF or other angiogenic growth factors such as bFGF, the angiopoietins and TWEAK [Lynch C N, Wang Y C, Lund J K, Chen Y W, Leal J A, Wiley S R. TWEAK induces angiogenesis and proliferation of endothelial cells. J Biol Chem. 1999 Mar 26;274(13):8455-9] can have therapeutic utility by inducing functional neovasculature.

### EXAMPLE 4

Biological Activities Induced By Hedgehog —Responsive Mesenchymal Cells

Hedgehog induces stromal fibroblasts and VEGF upregulation in the corneal model of angiogenesis

To determine the mechanism by which Shh induces angiogenesis both Shh and VEGF-stimulated corneas (see Example 3) were excised and X-gal stained as described in Example 1 after fixation of the whole eye for 1 hour in 1 % paraformaldehyde followed by enucleation and fixation of the corneal hemisphere in 1 % paraformaldehyde for 30 minutes. VEGF-induced corneas did not stain with X-gal, indicating that VEGF does not induce Ptc 1 expression during neovascularization. In contrast, strong X-gal staining was detected in the neovascular regions of Ptcl-lacZ corneas treated with Shh (data not presented here). Histologic analysis following paraffin embedding of X-gal-stained corneas and preparation of immunostained 5um sections with showed that the X-gal positive cells were not endothelial cells or smooth muscle cells, but fibroblasts surrounding the neovessels. Endothelial cell immunostaining was done with a rat monoclonal antibody against mouse CD-3 1 (Pharmigen, San Diego, Calif.) followed by a biotinylated goat anti-rat immunoglobulin secondary antibody. Smooth muscle cells and pericytes were identified with a mouse monoclonal antibody against SM a-actin conjugated with alkaline-phosphatase (Sigma, St. Louis, Mo.) and fibroblasts were identified using an anti-vimentin antibody (Sigma, St. Louis, Mo.).

We then immunostained the Shh-induced corneas with a rabbit polyclonal anti-VEGF antibody (Santa Cruz Biotechnology, Santa Cruz, Calif.) with a biotinylated goat anti-rabbit immunoglobulin as secondary antibody. The results show that VEGF protein is in the fibroblasts and matrix immediately adjacent to the neovascular area. These results suggested that hedgehog may induce resident fibroblasts in the cornea to produce angiogenic factors such as VEGF.

Fibroblasts in vitro respond to hedgehog stimulation by upregulation of Ptcl and angiogenic growth factors

To determine if hedgehog can directly induce fibroblasts to produce VEGF or other angiogenic factors , we treated normal human fibroblasts (CCD37) with Myr—Shh and the ability of fibroblasts to respond was evaluated by competitive RT-PCR for ptc 1 and several angiogenic growth factors. Total RNA was prepared from cells treated as described above using Trizol (Life Technologies, Rockville, Md.). Four micrograms of total RNA was used to prepare cDNA using the SuperScriptTM preamplification system (Cat. No. 18089-011, Life Technologies, Rockville, Md.). The PCR reaction using buffer reagents from the SuperScriptTM preamplification system (Life Technologies, Rockville, Md.) was quantitated with 20S rRNA competitive primers (Ambion). Primers for the amplification of Ptc 1 were 5′-TCAGGATGCATTTGACAGTGACTGG-3′ (SEQ ID NO: 38) and 5′-ACTCCGAGTCGGAGGAATCAGACCC-3′ (SEQ ID NO: 39) which are based on ptcl cDNA sequence (GenBank Accession Number U46155). All amplification for Ptcl were done with 25 cycles of 94° C. for 30 sec; 55° C. for 1 min; 72° C. for 1 min. The cDNA from the same cells was also used as a template for VEGF, bFGF, Angiopoietin 1, and Angiopoietin II amplification. The following primer pairs and PCR cycles were used: VEGF: 5′CGAAGTGGTGAAGTTCATGGATG3′ (SEQ ID NO: 40) and 5′TTCTGTATCAGTCTTTCCTGGTGAG3′ (SEQ ID NO: 41) which are based on the human VEGF cDNA sequence (GenBank Accession Number E15157). VEGF product was amplified with 30 cycles of 94° C. for 30 sec; 62° C. for 1 min; 72° C. for 1 min; bFGF: 5′TACAACTTCAAGCAGAAGAG3′ (SEQ ID NO: 42) and 5′CAGCTCTTAGCAGACATTGG3′ (SEQ ID NO: 43) which is based on the human bFGF cDNA sequence (GenBank Accession Number M27968). bFGF product was amplified with with 25 cycles of 94° C. for 30 sec; 62° C. for 1 min; 72° C. for 1 min; Angiopoietin1 5′CAACACAAACGCTCTGCAGAGAGA3′ (SEQ ID NO: 44) and 5′CTCCAGTTGCTGCTTCTGAAGGAC 3′ (SEQ ID NO: 45) which is based on human Angiopoietin1 cDNA sequence (GenBank Accession Number U83508). Angiopoietin I product was amplified with 25 cycles of 94° C. for 30 sec; 64° C. for 90 sec; Angiopoietin II: 5′AGCGACGTGAGGATGGCAGCGTT3′ (SEQ ID NO:46) and 5′ATTTCCTGGTTGGCTGATGCTGCTT3′ (SEQ ID NO: 47) which are based on human Angiopoietin II cDNA sequence (GenBank Accesion Number AB009865). Angiopoietin II product was amplified with with 32 cycles of 94° C. for 30 sec; 64° C. for 90 sec. As internal control for sample preparation, gel loading, and random variations in RT-PCR, 18S rRNA primers and 18S rRNA competimers (Ambion, Austin, Tex.), used to modify 18S cDNA amplification efficiency, were included in each PCR reaction with target gene-specific primers. The linear range of amplification and optimal 18S primer/Competimer ratio was determined for each target gene following the manufacturer's recommendations (Ambion, Austin, Tex.).

A time course of Shh induction shows that human fibroblast respond to Shh by upregulating the Ptcl gene (data not shown) indicating that these cells can respond via the known Hh signalling pathway. Neither human umbilical vein and microvascular endothelial cells respond to Hh (data not shown).

We next found that Hh can upregulate fibroblast expression of angiogenic growth factors, including VEGF, bFGF, Ang-1, and Ang-2 (data not shown). VEGF mRNA from human fibroblasts was significantly increased by Shh: all the three VEGF isoforms (VEGF121, 165, and 189) were strongly upregulated. VEGF 121, 165, and 189 upregulation began at 12 hours and was maximal after 48 hours of incubation of the cells with Shh. No bFGF upregulation was detectable at any time-points. Moreover, quantitative RT-PCR for Ang-1 and Ang-2 showed upregulation of both genes, with maximal increase after 36 hours stimulation. To show that the upregulation of VEGF mRNA correlated with an increase in protein production, the concentration of VEGF165 in cell media was measured by ELISA. Cells were stimulated with recombinant human myristolated Shh protein as described above. At harvest, the cell conditioned media was collected, centrifuged to remove cell debris (15 minutes at 1500×g) and production of VEGF165 protein was evaluated by using an ELISA kit (Quantikine human VEGF, R&D Systems, Minneapolis, Minn.). Total VEGF protein level underwent a progressive increase following Hh stimulation and a significant upregulation in the VEGF production was detectable at 72 hours (data not shown).

Smooth muscle cells upregulate ptcl and are induced to proliferate in vitro in response to hedgehog

We found that smooth muscle cells can also respond to Hh proteins in vitro. Eighty five percent confluent monolayers of vascular smooth muscle cells (PAC 1) were induced for 2 days with lug/ml of myrShh or an equivalent volume of vehicle in normal media (M 199 complete media with 10% fetal bovine serum). For comparison, primary normal human lung fibroblasts and normal prostate stromal cells were grown in complete FBM and similarly stimulated (Clonetics/Bio-Whittaker, Walkersville, Md.). The cells were harvested and RNA from the cells was prepared and analysed by RT-PCR as above. All of these cells showed increased ptc 1 expression following induction with myrShh, but not myrShh vehicle alone suggesting that each of these cell types are responsive to hedgehog (data not shown). In addition, hedgehog protein induced DNA synthesis in quiescent vascular SMCs and human fibroblasts. PAC-1 (Rothman et al., 1992), WKY (Lemire et al., 1994), primary pulmonary artery SMCs or aortic SMCs (Clonetics/Bio-Whittaker, Walkersville, Md.) were plated (5×103/well) in 96 well plates and allowed to adhere for 2-3 hours in 0.1 8ml of complete media (M 199 with 10% fetal bovine serum for PAC 1 cells, DMEM with 10% fetal bovine serum for WKY cells or smGM-2 for primary human pulmonary artery or aortic SMCs). The cells were then starved for 18-24 hours in complete media with 0.5% fetal bovine serum. Quiescent cells were stimulated with 0.1 to 40ug/ml of Hh proteins in 0.2 ml starvation media for 48 hours after which the cells were pulse labeled with 4.5uCi/ml 3H-thymidine (Amersham,) for 4-8 hours at 37° C. The media was then removed, the cells washed with PBS then trypsinized. 3H-thymidine uptake into cells was determined by scintillation counting using a 1205 Betaplate counter (Wallac, Gaithersburg, Md.). Vascular SMCs showed increased 3H-thymidine uptake 3 to 4-fold when induced by either myrShh (myristylated Sonic hedgehog) Dhh or basicFGF (obtained from Upstate Biotechnology, Lake Placid, N.Y.).

These results show that both SMCs and fibroblasts respond to hedgehog. Although no smooth muscle cells were found in the hedgehog-stimulated corneas (see Example 1 and 4), the responsiveness of SMCs to Hh in vitro correlates well to normal ptcl expression and increased ptcl in the response by normal vascular SMCs to systemically administered Hh protein (See Example 3).

### EXAMPLE 5

Hedgehog Improves Recovery from Ischemic Limb Injury

Peripheral vascular disease caused by atherosclerosis and/or diabetes can be modeled in rodents and rabbits by surgical ligation of the femoral artery and removal of a segment of the artery distal to the ligation (Takeshita et al., 1994 and 1996; Rivard et al., 1999; Couffinhal et al., 1999). The limb ischemia produced by the ligation also results in limb neuropathy (Schratzberger et al., 2000). Ischemic injury of healthy animals and humans activates a number of pathways which subsequently induce the regeneration and recovery of the damaged tissue. For example, VEGF is induced in response to hindlimb ischemia and can accelerate recovery when given pharmacologically following this ischemic insult (Schratzberger et al., 2000). We investigated the possibility that the hedgehog pathway is activated in response to limb ischemia in normal animals and is beneficial both in the endogenous and pharmacological settings to revascularization and recovery from ischemic neuropathy.

The expression of ptcl following hindlimb ischemia was investigated in 3-4 month old Ptcl-lacZ mice (Rivard et al., 1999). The mice were anesthetized with pentobarbital (1 60mg/kg i.p.) and an incision was made in the skin overlying the middle portion of the left hindlimb. Both the proximal end of the femoral artery and the distal portion of the saphenous artery were ligated and the artery and all side branches were dissected free and excised. The skin was closed with a surgical stapler and the animals were allowed to recover. The mice were either left untreated or injected daily or every other day i.m. in the ischemic limb with lmg/kg of II—Shh/mouse IgGI Fc fusion protein. Seven days after induction of ischemia, the animals were sacrificed and the upper hindlimb was isolated and whole mount stained with Xgal. Comparison of the contralateral upper hindlimbs (right) to the ischemic hindlimbs (left) shows a significant upregulation of ptcl expression (data not shown). Ischemia alone induced upregulation of ptcl expression in the ischemic limb and increasing frequency of hedgehog injection further increased ptcl expression in the ischemic limb muscle. Histological sections of the ischemic and control hindlimb muscle showed muscle fiber degeneration and edema in the ischemic versus nonischemic tissue (data not shown). In addition, the ischemic muscle has a number of ptcl-expressing (Xgal-stained) stromal cells in the interstitial areas between the muscle fibers. These cells which appear to be responding to hedgehog were shown to be fibroblasts identified by costaining with vimentin and X-gal or monocytes/macrophages identified by costaining with the moma2 antibody and X-gal (see Example 4 for Methods). These results show that the hedgehog pathway may be part of the normal response to ischemia which may be augmented by pharmacological administration of hedgehog protein.

The relevance of hedgehog upregulation following ischemia is determined by inhibiting hedgehog action with a blocking antibody to hedgehog. Unilateral hindlimb ischemia was induced in normal mice (C57BL6, 3-4months of age, female). The mice are treated with 10mg/kg daily 3 days prior to ischemia and 2.5-5mg/kg every 3 days following ischemia for 3 weeks with either the blocking antibody to hedgehog (SE1, Developmental Studies Hybridoma Bank, Karen Jensen, Department of Biological Sciences, The University of Iowa, 007 Biology Building East, Iowa City, IA 52242, tel: (319)335-3826, fax: (319)335-2077, 5E1 available for order on website: www.uiowa.edu/-dshbwww/l*ndex.html, e-mail: dshb@uiowa.edu) or an isotype matched control mouse monoclonal antibody.

The vascular perfusion of the ischemic vs contralateral limb is assessed at days 4, 7, 14, 21 and 28 days by lasar doppler (Lisca, Inc. laser Doppler perfusion imager system) (Rivard et al., 1999). Nerve vascular perfusion is determined by exposing the sciatic nerve and scanning the nerve surface area using lasar doppler or by injection of Fluoresceinated-BS 1 lectin (Vector Laboratories, Burlingame, Calif.) 30 minutes prior to sacrifice and visualizing the vaso nervorum by whole mount fluorescence microscopy postmortem (as described above). Vascular density is assessed at these times by histological staining for CD31 positive vasculature in sections (anti-murine CD31, Pharmingen, San Diego, Calif.) (Rivard et al., 1999). Neuropathy is assessed at these time points by nerve conduction measurements of the sciatic/peroneal nerves using standard orthodromic surface recording techniques and a Teca TD-10 portable recording system (Oxford Instruments, Concord, Mass.). Angiogenesis as measured both by vascular perfusion or vascular density is decreased in ischemic limbs of animals treated with hedgehog blocking antibody, 5E1, compared to ones treated with the isotype matched control, 1E6. Nerve conduction measurements are also decreased in 5E1-treated mice compared to control antibody-treated mice. Finally, nerve vascular perfusion is decreased in the 5E1-treated mice. These results suggest that the upregulation of the hedgehog pathway following ischemia is a beneficial compensatory response to ischemic injury.

The utility of treating ischemia by activating the hedgehog pathway is tested in aged mice (>2yrs old) or apoE null mice with surgically induced limb ischemia since these mice are deficient in their repair and regeneration processes following limb ischemia. These mice are made ischemic then injected (i.v., i.p., s.c. or i.m.) with doses ranging from 0-lOmg/kg of hedgehog protein or equivalent volumes of vehicle control or control protein beginning on the day of surgery and with a frequency of daily to 3 times per week. The vascular perfusion, vascular density and neuronal conduction and neuronal vascularity (vaso nervorum) of the ischemic vs contralateral limb are assessed at days 4, 7, 14, 21 and 28 postsurgery as described above. The results show that hedgehog-treated animals show significant improvements in vascular perfusion, vascular density as well as motor nerve conduction and their vaso nervorum compared to control treated animals (data not presented).

Hedgehog can also be delivered using gene therapy. Either full length or soluble Nterminal Shh adenovirus (10⁶ to 101¹⁰ particles) is injected i.m. at day 1 postinjury in the inguinal area of the upper hindlimb following surgery. Alternatively, the full length or soluble n-terminal Shh adenoassociated virus (AAV) or a control LacZ AAV is administered 4 weeks prior to surgery. Similar doses of adenovirus containing full length or n-terminal Shh or LacZ containing control adenovirus can be administered in place of AAV—Shh. Above endpoints for vascular and motor neuron conduction improvements are also seen with viral gene therapy.

Together these results show that the hedgehog pathway is a crucial component of the normal angiogenic response, tissue regeneration and recovery from ischemia injury and that hedgehog proteins can induce angiogenesis and improve recovery from ischemia when used pharmacologically.

### EXAMPLE 6

Hedgehog Induces Collateral Vessel formation and Improved Myocardial Function following Surgically Induced Myocardial Ischemia

Chronic myocardial ischemia and collateral vessel formation can be modeled in pigs through the placement of an ameroid constrictor around the left circumflex coronary artery. Treatment of these ischemic hearts with angiogenic proteins can increase myocardial vascularity, perfusion and function in the ischemic area as well as overall heart function. We determine that hedgehog protein or gene therapy can also improve these measures of cardiac perfusion, viability and function following ischemia in the following experiments.

Ameroid constrictors are placed around the left circumflex coronary artery (LCX) of anesthetized Yorkshire pigs (5-6 weeks old, 15-18kg, male or female) (Laham et al., 2000; Harada et al., 1994; Unger et al., 1994). The animals are allowed to recover for 3 weeks to allow time for ameroid closure. Either immediately after or 3 weeks post-ameroid placement, the animals are randomized into one of several groups (10 animals/group). Hedgehog or control is administered by one of the following routes:

1. direct injection of ischemic myocardium with hedgehog or saline

2. intrapericardial administration of hedgehog protein or saline

3. systemic administration of hedgehog protein or saline (s.c., i.m. or i.v. injection)

4. myocardial injection of hedgehog in (0.1-5mg) heparin or heparin alone following thoracotomy or via an injection catheter (Cordis-Webster)

5. intrapericardial injection of hedgehog in (0.1-5mg) heparin

6. intracoronary catheter delivery device

7. viral gene therapy via above methods using 10⁶-10¹² Particles of full length or n-terminal Shh adenovirus in a single or several bolus injections (0.lml-lml/injection). Heart muscle perfusion and function are monitored using several techniques immediately prior to the Hedgehog treatments and 2-4 weeks post-Hedgehog treatments. Coronary perfusion was determined by right and left coronary angiography.

To obtain a collateral index, left to left and right to left coronary collaterals are measured. Regional resting myocardial blood flow is measured using colored microspheres. Magnetic resonance imaging of wall thickening is used to determine global ventricular, ischemic/normal regional function and myocardial perfusion. Electromechanical left ventricular mapping is done using the NOGA system (Biosense, Johnson&Johnson, Warren, N.J.) to determine localized heart function (Vale et al., 1999, Komowski, Hong and Leon, 1998). In addition, complete autopsies and histopathology is done on each animal for coronary tissues (pericardium, epicardial coronary artery, myocardium in the left anterior descending artery distribution (normal tissue), left circumflex artery distribution, (ischemic tissue) and peripheral organs (gastrointestinal tract, lung, liver, kidney, bone, bone marrow)). Improvements in heart muscle perfusion and function as well as histological analysis of coronary tissue vascularization are assessed. Hedgehog treatments can show improvement in these parameters when compared to control treatments suggesting therapeutic utility for hedgehog treatments in myocardial infarction and coronary artery disease.

### EXAMPLE 7

Inhibition of Hedgehog (Anti-hedgehog blocking antibody) Decreases Tumor Growth Rate and/or Tumor Angiogenesis

To determine if tumor cell lines overexpress hedgehog protein, anti-hedgehog antibody was used to immunoprecipitate cell lysates of various tumor cell lines. We used gastrointestinal epithelial cell lines as an example: T84 (human colon epithelial carcinoma, CCL-284, ATCC, Manassas, Va.); Caco2 and SW480 (human colon epithelial adenocarcinomas, HTB-37 and CCL-228, ATCC, Manassas, Va.). Briefly, one milligram amounts of cell lysis supernatant were immunoprecipitated with either anti-hedgehog antibody, 5E1 (+) or an isotype matched control antibody, 9E10 (C). The immunoprecipitated samples were analysed by western blotting with an anti-hedgehog rabbit polyclonal antibody, r1200.

More specifically, confluent monolayers of each cell line in T 150 flasks were lysed in 3mL of cold lysis buffer (1% Triton X-100, 0.5% sodium deoxycholate, 0.% SDS, 150mM NaCl, lmM sodium vanadate, 10% glycerol, lOmM Tris—HCl, pH 8.0) containing a 2× concentration of Complete protease inhibitor cocktail (Boehringer Mannheim, Indianapolis, Ind.). The lysate was rocked for 30′ at 4° C. then scraped into a microfuge tube and debris pelleted in a microfuge for 10′. The supernatant was stored at −80° C. Protein concentration of the supernatants were determined using Bio—Rad Protein Assay reagent and equivalent milligram amounts of supernatant were used for each immunoprecipitation. Each sample was gently agitated overnight at 4° C. with 2.5 ug of either anti-hedgehog antibody, 5E I, or an isotype matched control antibody, 9E10 (anti-human c-myc, Calbiochem, San Diego, Calif. ) (Fan et al., 1998). Protein A conjugated Sepharose beads (30 microliters packed beads/sample) were added to each sample and the samples were gently agitated at 4° C. for 30-40 minutes. The beads and associated immune complexes were then spun down in a microfuge for 10 seconds and washed 4 times with 1 ml of ice cold lysis buffer. The buffer was then removed from the beads, reducing SDS-PAGE sample buffer was added, the samples were heated to 90° C. for 5 minutes then analyzed by SDS-PAGE (4-20% Tris-glycine gels, Novex, San Diego, Calif.). The proteins were transferred to nitrocellulose filters and western blot analysis was performed at room temperature.

The nitrocellulose filters was incubated with blocking solution (5% dry milk in Tris-buffered saline with 0.3% Tween-20) for 1 hour followed by blocking solution containing a 1:10,000 dilution of anti-hedgehog rabbit polyclonal, rl200, for 2-3 hours at room temperature or overnight incubation at 4° C. The nitrocellulose filters were washed 3 times with Tris-buffered saline with 0.3% Tween-20; incubated for 1 hour in 1:5000 dilution of horseradish peroxidase-conjugated goat anti-rabbit antibody (Jackson Immunoresearch) then visualized using ECL western blotting detection reagents (Amersham Pharmacia Biotech).

Hedgehog protein is overexpressed in several human gastrointestinal tumor cell lines compared to normal human gastrointestinal epithelial cells or fibroblasts (data not shown). The anti-hedgehog antibody immunoprecipitations show a hedgehog rabbit polyclonal antibody-reactive band at 19kD, the expected molecular weight for hedgehog protein. The control antibody (9E10) immunoprecipitation shows no hedgehog polyclonal antibody-reactive band comigrating with hedgehog protein standard at 19kD. Normal gastrointestinal epithelial also express a low level of hedgehog protein, but normal gastrointestinal fibroblasts do not show any expression. None of the epithelial cell lines tested respond to hedgehog (data not shown), but the hedgehog produced by these tumor cells may activate angiogenesis via induction of stromal tissue in the tumor.

The ability of hedgehog-blocking or hedgehog pathway-blocking reagents such as the anti-hedgehog blocking antibodies (5E I, ARG6, ALC9 or BH.E4) to inhibit tumor angiogenesis and tumor growth are determined in subcutaneously-implanted tumor models in athymic Swiss (Cr:NIH(S)-nu) or athymic random bred (NCr-nu) mice of a single sex (males>1 8 g or females>17 g, all within a 4g weight range). Carcinoma cell lines of gastrointestinal origin such as SW480, HT29 or T84 are passaged in nude mice as subcutaneous tumors or are passsaged in culture as cell monolayers. Either 2×10⁶ cells or tumor 20-40mg fragments of a passaged tumor are implanted subcutaneously in the axillary region of 6-10 athymic mice. Tumors were monitored frequently for progressive growth. Treatments are initiated when individual tumors range between 100 mg −700 mg. Mice are randomized into test and vehicle control groups and treated with either hedgehog blocking antibodies, control isotope-matched antibody, no treatement or cisplatinum. Antibodies were administered (25-100 mg/kg bolus i.p. injections) at a frequency of every day to 3 times a week for the follow-up period. Cisplatinum was administered subcutaneously three times a week ( 2 mg/kg). Body weights and tumor measurements (width and length) are recorded at 3-5 day intervals following treatment for 7-21 days. Tumors are collected on the final day for histological analysis. Mean tumor weight change and/or mean vascular density are decreased in the hedgehog blocking antibody-treated group compared to the control antibody-treated group. In addition, hedgehog blocking antibodies may be administered prior to tumor implantation and tumor growth rate is monitored as described to determine if early tumor growth rates are decreased by blocking hedgehog signalling.

### EXAMPLE 8

Production and Expression of HH-Ig fusions

MATERIALS AND METHODS

Construction ofpUB55, expression plasmidfor Sonic Hedgehog in Pichia pastoris:

pUB55 contains the N-terminal domain of human Sonic Hedgehog (SEQ ID NO: 21 in Table 4) with the alpha factor PrePro region as the secretion signal. pUB55 was constructed in pCCM73, a derivative of pPIC9 (obtained from Invitrogen, San Diego, Calif.) with the Kanamycin gene (HincII-Hinc l l fragment) of pUC4-K inserted at the Sphl site of pPIC9. The human Sonic hedgehog coding sequence from Earl—Notl was obtained from pEAG543 which has a stop codon and Not 1 site engineered following Gly 197 in the coding sequence. Plasmid pCCM73 was cut with XhoI and NotI and was ligated with the Earl—Notl fragment of pEAG543 (containing the Sonic Hedgehog coding sequence, Table 4) and oligonucleotides [5′TCG AGA AAA GAT GCG GAC CGG GCA GGG GGT 3′: SEQ ID NO: 36 and 5′CGA ACC CCC TGC CCG GTC CGC ATC TTT TC 3′: SEQ ID NO: 37] that form a XhoI-EarI fragment and create the appropriate coding sequence for placing Sonic hedgehog adjacent to the alpha factor leader sequence in frame.

Expression of Desert Hedgehog in Pichia pastoris and construction of KEX2 site mutations:

The Desert Hedgehog coding region in plasmid pEAG680 was modified to incorporate a BsrGI and an XmaI site site using the Stratagene QuikChange mutagenesis kit.

Expression of Indian Hedgehog in Pichia pastoris and construction of KEX2 site mutattions:

Plasmid pEAG657 is pBluescript with the Indian Hedgehog coding sequence with a stop codon following codon GlyXXX. pEAG658 is pBluescript with the Indian Hedgehog coding sequence and a Sall site engineered within residues suitable for fusing the Indian Hedgehog coding sequence (SEQ ID NO: 22) with Fc immunoglobulin coding sequences (SEQ ID NOS: 28-30) at the hinge region of immunoglobulins. To facilitate subsequent manipulations, Spe J and XmaI sites were introduced to pEAG658 by site-directed mutagenesis.

Table: DNA sequences of Hedgehog N-terminal domains and Immunoglobulin Fc

| | | |
|---|---|---|
| Regions: | | |
| Protein | DNA Sequence | |
| Human Sonic Hedgehog N- | TGCGGACCGGGCAGGGGGTTCGGGAAGAGGAGG | [SEQ ID NO:21] |
| terminal Domain | | |
| | CACCCCAAAAAGCTGACCCCTTTAGCCTACAAGC | |
| | AGTTTATCCCCAATGTGGCCGAGAAGACCCTAG | |
| | GCGCCAGCGGAAGGTATGAAGGGAAGATCTCCA | |
| | GAAACTCCGAGCGATTTAAGGAACTCACCCCCA | |
| | ATTACAACCCCGACATCATATTTAAGGATGAAG | |
| | AAAACACCGGAGCGGACAGGCTGATGACTCAGA | |
| | GGTGTAAGGACAAGTTGAACGCTTTGGCCATCTC | |
| | GGTGATGAACCAGTGGCCAGGAGTGAAACTGCG | |
| | GGTGACCGAGGGCTGGGACGAAGATGGCCACCA | |
| | CTCAGAGGAGTCTCTGCACTACGAGGGCCGCGC | |
| | AGTGGACATCACCACGTCTGACCGCGACCGCAG | |
| | CAAGTACGGCATGCTGGCCCGCCTGGCGGTGGA | |
| | GGCCGGCTTCGACTGGGTGTACTACGAGTCCAA | |
| | GGCACATATCCACTGCTCGGTGAAAGCAGAGAA | |
| | CTCGGTGGCGGCCAAATCGGGAGGC | |
| Human Indian Hedgehog | TGCGGGCCGGGTCGGGTGGTGGGCAGCCGCCGG | [SEQ ID NO:22] |
| N-terminal Domain | | |
| | CGAGGGCCACGCAAACTCGTGCCGCTCGCCTACA | |
| | AGCAGTTCAGCCCCAATGTGCCCGAGAAGACCC | |
| | TGGGCGCCAGCGGACGCTATGAAGGCAAGATCG | |
| | CTCGCAGCTCCGAGCGCTTCAAGGAGCTCACCCC | |
| | CAATTACAATCCAGACATCATCTTCAAGGACGA | |
| | GGAGAACACAGGCGCCGACCGCCTCATGACCCA | |
| | GCGCTGCAAGGACCGCCTGAACTCGCTGGCTATC | |
| | TCGGTGATGAACCAGTGGCCCGGTGTGAAGCTG | |
| | CGGGTGACCGAGGGCTGGGACGAGGACGGCCAC | |
| | CACTCAGAGGAGTCCCTGCATTATGAGGGCCGC | |
| | GCGGTGGACATCACCACATCAGACCGCGACCGC | |
| | AATAAGTATGGACTGCTGGCGCGCTTGGCAGTG | |
| | GAGGCCGGCTTTGACTGGGTGTATTACGAGTCAA | |
| | AGGCCCACGTGCATTGCTCCGTCAAGTCCGAGCA | |
| | CTCGGCCGCAGCCAAGACGGGCGGC | |
| Human Desert Hedgehog | TGCGGGCCGGGCCGGGGGCCGGTTGGCCGGCGC | [SEQ ID NO:27] |
| N-terminal Domain | | |
| | CGCTATGCGCGCAAGCAGCTCGTGCCGCTACTCT | |
| | ACAAGCAATTTGTGCCCGGCGTGCCAGAGCGGA | |
| | CCCTGGGCGCCAGTGGGCCAGCGGAGGGGAGGG | |
| | TGGCAAGGGGCTCCGAGCGCTTCCGGGACCTCG | |
| | TGCCCAACTACAACCCCGACATCATCTTCAAGGA | |
| | TGAGGAGAACAGTGGAGCCGACCGCCTGATGAC | |
| | CGAGCGTTGTAAGGAGCGGGTGAACGCTTTGGC | |
| | CATTGCCGTGATGAACATGTGGCCCGGAGTGCG | |
| | CCTACGAGTGACTGAGGGCTGGGACGAGGACGG | |
| | CCACCACGCTCAGGATTCACTCCACTACGAAGGC | |
| | GCAACAAGTATGGGTTGCTGGCGCGCCTCGCAG | |
| | TGGAAGCCGGCTTCGACTGGGTCTACTACGAGTC | |
| | CCGCAACCACGTCCACGTGTCGGTCAAAGCTGAT | |
| | AACTCACTGGCGGTCCGGGCGGGCGGC | |
| Fc region of human IgGI-- | GTCGACAAAACTCACACATGCCCACCGTGCCCA | [SEQ ID NO:28] |
| with Asn-Gln glycosylation | | |
| site mutation | GCACCTGAACTCCTGGGGGGACCGTCAGTCTTCC | |
| | TCTTCCCCCCAAAACCCAAGGACACCCTCATGAT | |
| | CTCCCGGACCCCTGAGGTCACATGCGTGGTGGTG | |
| | GACGTGAGCCACGAAGACCCTGAGGTCAAGTTC | |
| | AACTGGTACGTGGACGGCGTGGAGGTGCATAAT | |
| | GCCAAGACAAAGCCGcgggaggagcagtaccagagcacgtacc | |
| | gtgtggTCAGCGTCCTCACCGTCCTGCACCAGGACT | |
| | GGCTGAATGGCAAGGAGTACAAGTGCAAGGTCT | |
| | CCAACAAAGCCCTCCCAGCCCCCATCGAGAAAA | |
| | CCATCTCCAAAGCCAAAGGGCAGCCCCGAGAAC | |
| | CACAGGTGTACACCCTGCCCCCATCCCGGGATGA | |
| | GCTGACCAAGAACCAGGTCAGCCTGACCTGCCT | |
| | GGTCAAAGGCTTCTATCCCAGCGACATCGCCGTG | |
| | GAGTGGGAGAGCAATGGGCAGCCGGAGAACAA | |
| | CTACAAGACCACGCCTCCCGTGTTGGACTCCGAC | |
| | GGCTCCTTCTTCCTCTACAGCAAGCTCACCGTGG | |
| | ACAAGAGCAGGTGGCAGCAGGGGAACGTCTTCT | |
| | CATGCTCCGTGATGCATGAGGCTCTGCACAACCA | |
| | CTACACGCAGAAGAGCCTCTCCCTGTCTCCCGGG | |
| | AAA | |
| Fc region of murine IgGl-- | GTCGACGTGCCCAGGGATTGTGGTTGTAAGCCTT | [SEQ ID NO:29] |
| with Asn-Gln glycosylation | | |
| site mutation | GCATATGTACAGTCCCAGAAGTATCATCTGTCTT | |
| | CATCTTCCCCCCAAAGCCCAAGGATGTGCTCACC | |
| | ATTACTCTGACTCCTAAGGTCACGTGTGTTGTGG | |
| | TAGACATCAGCAAGGATGATCCCGAGGTCCAGT | |
| | TCAGCTGGTTTGTAGATGATGTGGAGGTGCACAC | |
| | AGCTCAGACGCAACCaCGGGaGAGCAGTTCCAA | |
| | AGCACTTTCCGCTCAGTCAGTGAATTCCCATCA | |
| | TGCACCAGGACTGGCTCAATGGCAAGGAGTTCA | |
| | AATGCAGGGTCAACAGTGCAGCTTTCCCTGCCCC | |
| | CATCGAGAAAACCATCTCCAAAACCAAAGGCAG | |
| | ACCGAAGGCTCCACAGGTGTACACCATTCCACCT | |
| | CCCAAGGAGCAGATGGCCAAGGATAAAGTCAGT | |
| | CTGACCTGCATGATAACAGACTTCTTCCCTGAAG | |
| | ACATTACTGTGGAGTGGCAGTGGAATGGGCAGC | |
| | CAGCGGAGAACTACAAGAACACTCAGCCCATCA | |
| | TGGACACAGATGGCTCTTACTTCGTCTACAGCAA | |
| | GCTCAATGTGCAGAAGAGCAACTGGGAGGCAGG | |
| | AAATACTTTCACCTGCTCTGTGTTACATGAGGGC | |
| | CTGCACAACCACCATACTGAGAAGAGCCTCTCCC | |
| | ACTCTCCTGGTAAA | |
| Fc region of murine IgG2a-- | GTCGACCCAGAGGGCCCACAATCAAGCCCTGT | [SEQ ID NO:30] |
| with Asn-Gln glycosylation | | |
| site mutation | CCTCCATGCAAATGCCCAGCACCTAACCTCTTGG | |
| | GTGGACCATCCGTCTTCATCTTCCCTCCAAAGAT | |
| | CAAGGATGTACTCATGATCTCCCTGAGCCCCATA | |
| | GTCACATGTGTGGTGGTGGATGTGAGCGAGGAT | |
| | GACCCAGATGTCCAGATCAGCTGGTTTGTGAACA | |
| | ACGTGGAAGTACACACAGCTCAGACACAAACCC | |
| | ATAGAGAGGATTACCAAAGTACaCTtCGGGTGGT | |
| | CAGTGCCCTCCCCATCCAGCACCAGGACTGGATG | |
| | AGTGGCAAGGAGTTCAAATGCAAGGTCAACAAC | |
| | AAAGACCTCCCAGCGCCCATCGAGAGAACCATC | |
| | TCAAAACCCAAAGGGTCAGTAAGAGCTCCACAG | |
| | GTATATGTCTTGCCTCCACCAGAAGAAGAGATG | |
| | ACTAAGAAACAGGTCACTCTGACCTGCATGGTG | |
| | ACAGACTTCATGCCTGAAGACATTTACGTGGAGT | |
| | GGACCAACAACGGGAAAACAGAGCTAAACTACA | |
| | AGAACACTGAACCAGTCCTGGACTCTGATGGTTC | |
| | TTACTTCATGTACAGCAAGCTGAGAGTGGAAAA | |
| | GAAGAACTGGGTGGAAAGAAATAGCTACTCCTG | |
| | TTCAGTGGTCCACGAGGGTCTGCACAATCACCAC | |
| | ACGACTAAGAGCTTCTCCCGGACTCCGGGTAAA | |
| Plasmid | DNA sequence | |
| PUB55 | GATCTAACATCCAAAGACGAAAGGTTGAATGAAACCTTTTTGCCAT | SEQ ID NO:31 |
| | CCGACATCCACAGGTCCATTCTCACACATAAGTGCCAAACGCAAC | |
| | AGGAGGGGATACACTAGCAGCAGACCGTTGCAAACGCAGGACCTC | |
| | CACTCCTCTTCTCCTCAACACCCACTTTTGCCATCGAAAAACCAGC | |
| | CCAGTTATTGGGCTTGATTGGAGCTCGCTCATTCCAATTCCTTCTAT | |
| | TAGGCTACTAACACCATGACTTTATTAGCCTGTCTATCCTGGCCCC | |
| | CCTGGCGAGGTTCATGTTTGTTTATTTCCGAATGCAACAAGCTCCG | |
| | CATTACACCCGAACATCACTCCAGATGAGGGCTTTCTGAGTGTGGG | |
| | GTCAAATAGTTTCATGTTCCCCAAATGGCCCAAAACTGACAGTTTA | |
| | AACGCTGTCTTGGAACCTAATATGACAAAAGCGTGATCTCATCCAA | |
| | GATGAACTAAGTTTGGTTCGTTGAAATGCTAACGGCCAGTTGGTCA | |
| | AAAAGAAACTTCCAAAAGTCGCCATACCGTTTGTCTTGTTTGGTAT | |
| | TGATTGACCGAATGCTCAAAAATAATCTCATTAATGCTTAGCGCAGT | |
| | CTCTCTATCGCTTCTGAACCCCGGTGCACCTGTGCCGAAACGCAAA | |
| | TGGGGAAACACCCGCTTTTTGGATGATTATGCATTGTCTCCACATT | |
| | GTATGCTTCCAAGATTCTGGTGGGAATACTGCTGATAGCCTAACGT | |
| | TCATGATCAAAATTTAACTGTTCTAACCCCTACTTGACAGCAATAT | |
| | ATAAACAGAAGGAAGCTGCCCTGTCTTAAACCTTTTTTTTTATCAT | |
| | CATTATTAGCTTACTTTCATAATTGCGACTGGTTCCAATTGACAAG | |
| | CTTTTGATTTTAACGACTTTTAACGACAACTTGAGAAGATCAAAAA | |
| | ACAACTAATTATTCGAAGGATCCAAACGATGAGATTTCCTTCAATT | |
| | TTTACTGCAGTTTTATTCGCAGCATCCTCCGCATTAGCTGCTCCAGT | |
| | CAACACTACAACAGAAGATGAAACGGCACAAATTCCGGCTGAAGC | |
| | TGTCATCGGTTACTCAGATTTAGAAGGGGATTTCGATGTTGCTGTT | |
| | TTGCCATTTTCCAACAGCACAAATAACGGGTTATTGTTTATAAATA | |
| | CTACTATTGCCAGCATTGCTGCTAAAGAAGAAGGGGTATCTCTCGA | |
| | GAAAAGATGCGGACCGGGCAGGGGGTTCGGGAAGAGGAGGCACC | |
| | CCAAAAAGCTGACCCCTTTAGCCTACAAGCAGTTTATCCCCAATGT | |
| | GGCCGAGAAGACCCTAGGCGCCAGCGGAAGGTATGAAGGGAAGA | |
| | TCTCCAGAAACTCCGAGCGATTTAAGGAACTCACCCCCAATTACAA | |
| | CCCCGACATCATATTTAAGGATGAAGAAAACACCGGAGCGGACAG | |
| | GCTGATGACTCAGAGGTGTAAGGACAAGTTGAACGCTTTGGCCAT | |
| | CTCGGTGATGAACCAGTGGCCAGGAGTGAAACTGCGGGTGACCGA | |
| | GGGCTGGGACGAAGATGGCCACCACTCAGAGGAGTCTCTGCACTA | |
| | CGAGGGCCGCGCAGTGGACATCACCACGTCTGACCGCGACCGCAG | |
| | CAAGTACGGCATGCTGGCCCGCCTGGCGGTGGAGGCCGGCTTCGA | |
| | CTGGGTGTACTACGAGTCCAAGGCACATATCCACTGCTCGGTGAA | |
| | AGCAGAGAACTCGGTGGCGGGCCAAATCGGGAGGCTGATTCGCGGC | |
| | CGCGAATTAATTCGCCTTAGACATGACTGTTCCTCAGTTCAAGTTG | |
| | GGCACTTACGAGAAGACCGGTCTTGCTAGATTCTAATCAAGAGGA | |
| | TGTCAGAATGCCATTTGCCTGAGAGATGCAGGCTTCATTTTTGATA | |
| | CTTTTTTATTTGTAACCTATATAGTATAGGATTTTTTTTGTCATTTTG | |
| | TTTCTTCTCGTACGAGCTTGCTCCTGATCAGCCTATCTCGCAGCTGA | |
| | TGAATATCTTGTGGTAGGGGTTTGGGAAAATCATTCGAGTTTGATG | |
| | TTTTTCTTGGTATTTCCCACTCCTCTTCAGAGTACAGAAGATTAAGT | |
| | GAGAAGTTCGTTTGTGCAAGCTTATCGATAAGCTTTAATGCGGTAG | |
| | TTTATCACAGTTAAATTGCTAACGCAGTCAGGCACCGTGTATGAAA | |
| | TCTAACAATGCGCTCATCGTCATCCTCGGCACCGTCACCCTGGATG | |
| | CTGTAGGCATAGGCTTGGTTATGCCGGTACTGCCGGGCCTCTTGCG | |
| | GGATATCGTCCATTCCGACAGCATCGCCAGTCACTATGGCGTGCTG | |
| | CTAGCGCTATATGCGTTGATGCAATTTCTATGCGCACCCGTTCTCG | |
| | GAGCACTGTCCGACCGCTTTGGCCGCCGCCCAGTCCTGCTCGCTTC | |
| | GCTACTTGGAGCCACTATCGACTACGCGATCATGGCGACCACACCC | |
| | GTCCTGTGGATCTATCGAATCTAAATGTAAGTTAAAATCTCTAAAT | |
| | AATTAAATAAGTCCCAGTTTCTCCATACGAACCTTAACAGCATTGC | |
| | GGTGAGCATCTAGACCTTCAACAGCAGCCAGATCCATCACTGCTTG | |
| | GCCAATATGTTTCAGTCCCTCAGGAGTTACGTCTTGTGAAGTGATG | |
| | AACTTCTGGAAGGTTGCAGTGTTAACTCCGCTGTATTGACGGGCAT | |
| | ATCCGTACGTTGGCAAAGTGTGGTTGGTACCGGAGGAGTAATCTCC | |
| | ACAACTCTCTGGAGAGTAGGCACCAACAAACACAGATCCAGCGTG | |
| | TTGTACTTGATCAACATAAGAAGAAGCATTCTCGATTTGCAGGATC | |
| | AAGTGTTCAGGAGCGTACTGATTGGACATTTCCAAAGCCTGCTCGT | |
| | AGGTTGCAACCGATAGGGTTGTAGAGTGTGCAATACACTTGCGTA | |
| | CAATTTCAACCCTTGGCAACTGCACAGCTTGGTTGTGAACAGCATC | |
| | TTCAATTCTGGCAAGCTCCTTGTCTGTCATATCGACAGCCAACAGA | |
| | ATCACCTGGGAATCAATACCATGTTCAGCTTGAGCAGAAGGTCTG | |
| | AGGCAACGAAATCTGGATCAGCGTATTTATCAGCAATAACTAGAA | |
| | CTTCAGAAGGCCCAGCAGGCATGTCAATACTACACAGGGCTGATG | |
| | TGTCATTTTGAACCATCATCTTGGCAGCAGTAACGAACTGGTTTCC | |
| | TGGACCAAATATTTTGTCACACTTAGGAACAGTTTCTGTTCCGTAA | |
| | GCCATAGCAGCTACTGCCTGGGCGCCTCCTGCTAGCACGATACACT | |
| | TAGCACCAACCTTGTGGGCAACGTAGATGACTTCTGGGGTAAGGG | |
| | TACCATCCTTCTTAGGTGGAGATGCAAAAACAATTTCTTTGCAACC | |
| | AGCAACTTTGGCAGGAACACCCAGCATCAGGGAAGTGGAAGGCAG | |
| | AATTGCGGTTCCACCAGGAATATAGAGGCCAACTTTCTCAATAGGT | |
| | CTTGCAAAACGAGAGCAGACTACACCAGGGCAAGTCTCAACTTGC | |
| | AACGTCTCCGTTAGTTGAGCTTCATGGAATTTCCTGACGTTATCTAT | |
| | AGAGAGATCAATGGCTCTCTTAACGTTATCTGGCAATTGCATAAGT | |
| | TCCTCTGGGAAAGGAGCTTCTAACACAGGTGTCTTCAAAGCGACTC | |
| | CATCAAACTTGGCAGTTAGTTCTAAAAGGGCTTTGTCACCATTTTG | |
| | ACGAACATTGTCGACAATTGGTTTGACTAATTCCATAATCTGTTCC | |
| | GTTTTCTGGATAGGACGACGAAGGGCATCTTCAATTTCTTGTGAGG | |
| | AGGCCTTAGAAACGTCAATTTTGCACAATTCAATACGACCTTCAGA | |
| | AGGGACTTCTTTAGGTTTGGATTCTTCTTTAGGTTGTTCCTTGGTGT | |
| | ATCCTGGCTTGGCATCTCCTTTCCTTCTAGTGACCTTTAGGGACTTC | |
| | ATATCCAGGTTTCTCTCCACCTCGTCCAACGTCACACCGTACTTGG | |
| | CACATCTAACTAATGCAAAATAAAATAAGTCAGCACATTCCCAGG | |
| | CTATATCTTCCTTGGATTTAGCTTCTGCAAGTTCATCAGCTTCCTCC | |
| | CTAATTTTAGCGTTCAAACAAAACTTCGTCGTCAAATAACCGTTTG | |
| | GTATAAGAACCTTCTGGAGCATTGCTCTTACGATCCCACAAGGTGC | |
| | TTCCATGGCTCTAAGACCCTTTGATTGGCCAAAACAGGAAGTGCGT | |
| | TCCAAGTGACAGAAACCAACACCTGTTTGTTCAACCACAAATTTCA | |
| | AGCAGTCTCCATCACAATCCAATTCGATACCCAGCAACTTTTGAGT | |
| | TCGTCCAGATGTAGCACCTTTATACCACAAACCGTGACGACGAGAT | |
| | TGGTAGACTCCAGTTTGTGTCCTTATAGCCTCCGGAATAGACTTTTT | |
| | GGACGAGTACACCAGGCCCAACGAGTAATTAGAAGAGTCAGCCAC | |
| | CAAAGTAGTGAATAGACCATCGGGGCGGTCAGTAGTCAAAGACGC | |
| | CAACAAAATTTCACTGACAGGGAACTTTTTGACATCTTCAGAAAGT | |
| | TCGTATTCAGTAGTCAATTGCCGAGCATCAATAATGGGGATTATAC | |
| | CAGAAGCAACAGTGGAAGTCACATCTACCAACTTTGCGGTCTCAG | |
| | AAAAAGCATAAACAGTTCTACTACCGCCATTAGTGAAACTTTTCAA | |
| | ATCGCCCAGTGGAGAAGAAAAAGGCACAGCGATACTAGCATTAGC | |
| | GGGCAAGGATGCAACTTTATCAACCAGGGTCCTATAGATAACCCT | |
| | AGCGCCTGGGATCATCCTTTGGACAACTCTTTCTGCCAAATCTAGG | |
| | TCCAAAATCACTTCATTGATACCATTATTGTACAACTTGAGCAAGT | |
| | TGTCGATCAGCTCCTCAAATTGGTCCTCTGTAACGGATGACTCAAC | |
| | TTGCACATTAACTTGAAGCTCAGTCGATTGAGTGAACTTGATCAGG | |
| | TTGTGCAGCTGGTCAGCAGCATAGGGAAACACGGCTTTTCCTACCA | |
| | AACTCAAGGAATTATCAAACTCTGCAACACTTGCGTATGCAGGTA | |
| | GCAAGGGAAATGTCATACTTGAAGTCGGACAGTGAGTGTAGTCTT | |
| | GAGAAATTCTGAAGCCGTATTTTTATTATCAGTGAGTCAGTCATCA | |
| | GGAGATCCTCTACGCCGGACGCATCGTGGCCGACCTGCAGGTCGG | |
| | CATCACCGGCGCCACAGGTGCGGTTGCTGGCGCCTATATCGCCGAC | |
| | ATCACCGATGGGGAAGATCGGGCTCGCCACTTCGGGCTCATGAGC | |
| | GCTTGTTTCGGCGTGGGTATGGTGGCAGGCCCCGTGGCCGGGGGA | |
| | CTGTTGGGCGCCATCTCCTTGGACCTGCAGGGGGGGGGGGGGAAA | |
| | GCCACGTTGTGTCTCAAAATCTCTGATGTTACATTGCACAAGATAA | |
| | AAATATATCATCATGAACAATAAAACTGTCTGCTTACATAAACAGT | |
| | AATACAAGGGGTGTTATGAGCCATATTCAACGGGAAACGTCTTGC | |
| | TCAAGGCCGCGATTAAATTCCAACATGGATGCTGATTTATATGGGT | |
| | ATAAATGGGCTCGCGATAATGTCGGGCAATCAGGTGCGACAATCT | |
| | ATCGATTGTATGGGAAGCCCGATGCGCCAGAGTTGTTTCTGAAACA | |
| | TGGCAAAGGTAGCGTTGCCAATGATGTTACAGATGAGATGGTCAG | |
| | ACTAAACTGGCTGACGGAATTTATGCCTCTTCCGACCATCAAGCAT | |
| | TTTATCCGTACTCCTGATGATGCATGGTTACTCACCACTGCGATCC | |
| | CCGGGAAAACAGCATTCCAGGTATTAGAAGAATATCCTGATTCAG | |
| | GTGAAAATATTGTTGATGCGCTGGCAGTGTTCCTGCGCCGGTTGCA | |
| | TTCGATTCCTGTTTGTAATTGTCCTTTTAACAGCGATCGCGTATTTC | |
| | GTCTCGCTCAGGCGCAATCACGAATGAATAACGGTTTGGTTGATGC | |
| | GAGTGATTTTGATGACGAGCGTAATGGCTGGCCTGTTGAACAAGTC | |
| | TGGAAAGAAATGCATAAGCTTTTGCCATTCTCACCGGATTCAGTCG | |
| | TCACTCATGGTGATTTCTCACTTGATAACCTTATTTTTGACGAGGG | |
| | GAAATTAATAGGTTGTATTGATGTTGGACGAGTCGGAATCGCAGA | |
| | CCGATACCAGGATCTTGCCATCCTATGGAACTGCCTCGGTGAGTTT | |
| | TCTCCTTCATTACAGAAACGGCTTTTTCAAAAATATGGTATTGATA | |
| | ATCCTGATATGAATAAATTGCAGTTTCATTTGATGCTCGATGAGTT | |
| | TTTCTAATCAGAATTGGTTAATTGGTTGTAACACTGGCAGAGCATT | |
| | ACGCTGACTTGACGGGACGGCGGCTTTGTTGAATAAATCGAACTTT | |
| | TGCTGAGTTGAAGGATCAGATCACGCATCTTCCCGACAACGCAGA | |
| | CCGTTCCGTGGCAAAGCAAAAGTTCAAAATCACCAACTGGTCCAC | |
| | CTACAACAAAGCTCTCATCAACCGTGGCTCCCTCACTTTCTGGCTG | |
| | GATGATGGGGCGATTCAGGCCTGGTATGAGTCAGCAACACCTTCTT | |
| | CACGAGGCAGACCTCAGCGCCCCCCCCCCCCTGCAGGTCCCACGG | |
| | CGGCGGTGCTCAACGGCCTCAACCTACTACTGGGCTGCTTCCTAAT | |
| | GCAGGAGTCGCATAAGGGAGAGCGTCGAGTATCTATGATTGGAAG | |
| | TATGGGAATGGTGATACCCGCATTCTTCAGTGTCTTGAGGTCTCCT | |
| | ATCAGATTATGCCCAACTAAAGCAACCGGAGGAGGAGATTTCATG | |
| | GTAAATTTCTCTGACTTTTGGTCATCAGTAGACTCGAACTGTGAGA | |
| | CTATCTCGGTTATGACAGCAGAAATGTCCTTCTTGGAGACAGTAAA | |
| | TGAAGTCCCACCAATAAAGAAATCCTTGTTATCAGGAACAAACTTC | |
| | TTGTTTCGAACTTTTTCGGTGCCTTGAACTATAAAATGTAGAGTGG | |
| | ATATGTCGGGTAGGAATGGAGCGGGCAAATGCTTACCTTCTGGAC | |
| | CTTCAAGAGGTATGTAGGGTTTGTAGATACTGATGCCAACTTCAGT | |
| | GACAACGTTGCTATTTCGTTCAAACCATTCCGAATCCAGAGAAATC | |
| | AAAGTTGTTTGTCTACTATTGATCCAAGCCAGTGCGGTCTTGAAAC | |
| | TGACAATAGTGTGCTCGTGTTTTGAGGTCATCTTTGTATGAATAAA | |
| | TCTAGTCTTTGATCTAAATAATCTTGACGAGCCAAGGCGATAAATA | |
| | CCCAAATCTAAAACTCTTTTAAAACGTTAAAAGGACAAGTATGTCT | |
| | GCCTGTATTAAACCCCAAATCAGCTCGTAGTCTGATCCTCATCAAC | |
| | TTGAGGGGCACTATCTTGTTTTAGAGAAATTTGCGGAGATGCGATA | |
| | TCGAGAAAAAGGTACGCTGATTTTAAACGTGAAATTTATCTCAAG | |
| | ATCTCTGCCTCGCGCGTTTCGGTGATGACGGTGAAAACCTCTGACA | |
| | CATGCAGCTCCCGGAGACGGTCACAGCTTGTCTGTAAGCGGATGC | |
| | CGGGAGCAGACAAGCCCGTCAGGGCGCGTCAGCGGGTGTTGGCGG | |
| | GTGTCGGGGCGCAGCCATGACCCAGTCACGTAGCGATAGCGGAGT | |
| | GTATACTGGCTTAACTATGCGGCATCAGAGCAGATTGTACTGAGA | |
| | GTGCACCATATGCGGTGTGAAATACCGCACAGATGCGTAAGGAGA | |
| | AAATACCGCATCAGGCGCTCTTCCGCTTCCTCGCTCACTGACTCGC | |
| | TGCGCTCGGTCGTTCGGCTGCGGCGAGCGGTATCAGCTCACTCAAA | |
| | GGCGGTAATACGGTTATCCACAGAATCAGGGGATAACGCAGGAAA | |
| | GAACATGTGAGCAAAAGGCCAGCAAAAGGCCAGGAACCGTAAAA | |
| | AGGCCGCGTTGCTGGCGTTTTTCCATAGGCTCCGCCCCCCTGACGA | |
| | GCATCACAAAAATCGACGCTCAAGTCAGAGGTGGCGAAACCCGAC | |
| | AGGACTATAAAGATACCAGGCGTTTCCCCCTGGAAGCTCCCTCGTG | |
| | CGCTCTCCTGTTCCGACCCTGCCGCTTACCGGATACCTGTCCGCCTT | |
| | TCTCCCTTCGGGAAGCGTGGCGCTTTCTCAATGCTCACGCTGTAGG | |
| | TATCTCAGTTCGGTGTAGGTCGTTCGCTCCAAGCTGGGCTGTGTGC | |
| | ACGAACCCCCCGTTCAGCCCGACCGCTGCGCCTTATCCGGTAACTA | |
| | TCGTCTTGAGTCCAACCCGGTAAGACACGACTTATCGCCACTGGCA | |
| | GCAGCCACTGGTAACAGGATTAGCAGAGCGAGGTATGTAGGCGGT | |
| | GCTACAGAGTTCTTGAAGTGGTGGCCTAACTACGGCTACACTAGA | |
| | AGGACAGTATTTGGTATCTGCGCTCTGCTGAAGCCAGTTACCTTCG | |
| | GAAAAAGAGTTGGTAGCTCTTGATCCGGCAAACAAACCACCGCTG | |
| | GTAGCGGTGGTTTTTTTGTTTGCAAGCAGCAGATTACGCGCAGAAA | |
| | AAAAGGATCTCAAGAAGATCCTTTGATCTTTTCTACGGGGTCTGAC | |
| | GCTCAGTGGAACGAAAACTCACGTTAAGGGATTTTGGTCATGAGA | |
| | TTATCAAAAAGGATCTTCACCTAGATCCTTTTAAATTAAAAATGAA | |
| | GTTTTAAATCAATCTAAAGTATATATGAGTAAACTTGGTCTGACAG | |
| | TTACCAATGCTTAATCAGTGAGGCACCTATCTCAGCGATCTGTCTA | |
| | TTTCGTTCATCCATAGTTGCCTGACTCCCCGTCGTGTAGATAACTAC | |
| | GATACGGGAGGGCTTACCATCTGGCCCCAGTGCTGCAATGATACC | |
| | GCGAGACCCACGCTCACCGGCTCCAGATTTATCAGCAATAAACCA | |
| | GCCAGCCGGAAGGGCCGAGCGCAGAAGTGGTCCTGCAACTTTATC | |
| | CGCCTCCATCCAGTCTATTAATTGTTGCCGGGAAGCTAGAGTAAGT | |
| | AGTTCGCCAGTTAATAGTTTGCGCAACGTTGTTGCCATTGCTGCAG | |
| | GCATCGTGGTGTCACGCTCGTCGTTTGGTATGGCTTCATTCAGCTC | |
| | CGGTTCCCAACGATCAAGGCGAGTTACATGATCCCCCATGTTGTGC | |
| | AAAAAAGCGGTTAGCTCCTTCGGTCCTCCGATCGTTGTCAGAAGTA | |
| | AGTTGGCCGCAGTGTTATCACTCATGGTTATGGCAGCACTGCATAA | |
| | TTCTCTTACTGTCATGCCATCCGTAAGATGCTTTTCTGTGACTGGTG | |
| | AGTACTCAACCAAGTCATTCTGAGAATAGTGTATGCGGCGACCGA | |
| | GTTGCTCTTGCCCGGCGTCAACACGGGATAATACCGCGCCACATAG | |
| | CAGAACTTTAAAAGTGCTCATCATTGGAAAACGTTCTTCGGGGCGA | |
| | AAACTCTCAAGGATCTTACCGCTGTTGAGATCCAGTTCGATGTAAC | |
| | CCACTCGTGCACCCAACTGATCTTCAGCATCTTTTACTTTCACCAGC | |
| | GTTTCTGGGTGAGCAAAAACAGGAAGGCAAAATGCCGCAAAAAA | |
| | GGGAATAAGGGCGACACGGAAATGTTGAATACTCATACTCTTCCTT | |
| | TTTCAATATTATTGAAGCATTTATCAGGGTTATTGTCTCATGAGCG | |
| | GATACATATTTGAATGTATTTAGAAAAATAAACAAATAGGGGTTC | |
| | CGCGCACATTTCCCCGAAAAGTGCCACCTGACGTCTAAGAAACCA | |
| | TTATTATCATGACATTAACCTATAAAAATAGGCGTATCACGAGGCC | |
| | CTTTCGTCTTCAAGAATTAATTCTCATGTTTGACAGCTTATCATCGA | |
| | TAAGCTGACTCATGTTGGTATTGTGAAATAGACGCAGATCGGGAA | |
| | CACTGAAAAATAACAGTTATTATTCGAGATC | |
| pUB114 | GATCTAACATCCAAAGACGAAAGGTTGAATGAAACCTTTTTGCCAT | SEQ ID NO:32 |
| | CCGACATCCACAGGTCCATTCTCACACATAAGTGCCAAACGCAAC | |
| | AGGAGGGGATACACTAGCAGCAGACCGTTGCAAACGCAGGACCTC | |
| | CACTCCTCTTCTCCTCAACACCCACTTTTGCCATCGAAAAACCAGC | |
| | CCAGTTATTGGGCTTGATTGGAGCTCGCTCATTCCAATTCCTTCTAT | |
| | TAGGCTACTAACACCATGACTTTATTAGCCTGTCTATCCTGGCCCC | |
| | CCTGGCGAGGTTCATGTTTGTTTATTTCCGAATGCAACAAGCTCCG | |
| | CATTACACCCGAACATCACTCCAGATGAGGGCTTTCTGAGTGTGGG | |
| | GTCAAATAGTTTCATGTTCCCCAAATGGCCCAAAACTGACAGTTTA | |
| | AACGCTGTCTTGGAACCTAATATGACAAAAGCGTGATCTCATCCAA | |
| | GATGAACTAAGTTTGGTTCGTTGAAATGCTAACGGCCAGTTGGTCA | |
| | AAAAGAAACTTCCAAAAGTCGCCATACCGTTTGTCTTGTTTGGTAT | |
| | TGATTGACGAATGCTCAAAAATAATCTCATTAATGCTTAGCGCAGT | |
| | CTCTCTATCGCTTCTGAACCCCGGTGCACCTGTGCCGAAACGCAAA | |
| | TGGGGAAACACCCGCTTTTTGGATGATTATGCATTGTCTCCACATT | |
| | GTATGCTTCCAAGATTCTGGTGGGAATACTGCTGATAGCCTAACGT | |
| | TCATGATCAAAATTTAACTGTTCTAACCCCTACTTGACAGCAATAT | |
| | ATAAACAGAAGGAAGCTGCCCTGTCTTAAACCTTTTITTTTATCAT | |
| | CATTATTAGCTTACTTTCATAATTGCGACTGGTTCCAATTGACAAG | |
| | CTTTTGATTTTAACGACTTTTAACGACAACTTGAGAAGATCAAAAA | |
| | ACAACTAATTATTCGAAGGATCCAAACGATGAGATTTCCTTCAATT | |
| | TTTACTGCAGTTTTATTCGCAGCATCCTCCGCATTAGCTGCTCCAGT | |
| | CAACACTACAACAGAAGATGAAACGGCACAAATTCCGGCTGAAGC | |
| | TGTCATCGGTTACTCAGATTTAGAAGGGGATTTCGATGTTGCTGTT | |
| | TTGCCATTTTCCAACAGCACAAATAACGGGTTATTGTTTATAAATA | |
| | CTACTATTGCCAGCATTGCTGCTAAAGAAGAAGGGGTATCTCTCGA | |
| | GAAAAGATGCGGACCGGGCAGGGGGTTCGGGAAGAGGAGGCACC | |
| | CCAAAAAGCTGACCCCTTTAGCCTACAAGCAGTTTATCCCCAATGT | |
| | GGCCGAGAAGACCCTAGGCGCCAGCGGAAGGTATGAAGGGAAGA | |
| | TCTCCAGAAACTCCGAGCGATTTAAGGAACTCACCCCCAATTACAA | |
| | CCCCGACATCATATTTAAGGATGAAGAAAACACCGGAGCGGACAG | |
| | GCTGATGACTCAGAGGTGTAAGGACAAGTTGAACGCTTTGGCCAT | |
| | CTCGGTGATGAACCAGTGGCCAGGAGTGAAACTGCGGGTGACCGA | |
| | GGGCTGGGACGAAGATGGCCACCACTCAGAGGAGTCTCTGCACTA | |
| | CGAGGGCCGCGCAGTGGACATCACCACGTCTGACCGCGACCGCAG | |
| | CAAGTACGGCATGCTGGCCCGCCTGGCGGTGGAGGCCGGCTTCGA | |
| | CTGGGTGTACTACGAGTCCAAGGCACATATCCACTGCTCGGTGAA | |
| | AGCAGAGAACTCGGTGGCGGCCAAATCGGGAGGCGTCGACGTGCC | |
| | CAGGGATTGTGGTTGTAAGCCTTGCATATGTACAGTCCCAGAAGTA | |
| | TCATCTGTCTTCATCTTCCCCCCAAAGCCCAAGGATGTGCTCACCA | |
| | TTACTCTGACTCCTAAGGTCACGTGTGTTGTGGTAGACATCAGCAA | |
| | GGATGATCCCGAGGTCCAGTTCAGCTGGTTTGTAGATGATGTGGAG | |
| | GTGCACACAGCTCAGACGCAACCaCGGGAaGAGCAGTTCCAAAGC | |
| | ACTTTCCGCTCAGTCAGTGAACTTCCCATCATGCACCAGGACTGGC | |
| | TCAATGGCAAGGAGTTCAAATGCAGGGTCAACAGTGCAGCTTTCC | |
| | CTGCCCCCATCGAGAAAACCATCTCCAAAACCAAAGGCAGACCGA | |
| | AGGCTCCACAGGTGTACACCATTCCACCTCCCAAGGAGCAGATGG | |
| | CCAAGGATAAAGTCAGTCTGACCTGCATGATAACAGACTTCTTCCC | |
| | TGAAGACATTACTGTGGAGTGGCAGTGGAATGGGCAGCCAGCGGA | |
| | GAACTACAAGAACACTCAGCCCATCATGGACACAGATGGCTCTTA | |
| | CTTCGTCTACAGCAAGCTCAATGTGCAGAAGAGCAACTGGGAGGC | |
| | AGGAAATACTTTCACCTGCTCTGTGTTACATGAGGGCCTGCACAAC | |
| | CACCATACTGAGAAGAGCCTCTCCCACTCTCCTGGTAAATGATCCC | |
| | AGTGTCCTTGGAGCCCTCTGGTCCTACAGCGGCCGCGAATTAATTC | |
| | GCCTTAGACATGACTGTTCCTCAGTTCAAGTTGGGCACTTACGAGA | |
| | AGACCGGTCTTGCTAGATTCTAATCAAGAGGATGTCAGAATGCCAT | |
| | TTGCCTGAGAGATGCAGGCTTCATTTTTGATACTTTTTTATTTGTAA | |
| | CCTATATAGTATAGGATTTTTTTTGTCATTTTGTTTCTTCTCGTACG | |
| | AGCTTGCTCCTGATCAGCCTATCTCGCAGCTGATGAATATCTTGTG | |
| | GTAGGGGTTTGGGAAAATCATTCGAGTTTGATGTTTTTCTTGGTAT | |
| | TTCCCACTCCTCTTCAGAGTACAGAAGATTAAGTGAGAAGTTCTTC | |
| | TGTGCAAGCTTATCGATAAGCTTTAATGCGGTAGTTTATCACATTC | |
| | AAATTGCTAACGCAGTCAGGCACCGTGTATGAAATCTAACAATGC | |
| | GCTCATCGTCATCCTCGGCACCGTCACCCTGGATGCTGTAGGCATA | |
| | GGCTTGGTTATGCCGGTACTGCCGGGCCTCTTGCGGGATATCGTCC | |
| | ATTCCGACAGCATCGCCAGTCACTATGGCGTGCTGCTAGCGCTATA | |
| | TGCGTTGATGCAATTTCTATGCGCACCCGTTCTCGGAGCACTGTCC | |
| | GACCGCTTTGGCCGCCGCCCAGTCCTGCTCGCTTCGCTACTTGGAG | |
| | CCACTATCGACTACGCGATCATGGCGACCACACCCGTCCTGTGGAT | |
| | CTATCGAATCTAAATGTAAGTTAAAATCTCTAAATAATTAAATAAG | |
| | TCCCAGTTTCTCCATACGAACCTTAACAGCATTGCGGTGAGCATCT | |
| | AGACCTTCAACAGCAGCCAGATCCATCACTGCTTGGCCAATATGTT | |
| | TCAGTCCCTCAGGAGTTACGTCTTGTGAAGTGATGAACTTCTGGAA | |
| | GGTTGCAGTGTTAACTCCGCTGTATTGACGGGCATATCCGTACGTT | |
| | GGCAAAGTGTGGTTGGTACCGGAGGAGTAATCTCCACAACTCTCT | |
| | GGAGAGTAGGCACCAACAAACACAGATCCAGCGTGTTGTACTTGA | |
| | TCAACATAAGAAGAAGCATTCTCGATTTGCAGGATCAAGTGTTCA | |
| | GGAGCGTACTGATTGGACATTTCCAAAGCCTGCTCGTAGGTTGCAA | |
| | CCGATAGGGTTGTAGAGTGTGCAATACACTTGCGTACAATTTCAAC | |
| | CCTTGGCAACTGCACAGCTTGGTTGTGAACAGCATCTTCAATTCTG | |
| | GCAAGCTCCTTGTCTGTCATATCGACAGCCAACAGAATCACCTGGG | |
| | AATCAATACCATGTTCAGCTTGAGCAGAAGGTCTGAGGCAACGAA | |
| | ATCTGGATCAGCGTATTTATCAGCAATAACTAGAACTTCAGAAGGC | |
| | CCAGCAGGCATGTCAATACTACACAGGGCTGATGTGTCATTTTGAA | |
| | CCATCATCTTGGCAGCAGTAACGAACTGGTTTCCTGGACCAAATAT | |
| | TTTGTCACACTTAGGAACAGTTTCTGTTCCGTAAGCCATAGCAGCT | |
| | ACTGCCTGGGCGCCTCCTGCTAGCACGATACACTTAGCACCAACCT | |
| | TGTGGGCAACGTAGATGACTTCTGGGGTAAGGGTACCATCCTTCTT | |
| | AGGTGGAGATGCAAAAACAATTTCTTTGCAACCAGCAACTTTGGC | |
| | AGGAACACCCAGCATCAGGGAAGTGGAAGGCAGAATTGCGGTTCC | |
| | ACCAGGAATATAGAGGCCAACTTTCTCAATAGGTCTTGCAAAACG | |
| | AGAGCAGACTACACCAGGGCAAGTCTCAACTTGCAACGTCTCCGT | |
| | TAGTTGAGCTTCATGGAATTTCCTGACGTTATCTATAGAGAGATCA | |
| | ATGGCTCTCTTAACGTTATCTGGCAATTGCATAAGTTCCTCTGGGA | |
| | AAGGAGCTTCTAACACAGGTGTCTTCAAAGCGACTCCATCAAACTT | |
| | GGCAGTTAGTTCTAAAAGGGCTTTGTCACCATTTTGACGAACATTG | |
| | TCGACAATTGGTTTGACTAATTCCATAATCTGTTCCGTTTTCTGGAT | |
| | AGGACGACGAAGGGCATCTTCAATTTCTTGTGAGGAGGCCTTAGA | |
| | AACGTCAATTTTGCACAATTCAATACGACCTTCAGAAGGGACTTCT | |
| | TTAGGTTTGGATTCTTCTTTAGGTTGTTCCTTGGTGTATCCTGGCTT | |
| | GGCATCTCCTTTCCTTCTAGTGACCTTTAGGGACTTCATATCCAGGT | |
| | TTCTCTCCACCTCGTCCAACGTCACACCGTACTTGGCACATCTAAC | |
| | TAATGCAAAATAAAATAAGTCAGCACATTCCCAGGCTATATCTTCC | |
| | TTGGATTTAGCTTCTGCAAGTTCATCAGCTTCCTCCCTAATTTTAGC | |
| | GTTCAAACAAAACTTCGTCGTCAAATAACCGTTTGGTATAAGAACC | |
| | TTCTGGAGCATTGCTCTTACGATCCCACAAGGTGCTTCCATGGCTC | |
| | TAAGACCCTTTGATTGGCCAAAACAGGAAGTGCGTTCCAAGTGAC | |
| | AGAAACCAACACCTGTTTGTTCAACCACAAATTTCAAGCAGTCTCC | |
| | ATCACAATCCAATTCGATACCCAGCAACTTTTGAGTTCGTCCAGAT | |
| | GTAGCACCTTTATACCACAAACCGTGACGACGAGATTGGTAGACT | |
| | CCAGTTTGTGTCCTTATAGCCTCCGGAATAGACTTTTTGGACGAGT | |
| | ACACCAGGCCCAACGAGTAATTAGAAGAGTCAGCCACCAAAGTAG | |
| | TGAATAGACCATCGGGGCGGTCAGTAGTCAAAGACGCCAACAAAA | |
| | TTTCACTGACAGGGAACTTTTTGACATCTTCAGAAAGTTCGTATTC | |
| | AGTAGTCAATTGCCGAGCATCAATAATGGGGATTATACCAGAAGC | |
| | AACAGTGGAAGTCACATCTACCAACTTTGCGGTCTCAGAAAAAGC | |
| | ATAAACAGTTCTACTACCGCCATTAGTGAAACTTTTCAAATCGCCC | |
| | AGTGGAGAAGAAAAAGGCACAGCGATACTAGCATTAGCGGGCAA | |
| | GGATGCAACTTTATCAACCAGGGTCCTATAGATAACCCTAGCGCCT | |
| | GGGATCATCCTTTGGACAACTCTTTCTGCCAAATCTAGGTCCAAAA | |
| | TCACTTCATTGATACCATTATTGTACAACTTGAGCAAGTTGTCGAT | |
| | CAGCTCCTCAAATTGGTCCTCTGTAACGGATGACTCAACTTGCACA | |
| | TTAACTTGAAGCTCAGTCGATTGAGTGAACTTGATCAGGTTGTGCA | |
| | GCTGGTCAGCAGCATAGGGAAACACGGCTTTTCCTACCAAACTCA | |
| | AGGAATTATCAAACTCTGCAACACTTGCGTATGCAGGTAGCAAGG | |
| | GAAATGTCATACTTGAAGTCGGACAGTGAGTGTAGTCTTGAGAAA | |
| | TTCTGAAGCCGTATTTTTATTATCAGTGAGTCAGTCATCAGGAGAT | |
| | CCTCTACGCCGGACGCATCGTGGCCGACCTGCAGGTCGGCATCACC | |
| | GGCGCCACAGGTGCGGTTGCTGGCGCCTATATCGCCGACATCACC | |
| | GATGGGGAAGATCGGGCTCGCCACTTCGGGCTCATGAGCGCTTGTT | |
| | TCGGCGTGGGTATGGTGGCAGGCCCCGTGGCCGGGGGACTGTTGG | |
| | GCGCCATCTCCTTGGACCTGCAGGGGGGGGGGGGGAAAGCCACGT | |
| | TGTGTCTCAAAATCTCTGATGTTACATTGCACAAGATAAAAATATA | |
| | TCATCATGAACAATAAAACTGTCTGCTTACATAAACAGTAATACAA | |
| | GGGGTGTTATGAGCCATATTCAACGGGAAACGTCTTGCTCAAGGC | |
| | CGCGATTAAATTCCAACATGGATGCTGATTTATATGGGTATAAATG | |
| | GGCTCGCGATAATGTCGGGCAATCAGGTGCGACAATCTATCGATT | |
| | GTATGGGAAGCCCGATGCGCCAGAGTTGTTTCTGAAACATGGCAA | |
| | AGGTAGCGTTGCCAATGATGTTACAGATGAGATGGTCAGACTAAA | |
| | CTGGCTGACGGAATTTATGCCTCTTCCGACCATCAAGCATTTTATC | |
| | CGTACTCCTGATGATGCATGGTTACTCACCACTGCGATCCCCGGGA | |
| | AAACAGCATTCCAGGTATTAGAAGAATATCCTGATTCAGGTGAAA | |
| | ATATTGTTGATGCGCTGGCAGTGTTCCTGCGCCGGTTGCATTCGAT | |
| | TCCTGTTTGTAATTGTCCTTTTAACAGCGATCGCGTATTTCGTCTCG | |
| | CTCAGGCGCAATCACGAATGAATAACGGTTTGGTTGATGCGAGTG | |
| | ATTTTGATGACGAGCGTAATGGCTGGCCTGTTGAACAAGTCTGGAA | |
| | AGAAATGCATAAGCTTTTGCCATTCTCACCGGATTCAGTCGTCACT | |
| | CATGGTGATTTCTCACTTGATAACCTTATTTTTGACGAGGGGAAAT | |
| | TAATAGGTTGTATTGATGTTGGACGAGTCGGAATCGCAGACCGAT | |
| | ACCAGGATCTTGCCATCCTATGGAACTGCCTCGGTGAGTTTTCTCC | |
| | TTCATTACAGAAACGGCTTTTTCAAAAATATGGTATTGATAATCCT | |
| | GATATGAATAAATTGCAGTTTCATTTGATGCTCGATGAGTTTTTCT | |
| | AATCAGAATTGGTTAATTGGTTGTAACACTGGCAGAGCATTACGCT | |
| | GACTTGACGGGACGGCGGCTTTGTTGAATAAATCGAACTTTTGCTG | |
| | AGTTGAAGGATCAGATCACGCATCTTCCCGACAACGCAGACCGTT | |
| | CCGTGGCAAAGCAAAAGTTCAAAATCACCAACTGGTCCACCTACA | |
| | ACAAAGCTCTCATCAACCGTGGCTCCCTCACTTTCTGGCTGGATGA | |
| | TGGGGCGATTCAGGCCTGGTATGAGTCAGCAACACCTTCTTCACGA | |
| | GGCAGACCTCAGCGCCCCCCCCCCCCTGCAGGTCCCACGGCGGCG | |
| | GTGCTCAACGGCCTCAACCTACTACTGGGCTGCTTCCTAATGCAGG | |
| | AGTCGCATAAGGGAGAGCGTCGAGTATCTATGATTGGAAGTATGG | |
| | GAATGGTGATACCCGCATTCTTCAGTGTCTTGAGGTCTCCTATCAG | |
| | ATTATGCCCAACTAAAGCAACCGGAGGAGGAGATTTCATGGTAAA | |
| | TTTCTCTGACTTTTGGTCATCAGTAGACTCGAACTGTGAGACTATCT | |
| | CGGTTATGACAGCAGAAATGTCCTTCTTGGAGACAGTAAATGAAG | |
| | TCCCACCAATAAAGAAATCCTTGTTATCAGGAACAAACTTCTTGTT | |
| | TCGAACTTTTTCGGTGCCTTGAACTATAAAATGTAGAGTGGATATG | |
| | TCGGGTAGGAATGGAGCGGGCAAATGCTTACCTTCTGGACCTTCA | |
| | AGAGGTATGTAGGGTTTGTAGATACTGATGCCAACTTCAGTGACA | |
| | ACGTTGCTATTTCGTTCAAACCATTCCGAATCCAGAGAAATCAAAG | |
| | TTGTTTGTCTACTATTGATCCAAGCCAGTGCGGTCTTGAAACTGAC | |
| | AATAGTGTGCTCGTGTTTTGAGGTCATCTTTGTATGAATAAATCTA | |
| | GTCTTTGATCTAAATAATCTTGACGAGCCAAGGCGATAAATACCCA | |
| | AATCTAAAACTCTTTTAAAACGTTAAAAGGACAAGTATGTCTGCCT | |
| | GTATTAAACCCCAAATCAGCTCGTAGTCTGATCCTCATCAACTTGA | |
| | GGGGCACTATCTTGTTTTAGAGAAATTTGCGGAGATGCGATATCGA | |
| | GAAAAAGGTACGCTGATTTTAAACGTGAAATTTATCTCAAGATCTC | |
| | TGCCTCGCGCGTTTCGGTGATGACGGTGAAAACCTCTGACACATGC | |
| | AGCTCCCGGAGACGGTCACAGCTTGTCTGTAAGCGGATGCCGGGA | |
| | GCAGACAAGCCCGTCAGGGCGCGTCAGCGGGTGTTGGCGGGTGTC | |
| | GGGGCGCAGCCATGACCCAGTCACGTAGCGATAGCGGAGTGTATA | |
| | CTGGCTTAACTATGCGGCATCAGAGCAGATTGTACTGAGAGTGCA | |
| | CCATATGCGGTGTGAAATACCGCACAGATGCGTAAGGAGAAAATA | |
| | CCGCATCAGGCGCTCTTCCGCTTCCTCGCTCACTGACTCGCTGCGC | |
| | TCGGTCGTTCGGCTGCGGCGAGCGGTATCAGCTCACTCAAAGGCG | |
| | GTAATACGGTTATCCACAGAATCAGGGGATAACGCAGGAAAGAAC | |
| | ATGTGAGCAAAAGGCCAGCAAAAGGCCAGGAACCGTAAAAAGGC | |
| | CGCGTTGCTGGCGTTTTTCCATAGGCTCCGCCCCCCTGACGAGCAT | |
| | CACAAAAATCGACGCTCAAGTCAGAGGTGGCGAAACCCGACAGGA | |
| | CTATAAAGATACCAGGCGTTTCCCCCTGGAAGCTCCCTCGTGCGCT | |
| | CTCCTGTTCCGACCCTGCCGCTTACCGGATACCTGTCCGCCTTTCTC | |
| | CCTTCGGGAAGCGTGGCGCTTTCTCAATGCTCACGCTGTAGGTATC | |
| | TCAGTTCGGTGTAGGTCGTTCGCTCCAAGCTGGGCTGTGTGCACGA | |
| | ACCCCCCGTTCAGCCCGACCGCTGCGCCTTATCCGGTAACTATCGT | |
| | CTTGAGTCCAACCCGGTAAGACACGACTTATCGCCACTGGCAGCA | |
| | GCCACTGGTAACAGGATTAGCAGAGCGAGGTATGTAGGCGGTGCT | |
| | ACAGAGTTCTTGAAGTGGTGGCCTAACTACGGCTACACTAGAAGG | |
| | ACAGTATTTGGTATCTGCGCTCTGCTGAAGCCAGTTACCTTCGGAA | |
| | AAAGAGTTGGTAGCTCTTGATCCGGCAAACAAACCACCGCTGGTA | |
| | GCGGTGGTTTTTTTGTTTGCAAGCAGCAGATTACGCGCAGAAAAAAA | |
| | AGGATCTCAAGAAGATCCTTTGATCTTTTCTACGGGGTCTGACGCT | |
| | CAGTGGAACGAAAACTCACGTTAAGGGATTTTGGTCATGAGATTA | |
| | TCAAAAAGGATCTTCACCTAGATCCTTTTAAATTAAAAATGAAGTT | |
| | TTAAATCAATCTAAAGTATATATGAGTAAACTTGGTCTGACAGTTA | |
| | CCAATGCTTAATCAGTGAGGCACCTATCTCAGCGATCTGTCTATTT | |
| | CGTTCATCCATAGTTGCCTGACTCCCCGTCGTGTAGATAACTACGA | |
| | TACGGGAGGGCTTACCATCTGGCCCCAGTGCTGCAATGATACCGC | |
| | GAGACCCACGCTCACCGGCTCCAGATTTATCAGCAATAAACCAGC | |
| | CAGCCGGAAGGGCCGAGCGCAGAAGTGGTCCTGCAACTTTATCCG | |
| | CCTCCATCCAGTCTATTAATTGTTGCCGGGAAGCTAGAGTAAGTAG | |
| | TTCGCCAGTTAATAGTTTGCGCAACGTTGTTGCCATTGCTGCAGGC | |
| | ATCGTGGTGTCACGCTCGTCGTTTGGTATGGCTTCATTCAGCTCCG | |
| | TTCCCCAACGATCAAGGCGAGTTACATGATCCCCCATGTTGTGCAA | |
| | AAAAGCGGTTAGCTCCTTCGGTCCTCCGATCGTTGTCAGAAGTAAG | |
| | TTGGCCGCAGTGTTATCACTCATGGTTATGGCAGCACTGCATAATT | |
| | CTCTTACTGTCATGCCATCCGTAAGATGCTTTTCTGTGACTGGTGA | |
| | GTACTCAACCAAGTCATTCTGAGAATAGTGTATGCGGCGACCGAG | |
| | TTGCTCTTGCCCGGCGTCAACACGGGATAATACCGCGCCACATAGC | |
| | AGAACTTTAAAAGTGCTCATCATTGGAAAACGTTCTTCGGGGCGA | |
| | AAACTCTCAAGGATCTTACCGCTGTTGAGATCCAGTTCGATGTAAC | |
| | CCACTCGTGCACCCAACTGATCTTCAGCATCTTTTACTTTCACCAGC | |
| | GTTCTCTGGGTGAGCAAAAACAGGAAGGCAAAATGCCGCAAAAAA | |
| | GGGAATAAGGGCGACACGGAAATGTTGAATACTCATACTCTTCCTT | |
| | TTTCAATATTATTGAAGCATTTATCAGGGTTATTGTCTCATGAGCG | |
| | GATACATATTTGAATGTATTTAGAAAAATAAACAAATAGGGGTTC | |
| | CGCGCACATTTCCCCGAAAAGTGCCACCTGACGTCTAAGAAACCA | |
| | TTATTATCATGACATTAACCTATAAAAATAGGCGTATCACGAGGCC | |
| | CTTTCGTCTTCAAGAATTAATTCTCATGTTTGACAGCTTATCATCGA | |
| | TAAGCTGACTCATGTTGGTATTGTGAAATAGACGCAGATCGGGAA | |
| | CACTGAAAAATAACAGTTATTATTCGAGATC | |
| pUB 115 | GATCTAACATCCAAAGACGAAAGGTTGAATGAAACCTTTTTGCCAT | SEQ ID NO:33 |
| | CCGACATCCACAGGTCCATTCTCACACATAAGTGCCAAACGCAAC | |
| | AGGAGGGGATACACTAGCAGCAGACCGTTGCAAACGCAGGACCTC | |
| | CACTCCTCTTCTCCTCAACACCCACTTTTGCCATCGAAAAACCAGC | |
| | CCAGTTATTGGGCTTGATTGGAGCTCGCTCATTCCAATTCCTTCTAT | |
| | TAGGCTACTAACACCATGACTTTATTAGCCTGTCTATCCTGGCCCC | |
| | CCTGGCGAGGTTCATGTTTGTTTATTTCCGAATGCAACAAGCTCCG | |
| | CATTACACCCGAACATCACTCCAGATGAGGGCTTTCTGAGTGTGGG | |
| | GTCAAATAGTTTCATGTTCCCCAAATGGCCCAAAACTGACAGTTTA | |
| | AACGCTGTCTTGGAACCTAATATGACAAAAGCGTGATCTCATCCAA | |
| | GATGAACTAAGTTTGGTTCGTTGAAATGCTAACGGCCAGTTGGTCA | |
| | AAAAGAAACTTCCAAAAGTCGCCATACCGTTTGTCTTGTTTGGTAT | |
| | TGATTGACGAATGCTCAAAAATAATCTCATTAATGCTTAGCGCAGT | |
| | CTCTCTATCGCTTCTGAACCCCGGTGCACCTGTGCCGAAACGCAAA | |
| | TGGGGAAACACCCGCTTTTTGGATGATTATGCATTGTCTCCACATT | |
| | GTATGCTTCCAAGATTCTGGTGGGAATACTGCTGATAGCCTAACGT | |
| | TCATGATCAAAATTTAACTGTTCTAACCCCTACTTGACAGCAATAT | |
| | ATAAACAGAAGGAAGCTGCCCTGTCTTAAACCTTTTTTTTTATCAT | |
| | CATTATTAGCTTACTTTCATAATTGCGACTGGTTCCAATTGACAAG | |
| | CTTTTGATTTTAACGACTTTTAACGACAACTTGAGAAGATCAAAAA | |
| | ACAACTAATTATTCGAAGGATCCAAACGATGAGATTTCCTTCAATT | |
| | TTTACTGCAGTTTTATTCGCAGCATCCTCCGCATTAGCTGCTCCAGT | |
| | CAACACTACAACAGAAGATGAAACGGCACAAATTCCGGCTGAAGC | |
| | TGTCATCGGTTACTCAGATTTAGAAGGGGATTTCGATGTTGCTGTT | |
| | TTGCCATTTTCCAACAGCACAAATAACGGGTTATTGTTTATAAATA | |
| | CTACTATTGCCAGCATTGCTGCTAAAGAAGAAGGGGTATCTCTCGA | |
| | GAAAAGATGCGGACCGGGCAGGGGGTTCGGGAAGAGGAGGCACC | |
| | CCAAAAAGCTGACCCCTTTAGCCTACAAGCAGTTTATCCCCAATGT | |
| | GGCCGAGAAGACCCTAGGCGCCAGCGGAAGGTATGAAGGGAAGA | |
| | TCTCCAGAAACTCCGAGCGATTTAAGGAACTCACCCCCAATTACAA | |
| | CCCCGACATCATATTTAAGGATGAAGAAAACACCGGAGCGGACAG | |
| | GCTGATGACTCAGAGGTGTAAGGACAAGTTGAACGCTTTGGCCAT | |
| | CTCGGTGATGAACCAGTGGCCAGGAGTGAAACTGCGGGTGACCGA | |
| | GGGCTGGGACGAAGATGGCCACCACTCAGAGGAGTCTCTGCACTA | |
| | CGAGGGCCGCGCAGTGGACATCACCACGTCTGACCGCGACCGCAG | |
| | CAAGTACGGCATGCTGGCCCGCCTGGCGGTGGAGGCCGGCTTCGA | |
| | CTGGGTGTACTACGAGTCCAAGGCACATATCCACTGCTCGGTGAA | |
| | AGCAGAGAACTCGGTGGCGGCCAAATCGGGAGGCGTCGACCCCAG | |
| | AGGGCCCACAATCAAGCCCTGTCCTCCATGCAAATGCCCAGCACCT | |
| | AACCTCTTGGGTGGACCATCCGTCTTCATCTTCCCTCCAAAGATCA | |
| | AGGATGTACTCATGATCTCCCTGAGCCCCATAGTCACATGTGTGGT | |
| | GGTGGATGTGAGCGAGGATGACCCAGATGTCCAGATCAGCTGGTT | |
| | TGTGAACAACGTGGAAGTACACACAGCTCAGACACAAACCCATAG | |
| | AGAGGATTACCAAAGTACaCTtCGGGTGGTCAGTGCCCTCCCCATCC | |
| | AGCACCAGGACTGGATGAGTGGCAAGGAGTTCAAATGCAAGGTCA | |
| | ACAACAAAGACCTCCCAGCGCCCATCGAGAGAACCATCTCAAAAC | |
| | CCAAAGGGTCAGTAAGAGCTCCACAGGTATATGTCTTGCCTCCACC | |
| | AGAAGAAGAGATGACTAAGAAACAGGTCACTCTGACCTGCATGGT | |
| | GACAGACTTCATGCCTGAAGACATTTACGTGGAGTGGACCAACAA | |
| | CGGGAAAACAGAGCTAAACTACAAGAACACTGAACCAGTCCTGGA | |
| | CTCTGATGGTTCTTACTTCATGTACAGCAAGCTGAGAGTGGAAAAG | |
| | AAGAACTGGGTGGAAAGAAATAGCTACTCCTGTTCAGTGGTCCAC | |
| | GAGGGTCTGCACAATCACCACACGACTAAGAGCTTCTCCCGGACT | |
| | CCGGGTAAATGAGCTCAGATCGATTCCATGGATCCTCACATCCCAA | |
| | TCCGCGGCCGCGAATTAATTCGCCTTAGACATGACTGTTCCTCAGT | |
| | TCAAGTTGGGCACTTACGAGAAGACCGGTCTTGCTAGATTCTAATC | |
| | AAGAGGATGTCAGAATGCCATTTGCCTGAGAGATGCAGGCTTCAT | |
| | TTTTGATACTTTTTTATTTGTAACCTATATAGTATAGGATTTTTTTTG | |
| | TCATTTTGTTTCTTCTCGTACGAGCTTGCTCCTGATCAGCCTATCTC | |
| | GCAGCTGATGAATATCTTGTGGTAGGGGTTTGGGAAAATCATTCGA | |
| | GTTCTGATGTTTTTCTTGGTATTTCCCACTCCTCTTCAGAGTACAGAA | |
| | GATTAAGTGAGAAGTTCGTTTGTGCAAGCTTATCGATAAGCTTTAA | |
| | TGCGGTAGTTTATCACAGTTAAATTGCTAACGCAGTCAGGCACCGT | |
| | GTATGAAATCTAACAATGCGCTCATCGTCATCCTCGGCACCGTCAC | |
| | CCTGGATGCTGTAGGCATAGGCTTGGTTATGCCGGTACTGCCGGGC | |
| | CTCTTGCGGGATATCGTCCATTCCGACAGCATCGCCAGTCACTATG | |
| | GCGTGCTGCTAGCGCTATATGCGTTGATGCAATTTCTATGCGCACC | |
| | CGTTCTCGGAGCACTGTCCGACCGCTTTGGCCGCCGCCCAGTCCTG | |
| | CTCGCTTCGCTACTTGGAGCCACTATCGACTACGCGATCATGGCGA | |
| | CCACACCCGTCCTGTGGATCTATCGAATCTAAATGTAAGTTAAAAT | |
| | CTCTAAATAATTAAATAAGTCCCAGTTTCTCCATACGAACCTTAAC | |
| | AGCATTGCGGTGAGCATCTAGACCTTCAACAGCAGCCAGATCCAT | |
| | CACTGCTTGGCCAATATGTTTCAGTCCCTCAGGAGTTACGTCTTGT | |
| | GAAGTGATGAACTTCTGGAAGGTTGCAGTGTTAACTCCGCTGTATT | |
| | GACGGGCATATCCGTACGTTGGCAAAGTGTGGTTGGTACCGGAGG | |
| | AGTAATCTCCACAACTCTCTGGAGAGTAGGCACCAACAAACACAG | |
| | ATCCAGCGTGTTGTACTTGATCAACATAAGAAGAAGCATTCTCGAT | |
| | TTGCAGGATCAAGTGTTCAGGAGCGTACTGATTGGACATTTCCAAA | |
| | GCCTGCTCGTAGGTTGCAACCGATAGGGTTGTAGAGTGTGCAATAC | |
| | ACTTGCGTACAATTTCAACCCTTGGCAACTGCACAGCTTGGTTGTG | |
| | AACAGCATCTTCAATTCTGGCAAGCTCCTTGTCTGTCATATCGACA | |
| | GCCAACAGAATCACCTGGGAATCAATACCATGTTCAGCTTGAGCA | |
| | GAAGGTCTGAGGCAACGAAATCTGGATCAGCGTATTTATCAGCAA | |
| | TAACTAGAACTTCAGAAGGCCCAGCAGGCATGTCAATACTACACA | |
| | GGGCTGATGTGTCATTTTGAACCATCATCTTGGCAGCAGTAACGAA | |
| | CTGGTTTCCTGGACCAAATATTTTGTCACACTTAGGAACAGTTTCT | |
| | GTTCCCGTAAGCCATAGCAGCTACTGCCTGGGCGCCTCCTGCTAGCA | |
| | CGATACACTTAGCACCAACCTTGTGGGCAACGTAGATGACTTCTGG | |
| | GGTAAGGGTACCATCCTTCTTAGGTGGAGATGCAAAAACAATTTCT | |
| | TTGCAACCAGCAACTTTGGCAGGAACACCCAGCATCAGGGAAGTG | |
| | GAAGGCAGAATTGCGGTTCCACCAGGAATATAGAGGCCAACTTTC | |
| | TCAATAGGTCTTGCAAAACGAGAGCAGACTACACCAGGGCAAGTC | |
| | TCAACTTGCAACGTCTCCGTTAGTTGAGCTTCATGGAATTTCCTGA | |
| | CGTTATCTATAGAGAGATCAATGGCTCTCTTAACGTTATCTGGCAA | |
| | TTGCATAAGTTCCTCTGGGAAAGGAGCTTCTAACACAGGTGTCTTC | |
| | AAAGCGACTCCATCAAACTTGGCAGTTAGTTCTAAAAGGGCTTTGT | |
| | CACCATTTTGACGAACATTGTCGACAATTGGTTTGACTAATTCCAT | |
| | AATCTGTTCCGTTTTCTGGATAGGACGACGAAGGGCATCTTCAATT | |
| | TCTTGTGAGGAGGCCTTAGAAACGTCAATTTTGCACAATTCAATAC | |
| | GACCTTCAGAAGGGACTTCTTTAGGTTTGGATTCTTCTTTAGGTTGT | |
| | TCCTTGGTGTATCCTGGCTTGGCATCTCCTTTCCTTCTAGTGACCTT | |
| | TAGGGACTTCATATCCAGGTTTCTCTCCACCTCGTCCAACGTCACA | |
| | CCGTACTTGGCACATCTAACTAATGCAAAATAAAATAAGTCAGCA | |
| | CATTCCCAGGCTATATCTTCCTTGGATTTAGCTTCTGCAAGTTCATC | |
| | AGCTTCCTCCCTAATTTTAGCGTTCAAACAAAACTTCGTCGTCAAA | |
| | TAACCGTTTGGTATAAGAACCTTCTGGAGCATTGCTCTTACGATCC | |
| | CACAAGGTGCTTCCATGGCTCTAAGACCCTTTGATTGGCCAAAACA | |
| | GGAAGTGCGTTCCAAGTGACAGAAACCAACACCTGTTTGTTCAAC | |
| | CACAAATTTCAAGCAGTCTCCATCACAATCCAATTCGATACCCAGC | |
| | AACTTTTGAGTTCGTCCAGATGTAGCACCTTTATACCACAAACCGT | |
| | GACGACGAGATTGGTAGACTCCAGTTTGTGTCCTTATAGCCTCCGG | |
| | AATAGACTTTTTGGACGAGTACACCAGGCCCAACGAGTAATTAGA | |
| | AGAGTCAGCCACCAAAGTAGTGAATAGACCATCGGGGCGGTCAGT | |
| | AGTCAAAGACGCCAACAAAATTTCACTGACAGGGAACTTTTTGAC | |
| | ATCTTCAGAAAGTTCGTATTCAGTAGTCAATTGCCGAGCATCAATA | |
| | ATGGGGATTATACCAGAAGCAACAGTGGAAGTCACATCTACCAAC | |
| | TTTGCGGTCTCAGAAAAAGCATAAACAGTTCTACTACCGCCATTAG | |
| | TGAAACTTTTCAAATCGCCCAGTGGAGAAGAAAAAGGCACAGCGA | |
| | TACTAGCATTAGCGGGCAAGGATGCAACTTTATCAACCAGGGTCCT | |
| | ATAGATAACCCTAGCGCCTGGGATCATCCTTTGGACAACTCTTTCT | |
| | GCCAAATCTAGGTCCAAAATCACTTCATTGATACCATTATTGTACA | |
| | ACTTGAGCAAGTTGTCGATCAGCTCCTCAAATTGGTCCTCTGTAAC | |
| | GGATGACTCAACTTGCACATTAACTTGAAGCTCAGTCGATTGAGTG | |
| | AACTTGATCAGGTTGTGCAGCTGGTCAGCAGCATAGGGAAACACG GC | |
| | TTTTCCTACCAAACTCAAGGAATTATCAAACTCTGCAACACTTG | |
| | CGTATGCAGGTAGCAAGGGAAATGTCATACTTGAAGTCGGACAGT | |
| | GAGTGTAGTCTTGAGAAATTCTGAAGCCGTATTTTTATTATCAGTG | |
| | AGTCAGTCATCAGGAGATCCTCTACGCCGGACGCATCGTGGCCGA | |
| | CCTGCAGGTCGGCATCACCGGCGCCACAGGTGCGGTTGCTGGCGC | |
| | CTATATCGCCGACATCACCGATGGGGAAGATCGGGCTCGCCACTTC | |
| | GGGCTCATGAGCGCTTGTTTCGGCGTGGGTATGGTGGCAGGCCCCG | |
| | TGGCCGGGGGACTGTTGGGCGCCATCTCCTTGGACCTGCAGGGGG | |
| | GGGGGGGGAAAGCCACGTTGTGTCTCAAAATCTCTGATGTTACATT | |
| | GCACAAGATAAAAATATATCATCATGAACAATAAAACTGTCTGCT | |
| | TACATAAACAGTAATACAAGGGGTGTTATGAGCCATATTCAACGG | |
| | GAAACGTCTTGCTCAAGGCCGCGATTAAATTCCAACATGGATGCTG | |
| | ATTTATATGGGTATAAATGGGCTCGCGATAATGTCGGGCAATCAG | |
| | GTGCGACAATCTATCGATTGTATGGGAAGCCCGATGCGCCAGAGT | |
| | TGTTTCTGAAACATGGCAAAGGTAGCGTTGCCAATGATGTTACAGA | |
| | TGAGATGGTCAGACTAAACTGGCTGACGGAATTTATGCCTCTTCCG | |
| | ACCATCAAGCATTTTATCCGTACTCCTGATGATGCATGGTTACTCA | |
| | CCACTGCGATCGCCGGGAAAACAGCATTCCAGGTATAGAAGAAT | |
| | ATCCTGATTCAGGTGAAAATATTGTTGATGCGCTGGCAGTGTTCCT | |
| | GCGCCGGTTGCATTCGATTCCTGTTTGTAATTGTCCTTTTAACAGCG | |
| | ATCGCGTATTTCGTCTCGCTCAGGCGCAATCACGAATGAATAACGG | |
| | TTTGGTTGATGCGAGTGATTTTGATGACGAGCGTAATGGCTGGCCT | |
| | GTTGAACAAGTCTGGAAAGAAATGCATAAGCTTTTGCCATTCTCAC | |
| | CGGATTCAGTCGTCACTCATGGTGATTTCTCACTTGATAACCTTATT | |
| | TTTGACGAGGGGAAATTAATAGGTTGTATTGATGTTGGACGAGTCG | |
| | GAATCGCAGACCGATACCAGGATCTTGCCATCCTATGGAACTGCCT | |
| | CGGTGAGTTTTCTCCTTCATTACAGAAACGGCTTTTTCAAAAATAT | |
| | GGTATTGATAATCCTGATATGAATAAATTGCAGTTTCATTTGATGC | |
| | TCGATGAGTTTTTCTAATCAGAATTGGTTAATTGGTTGTAACACTG | |
| | GCAGAGCATTACGCTGACTTGACGGGACGGCGGCTTTGTTGAATA | |
| | AATCGAACTTTTGCTGAGTTGAAGGATCAGATCACGCATCTTCCCG | |
| | ACAACGCAGACCGTTCCGTGGCAAAGCAAAAGTTCAAAATCACCA | |
| | ACTGGTCCACCTACAACAAAGCTCTCATCAACCGTGGCTCCCTCAC | |
| | TTTCTGGCTGGATGATGGGGCGATTCAGGCCTGGTATGAGTCAGCA | |
| | ACACCTTCTTCACGAGGCAGACCTCAGCGCCCCCCCCCCCCTGCAG | |
| | GTCCCACGGCGGCGGTGCTCAACGGCCTCAACCTACTACTGGGCTG | |
| | CTTCCTAATGCAGGAGTCGCATAAGGGAGAGCGTCGAGTATCTAT | |
| | GATTGGAAGTATGGGAATGGTGATACCCGCATTCTTCAGTGTCTTG | |
| | AGGTCTCCTATCAGATTATGCCCAACTAAAGCAACCGGAGGAGGA | |
| | GATTTCATGGTAAATTTCTCTGACTTTTGGTCATCAGTAGACTCGA | |
| | ACTGTGAGACTATCTCGGTTATGACAGCAGAAATGTCCTTCTTGGA | |
| | GACAGTAAATGAAGTCCCACCAATAAAGAAATCCTTGTTATCAGG | |
| | AACAAACTTCTTGTTTCGAACTTTTTCGGTGCCTTGAACTATAAAA | |
| | TGTAGAGTGGATATGTCGGGTAGGAATGGAGCGGGCAAATGCTTA | |
| | CCTTCTGGACCTTCAAGAGGTATGTAGGGTTTGTAGATACTGATGC | |
| | CAACTTCAGTGACAACGTTGCTATTTCGTTCAAACCATTCCGAATC | |
| | CAGAGAAATCAAAGTTGTTTGTCTACTATTGATCCAAGCCAGTGCG | |
| | GTCTTGAAACTGACAATAGTGTGCTCGTGTTTTGAGGTCATCTTTG | |
| | TATGAATAAATCTAGTCTTTGATCTAAATAATCTTGACGAGCCAAG | |
| | GCGATAAATACCCAAATCTAAAACTCTTTTAAAACGTTAAAAGGA | |
| | CAAGTATGTCTGCCTGTATTAAACCCCAAATCAGCTCGTAGTCTGA | |
| | TCCTCATCAACTTGAGGGGCACTATCTTGTTTTAGAGAAATTTGCG | |
| | GAGATGCGATATCGAGAAAAAGGTACGCTGATTTTAAACGTGAAA | |
| | TTTATCTCAAGATCTCTGCCTCGCGCGTTTCGGTGATGACGGTGAA | |
| | AACCTCTGACACATGCAGCTCCCGGAGACGGTCACAGCTTGTCTGT | |
| | AAGCGGATGCCGGGAGCAGACAAGCCCGTCAGGGCGCGTCAGCG | |
| | GGTGTTGGCGGGTGTCGGGGCGCAGCCATGACCCAGTCACGTAGC | |
| | GATAGCGGAGTGTATACTGGCTTAACTATGCGGCATCAGAGCAGA | |
| | TTGTACTGAGAGTGCACCATATGCGGTGTGAAATACCGCACAGAT | |
| | GCGTAAGGAGAAAATACCGCATCAGGCGCTCTTCCGCTTCCTCGCT | |
| | CACTGACTCGCTGCGCTCGGTCGTTCGGCTGCGGCGAGCGGTATCA | |
| | GCTCACTCAAAGGCGGTAATACGGTTATCCACAGAATCAGGGGAT | |
| | AACGCAGGAAAGAACATGTGAGCAAAAGGCCAGCAAAAGGCCAG | |
| | GAACCGTAAAAAGGCCGCGTTGCTGGCGTTTTTCCATAGGCTCCGC | |
| | CCCCCTGACGAGCATCACAAAAATCGACGCTCAAGTCAGAGGTGG | |
| | CGAAACCCGACAGGACTATAAAGATACCAGGCGTTTCCCCCTGGA | |
| | AGCTCCCTCGTGCGCTCTCCTGTTCCGACCCTGCCGCTTACCGGAT | |
| | ACCTGTCCGCCTTTCTCCCTTCGGGAAGCGTGGCGCTTTCTCAATG | |
| | CTCACGCTGTAGGTATCTCAGTTCGGTGTAGGTCGTTCGCTCCAAG | |
| | CTGGGCTGTGTGCACGAACCCCCCGTTCAGCCCGACCGCTGCGCCT | |
| | TATCCGGTAACTATCGTCTTGAGTCCAACCCGGTAAGACACGACTT | |
| | ATCGCCACTGGCAGCAGCCACTGGTAACAGGATTAGCAGAGCGAG | |
| | GTATGTAGGCGGTGCTACAGAGTTCTTGAAGTGGTGGCCTAACTAC | |
| | GGCTACACTAGAAGGACAGTATTTGGTATCTGCGCTCTGCTGAAGC | |
| | CAGTTACCTTCGGAAAAAGAGTTGGTAGCTCTTGATCCGGCAAAC | |
| | AAACCACCGCTGGTAGCGGTGGTTTTTTTGTTTGCAAGCAGCAGAT | |
| | TACGCGCAGAAAAAAAGGATCTCAAGAAGATCCTTTGATCTTTTCT | |
| | ACGGGGTCTGACGCTCAGTGGAACGAAAACTCACGTTAAGGGATT | |
| | TTGGTCATGAGATTATCAAAAAGGATCTTCACCTAGATCCTTTTAA | |
| | ATTAAAAATGAAGTTTTAAATCAATCTAAAGTATATATGAGTAAAC | |
| | TTGGTCTGACAGTTACCAATGCTTAATCAGTGAGGCACCTATCTCA | |
| | GCGATCTGTCTATTTCGTTCATCCATAGTTGCCTGACTCCCCGTCGT | |
| | GTAGATAACTACGATACGGGAGGGCTTACCATCTGGCCCCAGTGC | |
| | TGCAATGATACCGCGAGACCCACGCTCACCGGCTCCAGATTTATCA | |
| | GCAATAAACCAGCCAGCCGGAAGGGCCGAGCGCAGAAGTGGTCCT | |
| | GCAACTTTATCCGCCTCCATCCAGTCTATTAATTGTTGCCGGGAAG | |
| | CTAGAGTAAGTAGTTCGCCAGTTAATAGTTTGCGCAACGTTGTTGC | |
| | CATTGCTGCAGGCATCGTGGTGTCACGCTCGTCGTTTGGTATGGCT | |
| | TCATTCAGCTCCGGTTCCCAACGATCAAGGCGAGTTACATGATCCC | |
| | CCATGTTGTGCAAAAAAGCGGTTAGCTCCTTCGGTCCTCCGATCGT | |
| | TGTCAGAAGTAAGTTGGCCGCAGTGTTATCACTCATGGTTATGGCA | |
| | GCACTGCATAATTCTCTTACTGTCATGCCATCCGTAAGATGCTTTTC | |
| | TGTGACTGGTGAGTACTCAACCAAGTCATTCTGAGAATAGTGTATG | |
| | CGGCGACCGAGTTGCTCTTGCCCGGCGTCAACACGGGATAATACC | |
| | GCGCCACATAGCAGAACTTTAAAAGTGCTCATCATTGGAAAACGT | |
| | TCTTCGGGGCGAAAACTCTCAAGGATCTTACCGCTGTTGAGATCCA | |
| | GTTCGATGTAACCCACTCGTGCACCCAACTGATCTTCAGCATCTTT | |
| | TACTTTCACCAGCGTTTCTGGGTGAGCAAAAACAGGAAGGCAAAA | |
| | TGCCGCAAAAAAGGGAATAAGGGCGACACGGAAATGTTGAATACT | |
| | CATACTCTTCCTTTTTCAATATTATTGAAGCATTTATCAGGGTTATT | |
| | GTCTCATGAGCGGATACATATTTGAATGTATTTAGAAAAATAAACA | |
| | AATAGGGGTTCCGCGCACATTTCCCCGAAAAGTGCCACCTGACGTC | |
| | TAAGAAACCATTATTATCATGACATTAACCTATAAAAATAGGCGTA | |
| | TCACGAGGCCCTTTCGTCTTCAAGAATTAATTCTCATGTTTGACAG | |
| | CTTATCATCGATAAGCTGACTCATGTTGGTATTGTGAAATAGACGC | |
| | AGATCGGGAACACTGAAAAATAACAGTTATTATTCGAGATC | |
| pUB 116 | GATCTAACATCCAAAGACGAAAGGTTGAATGAAACCTTTTTGCCAT | SEQ ID NO:34 |
| | CCGACATCCACAGGTCCATTCTCACACATAAGTGCCAAACGCAAC | |
| | AGGAGGGGATACACTAGCAGCAGACCGTTGCAAACGCAGGACCTC | |
| | CACTCCTCTTCTCCTCAACACCCACTTTTGCCATCGAAAAACCAGC | |
| | CCAGTTATTGGGCTTGATTGGAGCTCGCTCATTCCAATTCCTTCTAT | |
| | TAGGCTACTAACACCATGACTTTATTAGCCTGTCTATCCTGGCCCC | |
| | CCTGGCGAGGTTCATGTTTGTTTATTTCCGAATGCAACAAGCTCCG | |
| | CATTACACCCGAACATCACTCCAGATGAGGGCTTTCTGAGTGTGGG | |
| | GTCAAATAGTTTCATGTTCCCCAAATGGCCCAAAACTGACAGTTTA | |
| | AACGCTGTCTTGGAACCTAATATGACAAAAGCGTGATCTCATCCAA | |
| | GATGAACTAAGTTTGGTTCGTTGAAATGCTAACGGCCAGTTGGTCA | |
| | AAAAGAAACTTCCAAAAGTCGCCATACCGTTTGTCTTGTTTGGTAT | |
| | TGATTGACGAATGCTCAAAAATAATCTCATTAATGCTTAGCGCAGT | |
| | CTCTCTATCGCTTCTGAACCCCGGTGCACCTGTGCCGAAACGCAAA | |
| | TGGGGAAACACCCGCTTTTTGGATGATTATGCATTGTCTCCACATT | |
| | GTATGCTTCCAAGATTCTGGTGGGAATACTGCTGATAGCCTAACGT | |
| | TCATGATCAAAATTTAACTGTTCTAACCCCTACTTGACAGCAATAT | |
| | ATAAACAGAAGGAAGCTGCCCTGTCTTAAACCTTTTTTTTTATCAT | |
| | CATTATTAGCTTACTTTCATAATTGCGACTGGTTCCAATTGACAAG | |
| | CTTTTGATTTTAACGACTTTTAACGACAACTTGAGAAGATCAAAAA | |
| | ACAACTAATTATTCGAAGGATCCAAACGATGAGATTTCCTTCAATT | |
| | TTTACTGCAGTTTTATTCGCAGCATCCTCCGCATTAGCTGCTCCAGT | |
| | CAACACTACAACAGAAGATGAAACGGCACAAATTCCGGCTGAAGC | |
| | TGTCATCGGTTACTCAGATTTAGAAGGGGATTTCGATGTTGCTGTT | |
| | TTGCCATTTTCCAACAGCACAAATAACGGGTTATTGTTTATAAATA | |
| | CTACTATTGCCAGCATTGCTGCTAAAGAAGAAGGGGTATCTCTCGA | |
| | GAAAAGATGCGGACCGGGCAGGGGGTTCGGGAAGAGGAGGCACC | |
| | CCAAAAAGCTGACCCCTTTAGCCTACAAGCAGTTTATCCCCAATGT | |
| | GGCCGAGAAGACCCTAGGCGCCAGCGGAAGGTATGAAGGGAAGA | |
| | TCTCCAGAAACTCCGAGCGATTTAAGGAACTCACCCCCAATTACAA | |
| | CCCCGACATCATATTTAAGGATGAAGAAAACACCGGAGCGGACAG | |
| | GCTGATGACTCAGAGGTGTAAGGACAAGTTGAACGCTTTGGCCAT | |
| | CTCGGTGATGAACCAGTGGCCAGGAGTGAAACTGCGGGTGACCGA | |
| | GGGCTGGGACGAAGATGGCCACCACTCAGAGGAGTCTCTGCACTA | |
| | CGAGGGCCGCGCAGTGGACATCACCACGTCTGACCGCGACCGCAG | |
| | CAAGTACGGCATGCTGGCCCGCCTGGCGGTGGAGGCCGGCTTCGA | |
| | CTGGGTGTACTACGAGTCCAAGGCACATATCCACTGCTCGGTGAA | |
| | AGCAGAGAACTCGGTGGCGGCCAAATCGGGAGGCGTCGACAAAA | |
| | CTCACACATGCCCACCGTGCCCAGCACCTGAACTCCTGGGGGGAC | |
| | CGTCAGTCTTCCTCTTCCCCCCAAAACCCAAGGACACCCTCATGAT | |
| | CTCCCGGACCCCTGAGGTCACATGCGTGGTGGTGGACGTGAGCCA | |
| | CGAAGACCCTGAGGTCAAGTTCAACTGGTACGTGGACGGCGTGGA | |
| | GGTGCATAATGCCAAGACAAAGCCGcgggaggagcagtaccagagcacgtaccgtg | |
| | tggTCAGCGTCCTCACCGTCCTGCACCAGGACTGGCTGAATGGCAAG | |
| | GAGTACAAGTGCAAGGTCTCCAACAAAGCCCTCCCAGCCCCCATC | |
| | GAGAAAACCATCTCCAAAGCCAAAGGGCAGCCCCGAGAACCACA | |
| | GGTGTACACCCTGCCCCCATCCCGGGATGAGCTGACCAAGAACCA | |
| | GGTCAGCCTGACCTGCCTGGTCAAAGGCTTCTATCCCAGCGACATC | |
| | GCCGTGGAGTGGGAGAGCAATGGGCAGCCGGAGAACAACTACAA | |
| | GACCACGCCTCCCGTGTTGGACTCCGACGGCTCCTTCTTCCTCTAC | |
| | AGCAAGCTCACCGTGGACAAGAGCAGGTGGCAGCAGGGGAACGT | |
| | CTTCTCATGCTCCGTGATGCATGAGGCTCTGCACAACCACTACACG | |
| | CAGAAGAGCCTCTCCCTGTCTCCCGGGAAATGAGTGCGGCGGCCG | |
| | CGAATTAATTCGCCTTAGACATGACTGTTCCTCAGTTCAAGTTGGG | |
| | CACTTACGAGAAGACCGGTCTTGCTAGATTCTAATCAAGAGGATGT | |
| | CAGAATGCCATTTGCCTGAGAGATGCAGGCTTCATTTTTGATACTT | |
| | TTTTATTTGTAACCTATATAGTATAGGATTTTTTTTGTCATTTTGTTT | |
| | CTTCTCGTACGAGCTTGCTCCTGATCAGCCTATCTCGCAGCTGATG | |
| | AATATCTTGTGGTAGGGGTTTGGGAAAATCATTCGAGTTTGATGTT | |
| | TTTCTTGGTATTTCCCACTCCTCTTCAGAGTACAGAAGATTAAGTG | |
| | AGAAGTTCGTTTGTGCAAGCTTATCGATAAGCTTTAATGCGGTAGT | |
| | TTATCACAGTTAAATTGCTAACGCAGTCAGGCACCGTGTATGAAAT | |
| | CTAACAATGCGCTCATCGTCATCCTCGGCACCGTCACCCTGGATGC | |
| | TGTAGGCATAGGCTTGGTTATGCCGGTACTGCCGGGCCTCTTGCGG | |
| | GATATCGTCCATTCCGACAGCATCGCCAGTCACTATGGCGTGCTGC | |
| | TAGCGCTATATGCGTTGATGCAATTTCTATGCGCACCCGTTCTCGG | |
| | AGCACTGTCCGACCGCTTTGGCCGCCGCCCAGTCCTGCTCGCTTCG | |
| | CTACTTGGAGCCACTATCGACTACGCGATCATGGCGACCACACCCG | |
| | TCCTGTGGATCTATCGAATCTAAATGTAAGTTAAAATCTCTAAATA | |
| | ATTAAATAAGTCCCAGTTTCTCCATACGAACCTTAACAGCATTGCG | |
| | GTGAGCATCTAGACCTTCAACAGCAGCCAGATCCATCACTGCTTGG | |
| | CCAATATGTTTCAGTCCCTCAGGAGTTACGTCTTGTGAAGTGATGA | |
| | ACTTCTGGAAGGTTGCAGTGTTAACTCCGCTGTATTGACGGGCATA | |
| | TCCGTACGTTGGCAAAGTGTGGTTGGTACCGGAGGAGTAATCTCCA | |
| | CAACTCTCTGGAGAGTAGGCACCAACAAACACAGATCCAGCGTGT | |
| | TGTACTTGATCAACATAAGAAGAAGCATTCTCGATTTGCAGGATCA | |
| | AGTGTTCAGGAGCGTACTGATTGGACATTTCCAAAGCCTGCTCGTA | |
| | GGTTGCAACCGATAGGGTTGTAGAGTGTGCAATACACTTGCGTAC | |
| | AATTTCAACCCTTGGCAACTGCACAGCTTGGTTGTGAACAGCATCT | |
| | TCAATTCTGGCAAGCTCCTTGTCTGTCATATCGACAGCCAACAGAA | |
| | TCACCTGGGAATCAATACCATGTTCAGCTTGAGCAGAAGGTCTGA | |
| | GGCAACGAAATCTGGATCAGCGTATTTATCAGCAATAACTAGAAC | |
| | TTCAGAAGGCCCAGCAGGCATGTCAATACTACACAGGGCTGATGT | |
| | GTCATTTTGAACCATCATCTTGGCAGCAGTAACGAACTGGTTTCCT | |
| | GGACCAAATATTTTGTCACACTTAGGAACAGTTTCTGTTCCGTAAG | |
| | CCATAGCAGCTACTGCCTGGGCGCCTCCTGCTAGCACGATACACTT | |
| | AGCACCAACCTTGTGGGCAACGTAGATGACTTCTGGGGTAAGGGT | |
| | ACCATCCTTCTTAGGTGGAGATGCAAAAACAATTTCTTTGCAACCA | |
| | GCAACTTTGGCAGGAACACCCAGCATCAGGGAAGTGGAAGGCAGA | |
| | ATTGCGGTTCCACCAGGAATATAGAGGCCAACTTTCTCAATAGGTC | |
| | TTGCAAAACGAGAGCAGACTACACCAGGGCAAGTCTCAACTTGCA | |
| | ACGTCTCCGTTAGTTGAGCTTCATGGAATTTCCTGACGTTATCTATA | |
| | GAGAGATCAATGGCTCTCTTAACGTTATCTGGCAATTGCATAAGTT | |
| | CCTCTGGGAAAGGAGCTTCTAACACAGGTGTCTTCAAAGCGACTCC | |
| | ATCAAACTTGGCAGTTAGTTCTAAAAGGGCTTTGTCACCATTTTGA | |
| | CGAACATTGTCGACAATTGGTTTGACTAATTCCATAATCTGTTCCG | |
| | TTTTCTGGATAGGACGACGAAGGGCATCTTCAATTTCTTGTGAGGA | |
| | GGCCTTAGAAACGTCAATTTTGCACAATTCAATACGACCTTCAGAA | |
| | GGGACTTCTTTAGGTTTGGATTCTTCTTTAGGTTGTTCCTTGGTGTA | |
| | TCCTGGCTTGGCATCTCCTTTCCTTCTAGTGACCTTTAGGGACTTCA | |
| | TATCCAGGTTTCTCTCCACCTCGTCCAACGTCACACCGTACTTGGC | |
| | ACATCTAACTAATGCAAAATAAAATAAGTCAGCACATTCCCAGGC | |
| | TATATCTTCCTTGGATTTAGCTTCTGCAAGTTCATCAGCTTCCTCCC | |
| | TAATTTTAGCGTTCAAACAAAACTTCGTCGTCAAATAACCGTTTGG | |
| | TATAAGAACCTTCTGGAGCATTGCTCTTACGATCCCACAAGGTGCT | |
| | TCCATGGCTCTAAGACCCTTTGATTGGCCAAAACAGGAAGTGCGTT | |
| | CCAAGTGACAGAAACCAACACCTGTTTGTTCAACCACAAATTTCAA | |
| | GCAGTCTCCATCACAATCCAATTCGATACCCAGCAACTTTTGAGTT | |
| | CGTCCAGATGTAGCACCTTTATACCACAAACCGTGACGACGAGATT | |
| | GGTAGACTCCAGTTTGTGTCCTTATAGCCTCCGGAATAGACTTTTT | |
| | GGACGAGTACACCAGGCCCAACGAGTAATTAGAAGAGTCAGCCAC | |
| | CAAAGTAGTGAATAGACCATCGGGGCGGTCAGTAGTCAAAGACGC | |
| | CAACAAAATTTCACTGACAGGGAACTTTTTGACATCTTCAGAAAGT | |
| | TCGTATTCAGTAGTCAATTGCCGAGCATCAATAATGGGGATTATAC | |
| | CAGAAGCAACAGTGGAAGTCACATCTACCAACTTTGCGGTCTCAG | |
| | AAAAAGCATAAACAGTTCTACTACCGCCATTAGTGAAACTTTTCAA | |
| | ATCGCCCAGTGGAGAAGAAAAAGGCACAGCGATACTAGCATTAGC | |
| | GGGCAAGGATGCAACTTTATCAACCAGGGTCCTATAGATAACCCT | |
| | AGCGCCTGGGATCATCCTTTGGACAACTCTTTCTGCCAAATCTAGG | |
| | TCCAAAATCACTTCATTGATACCATTATTGTACAACTTGAGCAAGT | |
| | TGTCGATCAGCTCCTCAAATTGGTCCTCTGTAACGGATGACTCAAC | |
| | TTGCACATTAACTTGAAGCTCAGTCGATTGAGTGAACTTGATCAGG | |
| | TTGTGCAGCTGGTCAGCAGCATAGGGAAACACGGCTTTTCCTACCA | |
| | AACTCAAGGAATTATCAAACTCTGCAACACTTGCGTATGCAGGTA | |
| | GCAAGGGAAATGTCATACTTGAAGTCGGACAGTGAGTGTAGTCTT | |
| | GAGAAATTCTGAAGCCGTATTTTTATTATCAGTGAGTCAGTCATCA | |
| | GGAGATCCTCTACGCCGGACGCATCGTGGCCGACCTGCAGGTCGG | |
| | CATCACCGGCGCCACAGGTGCGGTTGCTGGCGCCTATATCGCCGAC | |
| | ATCACCGATGGGGAAGATCGGGCTCGCCACTTCGGGCTCATGAGC | |
| | GCTTGTTTCGGCGTGGGTATGGTGGCAGGCCCCGTGGCCGGGGGA | |
| | CTGTTGGGCGCCATCTCCTTGGACCTGCAGGGGGGGGGGGGGAAA | |
| | GCCACGTTGTGTCTCAAAATCTCTGATGTTACATTGCACAAGATAA | |
| | AAATATATCATCATGAACAATAAAACTGTCTGCTTACATAAACAGT | |
| | AATACAAGGGGTGTTATGAGCCATATTCAACGGGAAACGTCTTGC | |
| | TCAAGGCCGCGATTAAATTCCAACATGGATGCTGATTTATATGGGT | |
| | ATAAATGGGCTCGCGATAATGTCGGGCAATCAGGTGCGACAATCT | |
| | ATCGATTGTATGGGAAGCCCGATGCGCCAGAGTTGTTTCTGAAACA | |
| | TGGCAAAGGTAGCGTTGCCAATGATGTTACAGATGAGATGGTCAG | |
| | ACTAAACTGGCTGACGGAATTTATGCCTCTTCCGACCATCAAGCAT | |
| | TTTATCCGTACTCCTGATGATGCATGGTTACTCACCACTGCGATCC | |
| | CCGGGAAAACAGCATTCCAGGTATTAGAAGAATATCCTGATTCAG | |
| | GTGAAAATATTGTTGATGCGCTGGCAGTGTTCCTGCGCCGGTTGCA | |
| | TTCGATTCCTGTTTGTAATTGTCCTTTTAACAGCGATCGCGTATTTC | |
| | GTCTCGCTCAGGCGCAATCACGAATGAATAACGGTTTGGTTGATGC | |
| | GAGTGATTTTGATGACGAGCGTAATGGCTGGCCTGTTGAACAAGTC | |
| | TGGAAAGAAATGCATAAGCTTTTGCCATTCTCACCGGATTCAGTCG | |
| | TCACTCATGGTGATTTCTCACTTGATAACCTTATTTTTGACGAGGG | |
| | GAAATTAATAGGTTGTATTGATGTTGGACGAGTCGGAATCGCAGA | |
| | CCGATACCAGGATCTTGCCATCCTATGGAACTGCCTCGGTGAGTTT | |
| | TCTCCTTCATTACAGAAACGGCTTTTTCAAAAATATGGTATTGATA | |
| | ATCCTGATATGAATAAATTGCAGTTTCATTTGATGCTCGATGAGTT | |
| | TTTCTAATCAGAATTGGTTAATTGGTTGTAACACTGGCAGAGCATT | |
| | ACGCTGACTTGACGGGACGGCGGCTTTGTTGAATAAATCGAACTTT | |
| | TGCTGAGTTGAAGGATCAGATCACGCATCTTCCCGACAACGCAGA | |
| | CCGTTCCGTGGCAAAGCAAAAGTTCAAAATCACCAACTGGTCCAC | |
| | CTACAACAAAGCTCTCATCAACCGTGGCTCCCTCACTTTCTGGCTG | |
| | GATGATGGGGCGATTCAGGCCTGGTATGAGTCAGCAACACCTTCTT | |
| | CACGAGGCAGACCTCAGCGCCCCCCCCCCCCTGCAGGTCCCACGG | |
| | CGGCGGTGCTCAACGGCCTCAACCTACTACTGGGCTGCTTCCTAAT | |
| | GCAGGAGTCGCATAAGGGAGAGCGTCGAGTATCTATGATTGGAAG | |
| | TATGGGAATGGTGATACCCGCATTCTTCAGTGTCTTGAGGTCTCCT | |
| | ATCAGATTATGCCCAACTAAAGCAACCGGAGGAGGAGATTTCATG | |
| | GTAAATTTCTCTGACTTTTGGTCATCAGTAGACTCGAACTGTGAGA | |
| | CTATCTCGGTTATGACAGCAGAAATGTCCTTCTTGGAGACAGTAAA | |
| | TGAAGTCCCACCAATAAAGAAATCCTTGTTATCAGGAACAAACTTC | |
| | TTGTTTCGAACTTTTTCGGTGCCTTGAACTATAAAATGTAGAGTGG | |
| | ATATGTCGGGTAGGAATGGAGCGGGCAAATGCTTACCTTCTGGAC | |
| | CTTCAAGAGGTATGTAGGGTTTGTAGATACTGATGCCAACTTCAGT | |
| | GACAACGTTGCTATTTCGTTCAAACCATTCCGAATCCAGAGAAATC | |
| | AAAGTTGTTTGTCTACTATTGATCCAAGCCAGTGCGGTCTTGAAAC | |
| | TGACAATAGTGTGCTCGTGTTTTGAGGTCATCTTTGTATGAATAAA | |
| | TCTAGTCTTTGATCTAAATAATCTTGACGAGCCAAGGCGATAAATA | |
| | CCCAAATCTAAAACTCTTTTAAAACGTTAAAAGGACAAGTATGTCT | |
| | GCCTGTATTAAACCCCAAATCAGCTCGTAGTCTGATCCTCATCAAC | |
| | TTGAGGGGCACTATCTTGTTTTAGAGAAATTTGCGGAGATGCGATA | |
| | TCGAGAAAAAGGTACGCTGATTTTAAACGTGAAATTTATCTCAAG | |
| | ATCTCTGCCTCGCGCGTTTCGGTGATGACGGTGAAAACCTCTGACA | |
| | CATGCAGCTCCCGGAGACGGTCACAGCTTGTCTGTAAGCGGATGC | |
| | CGGGAGCAGACAAGCCCGTCAGGGCGCGTCAGCGGGTGTTGGCGG | |
| | GTGTCGGGGCGCAGCCATGACCCAGTCACGTAGCGATAGCGGAGT | |
| | GTATACTGGCTTAACTATGCGGCATCAGAGCAGATTGTACTGAGA | |
| | GTGCACCATATGCGGTGTGAAATACCGCACAGATGCGTAAGGAGA | |
| | AAATACCGCATCAGGCGCTCTTCCGCTTCCTCGCTCACTGACTCGC | |
| | TGCGCTCGGTCGTTCGGCTGCGGCGAGCGGTATCAGCTCACTCAAA | |
| | GGCGGTAATACGGTTATCCACAGAATCAGGGGATAACGCAGGAAA | |
| | GAACATGTGAGCAAAAGGCCAGCAAAAGGCCAGGAACCGTAAAA | |
| | AGGCCGCGTTGCTGGCGTTTTTCCATAGGCTCCGCCCCCCTGACGA | |
| | GCATCACAAAAATCGACGCTCAAGTCAGAGGTGGCGAAACCCGAC | |
| | AGGACTATAAAGATACCAGGCGTTTCCCCCTGGAAGCTCCCTCGTG | |
| | CGCTCTCCTGTTCCGACCCTGCCGCTTACCGGATACCTGTCCGCCTT | |
| | TCTCCCTTCGGGAAGCGTGGCGCTTTCTCAATGCTCACGCTGTAGG | |
| | TATCTCAGTTCGGTGTAGGTCGTTCGCTCCAAGCTGGGCTGTGTGC | |
| | ACGAACCCCCCGTTCAGCCCGACCGCTGCGCCTTATCCGGTAACTA | |
| | TCGTCTTGAGTCCAACCCGGTAAGACACGACTTATCGCCACTGGCA | |
| | GCAGCCACTGGTAACAGGATTAGCAGAGCGAGGTATGTAGGCGGT | |
| | GCTACAGAGTTCTTGAAGTGGTGGCCTAACTACGGCTACACTAGA | |
| | AGGACAGTATTTGGTATCTGCGCTCTGCTGAAGCCAGTTACCTTCG | |
| | GAAAAAGAGTTGGTAGCTCTTGATCCGGCAAACAAACCACCGCTG | |
| | GTAGCGGTGGTTTTTTTGTTTGCAAGCAGCAGATTACGCGCAGAAA | |
| | AAAAGGATCTCAAGAAGATCCTTTGATCTTTTCTACGGGGTCTGAG | |
| | GCTCAGTGGAACGAAAACTCACGTTAAGGGATTTTGGTCATGAGA | |
| | TTATCAAAAAGGATCTTCACCTAGATCCTTTTAAATTAAAAATGAA | |
| | GTTTTAAATCAATCTAAAGTATATATGAGTAAACTTGGTCTGACAG | |
| | TTACCAATGCTTAATCAGTGAGGCACCTATCTCAGCGATCTGTCTA | |
| | TTTCGTTCATCCATAGTTGCCTGACTCCCCGTCGTGTAGATAACTAC | |
| | GATACGGGAGGGCTTACCATCTGGCCCCAGTGCTGCAATGATACC | |
| | GCGAGACCCACGCTCACCGGCTCCAGATTTATCAGCAATAAACCA | |
| | GCCAGCCGGAAGGGCCGAGCGCAGAAGTGGTCCTGCAACTTTATC | |
| | CGCCTCCATCCAGTCTATTAATTGTTGCCGGGAAGCTAGAGTAAGT | |
| | AGTTCGCCAGTTAATAGTTTGCGCAACGTTGTTGCCATTGCTGCAG | |
| | GCATCGTGGTGTCACGCTCGTCGTTTGGTATGGCTTCATTCAGCTC | |
| | CGGTTCCCAACGATCAAGGCGAGTTACATGATCCCCCATGTTGTGC | |
| | AAAAAAGCGGTTAGCTCCTTCGGTCCTCCGATCGTTGTCAGAAGTA | |
| | AGTTGGCCGCAGTGTTATCACTCATGGTTATGGCAGCACTGCATAA | |
| | TTCTCTTACTGTCATGCCATCCGTAAGATGCTTTTCTGTGACTGGTG | |
| | AGTACTCAACCAAGTCATTCTGAGAATAGTGTATGCGGCGACCGA | |
| | GTTGCTCTTGCCCGGCGTCAACACGGGATAATACCGCGCCACATAG | |
| | CAGAACTTTAAAAGTGCTCATCATTGGAAAACGTTCTTCGGGGCGA | |
| | AAACTCTCAAGGATCTTACCGCTGTTGAGATCCAGTTCGATGTAAC | |
| | CCACTCGTGCACCCAACTGATCTTCAGCATCTTTTACTTTCACCAGC | |
| | GTTTCTGGGTGAGCAAAAACAGGAAGGCAAAATGCCGCAAAAA | |
| | GGGAATAAGGGCGACACGGAAATGTTGAATACTCATACTCTTCCTT | |
| | TTTCAATATTATTGAAGCATTTATCAGGGTTATTGTCTCATGAGCG | |
| | GATACATATTTGAATGTATTTAGAAAAATAAACAAATAGGGGTTC | |
| | CGCGCACATTTCCCCGAAAAGTGCCACCTGACGTCTAAGAAACCA | |
| | TTATTATCATGACATTAACCTATAAAAATAGGCGTATCACGAGGCC | |
| | CTTTCGTCTTCAAGAATTAATTCTCATGTTTGACAGCTTATCATCGA | |
| | TAAGCTGACTCATGTTGGTATTGTGAAATAGACGCAGATCGGGAA | |
| | CACTGAAAAATAACAGTTATTATTCGAGATC | |
| PEAG657 | CTAAATTGTAAGCGTTAATATTTTGTTAAAATTCGCGTTAAATTTTT | SEQ ID NO:35 |
| | GTTAAATCAGCTCATTTTTTAACCAATAGGCCGAAATCGGCAAAAT | |
| | CCCTTATAAATCAAAAGAATAGACCGAGATAGGGTTGAGTGTTGT | |
| | TCCAGTTTGGAACAAGAGTCCACTATTAAAGAACGTGGACTCCAA | |
| | CGTCAAAGGGCGAAAAACCGTCTATCAGGGCGATGGCCCACTACG | |
| | TGAACCATCACCCTAATCAAGTTTTTTGGGGTCGAGGTGCCGTAAA | |
| | GCACTAAATCGGAACCCTAAAGGGAGCCCCCGATTTAGAGCTTGA | |
| | CGGGGAAAGCCGGCGAACGTGGCGAGAAAGGAAGGGAAGAAAGC | |
| | GAAAGGAGCGGGCGCTAGGGCGCTGGCAAGTGTAGCGGTCACGCT | |
| | GCGCGTAACCACCACACCCGCCGCGCTTAATGCGCCGCTACAGGG | |
| | CGCGTCCCATTCGCCATTCAGGCTGCGCAACTGTTGGGAAGGGCG | |
| | ATCGGTGCGGGCCTCTTCGCTATTACGCCAGCTGGCGAAAGGGGG | |
| | ATGTGCTGCAAGGCGATTAAGTTGGGTAACGCCAGGGTTTTCCCAG | |
| | TCACGACGTTGTAAAACGACGGCCAGTGAGCGCGCGTAATACGAC | |
| | TCACTATAGGGCGAATTGGGTACCGGGCCCTCTAGATCCTTTCAGC | |
| | TCCCTGCCCCGGACATGCCCAGTGGGTGGAAGCTGCCCTCTTCTAG | |
| | CAGGAGACGCCCCAGGCGGTAGAGCAGCTGGGGGTACCAATGCAC | |
| | ACCCTCCCCCGGaGTCCAGCTGCCCCATGCCAAGCTGTGAAAGAGT | |
| | CTCAGGGGCCAGAAGGCCAACTGAGCCAGGTGGTGGTCAGCCACG | |
| | GCCGCGAAGCAGGATGCCACCACATCCTCCACCACCAGTGTCCCA | |
| | TGCTTTGTGAGCGGGGCGTAGGCCCCGAGGGCCACGTGTGTAGAG | |
| | ACAGCTGCCACGCGGGCAGGCTGCAGGCCTGGCACCCCAGCCACC | |
| | AGCACGTACTGGCCAGGCTGCACGTGGCTGGCAAATGTGGCCCGG | |
| | AAGCGGGCTGCCGGCTCCGTGTGATTGTCAGCCGTAAAGAGCAGG | |
| | TGAGCGGGTGTGAGTGCCAGGCGGCGTGGGGGGTCCTGAGTCTCG | |
| | ATGACCTGGAAGGCTCTCAGCCTGTGGGGCTCGCGGTCCAGGAAA | |
| | ATGAGCACATCGCTGAAGGTGGGGCTCCCATCCTCCCCCATGGCCA | |
| | GCACACGGTCTCCCGGCCTCACGGCTGACAAGGCCACACGCGCCC | |
| | CACTCTccaggcgtacctgggctgcggccgcgaatcagccgcccgtcttggCTGCGGCCGAG | |
| | TGCTCGGACTTGACGGAGCAATGCACGTGGGCCTTTGACTCGTAAT | |
| | ACACCCAGTCAAAGCCGGCCTCCACTGCCAAGCGCGCCAGCAGTC | |
| | CATACTTATTGCGGTCGCGGTCTGATGTGGTGATGTCCACCGCGCG | |
| | GCCCTCATAATGCAGGGACTCCTCTGAGTGGTGGCCGTCCTCGTCC | |
| | CAGCCCTCGGTCACCCGCAGCTTCACACCGGGCCACTGGTTCATCA | |
| | CCGAGATAGCCAGCGAGTTCAGGCGGTCCTTGCAGCGCTGGGTCA | |
| | TGAGGCGGTCGGCGCCTGTGTTCTCCTCGTCCTTGAAGATGATGTC | |
| | TGGATTGTAATTGGGGGTGAGCTCCTTGAAGCGCTCGGAGCTGCG | |
| | AGCGATCTTGCCTTCATAGCGTCCGCTGGCGCCCAGGGTCTTCTCG | |
| | GGCACATTGGGGCTGAACTGCTTGTAGGCGAGCGGCACGAGTTTG | |
| | CGTGGCGGTCGCCGGCGGCTGCCCACCACCCGACCCGGCCCGCAG | |
| | CCCCATGCCGCcGGCACCACCAGCAGCAGCAACAGGACCAGGCAG | |
| | AAGTGCAGTCGGGGCCGGAGCCGggcgggagacatggcggccgcgacggtatcgata | |
| | agcTTGATATCGAATTCCTGCAGCCCGGGGGATCCACTAGTTCTAGA | |
| | GCGGCCGCCACCGCGGTGGAGCTCCAGCTTTTGTTCCCTTTAGTGA | |
| | GGGTTAATTGCGCGCTTGGCGTAATCATGGTCATAGCTGTTTCCTG | |
| | TGTGAAATTGTTATCCGCTCACAATTCCACACAACATACGAGCCGG | |
| | AAGCATAAAGTGTAAAGCCTGGGGTGCCTAATGAGTGAGCTAACT | |
| | CACATTAATTGCGTTGCGCTCACTGCCCGCTTTCCAGTCGGGAAAC | |
| | CTGTCGTGCCAGCTGCATTAATGAATCGGCCAACGCGCGGGGAGA | |
| | GGCGGTTTGCGTATTGGGCGCTCTTCCGCTTCCTCGCTCACTGACTC | |
| | GCTGCGCTCGGTCGTTCGGCTGCGGCGAGCGGTATCAGCTCACTCA | |
| | AAGGCGGTAATACGGTTATCCACAGAATCAGGGGATAACGCAGGA | |
| | AAGAACATGTGAGCAAAAGGCCAGCAAAAGGCCAGGAACCGTAA | |
| | AAAGGCCGCGTTGCTGGCGTTTTTCCATAGGCTCCGCCCCCCTGAC | |
| | GAGCATCACAAAAATCGACGCTCAAGTCAGAGGTGGCGAAACCCG | |
| | ACAGGACTATAAAGATACCAGGCGTTTCCCCCTGGAAGCTCCCTCG | |
| | TGCGCTCTCCTGTTCCGACCCTGCCGCTTACCGGATACCTGTCCGC | |
| | CTTTCTCCCTTCGGGAAGCGTGGCGCTTTCTCATAGCTCACGCTGT | |
| | AGGTATCTCAGTTCGGTGTAGGTCGTTCGCTCCAAGCTGGGCTGTG | |
| | TGCACGAACCCCCCGTTCAGCCCGACCGCTGCGCCTTATCCGGTAA | |
| | CTATCGTCTTGAGTCCAACCCGGTAAGACACGACTTATCGCCACTG | |
| | GCAGCAGCCACTGGTAACAGGATTAGCAGAGCGAGGTATGTAGGC | |
| | GGTGCTACAGAGTTCTTGAAGTGGTGGCCTAACTACGGCTACACTA | |
| | GAAGGACAGTATTTGGTATCTGCGCTCTGCTGAAGCCAGTTACCTT | |
| | CGGAAAAAGAGTTGGTAGCTCTTGATCCGGCAAACAAACCACCGC | |
| | TGGTAGCGGTGGTTTTTTTGTTTGCAAGCAGCAGATTACGCGCAGA | |
| | AAAAAAGGATCTCAAGAAGATCCTTTGATCTTTTCTACGGGGTCTG | |
| | ACGCTCAGTGGAACGAAAACTCACGTTAAGGGATTTTGGTCATGA | |
| | GATTATCAAAAAGGATCTTCACCTAGATCCTTTTAAATTAAAAATG | |
| | AAGTTTTAAATCAATCTAAAGTATATATGAGTAAACTTGGTCTGAC | |
| | AGTTACCAATGCTTAATCAGTGAGGCACCTATCTCAGCGATCTGTC | |
| | TATTTCGTTCATCCATAGTTGCCTGACTCCCCGTCGTGTAGATAACT | |
| | ACGATACGGGAGGGCTTACCATCTGGCCCCAGTGCTGCAATGATA | |
| | CCGCGAGACCCACGCTCACCGGCTCCAGATTTATCAGCAATAAAC | |
| | CAGCCAGCCGGAAGGGCCGAGCGCAGAAGTGGTCCTGCAACTTTA | |
| | TCCGCCTCCATCCAGTCTATTAATTGTTGCCGGGAAGCTAGAGTAA | |
| | GTAGTTCGCCAGTTAATAGTTTGCGCAACGTTGTTGCCATTGCTAC | |
| | AGGCATCGTGGTGTCACGCTCGTCGTTTGGTATGGCTTCATTCAGC | |
| | TCCGGTTCCCAACGATCAAGGCGAGTTACATGATCCCCCATGTTGT | |
| | GCAAAAAAGCGGTTAGCTCCTTCGGTCCTCCGATCGTTGTCAGAAG | |
| | TAAGTTGGCCGCAGTGTTATCACTCATGGTTATGGCAGCACTGCAT | |
| | AATTCTCTTACTGTCATGCCATCCGTAAGATGCTTTTCTGTGACTGG | |
| | TGACGCGTCAACCAAGTCATTCTGAGAATAGTGTATGCGGCGACC | |
| | GAGTTGCTCTTGCCCGGCGTCAATACGGGATAATACCGCGCCACAT | |
| | AGCAGAACTTTAAAAGTGCTCATCATTGGAAAACGTTCTTCGGGGC | |
| | GAAAACTCTCAAGGATCTTACCGCTGTTGAGATCCAGTTCGATGTA | |
| | ACCCACTCGTGCACCCAACTGATCTTCAGCATCTTTTACTTTCACCA | |
| | GCGTTTCTGGGTGAGCAAAAACAGGAAGGCAAAATGCCGCAAAAA | |
| | AGGGAATAAGGGCGACACGGAAATGTTGAATACTCATACTCTTCC | |
| | TTTTTCAATATTATTGAAGCATTTATCAGGGTTATTGTCTCATGAGC | |
| | GGATACATATTTGAATGTATTTAGAAAAATAAACAAATAGGGGTT | |
| | CCGCGCACATTTCCCCGAAAAGTGCCAC | |
| pEAG658 | CTAAATTGTAAGCGTTAATATTTTGTTAAAATTCGCGTTAAATTTTT | SEQ ID NO:36 |
| | GTTAAATCAGCTCATTTTTTAACCAATAGGCCGAAATCGGCAAAAT | |
| | CCCTTATAAATCAAAAGAATAGACCGAGATAGGGTTGAGTGTTGT | |
| | TCCAGTTTGGAACAAGAGTCCACTATTAAAGAACGTGGACTCCAA | |
| | CGTCAAAGGGCGAAAAACCGTCTATCAGGGCGATGGCCCACTACG | |
| | TGAACCATCACCCTAATCAAGTTTTTTGGGGTCGAGGTGCCGTAAA | |
| | GCACTAAATCGGAACCCTAAAGGGAGCCCCCGATTTAGAGCTTGA | |
| | CGGGGAAAGCCGGCGAACGTGGCGAGAAAGGAAGGGAAGAAAGC | |
| | GAAAGGAGCGGGCGCTAGGGCGCTGGCAAGTGTAGCGGTCACGCT | |
| | GCGCGTAACCACCACACCCGCCGCGCTTAATGCGCCGCTACAGGG | |
| | CGCGTCCCATTCGCCATTCAGGCTGCGCAACTGTTGGGAAGGGCG | |
| | ATCGGTGCGGGCCTCTTCGCTATTACGCCAGCTGGCGAAAGGGGG | |
| | ATGTGCTGCAAGGCGATTAAGTTGGGTAACGCCAGGGTTTTCCCAG | |
| | TCACGACGTTGTAAAACGACGGCCAGTGAGCGCGCGTAATACGAC | |
| | TCACTATAGGGCGAATTGGGTACCGGGCCCTCTAGATCCTTTCAGC | |
| | TCCCTGCCCCGGACATGCCCAGTGGGTGGAAGCTGCCCTCTTCTAG | |
| | CAGGAGACGCCCCAGGCGGTAGAGCAGCTGGGGGTACCAATGCAC | |
| | ACCCTCCCCCGGaGTCCAGCTGCCCCATGCCAAGCTGTGAAAGAGT | |
| | CTCAGGGGCCAGAAGGCCAACTGAGCCAGGTGGTGGTCAGCCACG | |
| | GCCGCGAAGCAGGATGCCACCACATCCTCCACCACCAGTGTCCCA | |
| | TGCTTTGTGAGCGGGGCGTAGGCCCCGAGGGCCACGTGTGTAGAG | |
| | ACAGCTGCCACGCGGGCAGGCTGCAGGCCTGGCACCCCAGCCACC | |
| | AGCACGTACTGGCCAGGCTGCACGTGGCTGGCAAATGTGGCCCGG | |
| | AAGCGGGCTGCCGGCTCCGTGTGATTGTCAGCCGTAAAGAGCAGG | |
| | TGAGCGGGTGTGAGTGCCAGGCGGCGTGGGGGGTCCTGAGTCTCG | |
| | ATGACCTGGAAGGCTCTCAGCCTGTGGGGCTCGCGGTCCAGGAAA | |
| | ATGAGCACATCGCTGAAGGTGGGGCTCCCATCCTCCCCCATGGCCA | |
| | GCACACGGTCTCCCGGCCTCACGGCTGACAAGGCCACACGCGCCC | |
| | CACTCTCCAGGCGTACCTgggctccggcagggtcgacgccgcccgtcttggCTGCGG | |
| | CCGAGTGCTCGGACTTGACGGAGCAATGCACGTGGGCCTTTGACTC | |
| | GTAATACACCCAGTCAAAGCCGGCCTCCACTGCCAAGCGCGCCAG | |
| | CAGTCCATACTTATTGCGGTCGCGGTCTGATGTGGTGATGTCCACC | |
| | GCGCGGCCCTCATAATGCAGGGACTCCTCTGAGTGGTGGCCGTCCTI | |
| | CGTCCCAGCCCTCGGTCACCCGCAGCTTCACACCGGGCCACTGGTT | |
| | CATCACCGAGATAGCCAGCGAGTTCAGGCGGTCCTTGCAGCGCTG | |
| | GGTCATGAGGCGGTCGGCGCCTGTGTTCTCCTCGTCCTTGAAGATG | |
| | ATGTCTGGATTGTAATTGGGGGTGAGCTCCTTGAAGCGCTCGGAGC | |
| | TGCGAGCGATCTTGCCTTCATAGCGTCCGCTGGCGCCCAGGGTCTT | |
| | CTCGGGCACATTGGGGCTGAACTGCTTGTAGGCGAGCGGCACGAG | |
| | TTTGCGTGGCGGTCGCCGGCGGCTGCCCACCACCCGACCCGGCCCG | |
| | CAGCCCCATGCCGCCGGCACCACCAGCAGCAGCAACAGGACCAGG | |
| | CAGAAGTGCAGTCGGGGCCGGAGCCGggcgggagacatggcggccgcgacggtat | |
| | cgataagcTTGATATCGAATTCCTGCAGCCCGGGGGATCCACTAGTTCT | |
| | AGAGCGGCCGCCACCGCGGTGGAGCTCCAGCTTTTGTTCCCTTTAG | |
| | TGAGGGTTAATTGCGCGCTTGGCGTAATCATGGTCATAGCTGTTTC | |
| | CTGTGTGAAATTGTTATCCGCTCACAATTCCACACAACATACGAGC | |
| | CGGAAGCATAAAGTGTAAAGCCTGGGGTGCCTAATGAGTGAGCTA | |
| | ACTCACATTAATTGCGTTGCGCTCACTGCCCGCTTTCCAGTCGGGA | |
| | AACCTGTCGTGCCAGCTGCATTAATGAATCGGCCAACGCGCGGGG | |
| | AGAGGCGGTTTGCGTATTGGGCGCTCTTCCGCTTCCTCGCTCACTG | |
| | ACTCGCTGCGCTCGGTCGTTCGGCTGCGGCGAGCGGTATCAGCTCA | |
| | CTCAAAGGCGGTAATACGGTTATCCACAGAATCAGGGGATAACGC | |
| | AGGAAAGAACATGTGAGCAAAAGGCCAGCAAAAGGCCAGGAACC | |
| | GTAAAAAGGCCGCGTTGCTGGCGTTTTTCCATAGGCTCCGCCCCCC | |
| | TGACGAGCATCACAAAAATCGACGCTCAAGTCAGAGGTGGCGAAA | |
| | CCCGACAGGACTATAAAGATACCAGGCGTTTCCCCCTGGAAGCTC | |
| | CCTCGTGCGCTCTCCTGTTCCGACCCTGCCGCTTACCGGATACCTGT | |
| | CCGCCTTTCTCCCTTCGGGAAGCGTGGCGCTTTCTCATAGCTCACG | |
| | CTGTAGGTATCTCAGTTCGGTGTAGGTCGTTCGCTCCAAGCTGGGC | |
| | TGTGTGCACGAACCCCCCGTTCAGCCCGACCGCTGCGCCTTATCCG | |
| | GTAACTATCGTCTTGAGTCCAACCCGGTAAGACACGACTTATCGCC | |
| | ACTGGCAGCAGCCACTGGTAACAGGATTAGCAGAGCGAGGTATGT | |
| | AGGCGGTGCTACAGAGTTCTTGAAGTGGTGGCCTAACTACGGCTA | |
| | CACTAGAAGGACAGTATTTGGTATCTGCGCTCTGCTGAAGCCAGTT | |
| | ACCTTCGGAAAAAGAGTTGGTAGCTCTTGATCCGGCAAACAAACC | |
| | ACCGCTGGTAGCGGTGGTTTTTTTGTTTGCAAGCAGCAGATTACGC | |
| | GCAGAAAAAAAGGATCTCAAGAAGATCCTTTGATCTTTTCTACGG | |
| | GGTCTGACGCTCAGTGGAACGAAAACTCACGTTAAGGGATTTTGG | |
| | TCATGAGATTATCAAAAAGGATCTTCACCTAGATCCTTTTAAATTA | |
| | AAAATGAAGTTTTAAATCAATCTAAAGTATATATGAGTAAACTTGG | |
| | TCTGACAGTTACCAATGCTTAATCAGTGAGGCACCTATCTCAGCGA | |
| | TCTGTCTATTTCGTTCATCCATAGTTGCCTGACTCCCCGTCGTGTAG | |
| | ATAACTACGATACGGGAGGGCTTACCATCTGGCCCCAGTGCTGCA | |
| | ATGATACCGCGAGACCCACGCTCACCGGCTCCAGATTTATCAGCA | |
| | ATAAACCAGCCAGCCGGAAGGGCCGAGCGCAGAAGTGGTCCTGCA | |
| | ACTTTATCCGCCTCCATCCAGTCTATTAATTGTTGCCGGGAAGCTA | |
| | GAGTAAGTAGTTCGCCAGTTAATAGTTTGCGCAACGTTGTTGCCAT | |
| | TGCTACAGGCATCGTGGTGTCACGCTCGTCGTTTGGTATGGCTTCA | |
| | TTCAGCTCCGGTTCCCAACGATCAAGGCGAGTTACATGATCCCCCA | |
| | TGTTGTGCAAAAAAGCGGTTAGCTCCTTCGGTCCTCCGATCGTTGT | |
| | CAGAAGTAAGTTGGCCGCAGTGTTATCACTCATGGTTATGGCAGCA | |
| | CTGCATAATTCTCTTACTGTCATGCCATCCGTAAGATGCTTTTCTGT | |
| | GACTGGTGACGCGTCAACCAAGTCATTCTGAGAATAGTGTATGCG | |
| | GCGACCGAGTTGCTCTTGCCCGGCGTCAATACGGGATAATACCGC | |
| | GCCACATAGCAGAACTTTAAAAGTGCTCATCATTGGAAAACGTTCT | |
| | TCGGGGCGAAAACTCTCAAGGATCTTACCGCTGTTGAGATCCAGTT | |
| | CGATGTAACCCACTCGTGCACCCAACTGATCTTCAGCATCTTTTAC | |
| | TTTCACCAGCGTTTCTGGGTGAGCAAAAACAGGAAGGCAAAATGC | |
| | CGCAAAAAAGGGAATAAGGGCGACACGGAAATGTTGAATACTCAT | |
| | ACTCTTCCTTTTTCAATATTATTGAAGCATTTATCAGGGTTATTGTC | |
| | TCATGAGCGGATACATATTTGAATGTATTTAGAAAAATAAACA | |
| | AATAGGGGTTCCGCGCACATTTCCCCGAAAAGTGCCAC | |

Construction ofHedgehog-Ig Fusion Proteins

Shh-Fc(muIgGl) plasmid pUB 114 (SEQ ID NO: 32), has the wild-type SHH domain (SEQ ID NO: 21 or 23) fused to the CH₂ and CH3 regions of murine IgGI (SEQ ID NO: 29).

The Fc region in pUBl14 contains a glycosylation site mutation [Asn297Gln]. Plasmid pUB55 (SEQ ID NO: 31) and pUB 114 plasmids are identical outside of the region coding for the Fc domain fused to SHH. Plasmids identical to pUB 114, but containing the human IgGI or murine IgG2a Fc region are pUB I15 (SEQ ID NO: 33) and pUB 116 (SEQ ID NO: 34), respectively.

For construction of yeast strains expressing protein, plasmids were digested with Stul and transformed into Pichia pastoris GS 115 by electroporation in 1 M Sorbitol >-=(Invitrogen) or by a Li salt transformation procedure (Frozen EZ Yeast Transformation kit, Zymo Research, Orange, Calif.). His+transformants were selected on MD agar. Colonies were purified on YPD agar and cultured for protein expression in 5 ml BMMY (2% Methanol) medium. BMMY culture supernatants were harvested at 1 or 2 days (1-day harvests were concentrated by TCA precipitation) and were analyzed by SDS-PAGE and Coomassie blue staining to distinguish clipped and unclipped SHE

Protein Purification

Large scale preparations of protein for purification were prepared as follows: An inoculum in BMGY (late log to stationary phase) was added to 1 L BMGY in a Fer nbach flask and incubated at 150 rpm for 2-3 days. The stationary phase BMGY culture was centrifuged and the cell pellet from 1 L was resuspended in BMMY(2% Methanol) and incubated in a Fer nbach flask at 30 C for 2-3 days. Pepstatin A (44 microM) was added to BMMY medium for expression of SHH-Fc fusion proteins.

A. Purification of Hedgehog-Ig fusion protein constructs

Pichia cells were removed from the conditioned medium by centrifugation before application to Protein A Fast Flow® (Pharmacia). Protein from constructs utilizing human IgGI (SEQ ID NO: 28) or murine IgG2A sequences (SEQ ID NO: 30) were applied directly to the Protein A. Constructs utilizing murine IgG 1 sequences were diluted ten-fold with water to reduce the salt concentration, re-concentrated using a 3K cutoff spiral filter (Amicon) and the pH adjusted with the addition of sodium borate buffer, pH 8.5 to a final concentration of 50 mM.

HHIg was eluted with 25 mM sodium phosphate, pH 2.8, and the fractions collected into tubes containing 0.1 volume of 0.5 M sodium phosphate pH 6 to readjust the pH. The Protein A eluant was then diluted eight-fold with 0.5 mM sodium phosphate, pH 6 and applied to a CM-Poros® column (Perseptive Biosystems) equilibrated with 50 mM sodium phosphate, pH 6.0. Elution with a gradient of 0-0.8 M NaCl separated two HH lg peaks.

The first is “one-armed” protein in which one of the HHIg polypeptides of the dimer is proteolytically cleaved at a sequence near the hinge and therefore this dimer contains only one HH N-terminal domain. The second peak is the dimer with two full-length HHIg chains. The peaks were pooled separately, reduced with 10 mM DTT and dialyzed against 5 mM sodium phosphate, pH 5.5, 150 mM NaCl and 0.5 mM DTT. No DTT was used when the N-terminal cysteine of the protein was replaced with other amino acids. These two purification steps achieve >95% purity. Purity was determined by SDS-PAGE on 4-20% gradient gels (Novex) stained with Coomassie Blue. Identity was confirmed by mass spectrometry, and potency was analyzed using a cell-based bioactivity assay (see above).

Mass spectrometry

The molecular masses of the purified proteins were determined by electrospray ionization mass spectroscopy (ESI-MS) on a Micromass Quattro II triple quadrupole mass spectrometer. Samples were desalted using an on-line Michrom Ultrafast Microprotein Analyzer system with a Reliasil© C4 column (1 mm ×5 cm). All electrospray mass spectral data were processed using the Micromass MassLynx data system.

References:

Apelqvist A, Ahlgren U, Edlund H. Sonic hedgehog directs specialised mesoderm differentiation in the intestine and pancreas. Curr Biol. Oct. 1, 1997 ;7(10):801-4.

Asahara, T, Chen D, Tomono, T, Fujikawa, K, Kearney, M, Magner, M, Yancopoulos, GD and Isner, JM. Tie2 receptor ligands, angiopoietin-1 and angiopoietin-2, modulate VEGF-Induced postnatal neovascularization. Circ. Res. 1997 83: 233-240.

Ballara S C, Miotla J M, Paleolog E M. New vessels, new approaches: angiogenesis as a therapeutic target in musculoskeletal disorders. Int J Exp Pathol. October 1999;80(5):235-50.

Banai S, Jaklitsch M T, Shou M, Lazarous D F, Scheinowitz M, Biro S, Epstein S E, Unger E F. Angiogenic-induced enhancement of collateral blood flow to ischemic myocardium by vascular endothelial growth factor in dogs. Circulation. May 1994;89(5):2183-9

Battler A, Scheinowitz M, Bor A, Hasdai D, Vered Z, Di Segni E, Varda-Bloom N, Nass D, Engelberg S, Eldar M, et al. Intracoronary injection of basic fibroblast growth factor enhances angiogenesis in infarcted swine myocardium. J Am Coll Cardiol. December 1993;22(7):2001-6.

Beck L Jr, D'Amore P A. Vascular development: cellular and molecular regulation. FASEB J. April 1997; 11(5):365-73

Bitgood M J, McMahon A P. Hedgehog and Bmp genes are coexpressed at many diverse sites of cell-cell interaction in the mouse embryo. Dev Biol. November 1995;172(1):126-38

Bitgood M J, Shen L, McMahon A P. (1996) Sertoli cell signaling by Desert hedgehog regulates the male germline. Curr Biol. 6(3):298-304.

Bhushan M, McLaughlin B, Weiss J B, Griffiths C E. Levels of endothelial cell stimulating angiogenesis factor and vascular endothelial growth factor are elevated in psoriasis. Br J Dermatol. December 1999;141(6):1054-60.

Buschmann 1, Schaper W. The pathophysiology of the collateral circulation (arteriogenesis). J Pathol. February 2000;190(3):338-42.

Carpenter D, Stone D M, Brush J, Ryan A, Armanini M, Frantz G, Rosenthal A, de Sauvage F J. Characterization of two patched receptors for the vertebrate hedgehog protein family. Proc Natl Acad Sci USA. Nov. 10, 1998;95(23):13630-4.

Chiang C, Litingtung Y, Lee E, Young K E, Corden J L, Westphal H, Beachy P A. cyclopia and defective axial patterning in mice lacking sonic hedgehog gene function. Nature 1996; 383:407-413.

Cherrington J M, Strawn L M, Shawver L K. New paradigms for the treatment of cancer: the role of anti-angiogenesis agents. Adv Cancer Res. 2000;79:1-38.

Couffinhal T, Silver M, Kearney M, Sullivan A, Witzenbichler B, Magner M, Annex B, Peters K, Isner J M. Impaired collateral vessel development associated with reduced expression of vascular endothelial growth factor in ApoE-/-mice. Circulation. 1999; 99: 3188-3198.

D'Amato R J, Adamis A P. Angiogenesis inhibition in age-related macular degeneration. Ophthalmology. September 1995;102(9):1261-2.

Ding Q, Fukami Si, Meng X, Nishizaki Y, Zhang X, Sasaki H, Dlugosz A, Nakafuku M, Hui Cc. Mouse suppressor of fused is a negative regulator of sonic hedgehog signaling and alters the subcellular distribution of Gli1. Curr Biol. Oct. 7, 1999;9(19):1119-22.

Dockter J L. Sclerotome induction and differentiation. Curr Top Dev Biol. 2000 48:77-127.

Dodd J, Jessell T M, Placzek M. The when and where of floor plate induction. Science 1998 282(5394):1654-7.

Ericson J, Muhr J, Jessell T M, Edlund T. Sonic hedgehog: a common signal for ventral patterning along the rostrocaudal axis of the neural tube. Int J Dev Biol. 1995 39(5):809-16.

Ericson J, Briscoe J, Rashbass P, van Heyningen V, Jessell T M. Graded sonic hedgehog signaling and the specification of cell fate in the ventral neural tube. Cold Spring Harb Symp Quant Biol. 1997 62:451-66

Engler D A. Use of vascular endothelial growth factor for therapeutic angiogenesis. Circulation. Oct. 1, 1996;94(7):1496-8.

Fan H, Villegas C, Chan A K, Wright J A. Myc-epitope tagged proteins detected with the 9E10 antibody in immunofluorescence and immunoprecipitation assays but not in western blot analysis. Biochem Cell Biol. 1998;76(1):125-8.

Folkman J, Shing Y. Angiogenesis. J Biol Chem. Jun. 5, 1992;267(16):10931-4

Fong T A, Shawver L K, Sun L, Tang C, App H, Powell T J, Kim Y H, Schreck R, Wang X, Risau W, Ullrich A, Hirth K P, McMahon G. SU5416 is a potent and selective inhibitor of the vascular endothelial growth factor receptor (Flk-1/KDR) that inhibits tyrosine kinase catalysis, tumor vascularization, and growth of multiple tumor types. Cancer Res. 1999 Jan 1;59(1):99-106.

Goodrich L V, Milenkovic L, Higgins K M, Scott M P. Altered neural cell fate and medulloblastoma in mouse patched mutants. Science 1997;277(5329):1109-1113.

Hammerschmidt M, Brook A, McMahon A P. The world according to hedgehog. Trends Genet. January 1997;13(l):14-21.

Harada K, Grossman W, Friedman M, Edelman E R, Prasad P V, Keighley C S, Manning W J, Sellke F W, Simons M. Basic fibroblast growth factor improves myocardial function in chronically ischemic porcine hearts. J Clin Invest. August 1994;94(2):623-30.

Hynes M, Ye W, Wang K, Stone D, Murone M, Sauvage Fd, Rosenthal A. The seven-transmembrane receptor smoothened cell-autonomously induces multiple ventral cell types. Nat Neurosci. January 2000;3(1):41-6.

Ingham P W. Signalling by hedgehog family proteins in Drosophila and vertebrate development. Curr Opin Genet Dev. 1995; 5:492-8.

Isner J M, Walsh K, Symes J, Pieczek A, Takeshita S, Lowry J, Rosenfield K, Weir L, Brogi E, Jurayj D. Arterial gene transfer for therapeutic angiogenesis in patients with peripheral artery disease. Hum Gene Ther. May 20, 1996;7(8):959-88

Iwamoto M, Enomoto-Iwamoto M, Kurisu K. Actions.of hedgehog proteins on skeletal cells. Crit Rev Oral Biol Med. 1999; 10:477-486.

Jensen A M, Wallace V A. Expression of Sonic hedgehog and its putative role as a precursor cell mitogen in the developing mouse retina. Development. January 1997;124(2):363-71.

Johnson R L, Tabin C J. Molecular models for vertebrate limb development. Cell. 1997; 90(6):979-90.

Karasek M A. Progress in our understanding of the biology of psoriasis. Cutis. November 1999;64(5):319-22.

Karp S J, Schipani E, St-Jacques B, Hunzelman J, Kronenberg H, McMahon A P. Indian hedgehog coordinates endochondral bone growth and morphogenesis via parathyroid hormone related-protein-dependent and -independent pathways. Development. 2000; 127(3):543-8.

Kenyon, B M, Voest, E E, Chen C C. Flynn, E., Folkman, J and D'Amato, R J. A model of angiogenesis in the mouse cornea. Investigative Ophthalmology & Visual Science 1996; 37: 1625-1632.

Klagsbrun M, D'Amore P A. Regulators of angiogenesis. Annu Rev Physiol. 1991;53:217-39

Klohs W D, Hamby J M Antiangiogenic agents. Curr Opin Biotechnol. December 1999;10(6):544-9.

Kornowski R, Hong M K, Leon M B. Comparison between left ventricular electromechanical mapping and radionuclide perfusion imaging for detection of myocardial viability. Circulation. Nov. 3, 1998;98(18):1837-41.

Komowski R, Fuchs S, Leon M B, Epstein S E. Delivery strategies to achieve therapeutic myocardial angiogenesis. Circulation. Feb. 1, 2000;101(4):454-8.

Laham R J, Rezaee M, Post M, Novicki D, Sellke F W, Pearlman J D, Simons M, Hung D. Intrapericardial delivery of fibroblast growth factor-2 induces neovascularization in a porcine model of chronic myocardial ischemia. J Pharmacol Exp Ther. February 2000;292(2):795-802.

Landau C, Jacobs A K, Haudenschild C C. Intrapericardial basic fibroblast growth factor induces myocardial angiogenesis in a rabbit model of chronic ischemia. Am Heart J. May 1995;129(5):924-31.

Lazarous D F, Shou M, Scheinowitz M, Hodge E, Thirumurti V, Kitsiou A N, Stiber J A, Lobo A D, Hunsberger S, Guetta E, Epstein S E, Unger E F. Comparative effects of basic fibroblast growth factor and vascular endothelial growth factor on coronary collateral development and the arterial response to injury. Circulation. Sep. 1, 1996;94(5): 1074-82

Lemire J M, Covin C W, Whit S, Giacelli C M, Schwartz S M. Characterization of cloned aortic smooth muscle cells from young rats. Am. J. Pathol. 1994; 144:1068-1081.

Litingtung Y, Lei L, Westphal H, Chiang C. Sonic hedgehog is essential to foregut development. Nat Genet. 1998; 20(l):58-61.

119

Magovem C J, Mack C A, Zhang J, Rosengart T K, Isom O W, Crystal R G. Regional angiogenesis induced in nonischemic tissue by an adenoviral vector expressing vascular endothelial growth factor. Hum Gene Ther. Jan. 20, 1997;8(2):215-27.

Majesky M W. A little VEGF goes a long way. Therapeutic angiogenesis by direct injection of vascular endothelial growth factor-encoding plasmid DNA. Circulation. Dec. 15, 1996;94(12):3062-4.

Mesri E A, Federoff H J, Brownlee M. Expression of vascular endothelial growth factor from a defective herpes simplex virus type 1 amplicon vector induces angiogenesis in mice. Circ Res. February 1995;76(2):161-7.

Motoyama J, Heng H, Crackower M A, Takabatake T, Takeshima K, Tsui L C, Hui C. Overlapping and non-overlapping Ptch2 expression with Shh during mouse embryogenesis. Mech Dev. November 1998;78(1-2):81-4.

Murone M, Rosenthal A, de Sauvage F J. Hedgehog signal transduction: from flies to vertebrates. Exp Cell Res. November 25, 1999a ;253(1):25-33

Murone M, Rosenthal A, de Sauvage F J. Sonic hedgehog signaling by the patched-smoothened receptor complex. Curr Biol. Jan. 28, 1999;9(2):76-84.

Ozaki H, Seo M S, Ozaki K, Yamada H, Yamada E, Okamoto N, Hofmann F, Wood J M, Campochiaro P A. Blockade of vascular endothelial cell growth factor receptor signaling is sufficient to completely prevent retinal neovascularization. Am J Pathol. February 2000; 156(2):697-707.

Parmantier E, Lynn B, Lawson D, Turmaine M, Namini S S, Chakrabarti L, McMahon A P, Jessen K R, Mirsky R. Schwann cell-derived Desert hedgehog controls the development of peripheral nerve sheaths. Neuron 1999; 23(4):713-24.

Passaniti, A, Taylor, R M, Pili, R, Guo, Y, Long, P V, Haney, F A, Pauly, R R, Grant, D S and Martin, G R. A simple, quantitative method for assessing angiogenesis and antiangiogenic agents using reconstituted basement membrane, heparin, and fibroblast growth factor. Lab. Invest. 1992 67: 519-528.

Peacock D J, Banquerigo M L, Brahn E. A novel angiogenesis inhibitor suppresses rat adjuvant arthritis. Cell Immunol. February 1995;160(2):178-84.

Pearlman J D, Hibberd M G, Chuang M L, Harada K, Lopez J J, Gladstone S R, Friedman M, Sellke F W, Simons M. Magnetic resonance mapping demonstrates benefits of VEGF-induced myocardial angiogenesis. Nat Med. October 1995;1(10): 1085-9.

Pearse R V 2nd, Collier L S, Scott M P, Tabin C J. Vertebrate homologs of Drosophila suppressor of fused interact with the gli family of transcriptional regulators. Dev Biol. Aug. 15, 1999;212(2):323-36.

Pepinsky R B, Zeng C, Wen D, Rayhom P, Baker D P, Williams K P, Bixler S A, Ambrose C M, Garber E A, Miatkowski K, Taylor, F R, Wang E A, Galdes A. Identification of a palmitic acid-modified form of human Sonic hedgehog. J Biol Chem 1998 273(22):14037-45.

Pepinsky R B, Rayhom P, Day E S, Dergay A, Williams K P, Galdes A, Taylor F R, Boriack-Sjodin A, Garber E A. Mapping sonic hedgehog-receptor interactions by steric interference. J. Biol. Chem. 2000 275:10995-11001.

Perrimon N. Hedgehog and beyond. Cell 1995; 80(4):517-20

Rivard A, Isner J M. Angiogenesis and vasculogenesis in treatment of cardiovascular disease. Mol Med. July 1998;4(7):429-40.

Rivard A, Fabre J E, Silver M, Chen D, Murohara T, Kearney M, Magner M, Asahara T, Isner J M. Age-dependent impairment of angiogenesis. Circulation. Jan. 5-12 1999;99(1):111-20.

Rothman A, Kulik, T J, Taubman, M B, Berk, B C, Smith C W J, nadal-Ginard, B. Development and characterization of a cloned rat pulmonary arterial smooth muscle cell line that maintains differentiated propoerties through multiple subcultures. Circulation. 1992; 86:1977-1986.

Sato N, Leopold P L, Crystal R G. Induction of the hair growth phase in postnatal mice by localized transient expression of Sonic hedgehog. J Clin Invest. October 199;104(7):855-64.

Schratzberger P, Schratzberger G, Silver M, Curry C, Kearney M, Magner M, Alroy J, Adelman L S, Weinberg D H, Ropper A H, Isner J M. Favorable effect of VEGF gene transfer on ischemic peripheral neuropathy. —Nat Med. April 2000;6(4):405-13.

Shou M, Thirumurti V, Rajanayagam S, Lazarous D F, Hodge E, Stiber J A, Pettiford M, Elliott E, Shah S M, Unger E F. Effect of basic fibroblast growth factor on myocardial angiogenesis in dogs with mature collateral vessels. J Am Coll Cardiol. April 1997;29(5): 1102-6.

St-Jacques B, Dassule H R, Karavanova I, Botchkarev V A, Li J, Danielian P S, McMahon J A, Lewis P M, Paus R, McMahon A P. Sonic hedgehog signaling is essential for hair development. Curr Biol. 1998; 8(19):1058-68

St-Jacques B, Hammerschmidt M, McMahon A P. Indian hedgehog signaling regulates proliferation and differentiation of chondrocytes and is essential for bone formation. Genes Dev. 1999; 13(16):2072-86.

Stone D M, Murone M, Luoh S, Ye W, Armanini M P, Gurney A, Phillips H, Brush J, Goddard A, de Sauvage, F J, Rosenthal A. Characterization of the human suppressor of fused, a negative regulator of the zinc-finger transcription factor Gli. J Cell Sci. December 1999;112 (Pt 23):4437-48.

Storgard C M, Stupack D G, Jonczyk A, Goodman S L, Fox R I, Cheresh D A. Decreased angiogenesis and arthritic disease in rabbits treated with an alphavbeta3 antagonist. J Clin Invest. Jaunary 1999;103(1):47-54.

Takeshita S, Pu L Q, Stein L A, Sniderman A D, Bunting S, Ferrara N, Isner J M, Symes J F. Intramuscular administration of vascular endothelial growth factor induces dose-dependent collateral artery augmentation in a rabbit model of chronic limb ischemia. Circulation. November 1994;90(5 Pt 2):II228-34.

Takeshita S, Weir L, Chen D, Zheng L P, Riessen R, Bauters C, Symes J F, Ferrara N, Isner J M. Therapeutic angiogenesis following arterial gene transfer of vascular endothelial growth factor in a rabbit model of hindlimb ischemia. Biochem Biophys Res Commun. Oct. 14, 1996;227(2):628-35.

Taylor F R, Wen D, Garber E A, Baker D P, Arduini R M, Williams K P, Weinreb P H, Rayhom P, Hronowski X, Whitty A, Day E S, Boriack-Sjodin A, Shapiro R, and Pepinsky R B. Enhanced potency of human sonic hedgehog by hydrophobic modification. Manuscript in prep.

Traiffort E, Charytoniuk D A, Faure H, Ruat M. Regional distribution of Sonic Hedgehog, patched, and smoothened mRNA in the adult rat brain. J Neurochem. March 1998;70(3):1327-30.

Traiffort E, Charytoniuk D, Watroba L, Faure H, Sales N, Ruat M. Discrete localizations of hedgehog signalling components in the developing and adult rat nervous system. Eur J Neurosci. September 1999;11(9):3199-214

Unger E F, Banai S, Shou M, Lazarous D F, Jaklitsch M T, Scheinowitz M, Correa R, Klingbeil C, Epstein S E. Basic fibroblast growth factor enhances myocardial collateral flow in a canine model. Am J Physiol. April 1994;266(4 Pt 2):H1588-95.

Vale P R, Losordo D W, Tkebuchava T, Chen D, Milliken C E, Isner J M. Catheter-based myocardial gene transfer utilizing nonfluoroscopic electromechanical left ventricular mapping. J Am Coll Cardiol. July 1999;34(1):246-54.

Valentini R P, Brookhiser W T, Park J, Yang T, Briggs J, Dressler G, Holzman L B. Post-translational processing and renal expression of mouse Indian hedgehog. J Biol Chem. Mar. 28, 1997;272(13):8466-73.

Walsh D A. Angiogenesis and arthritis. Rheumatology (Oxford). February 1999;38(2):103-12.

Wang L C, Liu Z Y, Gambardella L, Delacour A, Shapiro R, Yang J, Sizing I, Rayhom P, Garber E A, Benjamin C D, Williams K P, Taylor F R, Barrandon Y, Ling L, Burkly L C. Conditional Disruption of Hedgehog Signaling Pathway Defines its Critical Role in Hair Development and Regeneration. J Invest Dermatol. May 2000;114(5):901-908.

Wood J M, Bold G, Buchdunger E, Cozens R, Ferrari S, Frei J, Hofmann F, Mestan J, Mett H, O'Reilly T, Persohn E, Rosel J, Schnell C, Stover D, Theuer A, Towbin H, Wenger F, Woods-Cook K, Menrad A, Siemeister G, Schimer M, Thierauch K H, Schneider M R, Drevs J, Martiny-Baron G, Totzke F. PTK787/ZK 222584, a novel and potent inhibitor of vascular endothelial growth factor receptor tyrosine kinases, impairs vascular endothelial growth factor-induced responses and tumor growth after oral administration. Cancer Res. Apr. 15, 2000;60(8):2178-89.

Yancopoulos G D, Klagsbrun M, Folkman J. Vasculogenesis, angiogenesis, and growth factors: ephrins enter the fray at the border. Cell. May 29, 1998;93(5):661-4.

Yanagisawa-Miwa A, Uchida Y, Nakamura F, Tomaru T, Kido H, Kamijo T, Sugimoto T, Kaji K, Utsuyama M, Kurashima C, et al. Salvage of infarcted myocardium by angiogenic action of basic fibroblast growth factor. Science. Sep. 4, 1992;257(5075):1401-3.

Zhu Z, Witte L. Inhibition of tumor growth and metastasis by targeting tumor-associated angiogenesis with antagonists to the receptors of vascular endothelial growth factor. Invest New Drugs. 1999; 17(3):195-212.

We claim:

1. A method of promoting angiogenesis in a subject animal comprising administering to the subject an angiogenic amount of a hedgehog polypeptide or agonist thereof.

2. The method of claim 1, wherein the step of administering comprises contacting the hedgehog polypeptide or agonist with a mesenchymal cell of the subject.

3. The method of claim 1, comprising administering to the subject a polypeptide including a hedgehog amino acid sequence, which hedgehog sequence directs the binding of the polypeptide to a patched receptor polypeptide and/or induces alkaline phosphatase activity in C3H10T1/2 cells.

4. The method of claim 1, comprising administering to the subject a polypeptide including a hedgehog amino acid sequence having at least 60% amino acid identity with SEQ ID No. 10-18 or 20.

5. The method of claim 1, comprising administering to the subject a polypeptide including a hedgehog amino acid sequence encoded by a coding sequence which hybridizes under stringent conditions to any of SEQ ID No. 1-9 or 19.

6. The method of claim 1, comprising administering to the subject a polypeptide including a hedgehog amino acid sequence represented by SEQ ID No. 26.

7. The method of any of claims 3 -7, wherein the hedgehog sequence includes at least 50 resdiues of an extracellular domain of a hedgehog protein.

8. The method of any of claims 3 -7, wherein the polypeptide is derivatized with one or more chemical moieties.

9. The method of claim 8, wherein the chemical moiety is a polyalkylene glycol polymer.

10. The method of claim 8, wherein the chemical moiety is a hydrophobic moiety.

11. The method of claim 10, wherein the hydrophobic moiety is a lipid.

12. The method of claim 8, wherein the chemical moiety is one or more phosphate groups.

13. The method of claim 8, wherein the chemical moiety is one or more acetyl groups.

14. The method of claim 8, wherein the chemical moiety is one or more sugar or carbohydrate groups.

15. The method of claim 8, wherein the chemical moieties are any combination of phosphate, acetyl, sugar, carbohydrate, or hydrophobic moieties.

16. The method of claim 4, wherein the method further comprises administering an agent that enhances agonistic properties of the hedgehog therapeutic.

17. The method of claim 16, wherein the agent is an angiogenic factor selected from the group consisting of vascular endothelial growth factor (VEGF), hepatocyte growth factor (HGF), basic fibroblast growth factor (bFGF), angiopoietin 1, angiopoietin 2, monocyte chemotactic protein-1 (MCP-1).

18. A method of inhibiting angiogenesis in a subject animal comprising administering to the subject an antiangiogenic amount of a hedgehog antagonist.

19. The method of claim 18, comprising administering a polypeptide including one or more antigen binding domains which bind to and inhibit hedgehog signalling.

20. The method of claim 18, comprising administering a polypeptide including one or more antigen binding domains which bind to patched and inhibit hedgehog signalling.

21. The method of claim 18, comprising administering a polypeptide including one or more antigen binding domains which bind to smoothened and inhibit hedgehog signalling.

22. The method of claim 19, 20 or 21, wherein the antigen binding domain is part of a an antibody structure selected from the group consisting of a humanized antibody homology, a human antibody homolog, a chimeric antibody homolog and fragments thereof.

23. The method of claim 18, comprising administering a finctional antagonist of a hedgehog therapeutic.

24. The method of claim 18, or 20, wherein the subject has a condition selected from the group consisting of a malignant tumor, retinopathy, macular degeneration, a nonmalignant tumor, rheumatoid arthritis, osteoarthritis, neovascular glaucoma, keloids, Crohn's disease, ulcerative colitis, and psoriasis.

25. The method of claim 1, wherein the hedgehog agonist is a small organic molecule.

26. The method of claim 25, wherein the hedgehog agonist has a molecular weight less than 2500 amu.

27. The method of claim 25, wherein the hedgehog agonist is represented by general formula (XII):
Formula XII 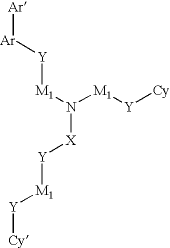
wherein, as valence and stability permit,
Ar and Ar′ independently represent substituted or unsubstituted aryl or heteroaryl rings;
Y, independently for each occurrence, may be absent or represent —N(R)—, —O—, —S—, or —Se—;
X can be selected from —C(═O)—, —C(═S)—, —S(O₂)—, —S(O)—, —C(═NCN)—, —P(═O)(OR₂)—, and a methylene group optionally substituted with 1-2 groups such as lower alky, alkenyl, or alkynyl groups;
M represents, independently for each occurrence, a substituted or unsubstituted methylene group, such as —CH₂—, —CHF—, —CHOH—, —CH(Me)—, —C(═O)—, etc., or two M taken together represent substituted or unsubstituted ethene or ethyne;
R represents, independently for each occurrence, H or substituted or unsubstituted aryl, heterocyclyl, heteroaryl, aralkyl, heteroaralkyl, alkynyl, alkenyl, or alkyl, or two R taken together may form a 4- to 8-membered ring, e.g., with N;
Cy and Cy′ independenly represent substituted or unsubstituted aryl, heterocyclyl, heteroaryl, or cycloalkyl, including polycyclic groups;
i represents, independently for each occurrence, an integer from 0 to 5, preferably from 0 to 2; and
n, individually for each occurence, represents an integer from 0 to 10, preferably from 0 to 5.

28. The method of any of claims 3-7, comprising administering a nucleic acid sequence encoding the polypeptide.

29. The method of claim 29, wherein the nucleic acid sequences encoding the polypeptide are introduced via a viral vector, via lipofection, and/or as naked DNA.

30. The method of claim 18, wherein the hedgehog antagonist is a small organic molecule.

31. The method of claim 30, wherein the hedgehog antagonist has a molecular weight less than 2500 amu.

32. The method of claim 30, wherein the hedgehog antagonist is represented by one or more of formulas I-XI.

33. The method of claim 30, wherein the hedgehog antagonist is represented by general formula (I): 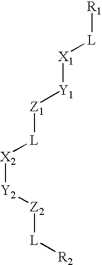
wherein, as valence and stability permit,
R₁ and R₂, independently for each occurrence, represent H, lower alkyl, aryl (e.g., substituted or unsubstituted), aralkyl (e.g., substituted or unsubstituted, e.g., —(CH₂)ₙaryl), or heteroaryl (e.g., substituted or unsubstituted), or heteroaralkyl (e.g., substituted or unsubstituted, e.g., —(CH₂)ₙheteroaralkyl-);
L, independently for each occurrence, is absent or represents —(CH₂)ₙ-alkyl, -alkenyl-, -alkynyl-, —(CH₂)ₙalkenyl-, —(CH₂)ₙalkynyl-, —(CH₂)ₙO(CH₂)ₚ—, —(CH₂)ₙNR₂(CH₂)ₚ—, —(CH₂)ₙS(CH₂)ₚ—, —(CH₂)ₙalkenyl(CH₂)ₚ—, —(CH₂)ₙalkynyl(CH₂)ₚ—, —O(CH₂)ₙ—, —NR₂(CH₂)ₙ—, or —S(CH₂)ₙ—;
X₁ and X₂ can be selected, independently, from —N(R₈)—, —O—, —S—, —Se—, —N═N—, —ON═CH—, —(R₈)N—N(R₈)—, —ON(R₈)—, a heterocycle, or a direct bond between L and Y₁ or Y₂, respectively;
Y₁ and Y₂ can be selected, independently, from —C(═O)—, —C(═S)—, —S(O₂)—, —S(O)—, —C(═NCN)—, —P(═O)(OR₂)—, a heteroaromatic group, or a direct bond between X₁ and Z₁ or X₂ and Z₂, respectively;
Z₁ and Z₂ can be selected, independently, from —N(R₈)—, —O—, —S—, —Se—, —N═N—, —ON═CH—, —R₈N—NR₈—, —ONR₈—, a heterocycle, or a direct bond between Y₁ or Y₂, respectively, and L;
R₈, independently for each occurrence, represents H, lower alkyl, —(CH₂)ₙaryl (e.g., substituted or unsubstituted), —(CH₂)ₙheteroaryl (e.g., substituted or unsubstituted), or two R₈ taken together may form a 4- to 8-membered ring, e.g., with X₁ and Z₁ or X₂ and Z₁, which ring may include one or more carbonyls;
p represents, independently for each occurrence, an integer from 0 to 10, preferably from 0 to 3; and
n, individually for each occurence, represents an integer from 0 to 10, preferably from 0 to 5.

34. The method of claim 30, wherein the hedgehog antagonist is represented by general formula (VI): 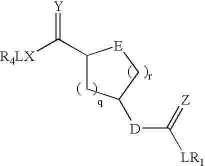
Formula VI
wherein, as valence and stability permit,
R₁, R₂, R₃, and R₄, independently for each occurrence, represent H, lower alkyl, —(CH₂)ₙaryl (e.g., substituted or unsubstituted), or —(CH₂)ₙheteroaryl (e.g., substituted or unsubstituted);
L, independently for each occurrence, is absent or represents —(CH₂)ₙ—, -alkenyl-, -alkynyl-, —(CH₂)ₙalkenyl-, —(CH₂)ₙalkynyl-, —(CH₂)ₙO(CH₂)ₚ—, —(CH₂)ₙNR₈(CH₂)ₚ—, —(CH₂)ₙS(CH₂)ₚ—, —(CH₂)ₙalkenyl(CH₂)ₚ—, —(CH₂)ₙalkynyl(CH₂)ₚ—, —O(CH₂)ₙ—, —NR₈(CH₂)ₙ—, or —S(CH₂)ₙ—;
X and D, independently, can be selected from —N(R₈)—, —O—, —S—, —(R₈)N—N(R₈)—, —ON(R₈)—, or a direct bond;
Y and Z, independently, can be selected from O or S;
E represents O, S, or NR₅, wherein R₅ represents LR₈ or —(C═O)LR₈.
R₈, independently for each occurrence, represents H, lower alkyl, —(CH₂)ₙaryl (e.g., substituted or unsubstituted), —(CH₂)ₙheteroaryl (e.g., substituted or unsubstituted), or two R₈ taken together may form a 4- to 8-membered ring;
p represents, independently for each occurrence, an integer from 0 to 10, preferably from 0 to 3;
n, individually for each occurrence, represents an integer from 0 to 10, preferably from 0 to 5; and
q and r represent, independently for each occurrence, an integer from 0-2.


SEQUENCE LISTING (48 sequences)

SEQ ID NO: 1 - DNA, 1277 bp, Gallus gallus
atggtcgaaatgctgctgttgacaagaattctcttggtgggcttcatctgcgctcttttagtctcctctgggctgacttgtggaccaggcaggggcattggaaaaaggaggcaccccaaaaagctgaccccgttagcctataagcagtttattcccaatgtggcagagaagaccctaggggccagtggaagatatgaagggaagatcacaagaaactccgagagatttaaagaactaaccccaaattacaaccctgacattatttttaaggatgaagagaacacgggagctgacagactgatgactcagcgctgcaaggacaagctgaatgccctggcgatctcggtgatgaaccagtggcccggggtgaagctgcgggtgaccgagggctgggacgaggatggccatcactccgaggaatcgctgcactacgagggtcgcgccgtggacatcaccacgtcggatcgggaccgcagcaagtacggaatgctggcccgcctcgccgtcgaggccggcttcgactgggtctactacgagtccaaggcgcacatccactgctccgtcaaagcagaaaactcagtggcagcgaaatcaggaggctgcttccctggctcagccacagtgcacctggagcatggaggcaccaagctggtgaaggacctgagccctggggaccgcgtgctggctgctgacgcggacggccggctgctctacagtgacttcctcaccttcctcgaccggatggacagctcccgaaagctcttctacgtcatcgagacgcggcagccccgggcccggctgctactgacggcggcccacctgctctttgtggccccccagcacaaccagtcggaggccacagggtccaccagtggccaggcgctcttcgccagcaacgtgaagcctggccaacgtgtctatgtgctgggcgagggcgggcagcagctgctgccggcgtctgtccacagcgtctcattgcgggaggaggcgtccggagcctacgccccactcaccgcccagggcaccatcctcatcaaccgggtgttggcctcctgctacgccgtcatcgaggagcacagttgggcccattgggccttcgcaccattccgcttggctcaggggctgctggccgccctctgcccagatggggccatccctactgccgccaccaccaccactggcatccattggtactcacggctcctctaccgcatcggcagctgggtgctggatggtgacgcgctgcatccgctgggcatggtggcaccggccagctg

SEQ ID NO: 2 - DNA, 1190 bp, Mus musculus
atggctctgccggccagtctgttgcccctgtgctgcttggcactcttggcactatctgcccagagctgcgggccgggccgaggaccggttggccggcggcgttatgtgcgcaagcaacttgtgcctctgctatacaagcagtttgtgcccagtatgcccgagcggaccctgggcgcgagtgggccagcggaggggagggtaacaagggggtcggagcgcttccgggacctcgtacccaactacaaccccgacataatcttcaaggatgaggagaacagcggcgcagaccgcctgatgacagagcgttgcaaagagcgggtgaacgctctagccatcgcggtgatgaacatgtggcccggagtacgcctacgtgtgactgaaggctgggacgaggacggccaccacgcacaggattcactccactacgaaggccgtgccttggacatcaccacgtctgaccgtgaccgtaataagtatggtttgttggcgcgcctagctgtggaagccggattcgactgggtctactacgagtcccgcaaccacatccacgtatcggtcaaagctgataactcactggcggtccgagccggaggctgctttccgggaaatgccacggtgcgcttgcggagcggcgaacggaaggggctgagggaactacatcgtggtgactgggtactggccgctgatgcagcgggccgagtggtacccacgccagtgctgctcttcctggaccgggatctgcagcgccgcgcctcgttcgtggctgtggagaccgagcggcctccgcgcaaactgttgctcacaccctggcatctggtgttcgctgctcgcgggccagcgcctgctccaggtgactttgcaccggtgttcgcgcgccgcttacgtgctggcgactcggtgctggctcccggcggggacgcgctccagccggcgcgcgtagcccgcgtggcgcgcgaggaagccgtgggcgtgttcgcaccgctcactgcgcacgggacgctgctggtcaacgacgtcctcgcctcctgctacgcggttctagagagtcaccagtgggcccaccgcgccttcgcccctttgcggctgctgcacgcgctcggggctctgctccctgggggtgcagtccagccgactggcatgcattggtactctcgcctcctttaccgcttggccgaggagttaatgggctg

SEQ ID NO: 3 - DNA, 1281 bp, Mus musculus
atgtctcccgcctggctccggccccgactgcggttctgtctgttcctgctgctgctgcttctggtgccggcggcgcggggctgcgggccgggccgggtggtgggcagccgccggaggccgcctcgcaagctcgtgcctcttgcctacaagcagttcagccccaacgtgccggagaagaccctgggcgccagcgggcgctacgaaggcaagatcgcgcgcagctctgagcgcttcaaagagctcacccccaactacaatcccgacatcatcttcaaggacgaggagaacacgggtgccgaccgcctcatgacccagcgctgcaaggaccgtctgaactcactggccatctctgtcatgaaccagtggcctggtgtgaaactgcgggtgaccgaaggccgggatgaagatggccatcactcagaggagtctttacactatgagggccgcgcggtggatatcaccacctcagaccgtgaccgaaataagtatggactgctggcgcgcttagcagtggaggccggcttcgactgggtgtattacgagtccaaggcccacgtgcattgctctgtcaagtctgagcattcggccgctgccaagacaggtggctgctttcctgccggagcccaggtgcgcctagagaacggggagcgtgtggccctgtcagctgtaaagccaggagaccgggtgctggccatgggggaggatgggacccccaccttcagtgatgtgcttattttcctggaccgcgagccaaaccggctgagagctttccaggtcatcgagactcaggatcctccgcgtcggctggcgctcacgcctgcccacctgctcttcattgcggacaatcatacagaaccagcagcccacttccgggccacatttgccagccatgtgcaaccaggccaatatgtgctggtatcaggggtaccaggcctccagcctgctcgggtggcagctgtctccacccacgtggcccttgggtcctatgctcctctcacaaggcatgggacacttgtggtggaggatgtggtggcctcctgctttgcagctgtggctgaccaccatctggctcagttggccttctggcccctgcgactgtttcccagtttggcatggggcagctggaccccaagtgagggtgttcactcctaccctcagatgctctaccgcctggggcgtctcttgctagaagagagcaccttccatccactgggcatgtctggggcaggaagctgaagggactctaaccactgccctcctggaactgctgtgcgtggatcc

SEQ ID NO: 4 - DNA, 1313 bp, Mus musculus
atgctgctgctgctggccagatgttttctggtgatccttgcttcctcgctgctggtgtgccccgggctggcctgtgggcccggcagggggtttggaaagaggcggcaccccaaaaagctgacccctttagcctacaagcagtttattcccaacgtagccgagaagaccctaggggccagcggcagatatgaagggaagatcacaagaaactccgaacgatttaaggaactcacccccaattacaaccccgacatcatatttaaggatgaggaaaacacgggagcagaccggctgatgactcagaggtgcaaagacaagttaaatgccttggccatctctgtgatgaaccagtggcctggagtgaggctgcgagtgaccgagggctgggatgaggacggccatcattcagaggagtctctacactatgagggtcgagcagtggacatcaccacgtccgaccgggaccgcagcaagtacggcatgctggctcgcctggctgtggaagcaggtttcgactgggtctactatgaatccaaagctcacatccactgttctgtgaaagcagagaactccgtggcggccaaatccggcggctgtttcccgggatccgccaccgtgcacctggagcagggcggcaccaagctggtgaaggacttacgtcccggagaccgcgtgctggcggctgacgaccagggccggctgctgtacagcgacttcctcaccttcctggaccgcgacgaaggcgccaagaaggtcttctacgtgatcgagacgctggagccgcgcgagcgcctgctgctcaccgccgcgcacctgctcttcgtggcgccgcacaacgactcggggcccacgcccgggccaagcgcgctctttgccagccgcgtgcgccccgggcagcgcgtgtacgtggtggctgaacgcggcggggaccgccggctgctgcccgccgcggtgcacagcgtgacgctgcgagaggaggaggcgggcgcgtacgcgccgctcacggcgcacggcaccattctcatcaaccgggtgctcgcctcgtgctacgctgtcatcgaggagcacagctgggcacaccgggccttcgcgcctttccgcctggcgcacgcgctgctggccgcgctggcacccgcccgcacggacggcgggggcgggggcagcatccctgcagcgcaatctgcaacggaagcgaggggcgcggagccgactgcgggcatccactggtactcgcagctgctctaccacattggcacctggctgttggacagcgagaccatgcatcccttgggaatggcggtcaagtccagctg

SEQ ID NO: 5 - DNA, 1256 bp, Brachydanio rerio
atgcggcttttgacgagagtgctgctggtgtctcttctcactctgtccttggtggtgtccggactggcctgcggtcctggcagaggctacggcagaagaagacatccgaagaagctgacacctctcgcctacaagcagttcatacctaatgtcgcggagaagaccttaggggccagcggcagatacgagggcaagataacgcgcaattcggagagatttaaagaacttactccaaattacaatcccgacattatctttaaggatgaggagaacacgggagcggacaggctcatgacacagagatgcaaagacaagctgaactcgctggccatctctgtaatgaaccactggccaggggttaagctgcgtgtgacagagggctgggatgaggacggtcaccattttgaagaatcactccactacgagggaagagctgttgatattaccacctctgaccgagacaagagcaaatacgggacactgtctcgcctagctgtggaggctggatttgactgggtctattacgagtccaaagcccacattcattgctctgtcaaagcagaaaattcggttgctgcgaaatctgggggctgtttcccaggttcggctctggtctcgctccaggacggaggacagaaggccgtgaaggacctgaaccccggagacaaggtgctggcggcagacagcgcgggaaacctggtgttcagcgacttcatcatgttcacagaccgagactccacgacgcgacgtgtgttttacgtcatagaaacgcaagaacccgttgaaaagatcaccctcaccgccgctcacctcctttttgtcctcgacaactcaacggaagatctccacaccatgaccgccgcgtatgccagcagtgtcagagccggacaaaaggtgatggttgttgatgatagcggtcagcttaaatctgtcatcgtgcagcggatatacacggaggagcagcggggctcgttcgcaccagtgactgcacatgggaccattgtggtcgacagaatactggcgtcctgttacgccgtaatagaggaccaggggcttgcgcatttggccttcgcgcccgccaggctctattattacgtgtcatcattcctgtcccccaaaactccagcagtcggtccaatgcgactttacaacaggagggggtccactggtactccaggctcctgtcatcaaatgggaacgtggcttttggacagcaacatgcttcatcctttggggatgtcagtaaactcaagctg

SEQ ID NO: 6 - DNA, 1425 bp, Homo sapiens
  modified_base (1387...1389): n=a, c, g, or t
atgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagcnnnagccggggggccgggggaggggcgcgggagggggcc

SEQ ID NO: 7 - DNA, 1622 bp, Homo sapiens
catcagcccaccaggagacctcgcccgccgctcccccgggctccccggccatgtctcccgcccggctccggccccgactgcacttctgcctggtcctgttgctgctgctggtggtgcccgcggcatggggctgcgggccgggtcgggtggtgggcagccgccggcgaccgccacgcaaactcgtgccgctcgcctacaagcagttcagccccaatgtgcccgagaagaccctgggcgccagcggacgctatgaaggcaagatcgctcgcagctccgagcgcttcaaggagctcacccccaattacaatccagacatcatcttcaaggacgaggagaacacaggcgccgaccgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagccccacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagctgaaaggactccaccgctgccctcctggaactgctgtactgggtccagaagcctctcagccaggagggagctggccctggaagggacctgagctgggggacactggctcctgccatctcctctgccatgaagatacaccattgagacttgactgggcaacaccagcgtcccccacccgcgtcgtggtgtagtcatagagctgcaagctgagctggcgaggggatggttgttgacccctctctcctagagaccttgaggctggcacggcgactcccaactcagcctgctctcactacgagttttcatactctgcctcccccattgggagggcccattccc

SEQ ID NO: 8 - DNA, 1191 bp, Homo sapiens
atggctctcctgaccaatctactgcccttgtgctgcttggcacttctggcgctgccagcccagagctgcgggccgggccgggggccggttggccggcgccgctatgcgcgcaagcagctcgtgccgctactctacaagcaatttgtgcccggcgtgccagagcggaccctgggcgccagtgggccagcggaggggagggtggcaaggggctccgagcgcttccgggacctcgtgcccaactacaaccccgacatcatcttcaaggatgaggagaacagtggagccgaccgcctgatgaccgagcgttgcaaggagagggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggctgctttccgggaaatgcaactgtgcgcctgtggagcggcgagcggaaagggctgcgggaactgcaccgcggagactgggttttggcggccgatgcgtcaggccgggtggtgcccacgccggtgctgctcttcctggaccgggacttgcagcgccgggcttcatttgtggctgtggagaccgagtggcctccacgcaaactgttgctcacgccctggcacctggtgtttgccgctcgagggccggcgcccgcgccaggcgactttgcaccggtgttcgcgcgccggctacgcgctggggactcggtgctggcgcccggcggggatgcgcttcggccagcgcgcgtggcccgtgtggcgcgggaggaagccgtgggcgtgttcgcgccgctcaccgcgcacgggacgctgctggtgaacgatgtcctggcctcttgctacgcggttctggagagtcaccagtgggcgcaccgcgcttttgcccccttgagactgctgcacgcgctaggggcgctgctccccggcggggccgtccagccgactggcatgcattggtactctcggctcctctaccgcttagcggaggagctactgggctga

SEQ ID NO: 9 - DNA, 1251 bp, Brachydanio rerio
atggacgtaaggctgcatctgaagcaatttgctttactgtgttttatcagcttgcttctgacgccttgtggattagcctgtggtcctggtagaggttatggaaaacgaagacacccaaagaaattaaccccgttggcttacaagcaattcatccccaacgttgctgagaaaacgcttggagccagcggcaaatacgaaggcaaaatcacaaggaattcagagagatttaaagagctgattccgaattataatcccgatatcatctttaaggacgaggaaaacacaaacgctgacaggctgatgaccaagcgctgtaaggacaagttaaattcgttggccatatccgtcatgaaccactggcccggcgtgaaactgcgcgtcactgaaggctgggatgaggatggtcaccatttagaagaatctttgcactatgagggacgggcagtggacatcactacctcagacagggataaaagcaagtatgggatgctatccaggcttgcagtggaggcaggattcgactgggtctattatgaatctaaagcccacatacactgctctgtcaaagcagaaaattcagtggctgctaaatcaggaggatgttttcctgggtctgggacggtgacacttggtgatgggacgaggaaacccatcaaagatcttaaagtgggcgaccgggttttggctgcagacgagaagggaaatgtcttaataagcgactttattatgtttatagaccacgatccgacaacgagaaggcaattcatcgtcatcgagacgtcagaacctttcaccaagctcaccctcactgccgcgcacctagttttcgttggaaactcttcagcagcttcgggtataacagcaacatttgccagcaacgtgaagcctggagatacagttttagtgtgggaagacacatgcgagagcctcaagagcgttacagtgaaaaggatttacactgaggagcacgagggctcttttgcgccagtcaccgcgcacggaaccataatagtggatcaggtgttggcatcgtgctacgcggtcattgagaaccacaaatgggcacattgggcttttgcgccggtcaggttgtgtcacaagctgatgacgtggctttttccggctcgtgaatcaaacgtcaattttcaggaggatggtatccactggtactcaaatatgctgtttcacatcggctcttggctgctggacagagactctttccatccactcgggattttacacttaagttga

SEQ ID NO: 10 - protein, 425 aa, Gallus gallus
MVEMLLLTRILLVGFICALLVSSGLTCGPGRGIGKRRHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEHGGTKLVKDLSPGDRVLAADADGRLLYSDFLTFLDRMDSSRKLFYVIETRQPRARLLLTAAHLLFVAPQHNQSEATGSTSGQALFASNVKPGQRVYVLGEGGQQLLPASVHSVSLREEASGAYAPLTAQGTILINRVLASCYAVIEEHSWAHWAFAPFRLAQGLLAALCPDGAIPTAATTTTGIHWYSRLLYRIGSWVLDGDALHPLGMVAPAS

SEQ ID NO: 11 - protein, 396 aa, Mus musculus [sequence omitted]

SEQ ID NO: 12 - protein, 411 aa, Mus musculus
MSPAWLRPRLRFCLFLLLLLLVPAARGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGRDEDGHHSEESLHYEGRAVDITTSDRDRNKYGLLARLAVEAGFDWVYYESKAHVHCSVKSEHSAAAKTGGCFPAGAQVRLENGERVALSAVKPGDRVLAMGEDGTPTFSDVLIFLDREPNRLRAFQVIETQDPPRRLALTPAHLLFIADNHTEPAAHFRATFASHVQPGQYVLVSGVPGLQPARVAAVSTHVALGSYAPLTRHGTLVVEDVVASCFAAVADHHLAQLAFWPLRLFPSLAWGSWTPSEGVHSYPQMLYRLGRLLLEESTFHPLGMSGAGS

SEQ ID NO: 13 - protein, 437 aa, Mus musculus
MLLLLARCFLVILASSLLVCPGLACGPGRGFGKRRHPKKLTPLAYKQFIPNVAEKTLGASGRYEGKITRNSERFKELTPNYNPDIIFKDEENTGADRLMTQRCKDKLNALAISVMNQWPGVRLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLRPGDRVLAADDQGRLLYSDFLTFLDRDEGAKKVFYVIETLEPRERLLLTAAHLLFVAPHNDSGPTPGPSALFASRVRPGQRVYVVAERGGDRRLLPAAVHSVTLREEEAGAYAPLTAHGTILINRVLASCYAVIEEHSWAHRAFAPFRLAHALLAALAPARTDGGGGGSIPAAQSATEARGAEPTAGIHWYSQLLYHIGTWLLDSETMHPLGMAVKSS

SEQ ID NO: 14 - protein, 418 aa, Brachydanio rerio
MRLLTRVLLVSLLTLSLVVSGLACGPGRGYGRRRHPKKLTPLAYKQFIPNVAEKTLGASGRYEGKITRNSERFKELTPNYNPDIIFKDEENTGADRLMTQRCKDKLNSLAISVMNHWPGVKLRVTEGWDEDGHHFEESLHYEGRAVDITTSDRDKSKYGTLSRLAVEAGFDWVYYESKAHIHCSVKAENSVAAKSGGCFPGSALVSLQDGGQKAVKDLNPGDKVLAADSAGNLVFSDFIMFTDRDSTTRRVFYVIETQEPVEKITLTAAHLLFVLDNSTEDLHTMTAAYASSVRAGQKVMVVDDSGQLKSVIVQRIYTEEQRGSFAPVTAHGTIVVDRILASCYAVIEDQGLAHLAFAPARLYYYVSSFLSPKTPAVGPMRLYNRRGSTGTPGSCHQMGTWLLDSNMLHPLGMSVNSS

SEQ ID NO: 15 - protein, 475 aa, Homo sapiens
  SITE (463): Xaa=unknown amino acid residue
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSSXSRGAGGGAREGA

SEQ ID NO: 16 - protein, 411 aa, Homo sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 17 - protein, 396 aa, Homo sapiens
MALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

SEQ ID NO: 18 - protein, 416 aa, Brachydanio rerio
MDVRLHLKQFALLCFISLLLTPCGLACGPGRGYGKRRHPKKLTPLAYKQFIPNVAEKTLGASGKYEGKITRNSERFKELIPNYNPDIIFKDEENTNADRLMTKRCKDKLNSLAISVMNHWPGVKLRVTEGWDEDGHHLEESLHYEGRAVDITTSDRDKSKYGMLSRLAVEAGFDWVYYESKAHIHCSVKAENSVAAKSGGCFPGSGTVTLGDGTRKPIKDLKVGDRVLAADEKGNVLISDFIMFIDHDPTTRRQFIVIETSEPFTKLTLTAAHLVFVGNSSAASGITATFASNVKPGDTVLVWEDTCESLKSVTVKRIYTEEHEGSFAPVTAHGTIIVDQVLASCYAVIENHKWAHWAFAPVRLCHKLMTWLFPARESNVNFQEDGIHWYSNMLFHIGSWLLDRDSFHPLGILHLS

SEQ ID NO: 19 - DNA, 1416 bp, Drosophila melanogaster
  CDS (1)..(1413)
atggataaccacagctcagtgccttgggccagtgccgccagtgtcacctgtctctccctgggatgccaaatgccacagttccagttccagttccagctccaaatccgcagcgagctccatctccgcaagcccgcaagaagaacgcaaacgatgcgccacattgcgcatacgcagcgttgcctcagcaggctgacctctctggtggccctgctgctgatcgtcttgccgatggtctttagcccggctcacagctgcggtcctggccgaggattgggtcgtcatagggcgcgcaacctgtatccgctggtcctcaagcagacaattcccaatctatccgagtacacgaacagcgcctccggacctctggagggtgtgatccgtcgggattcgcccaaattcaaggacctcgtgcccaactacaacagggacatccttttccgtgacgaggaaggcaccggagcggatggcttgatgagcaagcgctgcaaggagaagctaaacgtgctggcctactcggtgatgaacgaatggcccggcatccggctgctggtcaccgagagctgggacgaggactaccatcacggccaggagtcgctccactacgagggccgagcggtgaccattgccacctccgatcgcgaccagtccaaatacggcatgctcgctcgcctggccgtcgaggctggattcgattgggtctcctacgtcagcaggcgccacatctactgctccgtcaagtcagattcgtcgatcagttcccacgtgcacggctgcttcacgccggagagcacagcgctgctggagagtggagtccggaagccgctcggcgagctctctatcggagatcgtgttttgagcatgaccgccaacggacaggccgtctacagcgaagtgatcctcttcatggaccgcaacctcgagcagatgcaaaactttgtgcagctgcacacggacggtggagcagtgctcacggtgacgccggctcacctggttagcgtttggcagccggagagccagaagctcacgtttgtgtttgcgcatcgcatcgaggagaagaaccaggtgctcgtacgggatgtggagacgggcgagctgaggccccagcgagtggtcaagttgggcagtgtgcgcagtaagggcgtggtcgcgccgctgacccgcgagggcaccattgtggtcaactcggtggccgccagttgctatgcggtgatcaacagtcagtcgctggcccactggggactggctcccatgcgcctgctgtccacgctggaggcgtggctgcccgccaaggagcagttgcacagttcgccgaaggtggtgagctcggcgcagcagcagaatggcatccattggtatgccaatgcgctctacaaggtcaaggactacgtgctgccgcagagctggcgccacgattga

SEQ ID NO: 20 - protein, 471 aa, Drosophila melanogaster
MDNHSSVPWASAASVTCLSLGCQMPQFQFQFQLQIRSELHLRKPARRTQTMRHIAHTQRCLSRLTSLVALLLIVLPMVFSPAHSCGPGRGLGRHRARNLYPLVLKQTIPNLSEYTNSASGPLEGVIRRDSPKFKDLVPNYNRDILFRDEEGTGADGLMSKRCKEKLNVLAYSVMNEWPGIRLLVTESWDEDYHHGQESLHYEGRAVTIATSDRDQSKYGMLARLAVEAGFDWVSYVSRRHIYCSVKSDSSISSHVHGCFTPESTALLESGVRKPLGELSIGDRVLSMTANGQAVYSEVILFMDRNLEQMQNFVQLHTDGGAVLTVTPAHLVSVWQPESQKLTFVFAHRIEEKNQVLVRDVETGELRPQRVVKLGSVRSKGVVAPLTREGTIVVNSVAASCYAVINSQSLAHWGLAPMRLLSTLEAWLPAKEQLHSSPKVVSSAQQQNGIHWYANALYKVKDYVLPQSWRHD

SEQ ID NO: 21 - DNA, 522 bp, Homo sapiens
tgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggc

SEQ ID NO: 22 - DNA, 525 bp, Homo sapiens
tgcgggccgggtcgggtggtgggcagccgccggcgaccgccacgcaaactcgtgccgctcgcctacaagcagttcagccccaatgtgcccgagaagaccctgggcgccagcggacgctatgaaggcaagatcgctcgcagctccgagcgcttcaaggagctcacccccaattacaatccagacatcatcttcaaggacgaggagaacacaggcgccgaccgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacgggcggc

SEQ ID NO: 23 - protein, 175 aa, Homo sapiens
CGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGG

SEQ ID NO: 24 - protein, 174 aa, Homo sapiens
CGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGG

SEQ ID NO: 25 - protein, 176 aa, Homo sapiens
CGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGG

SEQ ID NO: 26 - protein, 175 aa, Artificial Sequence
  Description of Artificial Sequence Consensus sequence
XGPGRXXXXXRRXXXKXLXPLXYKQFXPXVXEKTLGASGRXEGKXXRXSERFKXLXPNYNPDIIFKDEENXGADRLMTXRCKXXXNSLAIXVMNXWPGVKLRVTEGWDEDGHHXXXSLHYEGRAVDITTSDRDRXKYGXLARLAVEAGFDWVYYESXXHXHXSVKXXXXAAXXGG

SEQ ID NO: 27 - DNA, 528 bp, Homo sapiens
tgcgggccgggccgggggccggttggccggcgccgctatgcgcgcaagcagctcgtgccgctactctacaagcaatttgtgcccggcgtgccagagcggaccctgggcgccagtgggccagcggaggggagggtggcaaggggctccgagcgcttccgggacctcgtgcccaactacaaccccgacatcatcttcaaggatgaggagaacagtggagccgaccgcctgatgaccgagcgttgtaaggagcgggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggc

SEQ ID NO: 28 - DNA, 684 bp, Homo sapiens
gtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaa

SEQ ID NO: 29 - DNA, 687 bp, Homo sapiens
gtcgacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccacgggaagagcagttccaaagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 30 - DNA, 702 bp, Homo sapiens
gtcgaccccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattaccaaagtacacttcgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtgacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 31 - DNA, 9776 bp, Plasmid P55
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagatgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgattcgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagcagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaaacaaaacttcgtcgtcaaataaccgtttggtataagaaccttctggagcattgctcttacgatcccacaaggtgcttccatggctctaagaccctttgattggccaaaacaggaagtgcgttccaagtgacagaaaccaacacctgtttgttcaaccacaaatttcaagcagtctccatcacaatccaattcgatacccagcaacttttgagttcgtccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttggacctgcagggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcaaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcccacggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcgagatc

SEQ ID NO: 32 - DNA, 10491 bp, Plasmid pUB114
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaaccttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagatgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggcgtcgacgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccacgggaagagcagttccaaagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgatcccagtgtccttggagccctctggtcctacagcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagcagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaaacaaaacttcgtcgtcaaataaccgtttggtataagaaccttctggagcattgctcttacgatcccacaaggtgcttccatggctctaagaccctttgattggccaaaacaggaagtgcgttccaagtgacagaaaccaacacctgtttgttcaaccacaaatttcaagcagtctccatcacaatccaattcgatacccagcaacttttgagttcgtccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttggacctgcagggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcaaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcccacggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcgagatc

SEQ ID NO: 33 - DNA, 10512 bp, Plasmid pUB115
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagatgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggcgtcgaccccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattaccaaagtacacttcgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtgacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagctcagatcgattccatggatcctcacatcccaatccgcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagcagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaaacaaaacttcgtcgtcaaataaccgtttggtataagaaccttctggagcattgctcttacgatcccacaaggtgcttccatggctctaagaccctttgattggccaaaacaggaagtgcgttccaagtgacagaaaccaacacctgtttgttcaaccacaaatttcaagcagtctccatcacaatccaattcgatacccagcaacttttgagttcgtccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttggacctgcagggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcaaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcccacggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcgagatc

SEQ ID NO: 34 - DNA, 10462 bp, Plasmid pUB116
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagatgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggcgtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggaaatgagtgcggcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagcagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaaacaaaacttcgtcgtcaaataaccgtttggtataagaaccttctggagcattgctcttacgatcccacaaggtgcttccatggctctaagaccctttgattggccaaaacaggaagtgcgttccaagtgacagaaaccaacacctgtttgttcaaccacaaatttcaagcagtctccatcacaatccaattcgatacccagcaacttttgagttcgtccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttggacctgcagggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcaaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcccacggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcgagatc

SEQ ID NO: 35 - DNA, 4205 bp, Plasmid pEAG657
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccctctagatcctttcagctccctgccccggacatgcccagtgggtggaagctgccctcttctagcaggagacgccccaggcggtagagcagctgggggtaccaatgcacaccctcccccggagtccagctgccccatgccaagctgtgaaagagtctcaggggccagaaggccaactgagccaggtggtggtcagccacggccgcgaagcaggatgccaccacatcctccaccaccagtgtcccatgctttgtgagcggggcgtaggccccgagggccacgtgtgtagagacagctgccacgcgggcaggctgcaggcctggcaccccagccaccagcacgtactggccaggctgcacgtggctggcaaatgtggcccggaagcgggctgccggctccgtgtgattgtcagccgtaaagagcaggtgagcgggtgtgagtgccaggcggcgtggggggtcctgagtctcgatgacctggaaggctctcagcctgtggggctcgcggtccaggaaaatgagcacatcgctgaaggtggggctcccatcctcccccatggccagcacacggtctcccggcctcacggctgacaaggccacacgcgccccactctccaggcgtacctgggctgcggccgcgaatcagccgcccgtcttggctgcggccgagtgctcggacttgacggagcaatgcacgtgggcctttgactcgtaatacacccagtcaaagccggcctccactgccaagcgcgccagcagtccatacttattgcggtcgcggtctgatgtggtgatgtccaccgcgcggccctcataatgcagggactcctctgagtggtggccgtcctcgtcccagccctcggtcacccgcagcttcacaccgggccactggttcatcaccgagatagccagcgagttcaggcggtccttgcagcgctgggtcatgaggcggtcggcgcctgtgttctcctcgtccttgaagatgatgtctggattgtaattgggggtgagctccttgaagcgctcggagctgcgagcgatcttgccttcatagcgtccgctggcgcccagggtcttctcgggcacattggggctgaactgcttgtaggcgagcggcacgagtttgcgtggcggtcgccggcggctgcccaccacccgacccggcccgcagccccatgccgccggcaccaccagcagcagcaacaggaccaggcagaagtgcagtcggggccggagccgggcgggagacatggcggccgcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgacgcgtcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 36 - DNA, 30 bp, Artificial Sequence
  Description of Artificial Sequence Oligonucleotide
tcgagaaaagatgcggaccgggcagggggt

SEQ ID NO: 37 - DNA, 29 bp, Artificial Sequence
  Description of Artificial Sequence Oligonucleotide
cgaaccccctgcccggtccgcatcttttc

SEQ ID NO: 38 - DNA, 25 bp, Artificial Sequence
  Description of Artificial Sequence Primer
tcaggatgcatttgacagtgactgg

SEQ ID NO: 39 - DNA, 25 bp, Artificial Sequence
  Description of Artificial Sequence Primer
actccgagtcggaggaatcagaccc

SEQ ID NO: 40 - DNA, 23 bp, Artificial Sequence
  Description of Artificial Sequence Primer
cgaagtggtgaagttcatggatg

SEQ ID NO: 41 - DNA, 25 bp, Artificial Sequence
  Description of Artificial Sequence Primer
ttctgtatcagtctttcctggtgag

SEQ ID NO: 42 - DNA, 20 bp, Artificial Sequence
  Description of Artificial Sequence Primer
tacaacttcaagcagaagag

SEQ ID NO: 43 - DNA, 20 bp, Artificial Sequence
  Description of Artificial Sequence Primer
cagctcttagcagacattgg

SEQ ID NO: 44 - DNA, 24 bp, Artificial Sequence
  Description of Artificial Sequence Primer
caacacaaacgctctgcagagaga

SEQ ID NO: 45 - DNA, 24 bp, Artificial Sequence
  Description of Artificial Sequence Primer
ctccagttgctgcttctgaaggac

SEQ ID NO: 46 - DNA, 23 bp, Artificial Sequence
  Description of Artificial Sequence Primer
agcgacgtgaggatggcagcgtt

SEQ ID NO: 47 - DNA, 25 bp, Artificial Sequence
  Description of Artificial Sequence Primer
atttcctggttggctgatgctgctt

SEQ ID NO: 48 - DNA, 4205 bp, Plasmid pEAG658
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccctctagatcctttcagctccctgccccggacatgcccagtgggtggaagctgccctcttctagcaggagacgccccaggcggtagagcagctgggggtaccaatgcacaccctcccccggagtccagctgccccatgccaagctgtgaaagagtctcaggggccagaaggccaactgagccaggtggtggtcagccacggccgcgaagcaggatgccaccacatcctccaccaccagtgtcccatgctttgtgagcggggcgtaggccccgagggccacgtgtgtagagacagctgccacgcgggcaggctgcaggcctggcaccccagccaccagcacgtactggccaggctgcacgtggctggcaaatgtggcccggaagcgggctgccggctccgtgtgattgtcagccgtaaagagcaggtgagcgggtgtgagtgccaggcggcgtggggggtcctgagtctcgatgacctggaaggctctcagcctgtggggctcgcggtccaggaaaatgagcacatcgctgaaggtggggctcccatcctcccccatggccagcacacggtctcccggcctcacggctgacaaggccacacgcgccccactctccaggcgtacctgggctccggcagggtcgacgccgcccgtcttggctgcggccgagtgctcggacttgacggagcaatgcacgtgggcctttgactcgtaatacacccagtcaaagccggcctccactgccaagcgcgccagcagtccatacttattgcggtcgcggtctgatgtggtgatgtccaccgcgcggccctcataatgcagggactcctctgagtggtggccgtcctcgtcccagccctcggtcacccgcagcttcacaccgggccactggttcatcaccgagatagccagcgagttcaggcggtccttgcagcgctgggtcatgaggcggtcggcgcctgtgttctcctcgtccttgaagatgatgtctggattgtaattgggggtgagctccttgaagcgctcggagctgcgagcgatcttgccttcatagcgtccgctggcgcccagggtcttctcgggcacattggggctgaactgcttgtaggcgagcggcacgagtttgcgtggcggtcgccggcggctgcccaccacccgacccggcccgcagccccatgccgccggcaccaccagcagcagcaacaggaccaggcagaagtgcagtcggggccggagccgggcgggagacatggcggccgcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgacgcgtcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac